(19) United States
(12) Patent Application Publication
(10) Pub. No.: US 2023/0233541 A1
(43) Pub. Date: Jul. 27, 2023

(54) SUBSTITUTED BENZAMIDES AS MODULATORS OF TREX1

(71) Applicant: Constellation Pharmaceuticals, Inc., Boston, MA (US)

(72) Inventors: Mary-Margaret Zablocki, Revere, MA (US); Kennedy Taveras, Lynn, MA (US); Jonathan E. Wilson, Arlington, MA (US); Aaron Coffin, Everett, MA (US); Julian R. Levell, Arlington, MA (US); Aravind Prasad Medikonda, Arlington, MA (US)

(21) Appl. No.: 17/926,849

(22) PCT Filed: May 27, 2021
(86) PCT No.: PCT/US2021/034488

§ 371 Date: Nov. 21, 2022

Related U.S. Application Data
(60) Provisional application No. 63/030,590, filed on May 27, 2020.

(51) Int. Cl. A61K 31/444; A61K 31/4245; A61K 31/4545; A61K 31/4725; A61P 35/00

(52) U.S. Cl. CPC A61K 31/444; A61K 31/4245; A61K 31/4545; A61K 31/4725; A61P 35/00

(57) ABSTRACT

Provided are compounds of Formula (I): (I) and pharmaceutically acceptable salts and compositions thereof, which are useful for treating a variety of conditions associated with TREX1.

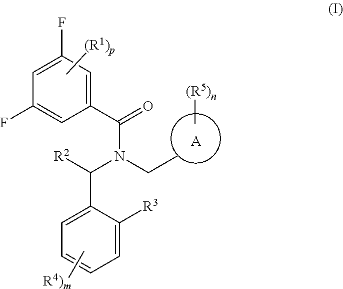

### RELATED APPLICATIONS

This application claims the benefit of priority to U.S. Provisional Application No. 63/030,590, filed May 27, 2020, the entire contents of which are incorporated herein by reference.

### BACKGROUND

A potential immune therapy is needed for cancers related to the innate immune system recognition of non-self, and to detect and protect against potential danger. Cancer cells differ antigenically from their normal counterparts and emit danger signals to alert the immune system similar to viral infection. These signals, which include damage-associated molecular patterns (DAMPs) and pathogen-associated molecular patterns (PAMPs), further activate the innate immune system resulting in the protection of the host from a variety of threats (Front. Cell Infect. Microbiol. 2012, 2, 168).

Ectopically expressed single stranded DNA (ssDNA) and double stranded DNA (dsDNA) are known PAMPs and/or DAMPs, which are being recognized by the cyclic GMP-AMP synthase (cGAS), a nucleic acid sensor (Nature 2011, 478, 515-518). Upon sensing of cytosolic DNA, cGAS catalyzes the generation of the cyclic dinucleotide 2′,3′-cGAMP, a potent second messenger and activator of the ER transmembrane adapter protein stimulator of interferon genes (STING) (Cell Rep. 2013, 3, 1355-1361). STING activation triggers phosphorylation of IRF3 via TBK1 which in turn leads to type I interferon production and activation of interferon stimulated genes (ISGs); a pre-requisite to the activation of innate immunity and initiation of adaptive immunity. Production of type I interferons thus constitutes a key bridge between the innate and adaptive immunity (Science 2013, 341, 903-906).

Excess type I IFN can be harmful to the host and induce autoimmunity, therefore, negative feedback mechanisms exist that keep type I IFN-mediated immune activation in check. Three prime repair exonuclease I (TREX1) is a 3′-5′ DNA exonuclease responsible for the removal of ectopically expressed ssDNA and dsDNA and is therefore a key repressor of the cGAS/STING pathway (PNAS 2015, 112, 5117-5122).

Type I interferons and downstream pro-inflammatory cytokine responses are critical to the development of immune responses and their effectiveness. Type I interferons enhance both the ability of dendritic cells and macrophages to take up, process, present, and cross-present antigens to T cells, and their potency to stimulate T cells by eliciting the up-regulation of the co-stimulatory molecules such as CD40, CD80 and CD86 (J. Exp. Med. 2011, 208, 2005-2016). Type I interferons also bind their own receptors and activate interferon responsive genes that contribute to activation of cells involved in adaptive immunity (EMBO Rep. 2015, 16, 202-212).

From a therapeutic perspective, type I interferons and compounds that can induce type I interferon production have potential for use in the treatment of human cancers (Nat. Rev Immunol. 2015, 15, 405-414). Interferons can inhibit human tumor cell proliferation directly. In addition, type I interferons can enhance anti-tumor immunity by triggering the activation of cells from both the innate and adaptive immune system. Importantly, the anti-tumor activity of PD-1 blockade requires pre-existing intratumoral T cells. By turning cold tumors into hot and thereby eliciting a spontaneous anti-tumor immunity, type I IFN-inducing therapies have the potential to expand the pool of patients responding to anti-PD-1 therapy as well as enhance the effectiveness of anti-PD1 therapy.

Human and mouse genetic studies suggest that TREX1 inhibition might be amenable to a systemic delivery route and therefore TREX1 inhibitory compounds could play an important role in the anti-tumor therapy landscape. TREX1 is a key determinant for the limited immunogenicity of cancer cells responding to radiation treatment [Trends in Cell Biol., 2017, 27(8), 543-4; Nature Commun., 2017, 8, 15618]. TREX1 is induced by genotoxic stress and involved in protection of glioma and melanoma cells to anticancer drugs [Biochim. Biophys. Acta, 2013, 1833, 1832-43]. STACT-TREX1 therapy shows robust anti-tumor efficacy in multiple murine cancer models [Glickman et al, Poster P235, 33^{rd} Annual Meeting of Society for Immunotherapy of Cancer, Washington D.C., Nov. 7-11, 2018]. (TREX1) expression correlates with cervical cancer cells growth in vitro and disease progression in vivo [Scientific Reports 1019, 9, 351]. Beyond oncology there is also support for agonists of the IFN pathway to be useful in antiviral therapy, for example STING agonists induce an innate antiviral immune response against Hepatitis B Virus via stimulation of the IFN pathway and upregulation of ISG's [Antimicrob. Agents Chemother. 2015, 59:1273-1281] and TREX1 inhibits the innate immune response to HIV type 1 [Nature Immunology, 2010, 11(11), 1005].

### SUMMARY

Provided herein are compounds having the Formula I:

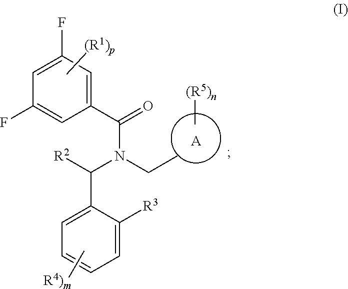

and pharmaceutically acceptable salts and compositions thereof, wherein R¹, R², R³, R⁴, R⁵, m, n, and p are as described herein. The disclosed compounds of Formula I, pharmaceutically acceptable salts, and compositions thereof, modulate TREX1, and are useful in a variety of therapeutic applications such as, for example, in treating cancer. As such, their uses for treating diseases responsive to the inhibition of TREX1 are included.

In addition to the uses of the compounds of Formula I, also provided are compounds and compositions, particularly for uses in treating diseases responsive to the inhibition of TREX1, having the Formula VI:

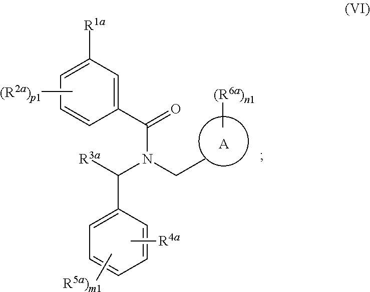

or a pharmaceutically acceptable salt thereof, wherein R^{1a}, R^{2a}, R^{3a}, R^{4a}, R^{5a}, R^{6a}, m1, n1, and p1 are as described herein.

### DETAILED DESCRIPTION

### 1. General Description of Compounds

In a first embodiment, provided herein is a compound of Formula I:

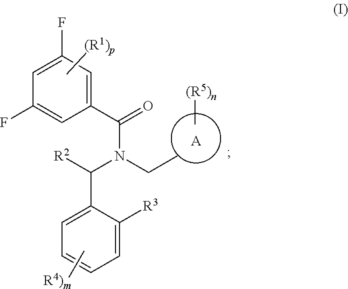

or a pharmaceutically acceptable salt thereof, wherein

R¹ is halo, hydroxy, (C₁-C₄)alkyl, halo(C₁-C₄)alkyl, (C₁-C₄)alkoxy, or halo(C₁-C₄)alkoxy;

R² is hydrogen, (C₁-C₄)alkyl, halo(C₁-C₄)alkyl, —(C₁-C₄)alkylOR^{a}, —C(O)R^{b}, —C(O)OR^{b}, —C(O)NR^{b}R^{c}, —C(O)NR^{b}R^{c}, 5- to 7-membered heteroaryl, or 4- to 7-membered heterocyclyl, wherein said heteroaryl and heterocyclyl are each optionally substituted with 1 to 3 groups selected from R⁶;

R³ is halo, (C₁-C₄)alkyl, halo(C₁-C₄)alkyl, (C₁-C₄)alkoxy, halo(C₁-C₄)alkoxy, —NR^{b}R^{c}, or CN;

R⁴ is halo, hydroxy, (C₁-C₄)alkyl, halo(C₁-C₄)alkyl, (C₁-C₄)alkoxy, halo(C₁-C₄)alkoxy, CN, —NR^{b}R^{c}, —C(O)R^{b}, —C(O)OR^{b}, —C(O)NR^{b}R^{c}, —SR^{b}, —C(O)NR^{b}R^{c}, —S(O)₂R^{b}, —S(O)R^{b}, —NR^{b}C(O)R^{c}, —NR^{b}C(O)OR^{c}, —NR^{b}C(S)OR^{c}, and —NR^{b}C(O)N^{c}R^{d};

Ring A is nitrogen-containing 5- to 7-membered heteroaryl;

R⁵ is halo, hydroxy, (C₁-C₄)alkyl, halo(C₁-C₄)alkyl, (C₁-C₄)alkoxy, halo(C₁-C₄)alkoxy, oxo, —(C₁-C₄)alkylOR^{d}, —(C₁-C₄)alkylC(O)R^{d}, —(C₁-C₄)alkylC(O)OR^{e}, —(C₁-C₄)alkylC(O)NR^{d}R^{e}, —C(O)R^{d}, —C(O)OR^{d}, —C(O)NR^{d}R^{e}, —C(O)NR^{d}SO₂(C₁-C₄)alkyl, CN, —NR^{d}R^{e}, —C(O)NR^{d}SO₃H, —NR^{d}C(O)R^{e}, —NR^{d}C(O)OR^{e}, —NR^{d}C(S)OR^{e}, —NR^{d}C(O)N^{e}R^{f}, —NR^{d}C(S)NR^{e}Rf^{c}, —NR^{d}S(O)₂NR^{e}R^{f}, —C(S)R^{d}, —S(O)₂R^{d}, —S(O)R^{d}, —C(S)OR^{d}, —C(S)NR^{d}R^{e}, —NR^{d}C(S)R^{e}, —SR^{d}, —(C₁-C₄)alkylC(O)NR^{d}R^{e}, —(C₁-C₄)alkylC(O)NR^{d}SO₂(C₁-C₄)alkyl, —(C₁-C₄)alkylNR^{d}R^{e}, —(C₁-C₄)alkylC(O)NR^{d}SO₃H, —(C₁-C₄)alkylNR^{d}C(O)R^{e}, —(C₁-C₄)alkylNR^{d}C(O)OR^{e}, —(C₁-C₄)alkylNR^{d}C(S)OR^{e}, —(C₁-C₄)alkylNR^{d}C(O)N^{e}R^{f}, —(C₁-C₄)alkylNR^{d}C(S)NR^{e}R^{f}, —(C₁-C₄)alkylNR^{d}S(O)₂NR^{e}R^{f}, —(C₁-C₄)alkylC(S)R^{d}, —(C₁-C₄)alkylS(O)₂R^{d}, —(C₁-C₄)alkylS(O)R^{d}, —(C₁-C₄)alkylC(S)OR^{d}, —(C₁-C₄)alkylC(S)NR^{d}R^{e}, —(C₁-C₄)alkylNRd^{b}C(S)R^{e}, —(C₁-C₄)alkylSR^{d}, 5- to 7-membered heteroaryl, or 4- to 7-membered heterocyclyl, wherein said heteroaryl and heterocyclyl are each optionally substituted with 1 to 3 groups selected from R⁷;

R^{a}, R^{b}, R^{c}, R^{d}, R^{e}, and R^{f} are each independently hydrogen, (C₁-C₄)alkyl, or halo(C₁-C₄)alkyl;

R⁶ and R⁷ are each independently halo, hydroxy, (C₁-C₄)alkyl, halo(C₁-C₄)alkyl, (C₁-C₄)alkoxy, halo(C₁-C₄)alkoxy, CN, or oxo;

m and n are each independently 0, 1, 2 or 3; and

p is 0 or 1.

In a second embodiment, provided herein, particularly for use in treating diseases responsive to the inhibition of TREX1, are compounds of the Formula VI:

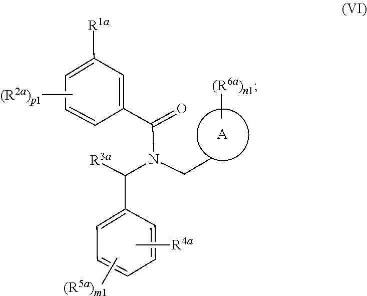

or a pharmaceutically acceptable salt thereof, wherein

R^{1a}, R^{2a}, R^{4a}, and R^{5a} are each independently halo, hydroxy, (C₁-C₄)alkyl, halo(C₁-C₄)alkyl, (C₁-C₄)alkoxy, halo(C₁-C₄)alkoxy, CN, —NR^{b1}R^{c1}, —C(O)R^{b1}, —C(O)OR^{b1}, —C(O)NR^{b1}R^{c1}, —SR^{b1}, —C(O)NR^{b1}R^{c1}, —S(O)₂R^{b1}, —S(O)R^{b1}, —NR^{b1}C(O)R^{c1}, —NR^{b1}C(O)OR^{c1}, —NR^{b1}C(S)OR^{c1}, and —NR^{b1}C(O)N^{c1}R^{d1};

R^{3a} is hydrogen, (C₁-C₄)alkyl, halo(C₁-C₄)alkyl, —(C₁-C₄)alkylOR^{a}, —C(O)R^{b}, —C(O)OR^{b}, —C(O)NR^{b}R^{c}, —C(O)NR^{b}R^{c}, 5- to 7-membered heteroaryl, or 5- to 7-membered heterocyclyl, wherein said heteroaryl and heterocyclyl are each optionally substituted with 1 to 3 groups selected from R^{7a};

Ring A is heteroaryl;

R^{6a} is halo, hydroxy, (C₁-C₄)alkyl, halo(C₁-C₄)alkyl, (C₁-C₄)alkoxy, halo(C₁-C₄)alkoxy, oxo, —(C₁-C₄)alkylOR^{d1}, —(C₁-C₄)alkylC(O)R^{d1}, —(C₁-C₄)alkylC(O)OR^{d1}, —(C₁-C₄)alkylC(O)NR^{d1}R^{e1}, —C(O)R^{d1}, —C(O)OR^{d1}, —C(O)NR^{d1}R^{e1}, —C(O)NR^{d1}SO₂(C₁-C₄)alkyl, CN, —NR^{d1}R^{e1}, —C(O)NR^{d1}SO₃H, —NR^{d1}C(O)R^{e1}, —NR^{d1}C(O)OR^{e1}, —NR^{d1}C(S)OR^{e1}, —NR^{d1}C(O)N^{e1}R^{f1}, —NR^{d1}C(S)NR^{e1}R^{f1}, —NR^{d1}S(O)₂NR^{e1}R^{f1}, —C(S)R^{d1}, —S(O)₂R^{d1}, —S(O)R^{d1}, —C(S)OR^{d1}, —C(S)NR^{d1}R^{e1}, —NR^{d1}C(S)R^{e1}, —SR^{d1}, —(C₁-C₄)alkylC(O)NR^{d1}R^{e1}, —(C₁-C₄)alkylC(O)NR^{d1}SO₂(C₁-C₄)alkyl, —(C₁-C₄)alkylNR^{d1}R^{e1}, —(C₁-C₄)alkylC(O)NR^{d1}SO₃H, —(C₁-C₄)alkylNR^{d1}C(O)R^{e1}, —(C₁-C₄)alkylNR^{d1}C(O)OR^{e1}, —(C₁-C₄)alkylNR^{d1}C(S)OR^{e1}, —(C₁-C₄)alkylNR^{d1}C(O)N^{e1}R^{f1}, —(C₁-C₄)alkylNR^{d1}C(S)NR^{e1}R^{f1}, —(C₁-C₄)alkylNR^{d1}S(O)₂NR^{e1}R^{f1}, —(C₁-C₄)alkylC(S)R^{d1}, —(C₁-C₄)alkylS(O)₂R^{d1}, —(C₁-C₄)alkylS(O)R^{d1}, —(C₁-C₄)alkylC(S)OR^{d1}, —(C₁-C₄)alkylC(S)NR^{d1}R^{e1}, —(C₁-C₄)alkylNR^{b1}C(S)R^{e1}, —(C₁-C₄)alkylSR^{d1}, 5- to 7-membered heteroaryl, or 4- to 7-membered heterocyclyl, wherein said heteroaryl and heterocyclyl are each optionally substituted with 1 to 3 groups selected from Ra;

R^{a1}, R^{b1}, R^{c1}, R^{d1}, R^{e1}, and R^{f1} are each independently hydrogen, (C₁-C₄)alkyl, or halo(C₁-C₄)alkyl;

R^{7a} and R^{8a} are each independently halo, hydroxy, (C₁-C₄)alkyl, halo(C₁-C₄)alkyl, (C₁-C₄)alkoxy, halo(C₁-C₄)alkoxy, CN, or oxo; and

m1, n1, and p1 are each independently 0, 1, 2, or 3.

### 2. Definitions

When used in connection to describe a chemical group that may have multiple points of attachment, a hyphen (-) designates the point of attachment of that group to the variable to which it is defined. For example, —NR^{b}C(O)OR^{c} and —NR^{b}C(S)OR^{c} mean that the point of attachment for this group occurs on the nitrogen atom.

The terms “halo” and “halogen” refer to an atom selected from fluorine (fluoro, —F), chlorine (chloro, —Cl), bromine (bromo, —Br), and iodine (iodo, —I).

The term “alkyl” when used alone or as part of a larger moiety, such as “haloalkyl”, and the like, means saturated straight-chain or branched monovalent hydrocarbon radical.

“Alkoxy” means an alkyl radical attached through an oxygen linking atom, represented by —O-alkyl. For example, “(C₁-C₄)alkoxy” includes methoxy, ethoxy, proproxy, and butoxy.

The term “haloalkyl” includes mono, poly, and perhaloalkyl groups where the halogens are independently selected from fluorine, chlorine, bromine, and iodine.

“Haloalkoxy” is a haloalkyl group which is attached to another moiety via an oxygen atom such as, e.g., —OCHF₂ or —OCF₃.

The term oxo means the group ═O.

The term “heteroaryl” used alone or as part of a larger moiety refers to a 5- to 12-membered (e.g., a 4- to 6-membered) aromatic radical containing 1-4 heteroatoms selected from N, O, and S. A heteroaryl group may be mono- or bi-cyclic as size permits. Monocyclic heteroaryl includes, for example, thienyl, furanyl, pyrrolyl, imidazolyl, pyrazolyl, triazolyl, tetrazolyl, oxazolyl, isoxazolyl, triazinyl, tetrazinyl, oxadiazolyl, thiazolyl, isothiazolyl, thiadiazolyl, pyridyl, pyridazinyl, pyrimidinyl, pyrazinyl, etc. Bi-cyclic heteroaryls include groups in which a monocyclic heteroaryl ring is fused to one or more aryl or heteroaryl rings. Nonlimiting examples include indolyl, imidazopyridinyl, benzooxazolyl, benzooxodiazolyl, indazolyl, benzimidazolyl, benzthiazolyl, quinolinyl, quinazolinyl, quinoxalinyl, pyrrolopyridinyl, pyrrolopyrimidinyl, pyrazolopyridinyl, thienopyridinyl, thienopyrimidinyl, indolizinyl, purinyl, naphthyridinyl, and pteridinyl. It will be understood that when specified, optional substituents on a heteroaryl group may be present on any substitutable position and, include, e.g., the position at which the heteroaryl is attached.

The term “heterocyclyl” means a 4- to 12-membered saturated or partially unsaturated heterocyclic ring containing 1 to 4 heteroatoms independently selected from N, O, and S. A heterocyclyl ring can be attached to its pendant group at any heteroatom or carbon atom that results in a stable structure. A heterocyclyl group may be mono- or bicyclic. Examples of monocyclic saturated or partially unsaturated heterocyclic radicals include, without limitation, tetrahydrofuranyl, tetrahydrothienyl, tetrahydropyranyl, pyrrolidinyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, and tetrahydropyrimidinyl. Bi-cyclic heterocyclyl groups include, e.g., unsaturated heterocyclic radicals fused to another unsaturated heterocyclic radical, cycloalkyl, or aromatic or heteroaryl ring, such as for example, benzodioxolyl, dihydrobenzooxazinyl, dihydrobenzodioxinyl, 6,7-dihydro-5H-pyrrolo[2,1-c][1,2,4]triazolyl, 5,6,7,8-tetrahydroimidazo[1,2-a]pyridinyl, 1,2-dihydroquinolinyl, dihydrobenzofuranyl, tetrahydronaphthyridine, indolinone, dihydropyrrolotriazole, quinolinone, chromanyl, and dioxaspirodecane. It will be understood that when specified, optional substituents on a heterocyclyl group may be present on any substitutable position and, include, e.g., the position at which the heterocyclyl is attached.

The term “spiro” refers to two rings that shares one ring atom (e.g., carbon).

The term “fused” refers to two rings that share two adjacent ring atoms with one another.

The term “bridged” refers to two rings that share three ring atoms with one another.

The term “TREX1” refers to three prime repair exonuclease 1 or DNA repair exonuclease 1, which is an enzyme that in humans is encoded by the TREX1 gene. Mazur D J, Perrino F W (August 1999). “Identification and expression of the TREX1 and TREX2 cDNA sequences encoding mammalian 3′-->5′ exonucleases”. J Biol Chem. 274 (28): 19655-60. doi:10.1074/jbc.274.28.19655. PMID 10391904; Hoss M, Robins P, Naven T J, Pappin D J, Sgouros J, Lindahl T (August 1999). “A human DNA editing enzyme homologous to the Escherichia coli DnaQ/MutD protein”. EMBO J. 18 (13): 3868-75. doi:10.1093/emboj/18.13.3868. PMC 1171463. PMID 10393201. This gene encodes the major 3′->5′ DNA exonuclease in human cells. The protein is a non-processive exonuclease that may serve a proofreading function for a human DNA polymerase. It is also a component of the SET complex, and acts to rapidly degrade 3′ ends of nicked DNA during granzyme A-mediated cell death. Cells lacking functional TREX1 show chronic DNA damage checkpoint activation and extra-nuclear accumulation of an endogenous single-strand DNA substrate. It appears that TREX1 protein normally acts on a single-stranded DNA polynucleotide species generated from processing aberrant replication intermediates. This action of TREX1 attenuates DNA damage checkpoint signaling and prevents pathological immune activation. TREX1 metabolizes reverse-transcribed single-stranded DNA of endogenous retroelements as a function of cell-intrinsic antiviral surveillance, resulting in a potent type I IFN response. TREX1 helps HIV-1 to evade cytosolic sensing by degrading viral cDNA in the cytoplasm.

The term “TREX2” refers to Three prime repair exonuclease 2 is an enzyme that in humans is encoded by the TREX2 gene. This gene encodes a nuclear protein with 3′ to 5′ exonuclease activity. The encoded protein participates in double-stranded DNA break repair, and may interact with DNA polymerase delta. Enzymes with this activity are involved in DNA replication, repair, and recombination. TREX2 is a 3′-exonuclease which is predominantly expressed in keratinocytes and contributes to the epidermal response to UVB-induced DNA damage. TREX2 biochemical and structural properties are similar to TREX1, although they are not identical. The two proteins share a dimeric structure and can process ssDNA and dsDNA substrates in vitro with almost identical km values. However, several features related to enzyme kinetics, structural domains, and subcellular distribution distinguish TREX2 from TREX1. TREX2 present a 10-fold lower affinity for DNA substrates in vitro compared with TREX1. In contrast with TREX1, TREX2 lacks a COOH-terminal domain that can mediate protein-protein interactions. TREX2 is localized in both the cytoplasm and nucleus, whereas TREX1 is found in the endoplasmic reticulum, and is mobilized to the nucleus during granzyme A-mediated cell death or after DNA damage.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The term “inhibit,” “inhibition” or “inhibiting” includes a decrease in the baseline activity of a biological activity or process.

As used herein, the terms “treatment,” “treat,” and “treating” refer to reversing, alleviating, delaying the onset of, or inhibiting the progress of a disease or disorder, or one or more symptoms thereof, as described herein. In some aspects, treatment may be administered after one or more symptoms have developed, i.e., therapeutic treatment. In other aspects, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “effective amount” or “therapeutically effective amount” refers to an amount of a compound described herein that will elicit a desired or beneficial biological or medical response of a subject e.g., a dosage of between 0.01-100 mg/kg body weight/day.

### 3. Compounds

In a second embodiment, R² in the compound of Formula I, or a pharmaceutically acceptable salt thereof, is hydrogen, (C₁-C₄)alkyl, halo(C₁-C₄)alkyl, —(C₁-C₄)alkylOR^{a}, 5- to 7-membered heteroaryl, or 4- to 7-membered heterocyclyl, wherein said heteroaryl and heterocyclyl are each optionally substituted with 1 to 3 groups selected from R⁶; R³ is halo, (C₁-C₄)alkyl, halo(C₁-C₄)alkyl, or CN; and m and n are each independently 0, 1, or 2, wherein the remaining variables are as described above for Formula I.

In a third embodiment, R³ in the compound of Formula I, or a pharmaceutically acceptable salt thereof, halo, halo(C₁-C₄)alkyl, (C₁-C₄)alkoxy, or halo(C₁-C₄)alkoxy; wherein the remaining features are as described above for Formula I or the second embodiment. Alternatively, as part of a third embodiment, R³ in the compound of Formula I, or a pharmaceutically acceptable salt thereof, is halo or halo(C₁-C₄)alkyl, wherein the remaining features are as described above for Formula I, or the second embodiment. In another alternative, as part of a third embodiment, R³ in the compound of Formula I, or a pharmaceutically acceptable salt thereof, is fluoro, chloro, methyl, methoxy, or CF₃, wherein the remaining features are as described above for Formula I, or the second embodiment. In another alternative, as part of a third embodiment, R³ in the compound of Formula I, or a pharmaceutically acceptable salt thereof, is fluoro, chloro, or CF₃, wherein the remaining features are as described above for Formula I, or the second embodiment.

In a fourth embodiment, the compound of Formula I is of the Formula II:

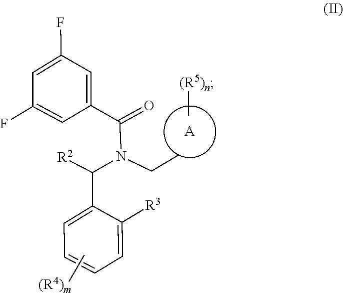

or a pharmaceutically acceptable salt thereof, wherein the variables are as described above for Formula I or the second embodiment or third embodiment.

In a fifth embodiment, R² in the compound of Formula I or II, or a pharmaceutically acceptable salt thereof is hydrogen or (C₁-C₄)alkyl, wherein the variables are as described above for Formula I or the second embodiment or third embodiment.

In a sixth embodiment, the compound of Formula I is of the Formula III:

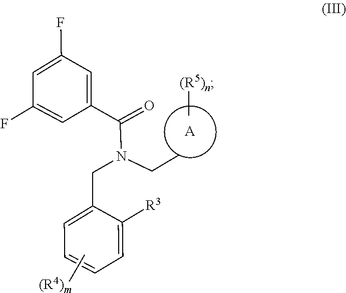

or a pharmaceutically acceptable salt thereof, wherein the variables are as described above for Formula I or the second or third embodiment.

In a seventh embodiment, R⁴ in the compound of Formula I, II, or III, or a pharmaceutically acceptable salt thereof, is halo, (C₁-C₄)alkyl, halo(C₁-C₄)alkyl, (C₁-C₄)alkoxy, or halo(C₁-C₄)alkoxy or CN, wherein the remaining variables are as described above for Formula I or the second, third, or fifth embodiment. Alternatively, as part of a seventh embodiment, R⁴ in the compound of Formula I, II, or III, or a pharmaceutically acceptable salt thereof, is chloro, methyl, or methoxy wherein the remaining variables are as described above for Formula I or the second, third, or fifth embodiment. In another alternative, as part of a seventh embodiment, R⁴ in the compound of Formula I, II, or III, or a pharmaceutically acceptable salt thereof, is halo wherein the remaining variables are as described above for Formula I or the second, third, or fifth embodiment. In another alternative, as part of a seventh embodiment, R⁴ in the compound of Formula I, II, or III, or a pharmaceutically acceptable salt thereof, is chloro wherein the remaining variables are as described above for Formula I or the second, third, or fifth embodiment.

In an eighth embodiment, m in the compound of Formula I, II, or III, or a pharmaceutically acceptable salt thereof, is 0, 1 or 2, wherein the remaining variables are as described above for Formula I or the second, third, fifth, or seventh embodiment. Alternatively, as part of an eighth embodiment, m in the compound of Formula I, II, or III, or a pharmaceutically acceptable salt thereof, is 0 or 1, wherein the remaining variables are as described above for Formula I or the second, third, fifth, or seventh embodiment.

In an ninth embodiment, the compound of Formula I is of the Formula IV:

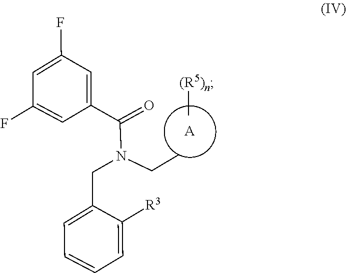

or a pharmaceutically acceptable salt thereof, wherein the remaining variables are as described above for Formula I or the second, third, fifth, seventh, or eighth embodiment.

In a tenth embodiment, Ring A in the compound of Formula I, II, III, or IV, or a pharmaceutically acceptable salt thereof, is pyridyl, pyrimidinyl, pyridazinyl, pyrazinyl, or pyrazolyl, wherein the remaining variables are as described above for Formula I or the second, third, fifth, seventh, or eighth embodiment. Alternatively, as part of a tenth embodiment, Ring A in the compound of Formula I, II, III, or IV, or a pharmaceutically acceptable salt thereof, is pyridyl, wherein the remaining variables are as described above for Formula I or the second, third, fifth, seventh, or eighth embodiment.

In an eleventh embodiment, the compound of Formula I is of the Formula V:

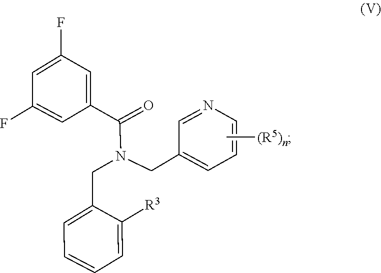

or a pharmaceutically acceptable salt thereof, wherein the variables are as described above for Formula I or the second or third embodiment.

In a twelfth embodiment, R⁵ in the compound of Formula I, II, I, IV, or V, or a pharmaceutically acceptable salt thereof, is in the para position with respect to the connection point for Ring A, wherein the remaining variables are as described above for Formula I or the second, third, fifth, seventh, eighth, or tenth embodiment.

In a thirteenth embodiment, R⁵ in the compound of Formula I, II, III, IV, or V, or a pharmaceutically acceptable salt thereof, is halo, oxo, (C₁-C₄)alkyl, halo(C₁-C₄)alkyl, —C(O)OR^{b}, —NR^{b}R^{c}, —C(O)NR^{b}R^{c}, —(C₁-C₄)alkylOR^{b}, —(C₁-C₄)alkylC(O)R^{b}, —(C₁-C₄)alkylC(O)NR^{b}R^{c}, —C(O)NR^{b}SO₂(C₁-C₄)alkyl, 5- to 6-membered heteroaryl, wherein said heteroaryl is optionally substituted with 1 to 3 groups selected from R⁷, wherein the remaining variables are as described above for Formula I or the second, third, fifth, seventh, eighth, tenth, or twelfth embodiment. Alternatively, as part of a thirteenth embodiment, R⁵ in the compound of Formula I, H, III, IV, or V, or a pharmaceutically acceptable salt thereof, is oxo, CF₃, CH₃, C(O)OCH₃, C(O)OH, CH₂COOH, NH₂, CONH₂, CH₂CONH₂, CONHCH₃, CH₂OH, CH₂CH₂OH, CONHSO₂CH₃, tetrazolyl, pyrazolyl, triazolyl, or pyrazolyl, wherein said pyrazolyl is optionally substituted with hydroxy, wherein the remaining variables are as described above for Formula I or the second, third, fifth, seventh, eighth, tenth, or twelfth embodiment.

In a fourteenth embodiment, n in the compound of Formula I, II, I, IV, or V. or a pharmaceutically acceptable salt thereof, is 0, 1 or 2, wherein the remaining variables are as described above for Formula I or the second, third, fifth, seventh, eighth, tenth, twelfth, or thirteenth embodiment. Alternatively, as part of a fourteenth embodiment, n in the compound of Formula I, II, III, IV, or V. or a pharmaceutically acceptable salt thereof, is 1 or 2, wherein the remaining variables are as described above for Formula I or the second, third, fifth, seventh, eighth, tenth, twelfth, or thirteenth embodiment. In another alternative, as part of a fourteenth embodiment, n in the compound of Formula I, II, I, IV, or V. or a pharmaceutically acceptable salt thereof, is 2, wherein the remaining variables are as described above for Formula I or the second, third, fifth, seventh, eighth, tenth, twelfth, or thirteenth embodiment.

In a fifteenth embodiment, R^{3a} in the compound of Formula VI, or a pharmaceutically acceptable salt thereof, is hydrogen, (C₁-C₄)alkyl, halo(C₁-C₄)alkyl, —(C₁-C₄)alkylOR^{a}, 5- to 7-membered heteroaryl, or 4- to 7-membered heterocyclyl, wherein said heteroaryl and heterocyclyl are each optionally substituted with 1 to 3 groups selected from R^{7a}; and m1, n1, and p1 are each independently 0, 1, or 2, wherein the remaining variables are as described above for Formula VI.

In a sixteenth embodiment, R^{1a} in the compound of Formula VI, or a pharmaceutically acceptable salt thereof, is halo, wherein the remaining variables are as described above for Formula VI or the fifteenth embodiment.

In a seventeenth embodiment, the compound of Formula VI is of the Formula VII:

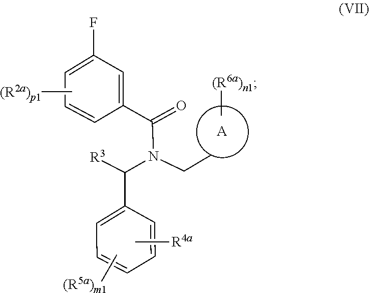

or a pharmaceutically acceptable salt thereof, wherein the variables are as described above for Formula VI or the fifteenth or sixteenth embodiment.

In an eighteenth embodiment, R^{2a} in the compound of Formula VI or VII, or a pharmaceutically acceptable salt thereof, is halo, hydroxy, (C₁-C₄)alkyl, halo(C₁-C₄)alkyl, or (C₁-C₄)alkoxy, wherein the variables are as described above for Formula VI or the fifteenth or sixteenth embodiment. Alternatively, as part of an eighteenth embodiment, R^{2a} in the compound of Formula VI or VII, or a pharmaceutically acceptable salt thereof, is halo, wherein the variables are as described above for Formula VI or the fifteenth or sixteenth embodiment.

In a nineteenth embodiment, the compound of Formula VI is of the Formula VIII:

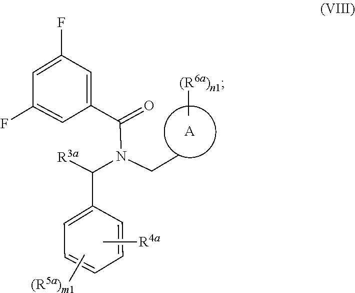

or a pharmaceutically acceptable salt thereof, wherein the variables are as described above for Formula VI or the fifteenth, sixteenth, or eighteenth embodiment.

In a twentieth embodiment, the compound of Formula VI is of the Formula IX:

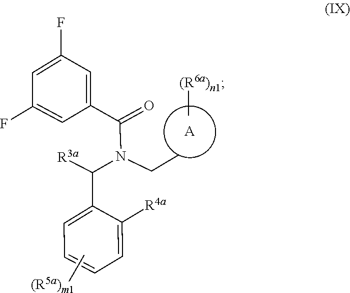

or a pharmaceutically acceptable salt thereof, wherein the variables are as described above for Formula VI or the fifteenth, sixteenth, or eighteenth embodiment.

In a twenty-first embodiment, R^{4a} in the compound of Formula VI, VII, VIII, or IX, or pharmaceutically acceptable salt thereof is halo, (C₁-C₄)alkyl, halo(C₁-C₄)alkyl, (C₁-C₄)alkoxy, or halo(C₁-C₄)alkoxy, wherein the variables are as described above for Formula VI or the fifteenth, sixteenth, or eighteenth embodiment. Alternatively, as part of a twenty-first embodiment, R^{4a} in the compound of Formula VI, VII, VIII, or IX, or pharmaceutically acceptable salt thereof is fluoro, chloro, methyl, methoxy, or CF₃, wherein the variables are as described above for Formula VI or the fifteenth, sixteenth, or eighteenth embodiment. In another alternative, as part of a twenty-first embodiment, R^{4a} in the compound of Formula VI, VII, VIII, or IX, or pharmaceutically acceptable salt thereof is halo(C₁-C₄)alkyl or halo, wherein the variables are as described above for Formula VI or the fifteenth, sixteenth, or eighteenth embodiment. In another alternative, as part of a twenty-first embodiment, R^{4a} in the compound of Formula VI, VII, VIII, or IX, or pharmaceutically acceptable salt thereof is fluoro, chloro or CF₃, wherein the variables are as described above for Formula VI or the fifteenth, sixteenth, or eighteenth embodiment.

In a twenty-second embodiment, R^{3a} in the compound of Formula VI, VII, VIII, or IX, or pharmaceutically acceptable salt thereof is hydrogen or (C₁-C₄)alkyl, wherein the variables are as described above for Formula VI or the fifteenth, sixteenth, eighteenth, or twenty-first embodiment.

In a twenty-third embodiment, the compound of Formula VI is of the Formula X:

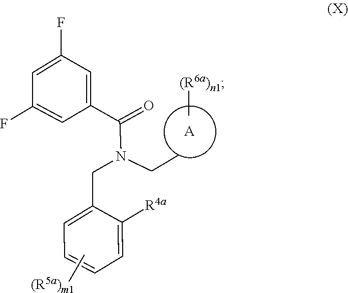

or a pharmaceutically acceptable salt thereof, wherein the variables are as described above for Formula VI or the fifteenth, sixteenth, or eighteenth embodiment.

In a twenty-fourth embodiment, R^{5a} in the compound of Formula VI, VII, VIII, IX, or X, or pharmaceutically acceptable salt thereof, is halo or (C₁-C₄)alkoxy, wherein the variables are as described above for Formula VI or the fifteenth, sixteenth, eighteenth, or twenty-first embodiment. Alternatively, as part of a twenty-fourth embodiment, R^{5a} in the compound of Formula VI, VII, VIII, IX, or X, or pharmaceutically acceptable salt thereof, is chloro, methyl, or methoxy, wherein the variables are as described above for Formula VI or the fifteenth, sixteenth, eighteenth, or twenty-first embodiment. In another alternative, as part of a twenty-fourth embodiment, R^{5a} in the compound of Formula VI, VII, VIII, IX, or X, or pharmaceutically acceptable salt thereof, is halo, wherein the variables are as described above for Formula VI or the fifteenth, sixteenth, eighteenth, or twenty-first embodiment. In another alternative, as part of a twenty-fourth embodiment, R^{5a} in the compound of Formula VI, VII, VIII, IX, or X, or pharmaceutically acceptable salt thereof, is chloro, wherein the variables are as described above for Formula VI or the fifteenth, sixteenth, eighteenth, or twenty-first embodiment.

In a twenty-fifth embodiment, m1 in the compound of Formula VI, VII, VIII, IX, or X, or pharmaceutically acceptable salt thereof, is 0, 1 or 2, wherein the variables are as described above for Formula VI or the fifteenth, sixteenth, eighteenth, twenty-first, or twenty-fourth embodiment.

In a twenty-sixth embodiment, Ring A in the compound of Formula VI, VII, VIII, IX, or X, or pharmaceutically acceptable salt thereof, is a 5-7 membered heteroaryl, wherein the variables are as described above for Formula VI or the fifteenth, sixteenth, eighteenth, twenty-first, twenty-fourth, or twenty-fifth embodiment. Alternatively, as part of a twenty-fourth embodiment, Ring A in the compound of Formula VI, VII, VIII, IX, or X, or pharmaceutically acceptable salt thereof, is a nitrogen-containing 5- to 7-membered heteroaryl, wherein the variables are as described above for Formula VI or the fifteenth, sixteenth, eighteenth, twenty-first, twenty-fourth, or twenty-fifth embodiment. In another alternative, as part of a twenty-fourth embodiment, Ring A in the compound of Formula VI, VII, VIII, IX, or X, or pharmaceutically acceptable salt thereof, is pyridyl, pyrimidinyl, pyradazinyl, pyrazinyl, or pyrazolyl, wherein the variables are as described above for Formula VI or the fifteenth, sixteenth, eighteenth, twenty-first, twenty-fourth, or twenty-fifth embodiment. In another alternative, as part of a twenty-fourth embodiment, Ring A in the compound of Formula VI, VII, VIII, IX, or X, or pharmaceutically acceptable salt thereof, is pyridyl, wherein the variables are as described above for Formula VI or the fifteenth, sixteenth, eighteenth, twenty-first, twenty-fourth, or twenty-fifth embodiment.

In a twenty-seventh embodiment, the compound of Formula VI is of the Formula XI:

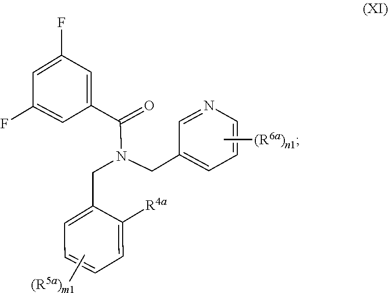

or a pharmaceutically acceptable salt thereof, wherein the variables are as described above for Formula VI or the twenty-first, twenty-fourth, twenty-fifth, or twenty-sixth embodiment.

In a twenty-eighth embodiment, R⁶ in the compound of Formula VI, VII, VIII, IX, X, or XI, or pharmaceutically acceptable salt thereof, is halo, oxo, (C₁-C₄)alkyl, halo(C₁-C₄)alkyl, —C(O)OR^{b1}, —NR^{b1}R^{c1}, —C(O)NR^{b1}R^{c1}, —(C₁-C₄)alkylOR^{b1}, —(C₁-C₄)alkylC(O)R^{b1}, —(C₁-C₄)alkylC(O)NR^{b1}R^{c1}, —C(O)NR^{b1}SO₂(C₁-C₄)alkyl, 5- to 6-membered heteroaryl, wherein said heteroaryl is optionally substituted with 1 to 3 groups selected from R^{7a}, wherein the variables are as described above for Formula VI or the fifteenth, sixteenth, eighteenth, twenty-first, twenty-fourth, twenty-fifth, or twenty-sixth embodiment. Alternatively, as part of a twenty-eighth embodiment, R^{6a} in the compound of Formula VI, VII, VIII, IX, X, or XI, or pharmaceutically acceptable salt thereof, is oxo, CF₃, CH₃, C(O)OCH₃, C(O)OH, CH₂COOH, NH₂, CONH₂, CH₂CONH₂, CONHCH₃, CH₂OH, CH₂CH₂OH, CONHSO₂CH₃, tetrazolyl, pyrazolyl, triazolyl, or pyrazolyl, wherein said pyrazolyl is optionally substituted with hydroxy, wherein the variables are as described above for Formula VI or the fifteenth, sixteenth, eighteenth, twenty-first, twenty-fourth, twenty-fifth, or twenty-sixth embodiment.

In a twenty-ninth embodiment, n1 in the compound of Formula VI, VII, VIII, IX, X, or XI, or pharmaceutically acceptable salt thereof is 0, 1 or 2, wherein the variables are as described above for Formula VI or the fifteenth, sixteenth, eighteenth, twenty-first, twenty-fourth, twenty-fifth, twenty-sixth, or twenty-eighth embodiment Alternatively, as part of a twenty-ninth embodiment, n1 in the compound of Formula VI, VII, VIII, IX, X, or XI, or pharmaceutically acceptable salt thereof is 1 or 2, wherein the variables are as described above for Formula VI or the fifteenth, sixteenth, eighteenth, twenty-first, twenty-fourth, twenty-fifth, twenty-sixth, or twenty-eighth embodiment. In another alternative, as part of a twenty-ninth embodiment, n1 in the compound of Formula VI, VII, VIII, IX, X, or XI, or pharmaceutically acceptable salt thereof is 0 or 1, wherein the variables are as described above for Formula VI or the fifteenth, sixteenth, eighteenth, twenty-first, twenty-fourth, twenty-fifth, twenty-sixth, or twenty-eighth embodiment.

Compounds having the Formula I and VI are further disclosed in the Exemplification and are included in the present disclosure. Pharmaceutically acceptable salts thereof as well as the neutral forms are included.

### 4. Uses, Formulation and Administration

The compounds and compositions described herein are generally useful for modulating the activity of TREX1. In some aspects, the compounds and pharmaceutical compositions described herein inhibit the activity TREX1.

In some aspects, the compounds and pharmaceutical compositions described herein are useful in treating a disorder associated with TREX1 function. Thus, provided herein are methods of treating a disorder associated with TREX1 function, comprising administering to a subject in need thereof, a therapeutically effective amount of a compound described herein, or a pharmaceutically acceptable salt thereof, or a pharmaceutical composition comprising a disclosed compound or pharmaceutically acceptable salt thereof. Also provided is the use of a compound described herein, or a pharmaceutically acceptable salt thereof, or a pharmaceutical composition comprising a disclosed compound or pharmaceutically acceptable salt thereof, for the manufacture of a medicament for treating a disorder associated with TREX1 function. Also provided is a compound described herein, or a pharmaceutically acceptable salt thereof, or a pharmaceutical composition comprising a disclosed compound or pharmaceutically acceptable salt thereof, for use in treating a disorder associated with TREX1.

In some aspects, the compounds and pharmaceutical compositions described herein are useful in treating cancer.

In some aspects, the cancer treated by the compounds and pharmaceutical compositions described herein is selected from colon cancer, gastric cancer, thyroid cancer, lung cancer, leukemia, pancreatic cancer, melanoma, multiple melanoma, brain cancer, CNS cancer, renal cancer, prostate cancer, ovarian cancer, leukemia, and breast cancer.

In some aspects, the cancer treated by the compounds and pharmaceutical compositions described herein is selected from lung cancer, breast cancer, pancreatic cancer, colorectal cancer, and melanoma.

In certain aspects, a pharmaceutical composition described herein is formulated for administration to a patient in need of such composition. Pharmaceutical compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the pharmaceutical compositions described herein may be aqueous or oleaginous suspension. These suspensions may be formulated according to techniques known in the art using suitable dispersing or wetting agents and suspending agents.

In some aspects, the pharmaceutical compositions are administered orally.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the pharmaceutical composition.

### EXEMPLIFICATION

### Chemical Synthesis

The representative examples that follow are intended to help illustrate the present disclosure, and are not intended to, nor should they be construed to, limit the scope of the invention.

General starting materials used were obtained from commercial sources or prepared in other examples, unless otherwise noted.

### Abbreviations

ACN Acetonitrile
AcOH Acetic acid
AIBN α,α′-Azoisobutyronitrile
DCE 1,2-dichloroethane
DCM Dichloromethane or methylene chloride
DEA Diethylamine
DIEA N,N-diisopropylethylamine
DMA N,N-Dimethylacetamide
DMAP N,N-Dimethylpyridin-4-amine
DMC 2-Chloro-1,3-dimethylimidazolinium chloride
DMF N,N-dimethylformamide
DMSO Dimethylsulfoxide
EtOAc Ethyl acetate
HATU 2-(3H-[1,2,3]triazolo[4,5-b]pyridin-3-yl)-1,1,3,3-tetramethyl-isouronium hexafluorophosphate
HCl hydrogen chloride
HPLC high performance liquid chromatography
IPA 2-Propanol
LAH Lithium aluminum hydride
LC/MS Liquid chromatography/mass spectrometry
K₂CO₃ Potassium carbonate
MeOH Methanol
MS Mass spectrometry
NaBH(OAc)₃ Sodium triacetoxyborohydride
NaCNBH₄ Sodium cyanoborohydride
NaOH Sodium hydroxide
NaHCO₃Sodium bicarbonate
Na₂SO₄ Sodium sulfate
RT Room temperature
TEA Triethylamine
TFA Trifluoroacetic acid
THF Tetrahydrofuran

The progress of reactions was often monitored by TLC or LC-MS. The LC-MS was recorded using one of the following methods.

### LCMS Method-1:

| Mobile Phase | (A) | 2 mM Ammonium acetate with 0.1% | | |
|---|---|---|---|---|
| | | Formic Acid in Water | | |
| | (B) | 0.1% Formic Acid in Acetonitrile | | |
| Column: | | BEH C18 (50*2.1 mm) 1.7 um | | |
| Column Flow: | | 0.55 ml/min | | |
| | | Time (min) | % A | % B |
| Gradient: | | 0.01 | 98 | 2 |
| | | 0.30 | 98 | 2 |
| | | 0.60 | 50 | 50 |
| | | 1.10 | 25 | 75 |
| | | 2.00 | 0 | 100 |
| | | 2.70 | 0 | 100 |
| | | 2.71 | 98 | 2 |
| | | 3.00 | 98 | 2 |

### LCMS Method-2:

| Mobile Phase | (A) | 5 mM Ammonium Acetate with 0.1% | | |
|---|---|---|---|---|
| | | Formic Acid in Water | | |
| | (B) | 0.1% Formic Acid in Acetonitrile | | |
| Column: | | BEH C18 (50*2.1 mm), 1.7 um or Equivalent | | |
| Column Flow: | | 0.45 ml/min | | |
| | | Time (min) | % A | % B |
| Gradient: | | 0.01 | 98 | 2 |
| | | 0.50 | 98 | 2 |
| | | 5.00 | 10 | 90 |
| | | 6.00 | 5 | 95 |
| | | 7.00 | 5 | 95 |
| | | 7.01 | 98 | 2 |
| | | 8.00 | 98 | 2 |

### LCMS Method-3:

| Mobile Phase | (A) | 5 mM Ammonium bicarbonate in Water | | |
|---|---|---|---|---|
| | (B) | Acetonitrile | | |
| Column: | | X-Bridge C18 (50*4.6 mm), 3.5 um | | |
| Column Flow: | | 1.0 ml/min | | |
| | | Time (min) | % A | % B |
| Gradient: | | 0.01 | 95 | 5 |
| | | 5.00 | 10 | 90 |
| | | 5.80 | 5 | 95 |
| | | 7.20 | 5 | 95 |
| | | 7.21 | 95 | 5 |
| | | 10.00 | 95 | 5 |

### LCMS Method-4:

| Mobile Phase | (A) | 10 mM Ammonium Acetate in Water | | |
|---|---|---|---|---|
| | (B) | 100% Acetonitrile | | |
| Column: | | X-Bridge C18 (150*4.6 mm), 5 um or Equivalent | | |
| Column Flow: | | 1.0 ml/min | | |
| | | Time (min) | % A | % B |
| Gradient: | | 0.01 | 90 | 10 |
| | | 5.00 | 10 | 90 |
| | | 7.00 | 0 | 100 |
| | | 11.00 | 0 | 100 |
| | | 11.01 | 90 | 10 |
| | | 12.00 | 90 | 10 |

### LCMS Method-5:

| Mobile Phase | (A) | 10 mM Ammonium Acetate in Water | | |
|---|---|---|---|---|
| | (B) | 100% Acetonitrile | | |
| Column: | | X-Bridge C18 (150*4.6 mm), 5 um or Equivalent | | |
| Column Flow: | | 1.0 ml/min | | |
| | | Time (min) | % A | % B |
| Gradient: | | 0.01 | 100 | 0 |
| | | 7.00 | 50 | 50 |
| | | 9.00 | 0 | 100 |
| | | 11.00 | 0 | 100 |
| | | 11.01 | 100 | 0 |
| | | 12.00 | 100 | 0 |

### LCMS Method-6:

| Mobile Phase | (A) | 0.1% Formic Acid in Water | | |
|---|---|---|---|---|
| | (B) | 0.1% Formic Acid in Acetonitrile | | |
| Column: | | Zorbax SB-C8 (4.5 × 75 mm), 3.5 μm | | |
| Column Flow: | | 1.5 ml/min | | |
| | | Time (min) | % A | % B |
| Gradient: | | 0.00 | 95 | 5 |
| | | 3.60 | 5 | 95 |
| | | 4.00 | 5 | 95 |
| | | 4.50 | 95 | 5 |

NMR was recorded at room temperature unless noted otherwise on Varian Inova 400 or 500 MHz spectrometers with the solvent peak used as the reference or on Bruker 300 or 400 MHz spectrometers with the TMS peak used as internal reference.

### General Key Intermediates Procedures

### Synthesis of Intermediate 1 [(3,5-dichlorophenyl)methyl][(pyridin-3-yl)methyl]amine hydrochloride

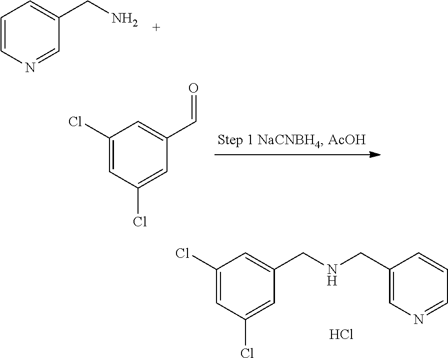 [SMILES: NCc1cccnc1 ; O=Cc1cc(Cl)cc(Cl)c1 ; Clc1cc(Cl)cc(CNCc2cccnc2)c1 ; Cl]

Step 1. Under an inert atmosphere 1-(pyridin-3-yl)methanamine (4.70 mL, 46.2 mmol) was combined in Methanol (83 mL) with 3,5-dichlorobenzaldehyde (4.04 g, 23.1 mmol). Sodium cyanoborohydride (2.90 g, 46.2 mmol), and Acetic acid (2.63 mL, 46.2 mmol) were added. The reaction mixture was stirred at room temperature for 4 hours. The reaction mixture was concentrated, taken up in ethyl acetate, and washed twice with saturated NaHCO₃ solution. The organic phase was concentrated under vacuum. The residue was dissolved in THF then HCl (4M in 1,4-dioxane) was added. The precipitated HCl salt was filtered, washed with THF, and dried under vacuum to afford [(3,5-dichlorophenyl)methyl][(pyridin-3-yl)methyl]amine hydrochloride (5.859 g, 40.5%) as a white solid. MS ES m/z: 267.1 [M+H]⁺.

### Synthesis of Intermediate 2 [(2,4-dichlorophenyl)methyl][(pyridin-3-yl)methyl]amine hydrochloride

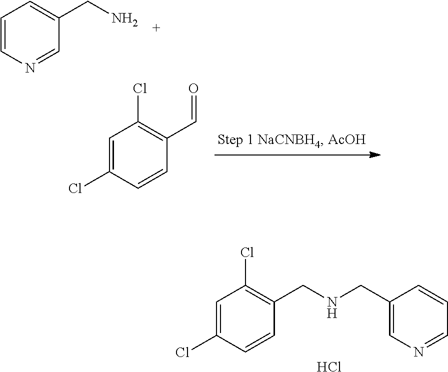 [SMILES: NCc1cccnc1 ; O=Cc1ccc(Cl)cc1Cl ; Clc1ccc(CNCc2cccnc2)c(Cl)c1 ; Cl]

Step 1. Under an inert atmosphere of N2, 1-(pyridin-3-yl)methanamine (4.70 mL, 46.2 mmol) was dissolved in Methanol (83 mL) followed by addition of 2,4-dichlorobenzaldehyde (4.04 g, 23.1 mmol), Sodium cyanoborohydride (2.90 g, 46.2 mmol), and Acetic acid (2.63 mL, 46.2 mmol). The reaction was stirred at room temperature for 4 hours. The reaction mixture was concentrated, taken up in ethyl acetate, and washed twice with saturated NaHCO₃ solution. The organic phase was concentrated. The residue was dissolved in THF then HCl (4M in 1,4-dioxane) was added. The precipitated HCl salt was filtered, washed with THF, and dried under vacuum to afford [(2,4-dichlorophenyl)methyl][(pyridin-3-yl)methyl]amine hydrochloride (5.692 g, 39.4%) as a white solid. MS ES m/z: 267.1 [M+H]⁺.

### Synthesis of Intermediate 3 (1-(2,3-dichlorophenyl)propan-1-amine)

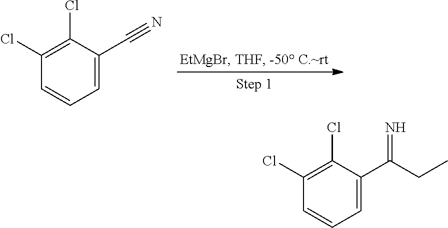

Step 1. To a stirred solution of 2,3-dichlorobenzonitrile (1.00 g, 5.814 mmol) and THF (10.00 mL) were added EtMgBr (1.55 g, 11.628 mmol) dropwise at −50° C. under argon atmosphere. The resulting mixture was stirred for 1 h at room temperature under argon atmosphere. The reaction was quenched with sat. NH₄Cl (aq.) at 0° C. The aqueous layer was extracted with EtOAc (3×20 mL). The resulting mixture was concentrated under vacuum to afford (1-(2,3-dichlorophenyl)propan-1-imine) (930 mg, 79%) as a yellow oil and used for next step directly.

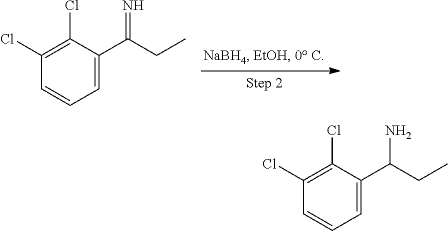

Step 2. To a stirred solution of (1-(2,3-dichlorophenyl)propan-1-imine) (900 mg, 4.454 mmol) and EtOH (60.00 mL) was added NaBH₄ (336.99 mg, 8.907 mmol) in portions at 0° C. The resulting mixture was stirred for 2 h at 0° C. The reaction was quenched with sat. NH₄Cl (aq.) at 0° C. The aqueous layer was extracted with EtOAc (3×100 mL). The resulting mixture was concentrated under vacuum to afford (1-(2,3-dichlorophenyl)propan-1-amine) (832 mg, 91%) as a yellow solid. ES MS m/z: 204.0 [M+H]⁺.

### Synthesis of 1-(2,4-Dichlorophenyl)-N-(pyridin-3-ylmethyl)-1-(1-(tetrahydro-2H-pyran-2-yl)-1H-pyrazol-4-yl)methanamine (Intermediate 4)

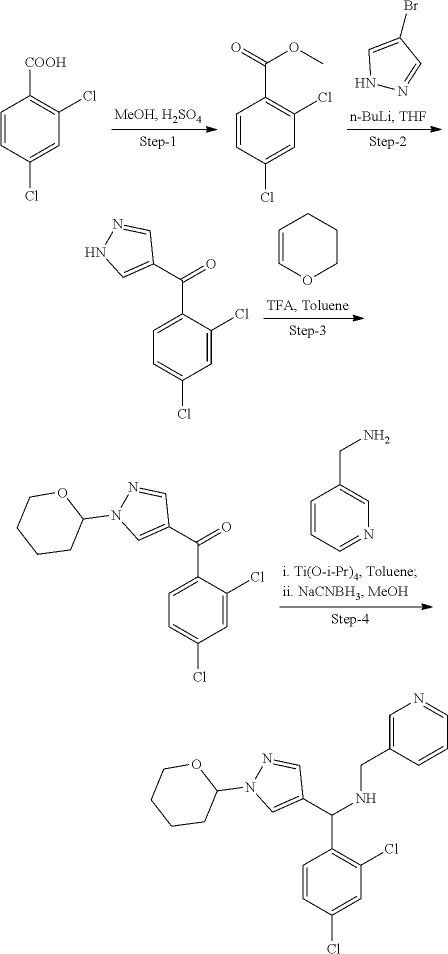

Step-1: Methyl 2,4-dichlorobenzoate: To the stirred solution of 2,4-dichlorobenzoic acid (1.5 g, 7.85 mmol) in MeOH (10 ml), concentrated sulfuric acid (1 ml) was added and the reaction mixture was heated at 80° C. for 16 hours. After completion of reaction (monitored by TLC), the reaction mixture was quenched with water (100 ml) and extracted with Ethyl acetate (3×100 ml). The combined organic layer was washed with brine (100 ml), dried over anhydrous Sodium sulphate, and concentrated under reduced pressure. The crude compound was purified by using combi-flash chromatography to obtain pure title compound (1.4 g, 87%). ¹H NMR (400 MHz, DMSO-d₆): δ 3.88 (s, 3H), 7.57 (dd, J=8.4 Hz, J=2.0 Hz, 1H), 7.79 (d, J=1.6 Hz, 1H), 7.86 (d, J=8.4 Hz, 1H).

Step-2: (2,4-Dichlorophenyl)(1H-pyrazol-4-yl)methanone: To a solution of 4-bromo-1H-pyrazole (1.0 g, 6.85 mmol) in THF (10 ml) at −78° C., n-BuLi (6.3 ml, 16.44 mmol) was added under N_{2(g)} atmosphere and stirred the reaction mixture at −78° C. for 5-10 minutes. Reaction mixture was stirred at room temperature for 1 hour. After 1 hour, the reaction mixture was cooled to −78° C. and methyl 2,4-dichlorobenzoate (1.4 g, 6.85 mmol) was added at −78° C. and allowed to come at room temperature and stirred at room temperature for 1 hour. After completion of the reaction (monitored by TLC), the reaction mixture was quenched with saturated solution of NH₄Cl (100 ml) and extracted with Ethyl acetate (3×100 ml). The combined organic layer was washed with brine (100 ml), dried over anhydrous Sodium sulphate, and concentrated under reduced pressure. The crude compound was purified by using combi-flash chromatography to obtain pure title compound (0.6 g, 51.3%). ES MS m/z: 239.0 [M+H]⁺. ¹H NMR (400 MHz, DMSO-d₆): δ 7.56 (bs, 2H), 7.91 (s, 1H), 8.29 (s, 1H), 13.65 (s, 1H).

Step-3: (2,4-Dichlorophenyl)(1-(tetrahydro-2H-pyran-2-yl)-1H-pyrazol-4-yl)methanone: To the solution of (2,4-dichlorophenyl)(1H-pyrazol-4-yl)methanone (0.900 g, 3.73 mmol) in Toluene (10 ml), 3,4-dihydropyran (0.470 g, 5.6 mmol) was added followed by the addition of TEA (0.1 ml) at room temperature. The reaction mixture was heated at 100° C. for 48 hours. After completion of reaction (monitored by TLC), reaction mixture was concentrated under vacuum to get crude compound which was used for next step without further purification (0.780 g, Crude).

Step-4: 1-(2,4-Dichlorophenyl)-N-(pyridin-3-ylmethyl)-1-(1-(tetrahydro-2H-pyran-2-yl)-1H-pyrazol-4-yl)methanamine: To the solution of (2,4-dichlorophenyl)(1-(tetrahydro-2H-pyran-2-yl)-1H-pyrazol-4-yl)methanone (0.780 g, 2.4 mmol) in Toluene (10 ml), pyridin-3-ylmethanamine (0.389 g, 3.6 mmol) was added followed by the addition of titanium(IV) isopropoxide (1.4 g, 4.8 mmol) and the reaction mixture was heated at 90° C. for 10 hours. NaCNBH₃ (0.223 g, 3.6 mmol) and MeOH (20 ml) were added and he reaction mixture was heated at 80° C. for another 16 hours. After completion of the reaction (monitored by TLC), to the reaction mixture reaction mixture water (50 ml) was added and stirred for 10 minutes. After 10 minutes, the reaction mixture was filtered and was washed with DCM (2×50 ml). Filtrate was concentrated to get the crude. Crude compound was used for next step without further purification (0.650 g, crude).

### Synthesis of Methyl 2-(5-(((2,4-dichlorobenzyl)amino)methyl)pyridin-2-yl)acetate (Intermediate 5)

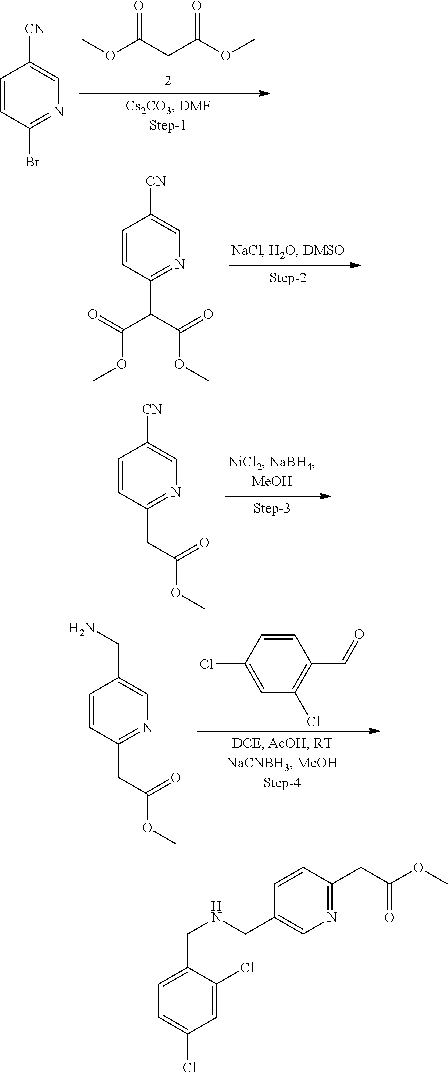

Step-1: Dimethyl 2-(5-cyanopyridin-2-yl)malonate: To a stirred solution of 6-bromonicotinonitrile (10.0 g, 54.641 mmol) and dimethyl malonate (10.82 g, 81.9 mmol) in DMF (100 ml) was added Cs₂CO₃ (53.40 g, 163.9 mmol) at room temperature. The reaction mixture was stirred at room temperature for overnight. After completion of reaction (monitored by TLC), the reaction mixture was poured on to ice cold Water (100 ml) and extracted with Ethyl acetate (3×100 ml). Combined organic layer was washed with brine (100 ml) and dried over Na₂SO₄. Evaporated under vacuum to obtain the crude compound. The crude compound was purified using column chromatography to obtain pure compound (9.2 g, 71%). ¹H NMR (400 MHz, DMSO-d₆): δ 3.81 (s, 6H), 5.04 (s, 1H), 7.68 (d, J=8.0 Hz, 1H), 8.01 (d, J=6.4 Hz, 1H), 8.85 (s, 1H).

Step-2: Methyl 2-(5-cyanopyridin-2-yl)acetate: To a stirred solution of dimethyl 2-(5-cyanopyridin-2-yl) malonate (8.0 g, 34.2 mmol) in DMSO (80 ml) was added aqueous solution of NaCl (2.19 g, 37.5 mmol) at room temperature. The reaction mixture was stirred at 130° C. for overnight. After completion of reaction (monitored by TLC), the reaction mixture was poured on to ice cold Water (100 ml) and extracted with Ethyl acetate (3×100 ml). Combined organic layer was washed with brine (100 ml) and dried over Na₂SO₄. evaporated under vacuum to obtain the crude compound. The crude compound was purified using column chromatography to obtain pure compound (2.7 g, 44%). ¹H NMR (400 MHz, DMSO-d₆): δ 3.78 (s, 3H), 3.97 (s, 2H), 7.49 (d, J=8.0 Hz, 1H), 7.97 (d, J=8.0 Hz, 1H), 8.87 (s, 1H).

Step-3: Methyl 2-(5-(aminomethyl)pyridin-2-yl)acetate: To a stirred solution of methyl 2-(5-cyanopyridin-2-yl)acetate (0.50 g, 2.8 mmol) in MeOH (5 ml) was added NiCl₂·6H₂O (0.067 g, 0.28 mmol) followed by NaBH₄ (0.750 g, 19.4 mmol) portion wise at 0° C. The reaction mixture was stirred at room temperature for overnight. After completion of reaction (monitored by TLC), the reaction mixture was filtered through celite and filtrate was evaporated under vacuum to obtain the crude compound. The crude compound was purified using RP-HPLC to obtain pure compound (0.400 g, 78%). ES MS m/z: 181.1 [M+H]⁺.

Step-4: Methyl 2-(5-(((2,4-dichlorobenzyl)amino)methyl)pyridin-2-yl)acetate: To a stirred solution of methyl 2-(5-(aminomethyl)pyridin-2-yl)acetate (0.300 g, 1.66 mmol) and 2,4-dichlorobenzaldehyde (0.291 g, 1.66 mmol) in 1,2-Dichloroethane (3 ml) was added AcOH (0.3 ml) at room temperature. Reaction mixture was stirred at room temperature for 1 hour. Sodium cyanoborohydride (0.156 g, 2.49 mmol) and Methanol (0.3 ml) were added and reaction mixture stirred at room temperature for another 1 hour. After completion of reaction (monitored by TLC), the reaction mixture was poured on to ice cold water (20 ml) and extracted with Ethyl acetate (2×20 ml). Combined organic layer was washed with brine (30 ml) and dried over Na₂SO₄. Evaporated under vacuum to obtain the crude compound. The crude compound was purified using column chromatography to obtain pure compound (0.200 g, 35%). ES MS m/z: 339.4 [M+H]⁺.

### Synthesis of Ethyl 3-((tert-butoxycarbonyl)amino)-5-(((2-chlorobenzyl)amino)methyl)picolinate (Intermediate 6)

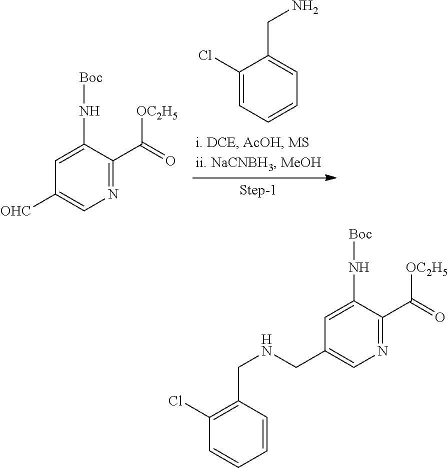 [SMILES: CCOC(=O)c1ncc(C=O)cc1NC(=O)OC(C)(C)C ; NCc1ccccc1Cl ; CCOC(=O)c1ncc(CNCc2ccccc2Cl)cc1NC(=O)OC(C)(C)C]

Step-1: Ethyl 3-((tert-butoxycarbonyl)amino)-5-(((2-chlorobenzyl)amino)methyl)picolinate: To the stirred solution of ethyl 3-((tert-butoxycarbonyl)amino)-5-formylpicolinate (0.6 g, 2.04 mmol) and (2-chlorophenyl)methanamine (0.35 g, 2.45 mmol) in 1,2-Dichloroethane (6.0 mL) was added AcOH (0.6 mL) and the reaction mixture was stirred at room temperature for 1 hour. NaCNBH₃ (0.190 g, 3.06 mmol) and MeOH (0.6 mL) were added and stirred at room temperature for another 1 hour. After completion of reaction (monitored by TLC), the reaction mixture was quenched with saturated solution of NaHCO₃ (50 ml) and was extracted with Dichloromethane (3×75 ml). The combined organic layer was washed with brine (50 ml), dried over anhydrous Sodium sulphate, and concentrated under reduced pressure to get the crude. The crude compound was used in next step without further purification. ES MS m/z: 420.35 [M+H]⁺.

### Synthesis of Methyl 5-(((2-chlorobenzyl)amino)methyl)pyrimidine-2-carboxylate (Intermediate 7)

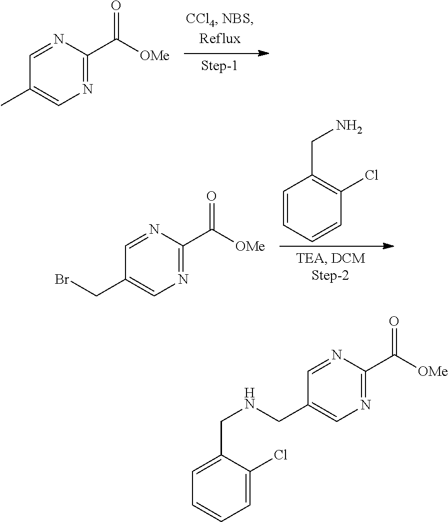 [SMILES: COC(=O)c1ncc(C)cn1 ; COC(=O)c1ncc(CBr)cn1 ; NCc1ccccc1Cl ; COC(=O)c1ncc(CNCc2ccccc2Cl)cn1]

Step-1: Methyl 5-(bromomethyl)pyrimidine-2-carboxylate: To a stirred solution of methyl 5-methylpyrimidine-2-carboxylate (0.2 g, 1.31 mmol), N-Bromosuccinimide (0.268 g, 1.51 mmol) and AIBN (0.086 g, 0.52 mmol) in CCl₄ (2 ml) were refluxed for 16 hours. After completion of reaction (monitored by TLC), the reaction mixture was concentrated to get crude compound. The crude compound was diluted by water (10 ml) and extracted with Ethyl acetate (2×15 ml). The combined organic layer was washed with brine (10 ml), dried over anhydrous Sodium sulphate, and concentrated under reduced pressure. The crude compound was purified by using combi-flash chromatography to obtain title compound (0.075 g, 24%). LCMS: m/z 231.1 [M+H]⁺.

Step-2: Methyl 5-(((2-chlorobenzyl)amino)methyl)pyrimidine-2-carboxylate: To a stirred solution of methyl 5-(bromomethyl)pyrimidine-2-carboxylate (0.2 g, 0.86 mmol) in DCM (2 ml), (2-chlorophenyl)methanamine (0.134 g, 0.95 mmol) was added. The reaction mixture was stirred at room temperature for 72 hours. After completion of reaction (monitored by TLC), the reaction mixture was concentrated. The crude compound was purified by using combi-flash chromatography to obtain title compound (0.060 g, 23%). LCMS: m/z 292.3 [M+H]⁺. ¹H NMR (400 MHz, CDCl3): δ 3.98 (s, 2H), 4.00 (s, 2H), 4.12 (s, 3H), 7.28-7.32 (m, 2H), 7.41-7.43 (m, 2H), 9.00 (s, 2H).

### Synthesis of Methyl 5-(((1-(3-methoxy-2-(trifluoromethyl)phenyl)propyl)amino)methyl)picolinate (Intermediate 8)

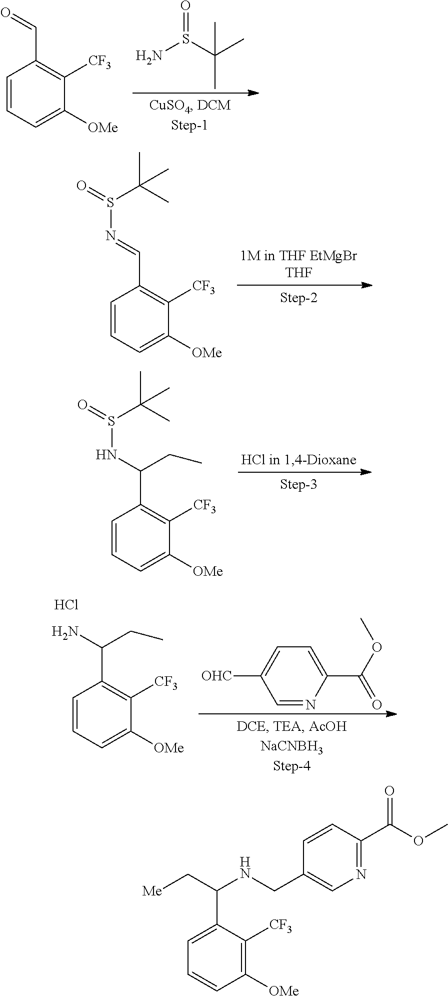

Step-1: (E)-N-(3-methoxy-2-(trifluoromethyl)benzylidene)-2-methylpropane-2-sulfinamide: To a stirred solution of 3-methoxy-2-(trifluoromethyl)benzaldehyde (0.3 g, 1.47 mmol) in dichloromethane (3 ml) was added 2-methylpropane-2-sulfinamide (0.267 g, 2.20 mmol) and copper sulphate (0.469 g, 2.93 mmol) and heated to reflux for 24 hours. After completion of reaction (monitored by TLC), the reaction mixture was quenched with Water (5 ml) and extracted with Ethyl acetate (3×10 ml). The combined organic layer was washed with brine (10 ml), dried over Na₂SO₄. and concentrated under reduced pressure. The crude compound was purified by using combi-flash chromatography to obtain title compound (0.3 g, 66%). LCMS: m/z 308.4 [M+H]⁺. 1H NMR (400 MHz, DMSO-d6): δ 1.19 (s, 9H), 3.94 (s, 3H), 7.48-7.51 (m, 2H), 7.73 (t, J=8.4 Hz, 1H), 8.82 (dd, J=7.2 Hz, J=3.6 Hz, 1H).

Step-2: N-(1-(3-methoxy-2-(trifluoromethyl)phenyl)propyl)-2-methylpropane-2-sulfinamide: To a solution of (E)-N-(3-methoxy-2-(trifluoromethyl)benzylidene)-2-methylpropane-2-sulfinamide (0.3 g, 0.976 mmol) in tetrahydrofuran (3 ml) was added ethyl magnesium bromide (1.0M in THF) (1.46 ml, 1.464 mmol) at 0° C. and reaction mixture stirred at room temperature for 1 hour. After completion of reaction (monitored by TLC), the reaction mixture was quenched with saturated ammonium chloride solution (5 ml) and extracted with Ethyl acetate (3×20 ml). The combined organic layer was washed with brine (20 ml), dried over Na₂SO₄. and concentrated under reduced pressure. The crude compound was purified by using combi-flash chromatography to obtain title compound (0.3 g, 91%). LCMS: m/z 338.2 [M+H]⁺. ¹H NMR (400 MHz, DMSO-d₆): δ 0.88 (t, 3H), 1.07 (s, 9H), 1.60-1.67 (m, 1H), 1.76-1.84 (m, 1H), 3.85 (s, 3H), 4.56-4.61 (m, 1H), 5.57 (d, J=6.8 Hz, 1H), 7.09 (d, J=8.4 Hz, 1H), 7.28 (d, J=8.4 Hz, 1H), 7.56-7.60 (t, J=8.0 Hz, 1H).

Step-3: 1-(3-Methoxy-2-(trifluoromethyl)phenyl)propan-1-amine HCl salt: To a solution of N-(1-(3-methoxy-2-(trifluoromethyl)phenyl)propyl)-2-methylpropane-2-sulfinamide (0.3 g, 0.89 mmol) in dichloromethane (3 ml) was added HCl in 1,4-dioaxane (1.5 ml) and reaction mixture stirred at room temperature for 2 hours. After completion of reaction (monitored by TLC), the reaction mixture was concentrated under reduced pressure. The crude compound was triturated with n-pentane to obtain title compound (0.230 g, 96%). LCMS: m/z 234.0 [M+H]⁺. 1H NMR (400 MHz, DMSO-d6): δ 0.78 (t, J=7.6 Hz, 3H), 1.78-1.85 (m, 1H), 1.96-2.03 (m, 1H), 3.89 (s, 3H), 4.50-4.54 (m, 1H), 7.30 (d, J=8.4 Hz, 1H), 7.41 (d, J=8.0 Hz, 1H), 7.72 (t, J=8.0 Hz, 1H), 8.66 (bs, 2H).

Step-4: Methyl 5-(((1-(3-methoxy-2-(trifluoromethyl)phenyl)propyl)amino)methyl)picolinate: To a mixture of 1-(3-methoxy-2-(trifluoromethyl)phenyl)propan-1-amine HCl salt (0.20 g, 0.741 mmol) in 1,2-dichloroethane (2 ml) was added Triethylamine (0.4 ml, 2.96 mmol) and stirred at room temperature for 30 min. Then molecular sieves, methyl-5-formylpicolinate (0.122 g, 0.741 mmol) and Acetic acid (0.5 ml, 8.89 mmol) were added and RM stirred for 1 hour. Then NaCNBH₃ (0.116 g, 1.85 mmol) was added and reaction mixture stirred at room temperature for 45 min. After completion of the reaction (monitored by TLC), the reaction mixture was filtered through celite bed and to the filtrate added saturated sodium bicarbonate solution (5 ml) and extracted with ethyl acetate (2×20 ml). The combined organic layer was washed with brine (20 ml), dried over Na₂SO₄. and concentrated under reduced pressure. The crude product was purified by column chromatography to afford pure title compound (0.140 g, 49%). LCMS: m/z 383.6 [M+H]⁺. 1H NMR (400 MHz, DMSO-d6): δ 0.86 (t, J=7.2 Hz, 3H), 1.50-1.66 (m, 2H), 3.04 (bs, 1H), 3.44 (d, J=14.8 Hz, 1H), 3.60 (d, J=14.8 Hz, 1H), 3.86 (s, 3H), 3.88 (s, 3H), 3.98 (bs, 1H), 7.11 (d, J=8.4 Hz, 1H), 7.48 (d, J=8.0 Hz, 1H), 7.60 (t, J=8.0 Hz, 1H), 7.87 (d, J=7.6 Hz, 1H), 7.99 (d, J=8.0 Hz, 1H), 8.57 (s, 1H).

### Synthesis of 1-(6-Bromopyridin-3-yl)-N-(2-chlorobenzyl)methanamine (Intermediate 64)

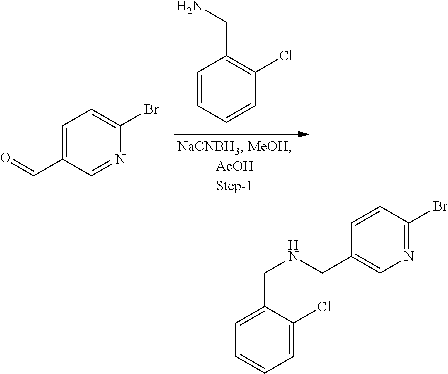 [SMILES: O=Cc1ccc(Br)nc1 ; NCc1ccccc1Cl ; Clc1ccccc1CNCc1ccc(Br)nc1]

Step-1: 1-(6-Bromopyridin-3-yl)-N-(2-chlorobenzyl)methanamine: A mixture 6-bromonicotinaldehyde (0.75 g, 4.07 mmol), (2-chlorophenyl)methanamine (0.574 g 4.07 mmol) and Acetic acid (1.16 ml, 20.35 mmol) in 1-2 dichloroethane (10 ml) was stirred for 2 hours at room temperature with Argon. Then NaCNBH₃ (0.384 g, 6.105 mmol) was added and reaction mixture was stirred at room temperature for another 16 hours. After completion of the reaction (monitored by TLC), Water (20 ml) was added and reaction mixture was extracted with Dichloromethane (2×30 ml). The combined organic layer was washed with brine (20 ml), dried over anhydrous Sodium sulphate and concentrated under vacuum. The crude product was purified by column chromatography to afford pure title compound (0.85 g, 67%) as white solid. LCMS: m/z 311.2 [M+H]⁺.

### Synthesis of Methyl 5-(((1-(2-chloro-3,4-dimethoxyphenyl)propyl)amino)methyl)picolinate (Intermediate 9)

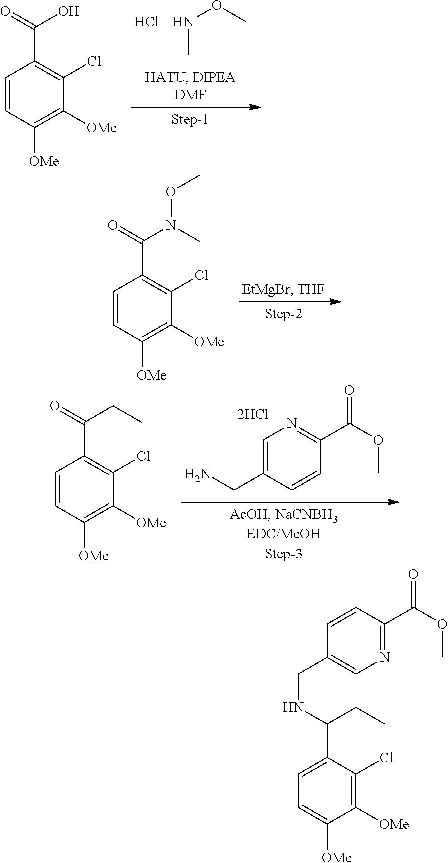

Step-1: 2-Chloro-N,3,4-trimethoxy-N-methylbenzamide: To a stirred solution of 2-chloro-3, 4-dimethoxybenzoic acid (3.0 g, 13.85 mmol) in dry DMF (30 ml) was added HATU (7.894 g, 20.77 mmol) at room temperature. Then DIPEA (4.903 ml, 27.70 mmol) was added drop wise. The reaction mixture was stirred at room temperature for 1 hour. N,O-dimethylhydroxylamine hydrochloride (2.026 g, 20.77 mmol) was added at room temperature and the reaction mixture was allowed to stir at room temperature for 4 hours. After completion of reaction (monitored by TLC), the reaction mixture was diluted with ice-cold water (40 ml) and aqueous layer was extracted with Ethyl acetate (2×80 ml). The combined organic layer was washed with brine (40 ml), dried over anhydrous Sodium sulphate, and concentrated under reduced pressure. The crude compound was purified by Combiflash column chromatography to give pure title compound (2.5 g, 69.5%). LCMS: m/z 260.27 [M+H]⁺.

### Step-2: 1-(2-Chloro-3,4-dimethoxyphenyl)propan-1-one

To the stirred solution of 2-chloro-N, 3, 4-trimethoxy-N-methylbenzamide (1.0 g, 3.85 mmol) in dry THF (10 ml) was added Ethyl magnesium bromide (6.40 ml, 3M solution in Diethyl ether, 19.25 mmol) drop wise at 0° C. under the Nitrogen gas atmosphere and the reaction mixture was stirred at room temperature for 1.5 hours. After completion of reaction (monitored by TLC), the reaction mixture was quenched with aqueous saturated Ammonium chloride solution (10 ml) slowly. The aqueous layer was extracted with Ethyl acetate (3×20 ml). The combined organic layer was washed with brine (10 ml), dried over anhydrous Sodium sulphate and concentrated under reduced pressure to get crude compound. The crude compound was purified by Combiflash column chromatography to give pure title compound (0.600 g, 68%). LCMS: m/z 229 [M+H]⁺. ¹H NMR (400 MHz, CDCl₃): δ 1.21 (t, J=7.2 Hz, 3H), 2.97 (q, J=7.2 Hz, 2H), 3.89 (s, 3H), 3.98 (s, 3H), 6.87 (d, J=8.8 Hz, 1H), 7.33 (d, J=8.8 Hz, 1H).

Step-3: Methyl 5-(((1-(2-chloro-3,4-dimethoxyphenyl)propyl)amino)methyl)picolinate: To the stirred solution of methyl 5-(aminomethyl)picolinate dihydrochloride salt (0.250 g, 1.04 mmol) in DCE (2.5 ml) was added Et₃N (0.436 ml, 3.13 mmol) drop wise under the Nitrogen gas atmosphere. The reaction mixture was stirred at room temperature for 2 hours to liberate free amine. 1-(2-chloro-3,4-dimethoxyphenyl)propan-1-one (0.191 g, 0.83 mmol), glacial acetic acid (0.1 ml), and 4 A° molecular sieves (0.500 g) were added and the reaction mixture was stirred at 60° C. for 16 hours. After completion of reaction (monitored by TLC), the reaction mixture was cooled to room temperature. Sodium cyanoborohydride (0.131 g, 2.09 mmol) was added and the reaction mixture was stirred at 60° C. for 3 hours. After completion of reaction (monitored by TLC), the reaction mixture was quenched by aqueous saturated Sodium bicarbonate (5 ml) solution. The aqueous layer was extracted with Ethyl acetate (3×10 ml). The combined organic layer was washed with brine (5 ml), dried over anhydrous Sodium sulphate and concentrated under reduced pressure to get crude compound. The crude compound was purified by Combiflash column chromatography to give pure title compound (0.170 g, 53.8%). LCMS: m/z 379.3 [M+H]⁺. ¹H NMR (400 MHz, DMSO-d₆): δ 0.80 (t, J=7.4 Hz, 3H), 1.53-1.61 (m, 2H), 2.95 (bs, 1H), 3.49 (d, J=14.4 Hz, 1H), 3.64 (d, J=15.6 Hz, 1H), 3.72 (s, 3H), 3.82 (s, 3H), 3.86 (s, 3H), 5.76 (s, 1H), 7.08 (d, J=8.4 Hz, 1H), 7.32 (d, J=8.8 Hz, 1H), 7.87 (d, J=7.6 Hz, 1H), 7.99 (d, J=7.6 Hz, 1H), 8.56 (s, 1H).

### Synthesis of Methyl 5-(((1-(4-chlorobenzo[d][1, 3]dioxol-5-yl)propyl)amino)methyl)picolinate (Intermediate 10)

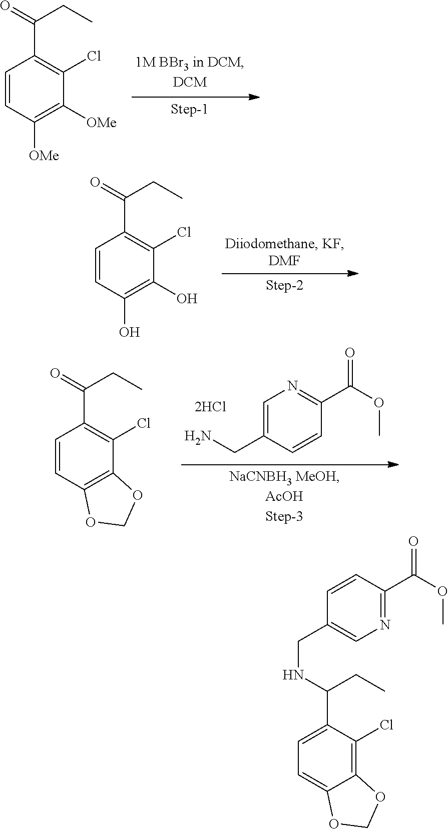

Step-1: 1-(2-Chloro-3, 4-dihydroxyphenyl)propan-1-one: 1-(2-chloro-3, 4-dimethoxyphenyl)propan-1-one (1.00 g, 4.37 mmol) was dissolved in DCM (10 ml) and 1M BBr₃ in DCM (8.74 ml, 1.0 M in DCM, 8.75 mmol) was added drop wise at −78° C. The reaction mixture was then stirred at −78° C. for 1 hour and then at room temperature for 2.5 hours. Reaction was monitored by the TLC (5% Methanol in DCM). After completion of the reaction, the reaction mixture quenched with 30% aqueous Ammonia and concentrated. Methanol (1 ml) was added and reaction mixture was concentrated to dryness. Reaction mixture was azeotroped by Methanol (1 ml) twice. The crude compound was loaded on Celite and purified by RP Gold column chromatography using Acetonitrile and 0.1% formic acid in water to give pure product (0.420 g, 47.9%). LCMS: m/z 199.2 [M+H]⁺. ¹H NMR (400 MHz, DMSO-d₆): δ 1.04 (t, J=7.0 Hz, 3H), 2.86 (q, J=7.2 Hz, 2H), 6.79 (d, J=8.4 Hz, 1H), 7.07 (d, J=8.4 Hz, 1H), 9.49 (s, 1H), 10.19 (s, 1H).

Step-2: 1-(4-Chlorobenzo[d][1, 3]dioxol-5-yl)propan-1-one: To a stirred solution of 1-(2-chloro-3, 4-dihydroxyphenyl)propan-1-one (0.40 g, 1.98 mmol) in dry DMF (6.0 ml) was added Potassium fluoride (0.578 g, 9.96 mmol). The reaction mixture was stirred at room temperature for 30 minutes. Then Diiodomethane (0.20 ml, 2.38 mmol) was added drop wise at room temperature and reaction mixture was stirred at 100° C. for 2 hours. After completion of reaction (monitored by TLC), the reaction mixture was diluted with water (10 ml) and aqueous layer was extracted with Ethyl acetate (2×20 ml). The combined organic layer was dried over anhydrous Sodium sulphate and concentrated under reduced pressure. The crude compound was purified by Combiflash column chromatography to give pure title compound (0.200 g, 47.2%). LCMS: m/z 213.12 [M⁺+1] and 215.1 [M+2H]⁺. ¹H NMR (400 MHz, DMSO-d₆): δ 1.04 (t, J=7.2 Hz, 3H), 2.93 (q, J=6.8 Hz, 2H), 6.21 (s, 2H), 7.00 (d, J=8.4 Hz, 1H), 7.39 (d, J=8.4 Hz, 1H).

Step-3: Methyl 5-(((1-(4-chlorobenzo[d][1, 3]dioxol-5-yl)propyl)amino)methyl)picolinate: To the stirred solution of methyl 5-(aminomethyl)picolinate dihydrochloride salt (0.185 g, 0.78 mmol) in DCE (4 ml) was added Et₃N (0.323 mL, 2.32 mmol) drop wise under the Nitrogen gas atmosphere. The reaction mixture was stirred at room temperature for 2 hours to liberate free amine. 1-(4-chlorobenzo[d] [1, 3]dioxol-5-yl)propan-1-one (0.132 g, 0.62 mmol), glacial acetic acid (0.1 mL), and 4 A° molecular sieves (0.300 g) were added and the reaction mixture was stirred at 60° C. for 16 hours. After completion of reaction (monitored by TLC), the reaction mixture was cooled to room temperature. Sodium cyanoborohydride (0.097 g, 1.55 mmol) and Methanol (5 drops) was added. The reaction mixture was stirred at 60° C. for 3 hours. After completion of reaction (monitored by TLC), the reaction mixture was quenched with aqueous saturated Sodium bicarbonate (5 ml) solution. The aqueous layer was extracted with Ethyl acetate (3×10 ml). The combined organic layer was washed with brine (5 ml), dried over anhydrous Sodium sulphate and concentrated under reduced pressure to get crude compound. The crude compound was purified by Combiflash column chromatography to give pure title compound (0.088 g, 39.1%). LCMS: m/z 363.3 [M+H]⁺. ¹H NMR (400 MHz, DMSO-d₆): δ 0.81 (t, J=7.2 Hz, 3H), 1.56-1.64 (m, 2H), 3.51 (d, J=14.8 Hz, 1H), 3.67 (d, J=13.6 Hz, 1H), 3.84 (s, 1H), 3.88 (s, 3H), 6.13 (s, 2H), 6.96 (d, J=8.0 Hz, 1H), 7.11 (d, J=8.4 Hz, 1H), 7.89 (d, J=8.0 Hz, 1H), 8.01 (d, J=7.6 Hz, 1H), 8.58 (s, 1H).

### Synthesis of Methyl 5-(((1-(2-chloro-3-ethoxyphenyl)propyl)amino)methyl)picolinate (Intermediate 11)

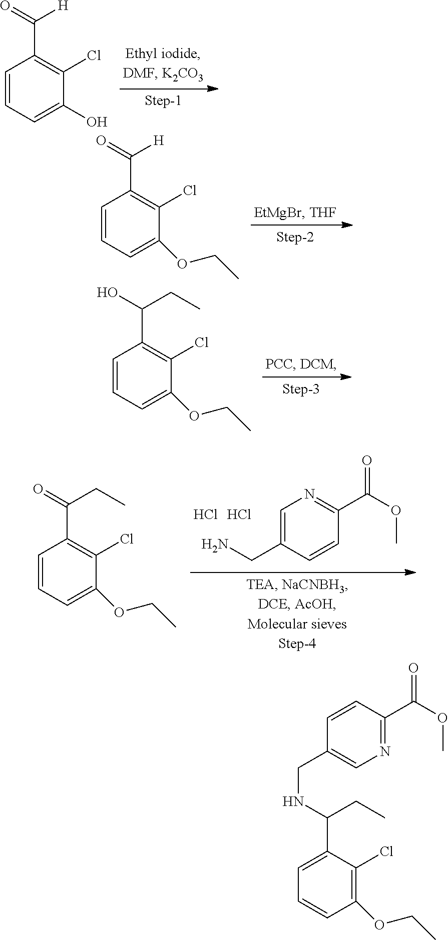

Step-1: 2-Chloro-3-ethoxybenzaldehyde: To a stirred solution of 2-chloro-3-hydroxybenzaldehyde (1.00 g, 6.38 mmol) in DMF (15 ml) was added Potassium carbonate (1.76 g, 12.77 mmol) at room temperature. The reaction mixture was stirred at room temperature for 30 minutes. Ethyl iodide (1.99 g, 12.77 mmol) was added dropwise and the reaction mixture was stirred at room temperature for 1.5 hours. After completion of reaction (monitored by TLC), the reaction mixture was diluted with Ethyl acetate (50 ml) and washed with ice-cold water (50 ml) followed by washing with brine (30 ml). The combined organic layer was dried over anhydrous Sodium sulphate and concentrated under reduced pressure to get crude compound. The crude compound was purified by Combiflash column chromatography to give pure title compound (1.18 g, 100%). ¹H NMR (400 MHz, DMSO-d₆): δ 1.43 (t, J=7.0 Hz, 3H), 4.19 (q, J=7.2 Hz, 2H), 7.42-7.48 (m, 3H), 10.38 (s, 1H).

Step-2: 1-(2-Chloro-3-ethoxyphenyl)propan-1-ol: To the stirred solution of 2-chloro-3-ethoxybenzaldehyde (1.10 g, 5.95 mmol) in dry THF (25 ml) was added Ethyl magnesium bromide (2.38 ml, 3M solution in Diethyl ether, 7.14 mmol) drop wise at −78° C. under the Nitrogen gas atmosphere. The reaction mixture was stirred at −78° C. for 2 hours and continued stirring at room temperature for 13 hours. After completion of reaction (monitored by TLC), the reaction mixture was quenched by aqueous saturated Ammonium chloride solution (20 ml) slowly. The aqueous layer was extracted with Ethyl acetate (3×30 ml). The combined organic layer was washed with brine (20 ml), dried over anhydrous Sodium sulphate, and concentrated under reduced pressure to get crude compound. The crude compound was purified by Combiflash column chromatography to give pure title compound (0.698 g, 54%). ¹H NMR (400 MHz, DMSO-d₆): δ 0.88 (t, J=7.4 Hz, 3H), 1.35 (t, J=7.0 Hz, 3H), 1.43-1.52 (m, 1H), 1.60-1.67 (m, 1H), 4.09 (q, J=6.8 Hz, 2H), 4.80-4.84 (m, 1H), 5.27 (d, J=4.4 Hz, 1H), 6.98 (d, J=7.2 Hz, 1H), 7.14 (d, J=7.2 Hz, 1H), 7.27 (d, J=7.8 Hz, 1H).

Step-3: 1-(2-Chloro-3-ethoxyphenyl)propan-1-one: To the stirred solution of 1-(2-chloro-3-ethoxyphenyl)propan-1-ol (0.690 g, 3.21 mmol) in dry DCM (15 ml) under Nitrogen gas atmosphere, was added Pyridinium chlorochromate (1.380 g, 6.43 mmol) at 0° C. and reaction mixture was stirred at room temperature for 13 hours. After completion of reaction (monitored by TLC), the reaction mixture was filtered through Celite bed and the filtrate was concentrated under reduced pressure to get crude compound. The crude compound was purified by Combiflash column chromatography to give pure title compound (0.681 g, 99%). LCMS: m/z 213.2 [M⁺+1] and 215.3 [M+2H]⁺. ¹H NMR (400 MHz, DMSO-d₆): δ 1.06 (t, J=7.2 Hz, 3H), 1.36 (t, J=7.0 Hz, 3H), 2.87 (q, J=7.2 Hz, 2H), 4.14 (q, J=6.8 Hz, 2H), 7.08 (d, J=6.8 Hz, 1H), 7.23 (d, J=7.6 Hz, 1H), 7.37 (d, J=7.8 Hz, 1H).

Step-4: Methyl 5-(((1-(2-chloro-3-ethoxyphenyl)propyl)amino)methyl)picolinate: To the stirred solution of methyl 5-(aminomethyl)picolinate dihydrochloride salt (0.365 g, 1.53 mmol) in DCE (4 ml) was added Et₃N (0.48 ml, 3.53 mmol) drop wise under the Nitrogen gas atmosphere. The reaction mixture was stirred at room temperature for 3 hours to liberate free amine. 1-(2-chloro-3-ethoxyphenyl)propan-1-one (0.250 g, 1.17 mmol), glacial acetic acid (0.28 ml), and 4 A° molecular sieves (0.50 g) were added and the reaction mixture was stirred at 70° C. for 16 hours. After completion of reaction (monitored by TLC), the reaction mixture was cooled to room temperature. Sodium cyanoborohydride (0.147 g, 2.35 mmol) was added and the reaction mixture was stirred at 70° C. for 16 hours. After completion of reaction (monitored by TLC), the reaction mixture was quenched by aqueous saturated Sodium bicarbonate (5 ml) solution. The aqueous layer was extracted with Ethyl acetate (3×10 ml). The combined organic layer was washed with brine (5 ml), dried over anhydrous Sodium sulphate, and concentrated under reduced pressure to get crude compound. The crude compound was purified by Combiflash column chromatography to give pure title compound (0.240 g, 56%). LCMS: m/z 363.35 [M+H]⁺. ¹H NMR (400 MHz, DMSO-d₆): δ 0.81 (t, J=7.4 Hz, 3H), 1.36 (t, J=6.8 Hz, 3H), 1.52-1.67 (m, 2H), 3.49 (d, J=14.8 Hz, 1H), 3.65 (d, J=14.8 Hz, 1H), 3.87 (s, 3H), 4.00 (bs, 1H), 4.07 (q, J=6.8 Hz, 2H), 6.99 (d, J=8.0 Hz, 1H), 7.21 (d, J=7.6 Hz, 1H), 7.30 (d, J=8.0 Hz, 1H), 7.88 (d, J=7.6 Hz, 1H), 8.00 (d, J=7.6 Hz, 1H), 8.57 (s, 1H).

### Synthesis of Methyl 5-(((1-(2-chloro-3-(oxetan-3-yloxy)phenyl)propyl)amino)methyl)picolinate (Intermediate 12)

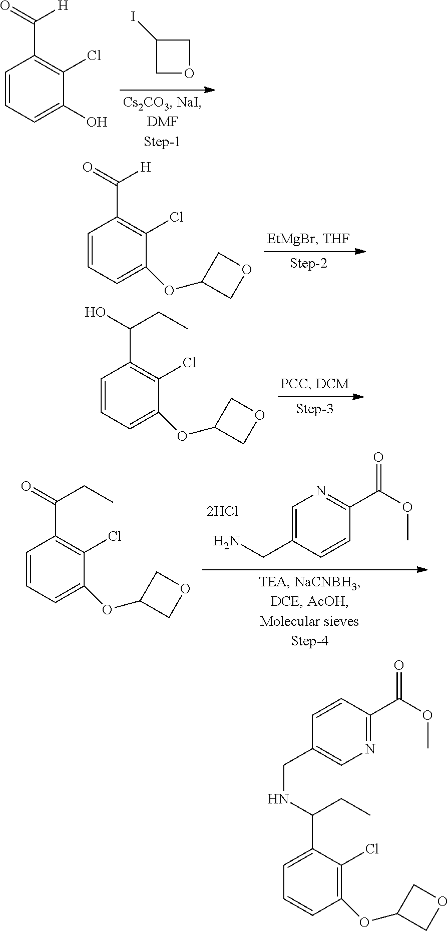

Step-1: 2-Chloro-3-(oxetan-3-yloxy)benzaldehyde: To a stirred solution of 2-chloro-3-hydroxybenzaldehyde (1.0 g, 6.38 mmol) in dry DMF (10 ml) was Cesium carbonate (4.16 g, 12.77 mmol) and NaI (0.478 g, 3.19 mmol) at room temperature. After 15 minutes 3-iodooxetanein (4.70 g, 25.54 mmol) in DMF (5 ml) was added and reaction mixture was heated at 60° C. for 16 hours. After completion of reaction (monitored by TLC), water (50 ml) was added, and the reaction mixture was extracted with Ethyl acetate (2×100 ml). The combined organic layer was washed with brine (50 ml), dried over anhydrous Sodium sulphate, and concentrated under reduced pressure. The crude compound was purified by using combi-flash chromatography to obtain pure title compound (0.4 g, 29%). ¹H NMR (400 MHz, DMSO-d₆): δ 4.60-4.63 (m, 2H), 4.97-5.00 (m, 2H), 5.43-5.45 (m, 1H), 7.11-7.14 (m, 1H), 7.43-7.50 (m, 2H), 10.39 (s, 1H).

Step-2: 1-(2-Chloro-3-(oxetan-3-yloxy)phenyl)propan-1-ol: To a stirred solution of 2-chloro-3-(oxetan-3-yloxy)benzaldehyde (1.0 g, 4.71 mmol) in dry THF (25 ml) was added Ethyl magnesium bromide solution (2.04 ml, 3M in diethyl ether, 6.13 mmol) dropwise at 0° C. and reaction mixture was stirred at 0° C. for 3 hours. After completion of reaction (monitored by TLC), water (20 ml) was added, and the reaction mixture was extracted with Ethyl acetate (2×50 ml). The combined organic layer was washed with brine (50 ml), dried over anhydrous Sodium sulphate, and concentrated under reduced pressure. The crude compound was purified by using combi-flash chromatography to obtain pure title compound (0.6 g, 52%). ¹H NMR (400 MHz, DMSO-d₆): δ 0.90 (t, J=7.6 Hz, 3H), 1.46-1.52 (m, 1H), 1.62-1.69 (m, 1H), 4.55-4.61 (m, 2H), 4.82-4.87 (m, 1H), 4.94-4.97 (m, 2H), 5.31-5.35 (m, 2H), 6.63-6.65 (m, 1H), 7.19-7.28 (m, 2H).

Step-3: 1-(2-Chloro-3-(oxetan-3-yloxy)phenyl)propan-1-one: To a stirred solution of 1-(2-chloro-3-(oxetan-3-yloxy)phenyl)propan-1-ol (0.606 g, 2.49 mmol) in dry DCM (9 ml), PCC (1.07 g, 4.99 mmol) was added at 0° C. Reaction mixture was stirred at room temperature for 6 hours. After completion of reaction (monitored by TLC), water (20 ml) was added, and reaction mixture was extracted with Ethyl acetate (2×50 ml). The combined organic layer was washed with brine (50 ml), dried over anhydrous Sodium sulphate, and concentrated under reduced pressure. The crude compound was purified by using combi-flash chromatography to obtain pure title compound (0.5 g, 83%). LCMS: m/z 241.1 [M+H]⁺. ¹H NMR (400 MHz, DMSO-d₆): δ 1.07 (t, J=7.2 Hz, 3H), 2.89 (t, J=7.2 Hz, 2H), 4.56-4.62 (m, 2H), 4.94-4.99 (m, 2H), 5.36-5.41 (m, 1H), 6.88 (d, J=8.8 Hz, 1H), 7.11-7.16 (m, 1H), 7.35 (d, J=8.0 Hz, 1H).

Step-4: Methyl 5-(((1-(2-chloro-3-(oxetan-3-yloxy)phenyl)propyl)amino)methyl)picolinate: To a stirred solution of methyl 5-(aminomethyl)picolinate dihydrochloride salt (0.079 g, 0.33 mmol) in DCE (2 ml) was added TEA (0.139 ml, 0.99 mmol) and reaction mixture was stirred for 2 hours. To this 1-(2-chloro-3-(oxetan-3-yloxy)phenyl)propan-1-one (0.080 g, 0.26 mmol), Acetic acid (0.15 ml) and molecular sieves (200 mg) was added. The reaction mixture was heated at 60° C. for 16 hours. Reaction mixture was cooled to room temperature and Sodium cyanoborohydride (0.041 g, 0.66 mmol) was added. The reaction mixture was heated at 60° C. for 3 hours. After completion of reaction (monitored by TLC), Sat. solution of NaHCO₃ (5 ml) was added and reaction mixture was extracted with Ethyl acetate (2×10 ml). Combined organic layer was washed with brine (10 ml) dried over Na2SO4, and evaporated under vacuum to obtain the crude compound. The crude compound was purified using column chromatography to obtain pure compound (60 g, 46%). LCMS: m/z 391.6 [M+H]⁺.

### Synthesis of Methyl 5-(((2,3-dichlorobenzyl)amino)methyl)-4-methoxypicolinate (Intermediate 13)

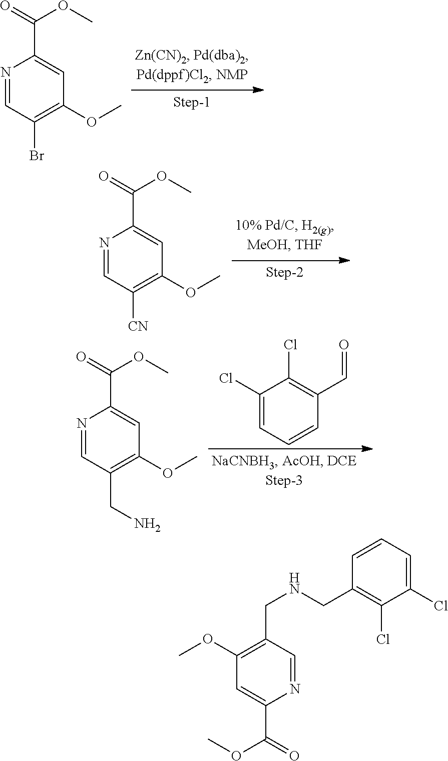

Step-1: Methyl 5-cyano-4-methoxypicolinate: To a stirred solution of methyl 5-bromo-4-methoxypicolinate (1.0 g, 4.06 mmol) in NMP (10 ml) was added Zn(CN)₂ (1.19 g, 10.10 mmol) and the mixture was degassed for 10 minutes with Ar_{(g)}. To the above mixture was added Pd(dba)₂ (0.233 g, 0.40 mmol) followed by Pd(dppf)Cl₂ and reaction mixture was purged with Ar_{(g)}. for 10 minutes. Reaction mixture was heated on oil bath at 100° C. for 4 hours. After completion of reaction (monitored by TLC), the reaction mixture was diluted with Ethyl acetate (30 ml) and cold water was added. Reaction mixture was extracted with Ethyl acetate (2×30 ml). The combined organic layer was washed with Brine (50 ml), dried over Na₂SO₄. and concentrated under reduced pressure to get desired compound as light brown oil. The crude compound was purified by combi flash chromatography to obtained pure compound (0.24 g, 30%). LCMS: m/z 193.1 [M+H]⁺. ¹H NMR (400 MHz, CDCl₃): δ 8.80 (s, 1H), 7.80 (s, 1H), 4.12 (s, 3H), 4.07 (s, 3H).

Step-2: Methyl 5-(aminomethyl)-4-methoxypicolinate: To a stirred solution of methyl 5-cyano-4-methoxypicolinate (0.65 g, 3.3 mmol) in MeOH (10 ml) in autoclave was added Pd/C (0.065 g) and the reaction mixture was purged with H_{2(g)}. Sealed and the reaction mixture was stirred at room temperature for 16 hours. After completion of reaction (monitored by TLC), the reaction mixture was filtered through celite bed, washed with MeOH and concentrated under reduced pressure to get crude compound which was used for the next step without further purification (0.40 g, 60%). %). LCMS: m/z 197.1 [M+H]⁺.

Step-3: Methyl 5-(((2,3-dichlorobenzyl)amino)methyl)-4-methoxypicolinate: To a stirred solution of methyl 5-(aminomethyl)-4-methoxypicolinate (0.08 g, 0.40 mmol) and 2,3-dichlorobenzaldehyde (0.084 g, 0.48 mmol) in DCE (10 ml) was added Acetic acid (0.1 ml) dropwise followed by addition of powdered molecular sieves. The reaction mixture was stirred at room temperature for 1 hour. Sodium cyanoborohydride (0.05 g, 0.80 mmol) was added to the reaction mixture. The reaction mixture was stirred at room temperature for 2 hours. After completion of the reaction (monitored by TLC), the reaction mixture was quenched by saturated Sodium bicarbonate (5 mL). The aqueous layer was extracted with Ethyl acetate (2×10 mL). The combined organic layer was washed with Brine (10 ml), dried over Na₂SO₄. and concentrated under reduced pressure to get crude compound. The crude compound was purified by combi flash chromatography to obtained pure ethyl 2-((4-cyano-2,6-difluorophenethyl)amino)-2-phenylacetate (0.082 g, 56%). ¹H NMR (400 MHz, CDCl₃): δ 8.55 (s, 1H), 7.67 (s, 1H), 7.49-7.42 (m, 2H), 7.22 (t, J=8 Hz, 1H), 4.03-3.89 (m, 10H).

### Synthesis of Methyl 5-(((2,3-dichlorobenzyl)amino)methyl)-4-(dimethylamino)picolinate (Intermediate 14)

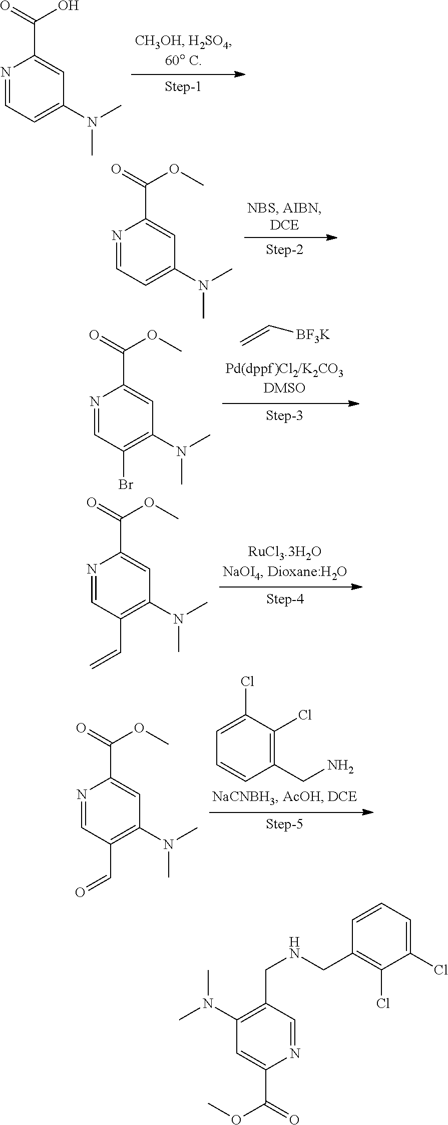

Step-1: Methyl 4-(dimethylamino)picolinate: To a stirred solution of 4-(dimethylamino)picolinic acid (3.0 g, 18.05 mmol) in Methanol (60 ml) was added conc. H₂SO₄ (1.5 ml) drop wise at room temperature. The reaction mixture was stirred at 60° C. for 24 hours. After completion of reaction (monitored by TLC), the reaction mixture was concentrated under reduced pressure to obtain residue which was taken in Dichloromethane (2×50 ml) and the combined organic layer was washed with saturated Sodium bicarbonate solution (50 ml) followed by brine (50 ml). Organic layer was dried over anhydrous Na₂SO₄ and concentrated under reduced pressure to obtain crude compound which was triturated with n-pentane to obtain title compound (2.2 g, 67%). LCMS: m/z 181.0 [M+H]⁺. 1H NMR (400 MHz, DMSO-d₆): δ 63.02 (s, 6H), 3.84 (s, 3H), 6.78-6.80 (m, 1H), 7.24 (d, J=2.4 Hz, 1H), 8.20 (d, J=5.6 Hz, 1H).

Step-2: Methyl 5-bromo-4-(dimethylamino)picolinate: To a stirred solution of methyl 4-(dimethylamino)picolinate (2.2 g, 12.21 mmol) in DCE (22 ml), N-Bromosuccinimide (2.17 g, 12.19 mmol) was added followed by AIBN (0.4 g, 2.44 mmol) and the reaction mixture was stirred at room temperature for 10 minutes then heated at 90° C. for 2 hours. After completion of reaction (monitored by TLC), the reaction mixture was concentrated to get crude compound. The crude compound was purified by combi flash to obtain the title compound (2.2 g, 69%). LCMS: m/z 259.2 [M+H]⁺. 1H NMR (400 MHz, DMSO-d6): δ 2.97 (s, 6H), 3.86 (s, 3H), 7.52 (s, 1H), 8.54 (s, 1H).

Step-3: Methyl 4-(dimethylamino)-5-vinylpicolinate: To a stirred solution of methyl 5-bromo-4-(dimethylamino)picolinate (2.2 g, 8.49 mmol) in DMSO (22 ml) was added trifluorovinylborane potassium salt (3.41 g, 25.45 mmol) portion wise at room temperature. The reaction mixture was degassed with Ar_{(g)} for 10 minutes with stirring. To the above mixture was added K₂CO₃ (3.51 g, 25.39 mmol) and again degassed with Ar_{(S)} for 10 minutes then Pd(dppf)Cl₂ (1.24 g, 1.69 mmol) was added and the reaction mixture was stirred at 80° C. for 1 hour. After completion of reaction (monitored by TLC), the reaction mixture was cooled to room temperature, diluted with cold water and extracted with ethyl acetate (2×50 ml). Combined organic layer washed with brine (50 ml), dried over anhydrous Na₂SO₄ and concentrated under reduced pressure to obtain crude compound, which was purified by column chromatography to obtain the title compound (1.4 g, 79%). LCMS: m/z 206.5 [M+H]⁺. 1H NMR (400 MHz, DMSO-d6): δ 2.88 (s, 6H), 3.85 (s, 3H), 5.41-5.44 (d, J=11.6 Hz, 1H), 5.80-5.85 (m, 1H), 6.75-6.82 (m, 1H), 7.45 (s, 1H), 8.44 (s, 1H).

Step-4: Methyl 4-(dimethylamino)-5-formylpicolinate: To a stirred solution of methyl 4-(dimethylamino)-5-vinylpicolinate (1.4 g, 6.79 mmol) in 1,4-dioxane (42 ml) at 0° C. was added RuCl₃·3H₂O (0.017 g, 0.067 mmol) followed by addition of sodium periodate solution (5.78 g, 27.16 mmol) in water (14 mL) dropwise and the reaction was stirred at room temperature for 1 hour. After completion of reaction (monitored by TLC), the reaction mixture was diluted with water and extracted with ethyl acetate (2×50 ml). Combined organic layer washed with brine (50 ml), dried over anhydrous Na₂SO₄ and concentrated under reduced pressure to obtain crude compound which was purified by column chromatography to obtain the title compound (0.6 g, 42%). LCMS: m/z 209.0 [M+H]⁺. 1H NMR (400 MHz, DMSO-d6): δ 3.08 (s, 6H), 3.88 (s, 3H), 7.49 (s, 1H), 8.69 (s, 1H), 10.04 (s, 1H).

Step-5: Methyl 5-(((2,3-dichlorobenzyl)amino)methyl)-4-(dimethylamino)picolinate To a solution of methyl 4-(dimethylamino)-5-formylpicolinate (0.600 g, 2.88 mmol) in 1,2-dichloroethane (9 ml) was added molecular sieves followed by (2,3-dichlorophenyl)methanamine (0.507 g, 2.88 mmol) and Acetic acid (2.0 ml, 34.56 mmol) RM stirred for 2 hours. Then sodium cyanoborohydride (0.271 g, 4.32 mmol) was added followed by methanol (6 ml) and reaction mixture stirred at room temperature for 24 hours. After completion of the reaction (monitored by TLC), the reaction mixture was passed through celite bed. To the filtrate added saturated sodium bicarbonate solution (10 ml) and extracted with ethyl acetate (2×20 ml). The combined organic layer was washed with brine (20 ml), dried over anhydrous Sodium sulphate, and concentrated under reduced pressure to obtain title compound (0.450 g, 42%). LCMS: m/z 368.3 [M+H]⁺. Crude intermediate was used for next step without further purification.

### Synthesis of 3-(2,3-dichlorophenyl)-3-((pyridin-3-ylmethyl)amino)propanenitrile (Intermediate 15)

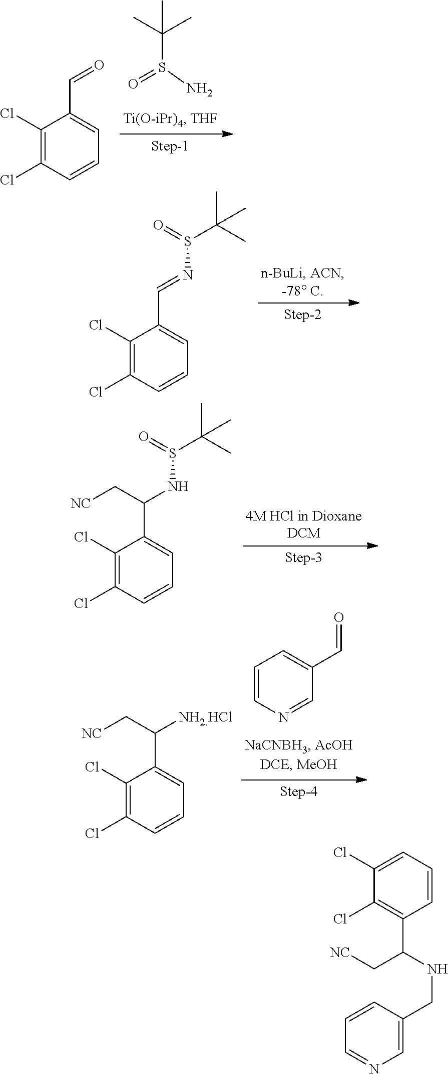

Step-1: (R,E)-N-(2,3-dichlorobenzylidene)-2-methylpropane-2-sulfinamide: To a stirred solution of 2,3-dichlorobenzaldehyde (3.0 g, 17.14 mmol), in THF (60 ml) was 2-methylpropane-2-sulfinamide (2.49 g, 20.53 mmol) at room temperature and the reaction mixture was stirred for 15 min then Ti(O-iPr)₄ (9.15 g, 32.22 mmol) was added slowly at 0° C. Reaction mixture was stirred at room temperature for 16 hours under N_{2(g)} atmosphere. After completion of reaction (monitored by TLC), the reaction mixture was quenched with aqueous Ammonium chloride (30 ml), diluted with Ethyl acetate (30 ml), passed through celite and washed with Ethyl acetate (2×30 ml). Combined organic layer was further washed with water followed by brine (30 ml). Organic layer was dried over Na₂SO₄ and concentrated under reduced pressure to get crude compound which was further purified by column chromatography using to obtain the title compound (2.8 g, 59%). LCMS: min; m/z 278.3 [M+H]⁺.

Step-2: (R)—N-(2-cyano-1-(2,3-dichlorophenyl)ethyl)-2-methylpropane-2-sulfinamide: To a stirred solution of n-BuLi (2.5M in THF) (7.4 mL, 18.60 mmol), acetonitrile (0.682 g, 16.54 mmol) was added dropwise at −78° C. and the reaction mixture was stirred for 1 hour under N_{2(g)} atmosphere. A solution of (R,E)-N-(2,3-dichlorobenzylidene)-2-methylpropane-2-sulfinamide (2.3 g, 8.27 mmol) in tetrahydrofuran (60 ml) was added at −78° C. and the reaction mixture was further stirred 1.5 hour. After completion of reaction (monitored by TLC), the reaction mixture was quenched with dilute HCl solution (5 ml) and extracted with Ethyl acetate (3×50 ml). The combined organic layer was washed with brine (50 ml), dried over Na₂SO₄. and concentrated under reduced pressure. The crude compound was purified by using reverse phase combi-flash chromatography using ACN and 0.1% formic acid in water to obtain title compound (1.8 g, 68%). LCMS: m/z 319.2 [M+H]⁺. ¹H NMR (400 MHz, DMSO-d₆): δ 1.26 (s, 9H), 2.96-3.08 (m, 2H), 4.97-5.03 (m, 1H), 6.24 (d, J=9.2 Hz, 1H), 7.44 (t, J=8.0 Hz, 1H), 7.63-7.67 (m, 2H).

Step-3: 3-amino-3-(2,3-dichlorophenyl)propanenitrile HCl salt: To a stirred solution of (R)—N-(2-cyano-1-(2,3-dichlorophenyl)ethyl)-2-methylpropane-2-sulfinamide (1.8 g, 5.64 mmol) in Dichloromethane (20 ml) was added 4M HCl in 1,4-Dioaxane (8.5 ml, 33.84 mmol) and reaction mixture stirred at room temperature for 2 hours. After completion of reaction (monitored by TLC), the reaction mixture was concentrated under reduced pressure. The crude compound was triturated with n-pentane to obtain title compound (1.4 g, 98%). LCMS: m/z 214.9 [M+H]⁺. ¹H NMR (400 MHz, DMSO-d₆): δ 3.41-3.43 (m, 2H), 5.08-5.12 (m, 1H), 7.56 (t, J=8.0 Hz, 1H), 7.78-7.81 (dd, J=1.2 Hz, 8.0 Hz, 1H), 7.90-7.92 (dd, J=1.2 Hz, 8.0 Hz, 1H), 9.14 (bs, 3H).

Step-4: 3-(2,3-dichlorophenyl)-3-((pyridin-3-ylmethyl)amino)propanenitrile: To a mixture of 3-amino-3-(2,3-dichlorophenyl)propanenitrile HCl salt (1.6 g, 7.44 mmol) in 1,2-Dichloroethane (20 ml) and Methanol (1 mL) was added triethylamine (3.1 ml, 22.32 mmol) and stirred at room temperature for 30 min. Then molecular sieves (3.2 g), nicotinaldehyde (0.796 g, 7.44 mmol) and Acetic acid (1.78 g, 29.76 mmol) were added and reaction mixture stirred for 2 hours. Then NaCNBH₃ (0.701 g, 11.16 mmol) was added and reaction mixture stirred at room temperature for 16 hours. After completion of the reaction (monitored by TLC), the reaction mixture was passed through celite bed. To the filtrate added saturated sodium bicarbonate solution (10 ml) and extracted with ethyl acetate (2×20 ml). The combined organic layer was washed with brine, dried over anhydrous Sodium sulphate, and concentrated under reduced pressure. The crude product was purified by RP-purification using ACN and 0.1% formic acid in water to obtain title compound (1.55 g, 68%). LCMS: m/z 306.3 [M+H]⁺. ¹H NMR (400 MHz, DMSO-d₆): δ 2.87-2.99 (m, 2H), 3.49 (d, J=14 Hz, 1H), 3.64 (d, J=13.6 Hz, 1H), 4.38 (t, J=6.0 Hz, 1H), 7.34-7.37 (m, 1H), 7.47 (t, J=8.0 Hz, 1H), 7.62 (dd, J=1.2 Hz, 8.0 Hz, 1H), 7.72-7.75 (m, 2H), 8.45 (d, J=3.6 Hz, 1H), 8.48 (bs, 1H).

### Synthesis of 4-(2-Chlorophenyl)-4-((pyridin-3-ylmethyl)amino)butanenitrile (Intermediate 16)

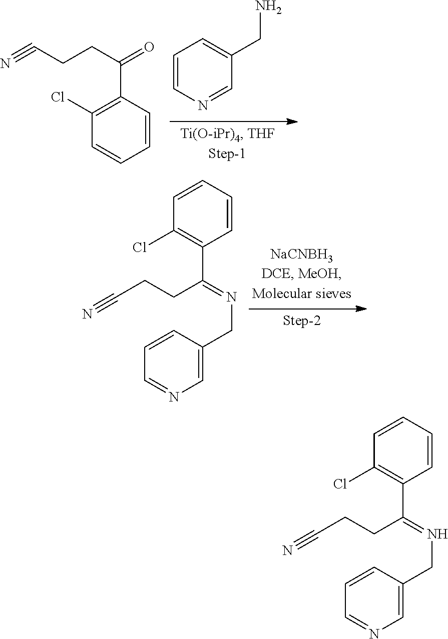

Step-1: (E)-4-(2-Chlorophenyl)-4-((pyridin-3-ylmethyl)imino)butanenitrile: To the solution of 4-(2-chlorophenyl)-4-oxobutanenitrile (0.3 g, 1.54 mmol) in THF (6 ml), pyridin-3-ylmethanamine (0.217 g, 2.01 mmol) was added at room temperature followed by the addition of Titanium(IV) isopropoxide (0.827 g, 2.91 mmol) dropwise at 0° C. Reaction mixture was allowed to stir at room temperature for 16 hours. After completion of reaction (monitored by TLC), the reaction mixture was quenched with sat. NH₄Cl (15 ml) and diluted with Ethyl acetate (20 ml). The reaction mixture was filtered through celite bed and washed with ethyl acetate (20 ml). The organic layer was separated, and aqueous layer was extracted with Ethyl acetate (2×20 ml). The combined organic layer was washed with brine (50 ml), dried over anhydrous Sodium sulphate, and concentrated under reduced pressure. The crude compound used for next step without further purification (0.5 g, Crude). LCMS: m/z 284.3 [M+H]⁺.

Step-2: 4-(2-Chlorophenyl)-4-((pyridin-3-ylmethyl)amino)butanenitrile: To a stirred solution of (E)-4-(2-chlorophenyl)-4-((pyridin-3-ylmethyl)imino)butanenitrile (0.5 g, 1.76 mmol) in 1,2-Dichloroethane (6 ml) and Ethanol (2 ml), Molecular sieves (1 g) was added. The reaction mixture and stirred at room temperature for 10 minutes. Sodium cyanoborohydride (0.166 g, 2.64 mmol) was added at 0° C. and reaction mixture was stirred at room temperature for 16 hours. After completion of reaction (monitored by TLC), the reaction mixture was diluted with 10% Methanol in DCM (50 ml) and filtered through celite bed. Filtrate was concentrated to afford crude compound which was used for next step without further purification. LCMS: m/z 286.3 [M+H]⁺.

### Synthesis of Ethyl 3-((tert-butoxycarbonyl)amino)-5-(((1-(2,3-dichlorophenyl)propyl)amino)methyl)picolinate (Intermediate 17)

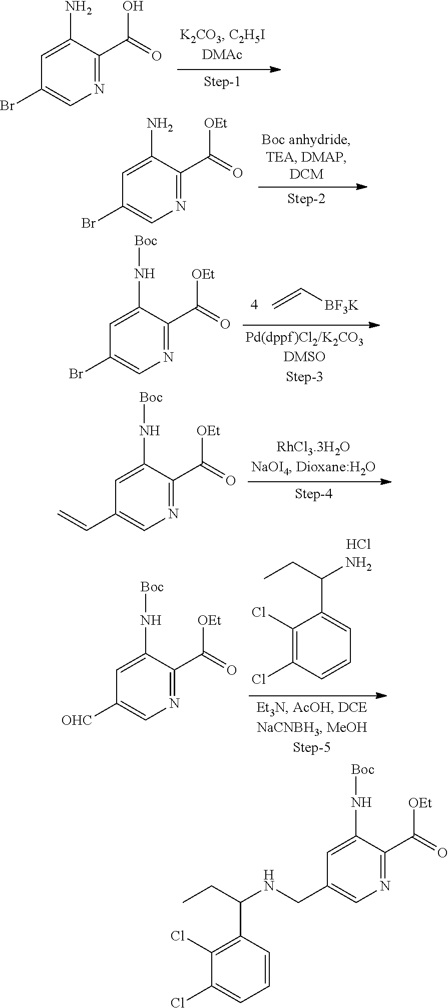

Step-1: Ethyl 3-amino-5-bromopicolinate: A solution of 3-amino-5-bromopicolinic acid (10 g, 46.08 mmol), Potassium carbonate (6.36 g, 46.08 mmol) and Ethyl iodide (3.70 ml, 46.08 mmol) in Dimethyl acetamide (100 ml) was stirred at room temperature for 16 hours. After completion of reaction (monitored by TLC), the reaction mixture was poured on to ice cold water, filtered and dried to get pure solid compound (7 g, 62%). LCMS: m/z 244.9 [M+H]⁺.

Step-2: Ethyl 5-bromo-3-((tert-butoxycarbonyl)amino)picolinate: To a stirred solution of ethyl 3-amino-5-bromopicolinate (3 g, 12.24 mmol) in Dichloromethane was added triethyl amine (5.11 ml, 36.72 mmol) followed by Di-tert-butyl dicarbonate (4.0 g, 18.36 mmol) and DMAP (0.299 g, 2.45 mmol) at 0° C. Reaction mixture was stirred at room temperature for 16 hours. After completion of reaction (monitored by TLC), the reaction mixture was poured on to ice cold water (30 ml) and extracted using Dichloromethane (2×30 ml). Combined organic layer was washed with brine (50 ml) and dried over Na₂SO₄. Evaporated under vacuum to obtain the crude compound. The crude compound was purified using column chromatography to obtain pure compound (2.8 g, 66%). LCMS: m/z 345.3 [M+H]⁺.

Step-3: Ethyl 3-((tert-butoxycarbonyl)amino)-5-vinylpicolinate: To a stirred solution of ethyl 5-bromo-3-((tert-butoxycarbonyl)amino)picolinate (2.6 g, 7.53 mmol) and trifluoro(vinyl)borane potassium salt (3.02 g, 22.55 mmol) in Dimethyl sulfoxide (26 ml) was added potassium carbonate (3.12 g, 22.6 mmol) followed by PdCl₂(dppf) (1.10 g, 1.50 mmol) at room temperature. Reaction mixture was purged with Ar_{(g)} and stirred at 80° C. for 1 hour. After completion of reaction (monitored by TLC), the reaction mixture was poured on to ice cold water (30 ml) and extracted with Ethyl acetate (2×30 ml). Combined organic layer was washed with brine (50 ml) and dried over Na₂SO₄. Evaporated under vacuum to obtain the crude compound. The crude compound was purified using column chromatography to obtain pure compound (1.8 g, 81%). LCMS: m/z 293.0 [M+H]⁺. ¹H NMR (400 MHz, DMSO-d): 1.34 (t, J=7.2 Hz, 3H), 1.50 (s, 9H), 4.34 (q, J=6.8 Hz, 2H), 5.57 (d, J=10.8 Hz, 1H), 6.05 (d, J=17.6 Hz, 1H), 6.83-6.90 (m, 1H), 8.49 (s, 1H), 8.54 (s, 1H), 10.02 (s, 1H).

Step-4: Ethyl 3-((tert-butoxycarbonyl)amino)-5-formylpicolinate: To a stirred solution of ethyl 3-((tert-butoxycarbonyl)amino)-5-vinylpicolinate (1.8 g, 6.16 mmol) in 1,4-Dioxane (18 ml) was added Ruthenium(III)chloride (0.012 g, 0.06 mmol) followed by a solution of Sodium periodate (5.26 g, 24.56 mmol) in water (54 ml) dropwise at 0° C. Reaction mixture was allowed to stirred at room temperature for 1 hour. After completion of reaction (monitored by TLC), the reaction mixture was poured on to ice cold water (20 ml) and extracted with Ethyl acetate (2×20 ml). Combined organic layer organic layer was washed with brine (30 ml) and dried over Na₂SO₄. Evaporated under vacuum to obtain the crude compound. The crude compound was purified using column chromatography to obtain pure compound (0.600 g, 33%). LCMS: m/z 295.3 [M+H]⁺. ¹H NMR (400 MHz, DMSO-d₆): δ 1.34 (t, J=6.8 Hz, 3H), 1.50 (s, 9H), 4.35 (q, J=6.8 Hz, 2H), 8.79 (s, 1H), 8.80 (s, 1H), 10.02 (s, 1H), 10.16 (s, 1H).

Step-5: Ethyl 3-((tert-butoxycarbonyl)amino)-5-(((1-(2,3-dichlorophenyl)propyl)amino)methyl)picolinate: To a stirred solution of 1-(2,3-dichlorophenyl)propan-1-amine hydrochloride (0.490 g, 2.04 mmol) in 1,2-Dichloroethane (6 ml) was added triethyl amine (0.71 ml, 5.093 mmol) and stirred for 1 hour at room temperature. The solution of ethyl 3-((tert-butoxycarbonyl)amino)-5-formylpicolinate (0.500 g, 1.69 mmol) in 1,2-Dichloroethane (6 ml) and Acetic acid (0.1 ml) was added to the reaction mixture and stirred at room temperature for another 1 hour. Sodium cyanoborohydride (0.160 g, 2.55 mmol) and Methanol (2 ml) was added and reaction mixture was stirred at room temperature for another 1 hour. After completion of reaction (monitored by TLC), the reaction mixture was poured on to ice cold water (20 ml) and extracted using Ethyl acetate (2×20 ml). Organic layer was washed with brine (30 ml) and combined organic layer was evaporated under vacuum to obtain the crude compound. The crude compound was purified using column chromatography to obtain pure compound (0.500 g, 61%). LCMS: m/z 484.0 [M+2H]⁺. ¹H NMR (400 MHz, DMSO-d₆): δ 0.85 (t, J=7.6 Hz, 3H), 1.32 (t, J=8 Hz, 3H), 1.49 (s, 9H), 3.49 (d, J=14.8 Hz, 1H), 3.66 (d, J=14.4 Hz, 1H), 4.32 (q, J=6.8 Hz, 2H), 7.39 (t, J=3.2 Hz, 1H), 7.52 (d, J=6.4 Hz, 1H), 7.63 (d, J=6 Hz, 1H), 8.21 (s, 1H), 8.43 (s, 1H), 9.99 (s, 1H).

### Synthesis of Ethyl 3-((tert-butoxycarbonyl)amino)-5-(((2,3-dichlorobenzyl)amino)methyl)picolinate (Intermediate 18)

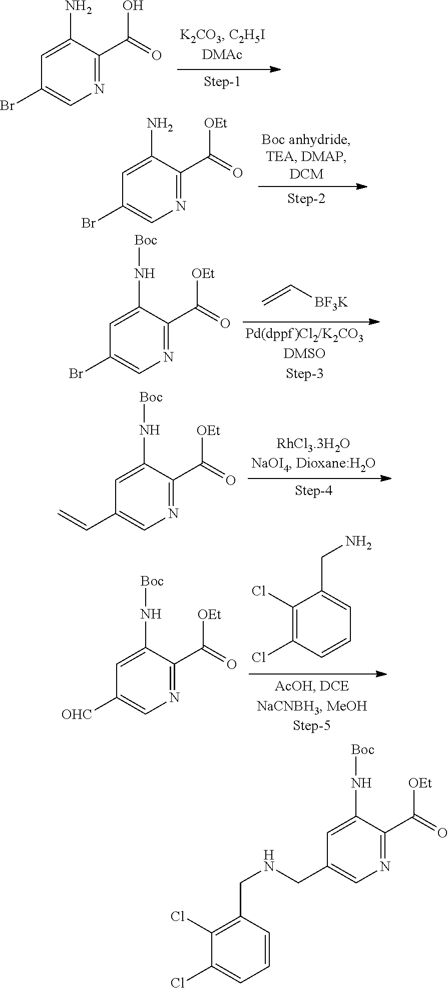

Step-1: Ethyl 3-amino-5-bromopicolinate: A solution of 3-amino-5-bromopicolinic acid (10 g, 46.08 mmol), Potassium carbonate (6.36 g, 46.0 8 mmol) and Ethyl iodide (3.70 ml, 46.08 mmol) in Dimethyl acetamide (100 ml) was stirred at room temperature for 16 hours. After completion of reaction (monitored by TLC), the reaction mixture was poured on to ice cold water, filtered and dried to get pure solid compound (7 g, 62%). LCMS: n/z 244.9 [M+H]⁺.

Step-2: Ethyl 5-bromo-3-((tert-butoxycarbonyl)amino)picolinate: To a stirred solution of ethyl 3-amino-5-bromopicolinate (3 g, 12.24 mmol) in Dichloromethane was added triethyl amine (5.11 ml, 36.722 mmol) followed by Di-tert-butyl dicarbonate (4.0 g, 18.36 mmol) and DMAP (0.299 g, 2.45 mmol) at 0° C. Reaction mixture was stirred at room temperature for 16 hours. After completion of reaction (monitored by TLC), the reaction mixture was poured on to ice cold water (30 ml) and extracted using Dichloromethane (2×30 ml). Combined organic layer was washed with brine (50 ml) and dried over Na₂SO₄. Evaporated under vacuum to obtain the crude compound. The crude compound was purified using column chromatography to obtain pure compound (2.8 g, 66%). LCMS: n/z 345.3 [M+H]⁺.

Step-3: Ethyl 3-((tert-butoxycarbonyl)amino)-5-vinylpicolinate: To a stirred solution of ethyl 5-bromo-3-((tert-butoxycarbonyl)amino)picolinate (2.6 g, 7.53 mmol) and trifluoro(vinyl)borane potassium salt (3.02 g, 22.55 mmol) in Dimethyl sulfoxide (26 ml) was added potassium carbonate (3.12 g, 22.59 mmol) followed by PdCl₂(dppf) (1.10 g, 1.50 mmol) at room temperature. Reaction mixture was purged with Ar_{(g)} and stirred at 80° C. for 1 hour. After completion of reaction (monitored by TLC), the reaction mixture was poured on to ice cold water (30 ml) and extracted with Ethyl acetate (2×30 ml). Combined organic layer was washed with brine (50 ml) and dried over Na₂SO₄. Evaporated under vacuum to obtain the crude compound. The crude compound was purified using column chromatography to obtain pure compound (1.8 g, 81%). LCMS: n/z 293.0 [M+H]⁺. ¹H NMR (400 MHz, DMSO-d₆): δ 1.34 (t, J=7.2 Hz, 3H), 1.50 (s, 9H), 4.34 (q, J=6.8 Hz, 2H), 5.57 (d, J=10.8 Hz, 1H), 6.05 (d, J=17.6 Hz, 1H), 6.83-6.90 (m, 1H), 8.49 (s, 1H), 8.54 (s, 1H), 10.02 (s, 1H).

Step-4: Ethyl 3-((tert-butoxycarbonyl)amino)-5-formylpicolinate: To a stirred solution of ethyl 3-((tert-butoxycarbonyl)amino)-5-vinylpicolinate (1.8 g, 6.16 mmol) in 1,4-Dioxane (18 ml) was added Ruthenium(III)chloride (0.012 g, 0.057 mmol) followed by a solution of Sodium periodate (5.26 g, 24.56 mmol) in water (54 ml) dropwise at 0° C. Reaction mixture was allowed to stirred at room temperature for 1 hour. After completion of reaction (monitored by TLC), the reaction mixture was poured on to ice cold water (20 ml) and extracted with Ethyl acetate (2×20 ml). Combined organic layer organic layer was washed with brine (30 ml) and dried over Na₂SO₄. Evaporated under vacuum to obtain the crude compound. The crude compound was purified using column chromatography to obtain pure compound (0.600 g, 33%). LCMS: m/z 295.3 [M+H]⁺. ¹H NMR (400 MHz, DMSO-d₆): δ 1.34 (t, J=6.8 Hz, 3H), 1.50 (s, 9H), 4.35 (q, J=6.8 Hz, 2H), 8.79 (s, 1H), 8.80 (s, 1H), 10.02 (s, 1H), 10.16 (s, 1H).

Step-5: Ethyl 3-((tert-butoxycarbonyl)amino)-5-(((2,3-dichlorobenzyl)amino)methyl)picolinate: To a stirred solution of ethyl 3-((tert-butoxycarbonyl)amino)-5-formylpicolinate (0.200 g, 0.68 mmol) and (2,3-dichlorophenyl)methanamine (0.143 g, 0.81 mmol) in 1,2-Dichloroethane (5 ml) was added Acetic acid (0.05 ml). The reaction mixture and stirred at room temperature for 1 hour. Sodium cyanoborohydride (0.064 g, 1.02 mmol) and Methanol (1 ml) was added and reaction mixture was stirred at room temperature for another 1 hour. After completion of reaction (monitored by TLC), the reaction mixture was poured on to ice cold water (20 ml) and extracted with Ethyl acetate (2×20 ml). Combined organic layer was washed with brine (30 ml) and dried over Na₂SO₄. Evaporated under vacuum to obtain the crude compound. The crude compound was purified using column chromatography to obtain pure compound (0.190 g, 61%). LCMS: m/z 454.3 [M+H]⁺. ¹H NMR (400 MHz, DMSO-d₆): δ 0.87 (t, J=7.2 Hz, 3H), 1.49 (s, 9H), 3.83 (s, 4H), 4.33 (q, 2H, J=7.2 Hz), 7.37 (t, J=7.6 Hz, 1H), 7.55 (t, J=6.4 Hz, 2H), 8.32 (s, 1H), 8.53 (s, 1H), 10.00 (s, 1H).

The Following Compounds in Table 1 were Prepared in a Similar Manner to Intermediates I-III Procedures:

TABLE 1

| ID | Structure Name | Exact Mass |
|---|---|---|
| Intermediate 20 | 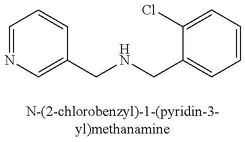 [SMILES: Clc1ccccc1CNCc1cccnc1] | 232.08 |
| Intermediate 211 | 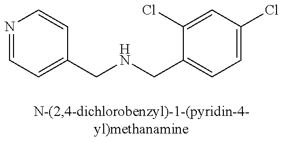 [SMILES: Clc1ccc(CNCc2ccncc2)c(Cl)c1] | 266.04 |
| Intermediate 22 | 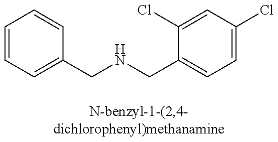 [SMILES: Clc1ccc(CNCc2ccccc2)c(Cl)c1] | 265.04 |
| Intermediate 23 | 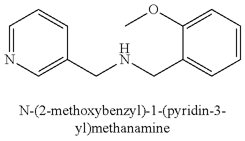 [SMILES: COc1ccccc1CNCc1cccnc1] | 228.13 |
| Intermediate 24 | 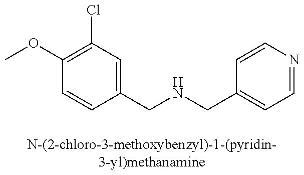 [SMILES: COc1ccc(CNCc2ccncc2)cc1Cl] | 262.09 |
| Intermediate 25 | 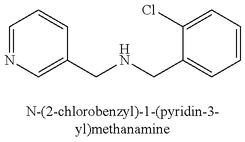 [SMILES: Clc1ccccc1CNCc1cccnc1] | 232.08 |
| Intermediate 26 | 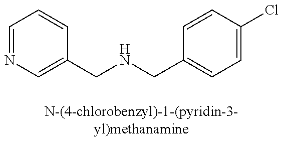 [SMILES: Clc1ccc(CNCc2cccnc2)cc1] | 232.08 |
| Intermediate 27 | 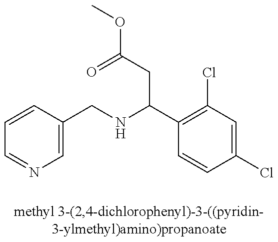 [SMILES: COC(=O)CC(NCc1cccnc1)c1ccc(Cl)cc1Cl] | 338.06 |
| Intermediate 28 | 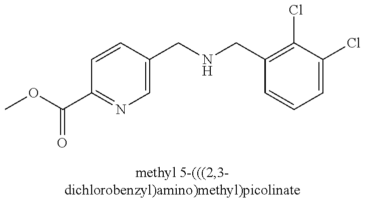 [SMILES: COC(=O)c1ccc(CNCc2cccc(Cl)c2Cl)cn1] | 324.04 |
| Intermediate 29 | 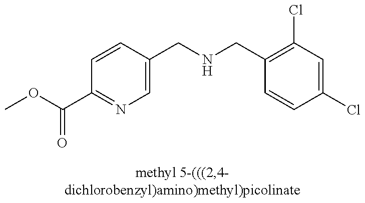 [SMILES: COC(=O)c1ccc(CNCc2ccc(Cl)cc2Cl)cn1] | 324.04 |
| Intermediate 30 | 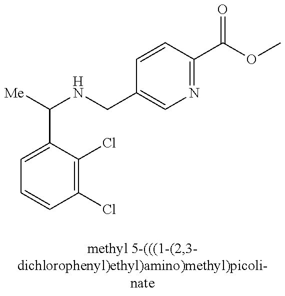 [SMILES: COC(=O)c1ccc(CNC(C)c2cccc(Cl)c2Cl)cn1] | 338.06 |
| Intermediate 31 | 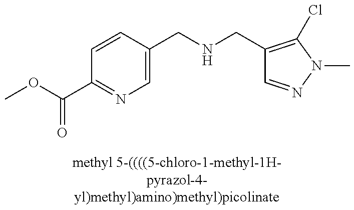 | 294.09 |
| Intermediate 32 | 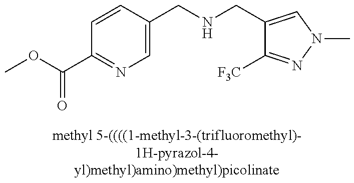 | 328.11 |
| Intermediate 33 | 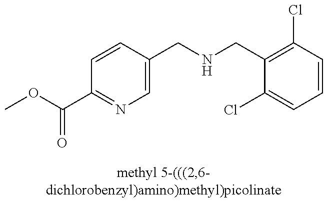 [SMILES: COC(=O)c1ccc(CNCc2c(Cl)cccc2Cl)cn1] | 324.04 |
| Intermediate 34 | 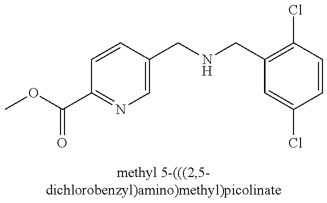 [SMILES: COC(=O)c1ccc(CNCc2cc(Cl)ccc2Cl)cn1] | 324.04 |
| Intermediate 35 | 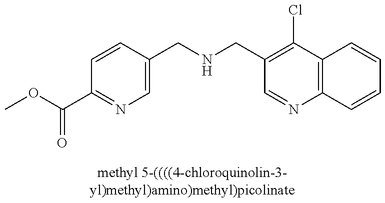 [SMILES: COC(=O)c1ccc(CNCc2cnc3ccccc3c2Cl)cn1] | 341.09 |
| Intermediate 36 | 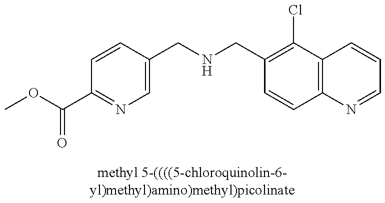 [SMILES: COC(=O)c1ccc(CNCc2ccc3ncccc3c2Cl)cn1] | 341.09 |
| Intermediate 37 | 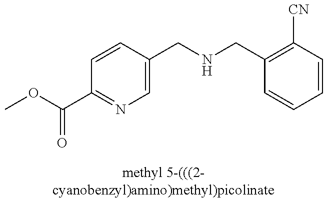 [SMILES: COC(=O)c1ccc(CNCc2ccccc2C#N)cn1] | 281.12 |
| Intermediate 38 | 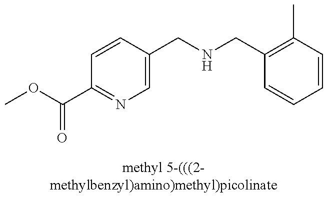 [SMILES: COC(=O)c1ccc(CNCc2ccccc2C)cn1] | 270.14 |
| Intermediate 39 | 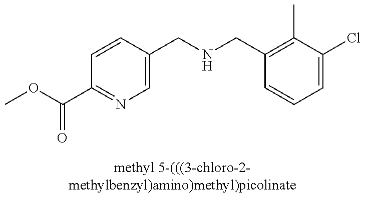 [SMILES: COC(=O)c1ccc(CNCc2cccc(Cl)c2C)cn1] | 304.10 |
| Intermediate 40 | 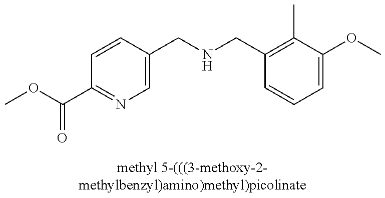 | 300.15 |
| Intermediate 41 | 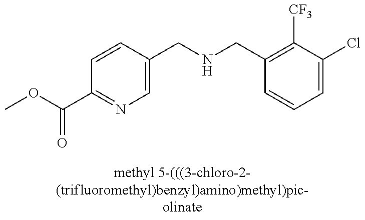 [SMILES: COC(=O)c1ccc(CNCc2cccc(Cl)c2C(F)(F)F)cn1] | 358.07 |
| Intermediate 42 | 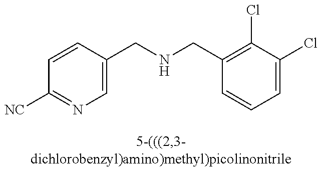 [SMILES: N#Cc1ccc(CNCc2cccc(Cl)c2Cl)cn1] | 291.03 |
| Intermediate 43 | 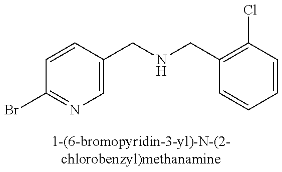 [SMILES: Clc1ccccc1CNCc1ccc(Br)nc1] | 309.99 |
| Intermediate 44 | 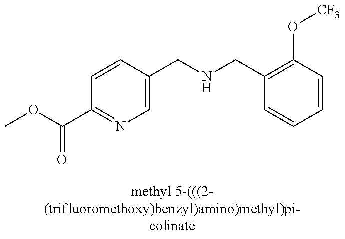 [SMILES: COC(=O)c1ccc(CNCc2ccccc2OC(F)(F)F)cn1] | 340.10 |
| Intermediate 45 | 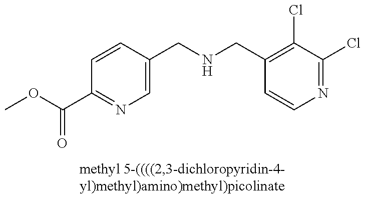 [SMILES: COC(=O)c1ccc(CNCc2ccnc(Cl)c2Cl)cn1] | 325.04 |
| Intermediate 46 | 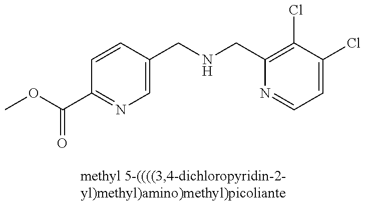 [SMILES: COC(=O)c1ccc(CNCc2nccc(Cl)c2Cl)cn1] | 325.04 |
| Intermediate 47 | 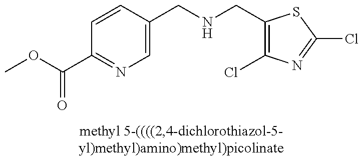 [SMILES: COC(=O)c1ccc(CNCc2sc(Cl)nc2Cl)cn1] | 330.99 |
| Intermediate 48 | 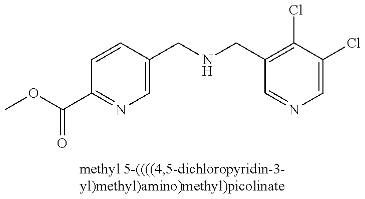 [SMILES: COC(=O)c1ccc(CNCc2cncc(Cl)c2Cl)cn1] | 325.04 |
| Intermediate 49 | 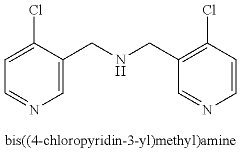 [SMILES: Clc1ccncc1CNCc1cnccc1Cl] | 267.03 |
| Intermediate 50 | 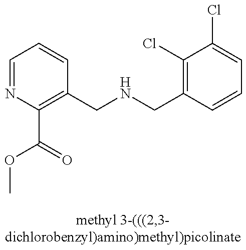 [SMILES: COC(=O)c1ncccc1CNCc1cccc(Cl)c1Cl] | 324.04 |
| Intermediate 51 | 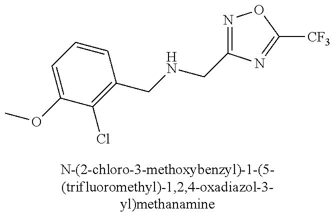 [SMILES: COc1cccc(CNCc2noc(C(F)(F)F)n2)c1Cl] | 321.05 |
| Intermediate 52 | 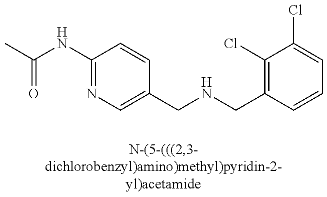 [SMILES: CC(=O)Nc1ccc(CNCc2cccc(Cl)c2Cl)cn1] | 323.06 |
| Intermediate 53 | 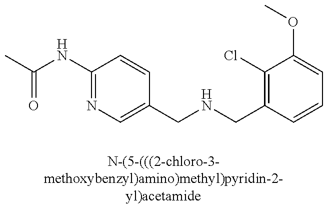 [SMILES: COc1cccc(CNCc2ccc(NC(C)=O)nc2)c1Cl] | 319.11 |
| Intermediate 54 | 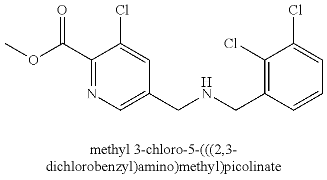 [SMILES: COC(=O)c1ncc(CNCc2cccc(Cl)c2Cl)cc1Cl] | 358.00 |
| Intermediate 55 | 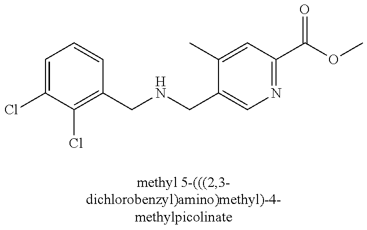 [SMILES: COC(=O)c1cc(C)c(CNCc2cccc(Cl)c2Cl)cn1] | 338.06 |
| Intermediate 56 | 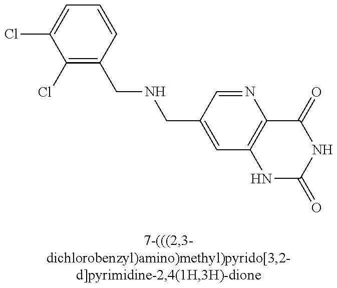 [SMILES: O=c1[nH]c(=O)c2ncc(CNCc3cccc(Cl)c3Cl)cc2[nH]1] | 350.0 |
| Intermediate 57 | 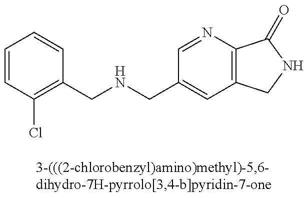 [SMILES: O=C1NCc2cc(CNCc3ccccc3Cl)cnc21] | 287.1 |
| Intermediate 58 | 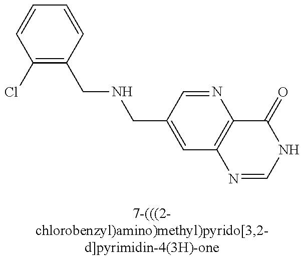 [SMILES: O=c1[nH]cnc2cc(CNCc3ccccc3Cl)cnc12] | 300.1 |
| Intermediate 59 | 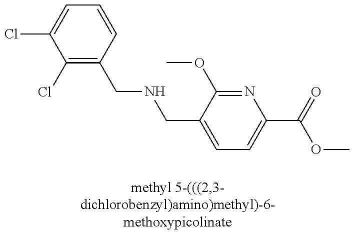 [SMILES: COC(=O)c1ccc(CNCc2cccc(Cl)c2Cl)c(OC)n1] | 354.1 |
| Intermediate 60 | 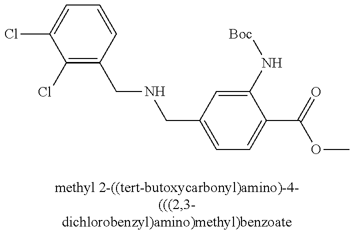 [SMILES: COC(=O)c1ccc(CNCc2cccc(Cl)c2Cl)cc1NC(=O)OC(C)(C)C] | 438.1 |
| Intermediate 61 | 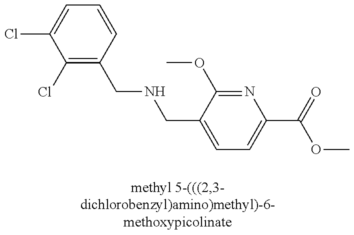 [SMILES: COC(=O)c1ccc(CNCc2cccc(Cl)c2Cl)c(OC)n1] | 354.05 |
| Intermediate 62 | 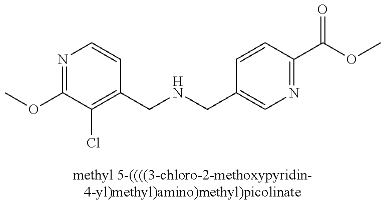 | 321.09 |
| Intermediate 63 | 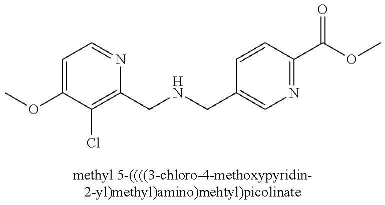 | 321.09 |

General Library Procedure I

### Synthesis of N-[(3,5-dichlorophenyl)methyl]-N-[(pyridin-3-yl)methyl]benzamide (Compound 1)

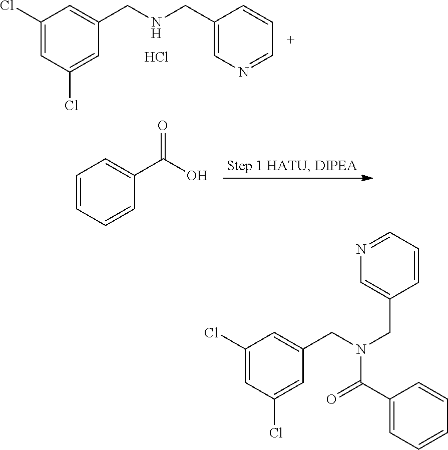 [SMILES: Clc1cc(Cl)cc(CNCc2cccnc2)c1 ; Cl ; O=C(O)c1ccccc1 ; O=C(c1ccccc1)N(Cc1cccnc1)Cc1cc(Cl)cc(Cl)c1]

Step 1. Benzoic acid (30 mg, 245 μmol) and HATU (111 mg, 294 μmol) were combined in DMF and stirred at room temperature for 10 minutes. This solution was added to a flask containing Intermediate I [(3,5-dichlorophenyl)methyl][(pyridin-3-yl)methyl]amine hydrochloride (89.2 mg, 294 μmol) and then N,N-diisopropylethylamine (211 μL, 1.22 mmol) was added. The reaction was stirred at room temperature overnight. The reaction mixture was directly subjected to mass directed Prep HPLC to afford N-[(3,5-dichlorophenyl)methyl]-N-[(pyridin-3-yl)methyl]benzamide (14.9 mg, 16.1%). MS ES m/z: 371.0 [M+H]⁺.

The Following Compounds in Table 2 were Prepared in a Similar Manner to General Library Procedure I:

TABLE 2

| | | (ES, m/z) |
|---|---|---|
| ID | Structure | [M + H]⁺ |
| Compound 2 | 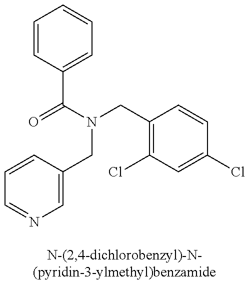 [SMILES: O=C(c1ccccc1)N(Cc1cccnc1)Cc1ccc(Cl)cc1Cl] | |
| Compound 3 | 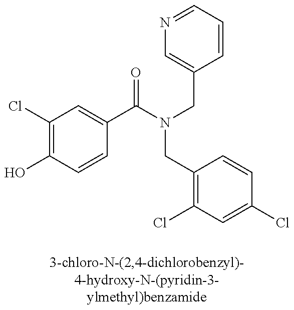 [SMILES: O=C(c1ccc(O)c(Cl)c1)N(Cc1cccnc1)Cc1ccc(Cl)cc1Cl] | 421.2 |
| Compound 4 | 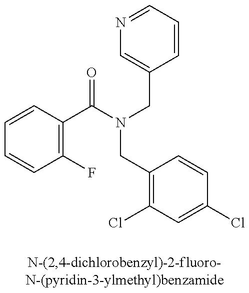 [SMILES: O=C(c1ccccc1F)N(Cc1cccnc1)Cc1ccc(Cl)cc1Cl] | 389.1 |
| Compound 5 | 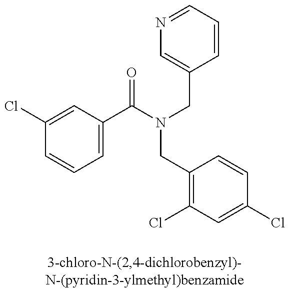 [SMILES: O=C(c1cccc(Cl)c1)N(Cc1cccnc1)Cc1ccc(Cl)cc1Cl] | 405.0 |
| Compound 6 | 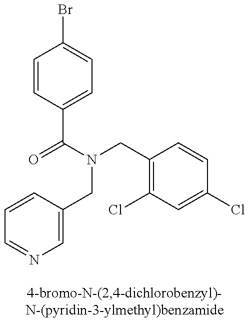 [SMILES: O=C(c1ccc(Br)cc1)N(Cc1cccnc1)Cc1ccc(Cl)cc1Cl] | 451.2 |
| Compound 7 | 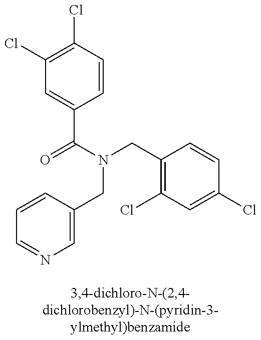 [SMILES: O=C(c1ccc(Cl)c(Cl)c1)N(Cc1cccnc1)Cc1ccc(Cl)cc1Cl] | 441.0 |
| Compound 8 | 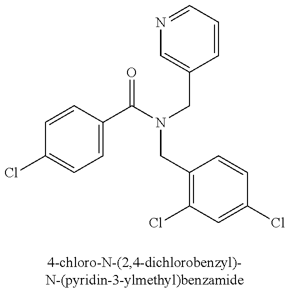 [SMILES: O=C(c1ccc(Cl)cc1)N(Cc1cccnc1)Cc1ccc(Cl)cc1Cl] | 405.0 |
| Compound 9 | 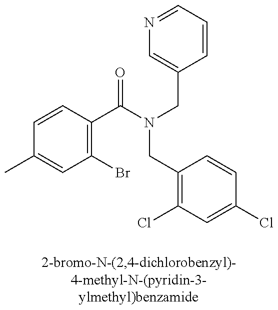 [SMILES: Cc1ccc(C(=O)N(Cc2cccnc2)Cc2ccc(Cl)cc2Cl)c(Br)c1] | 465.2 |
| Compound 10 | 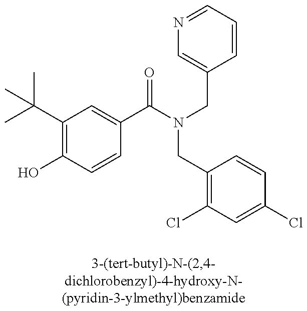 [SMILES: CC(C)(C)c1cc(C(=O)N(Cc2cccnc2)Cc2ccc(Cl)cc2Cl)ccc1O] | 455.3 |
| Compound 11 | 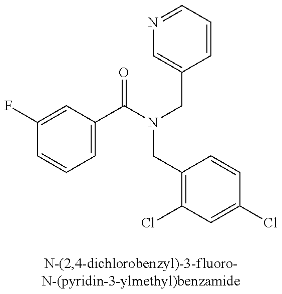 [SMILES: O=C(c1cccc(F)c1)N(Cc1cccnc1)Cc1ccc(Cl)cc1Cl] | 389.1 |
| Compound 12 | 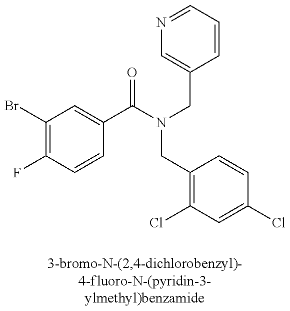 [SMILES: O=C(c1ccc(F)c(Br)c1)N(Cc1cccnc1)Cc1ccc(Cl)cc1Cl] | 469.1 |
| Compound 13 | 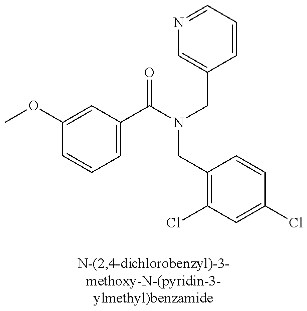 [SMILES: COc1cccc(C(=O)N(Cc2cccnc2)Cc2ccc(Cl)cc2Cl)c1] | 401.1 |
| Compound 14 | 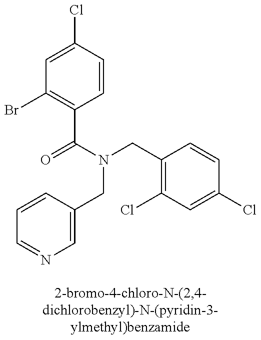 [SMILES: O=C(c1ccc(Cl)cc1Br)N(Cc1cccnc1)Cc1ccc(Cl)cc1Cl] | 485.0 |
| Compound 15 | 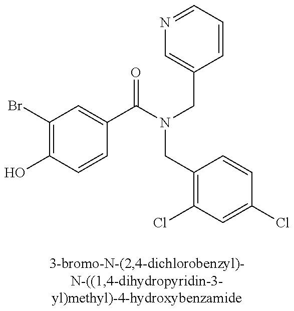 [SMILES: O=C(c1ccc(O)c(Br)c1)N(Cc1cccnc1)Cc1ccc(Cl)cc1Cl] | 467.2 |
| Compound 16 | 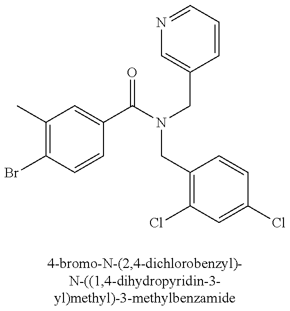 [SMILES: Cc1cc(C(=O)N(Cc2cccnc2)Cc2ccc(Cl)cc2Cl)ccc1Br] | 465.2 |
| Compound 17 | 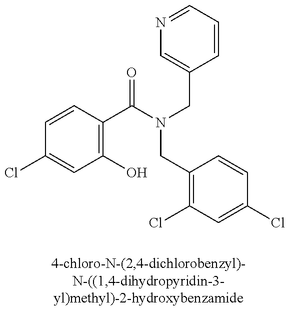 [SMILES: O=C(c1ccc(Cl)cc1O)N(Cc1cccnc1)Cc1ccc(Cl)cc1Cl] | 421.2 |
| Compound 18 | 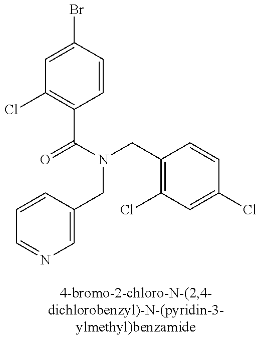 [SMILES: O=C(c1ccc(Br)cc1Cl)N(Cc1cccnc1)Cc1ccc(Cl)cc1Cl] | 484.9 |
| Compound 19 | 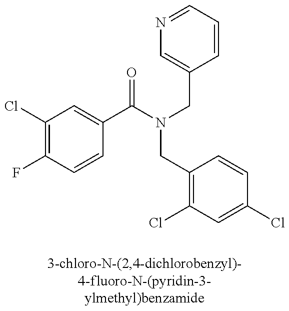 [SMILES: O=C(c1ccc(F)c(Cl)c1)N(Cc1cccnc1)Cc1ccc(Cl)cc1Cl] | 423.0 |
| Compound 20 | 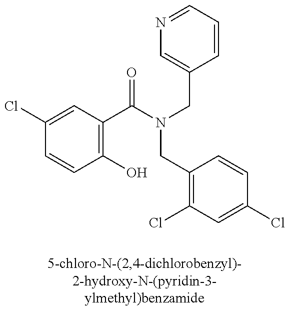 [SMILES: O=C(c1cc(Cl)ccc1O)N(Cc1cccnc1)Cc1ccc(Cl)cc1Cl] | 421.2 |
| Compound 21 | 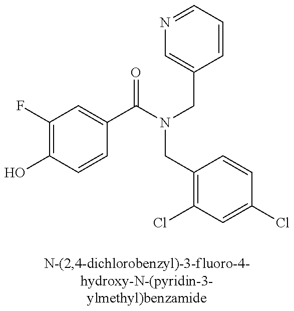 [SMILES: O=C(c1ccc(O)c(F)c1)N(Cc1cccnc1)Cc1ccc(Cl)cc1Cl] | 405.3 |
| Compound 22 | 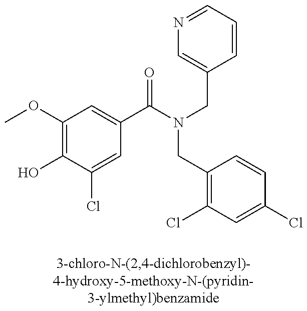 [SMILES: COc1cc(C(=O)N(Cc2cccnc2)Cc2ccc(Cl)cc2Cl)cc(Cl)c1O] | 453.0 |
| Compound 23 | 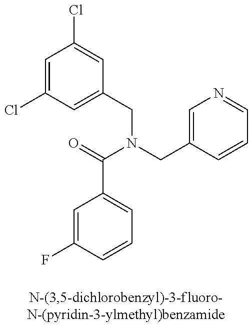 [SMILES: O=C(c1cccc(F)c1)N(Cc1cccnc1)Cc1cc(Cl)cc(Cl)c1] | 389.0 |
| Compound 24 | 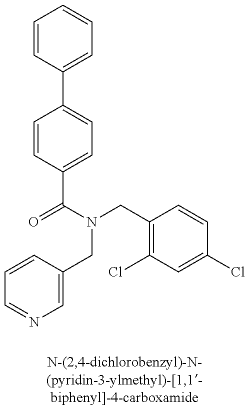 [SMILES: O=C(c1ccc(-c2ccccc2)cc1)N(Cc1cccnc1)Cc1ccc(Cl)cc1Cl] | 447.1 |
| Compound 25 | 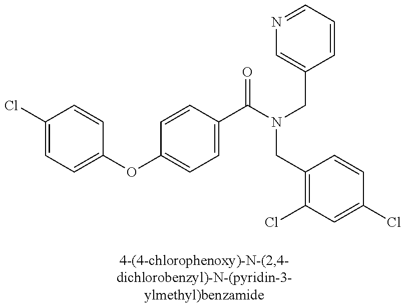 [SMILES: O=C(c1ccc(Oc2ccc(Cl)cc2)cc1)N(Cc1cccnc1)Cc1ccc(Cl)cc1Cl] | 497.0 |
| Compound 26 | 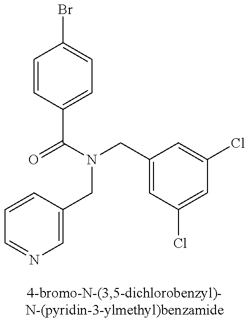 [SMILES: O=C(c1ccc(Br)cc1)N(Cc1cccnc1)Cc1cc(Cl)cc(Cl)c1] | 450.9 |
| Compound 27 | 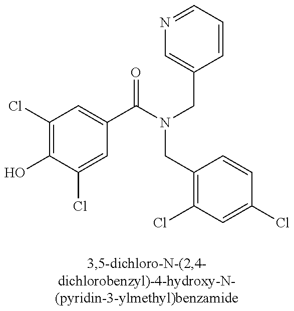 [SMILES: O=C(c1cc(Cl)c(O)c(Cl)c1)N(Cc1cccnc1)Cc1ccc(Cl)cc1Cl] | 455.0 |
| Compound 28 | 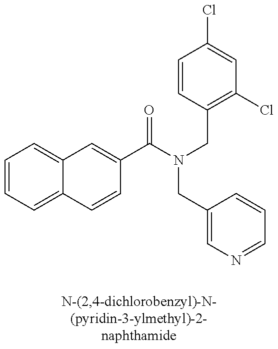 [SMILES: O=C(c1ccc2ccccc2c1)N(Cc1cccnc1)Cc1ccc(Cl)cc1Cl] | 421.1 |
| Compound 29 | 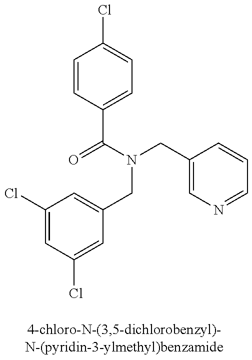 [SMILES: O=C(c1ccc(Cl)cc1)N(Cc1cccnc1)Cc1cc(Cl)cc(Cl)c1] | 407.0 |
| Compound 30 | 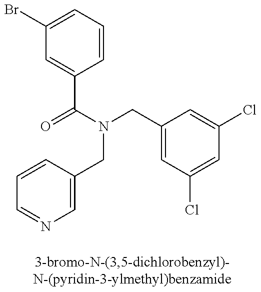 [SMILES: O=C(c1cccc(Br)c1)N(Cc1cccnc1)Cc1cc(Cl)cc(Cl)c1] | 451.0 |
| Compound 31 | 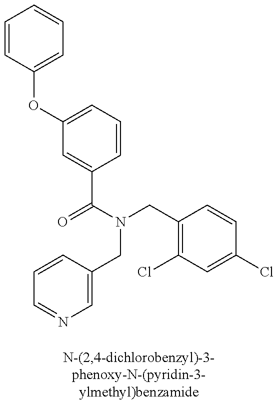 [SMILES: O=C(c1cccc(Oc2ccccc2)c1)N(Cc1cccnc1)Cc1ccc(Cl)cc1Cl] | 463.1 |
| Compound 32 | 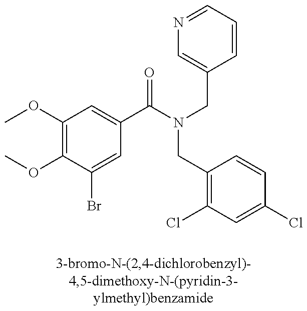 [SMILES: COc1cc(C(=O)N(Cc2cccnc2)Cc2ccc(Cl)cc2Cl)cc(Br)c1OC] | 511.0 |
| Compound 33 | 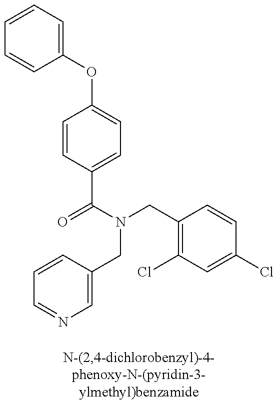 [SMILES: O=C(c1ccc(Oc2ccccc2)cc1)N(Cc1cccnc1)Cc1ccc(Cl)cc1Cl] | 463.1 |
| Compound 34 | 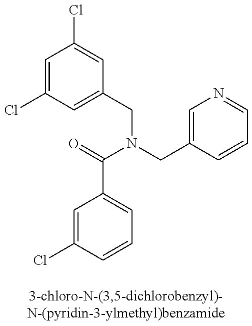 [SMILES: O=C(c1cccc(Cl)c1)N(Cc1cccnc1)Cc1cc(Cl)cc(Cl)c1] | 407.0 |
| Compound 35 | 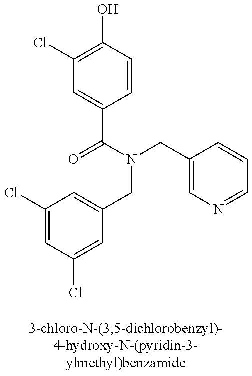 [SMILES: O=C(c1ccc(O)c(Cl)c1)N(Cc1cccnc1)Cc1cc(Cl)cc(Cl)c1] | 423.0 |
| Compound 36 | 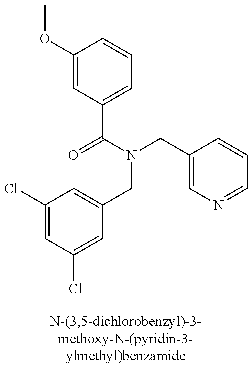 [SMILES: COc1cccc(C(=O)N(Cc2cccnc2)Cc2cc(Cl)cc(Cl)c2)c1] | 400.9 |
| Compound 37 | 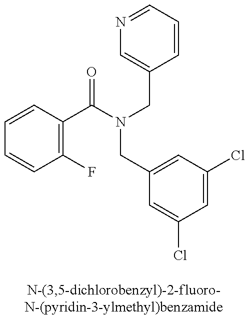 [SMILES: O=C(c1ccccc1F)N(Cc1cccnc1)Cc1cc(Cl)cc(Cl)c1] | 389.0 |
| Compound 38 | 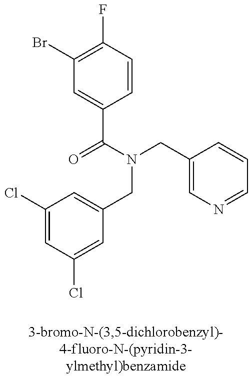 [SMILES: O=C(c1ccc(F)c(Br)c1)N(Cc1cccnc1)Cc1cc(Cl)cc(Cl)c1] | 469.0 |
| Compound 39 | 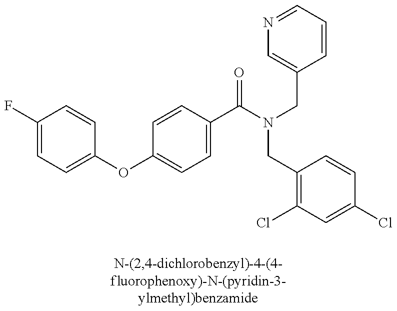 [SMILES: O=C(c1ccc(Oc2ccc(F)cc2)cc1)N(Cc1cccnc1)Cc1ccc(Cl)cc1Cl] | 481.1 |
| Compound 40 | 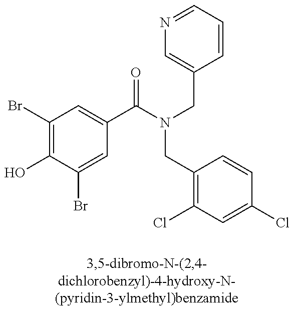 [SMILES: O=C(c1cc(Br)c(O)c(Br)c1)N(Cc1cccnc1)Cc1ccc(Cl)cc1Cl] | 546.9 |
| Compound 41 | 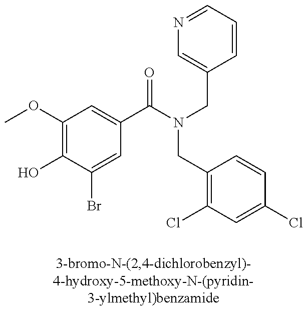 [SMILES: COc1cc(C(=O)N(Cc2cccnc2)Cc2ccc(Cl)cc2Cl)cc(Br)c1O] | 497.0 |
| Compound 42 | 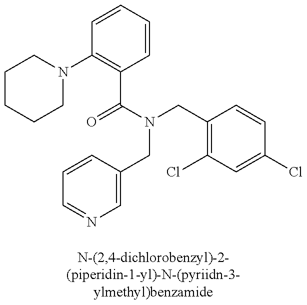 [SMILES: O=C(c1ccccc1N1CCCCC1)N(Cc1cccnc1)Cc1ccc(Cl)cc1Cl] | 454.1 |
| Compound 43 | 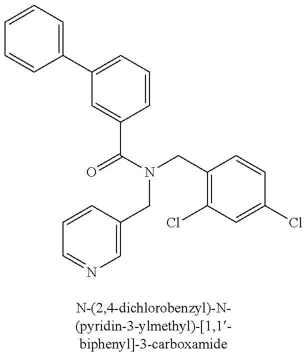 [SMILES: O=C(c1cccc(-c2ccccc2)c1)N(Cc1cccnc1)Cc1ccc(Cl)cc1Cl] | 447.1 |
| Compound 44 | 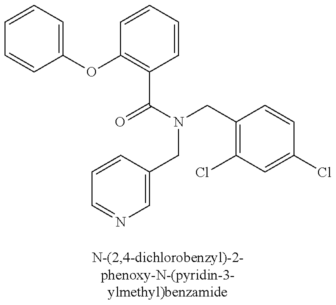 [SMILES: O=C(c1ccccc1Oc1ccccc1)N(Cc1cccnc1)Cc1ccc(Cl)cc1Cl] | 463.1 |
| Compound 45 | 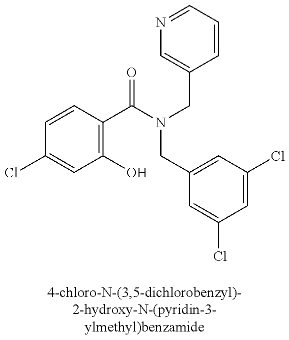 [SMILES: O=C(c1ccc(Cl)cc1O)N(Cc1cccnc1)Cc1cc(Cl)cc(Cl)c1] | 420.9 |
| Compound 46 | 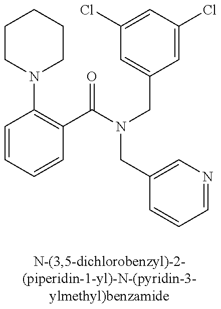 [SMILES: O=C(c1ccccc1N1CCCCC1)N(Cc1cccnc1)Cc1cc(Cl)cc(Cl)c1] | 454.1 |
| Compound 47 | 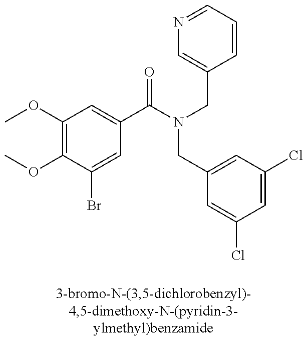 [SMILES: COc1cc(C(=O)N(Cc2cccnc2)Cc2cc(Cl)cc(Cl)c2)cc(Br)c1OC] | 510.9 |
| Compound 48 | 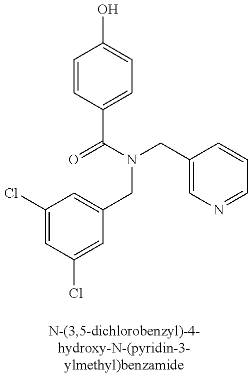 [SMILES: O=C(c1ccc(O)cc1)N(Cc1cccnc1)Cc1cc(Cl)cc(Cl)c1] | 387.0 |
| Compound 49 | 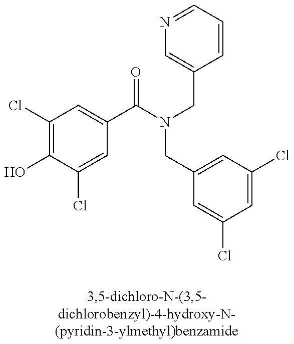 [SMILES: O=C(c1cc(Cl)c(O)c(Cl)c1)N(Cc1cccnc1)Cc1cc(Cl)cc(Cl)c1] | 456.9 |
| Compound 50 | 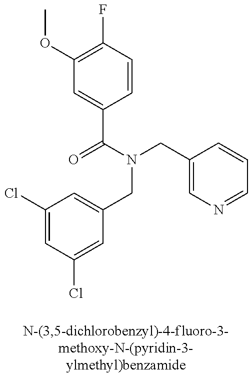 [SMILES: COc1cc(C(=O)N(Cc2cccnc2)Cc2cc(Cl)cc(Cl)c2)ccc1F] | 419.1 |
| Compound 51 | 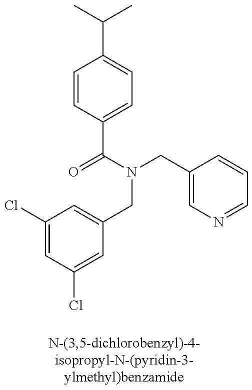 [SMILES: CC(C)c1ccc(C(=O)N(Cc2cccnc2)Cc2cc(Cl)cc(Cl)c2)cc1] | 413.2 |
| Compound 52 | 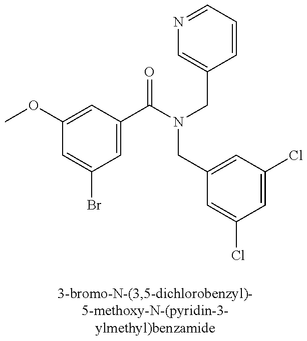 [SMILES: COc1cc(Br)cc(C(=O)N(Cc2cccnc2)Cc2cc(Cl)cc(Cl)c2)c1] | 481.1 |
| Compound 53 | 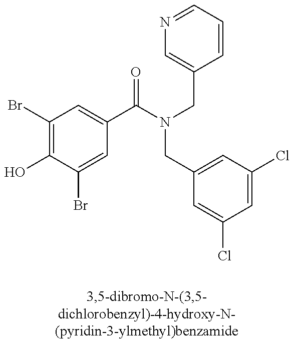 [SMILES: O=C(c1cc(Br)c(O)c(Br)c1)N(Cc1cccnc1)Cc1cc(Cl)cc(Cl)c1] | 544.8 |
| Compound 54 | 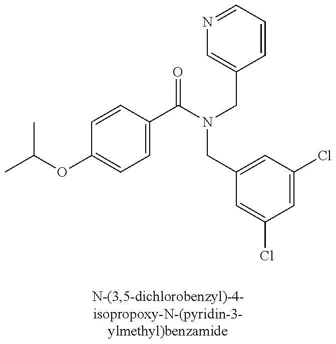 [SMILES: CC(C)Oc1ccc(C(=O)N(Cc2cccnc2)Cc2cc(Cl)cc(Cl)c2)cc1] | 429.0 |
| Compound 55 | 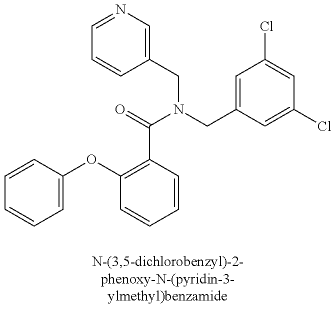 [SMILES: O=C(c1ccccc1Oc1ccccc1)N(Cc1cccnc1)Cc1cc(Cl)cc(Cl)c1] | 463.0 |
| Compound 56 | 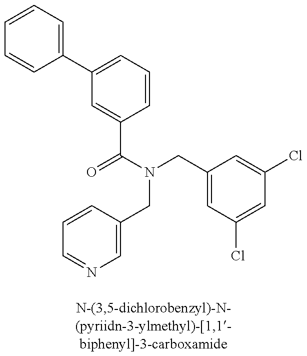 [SMILES: O=C(c1cccc(-c2ccccc2)c1)N(Cc1cccnc1)Cc1cc(Cl)cc(Cl)c1] | 447.0 |
| Compound 57 | 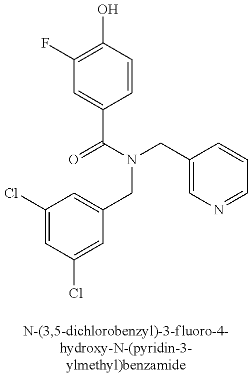 [SMILES: O=C(c1ccc(O)c(F)c1)N(Cc1cccnc1)Cc1cc(Cl)cc(Cl)c1] | 405.0 |
| Compound 58 | 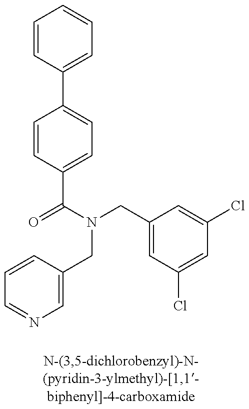 [SMILES: O=C(c1ccc(-c2ccccc2)cc1)N(Cc1cccnc1)Cc1cc(Cl)cc(Cl)c1] | 447.0 |
| Compound 59 | 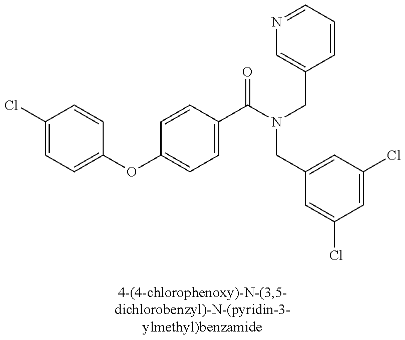 [SMILES: O=C(c1ccc(Oc2ccc(Cl)cc2)cc1)N(Cc1cccnc1)Cc1cc(Cl)cc(Cl)c1] | 498.9 |
| Compound 60 | 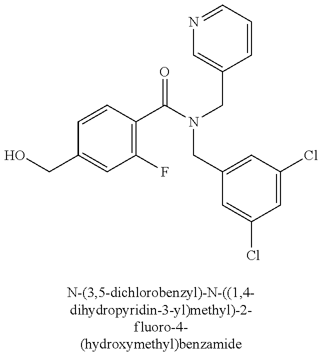 [SMILES: O=C(c1ccc(CO)cc1F)N(Cc1cccnc1)Cc1cc(Cl)cc(Cl)c1] | 419.0 |
| Compound 61 | 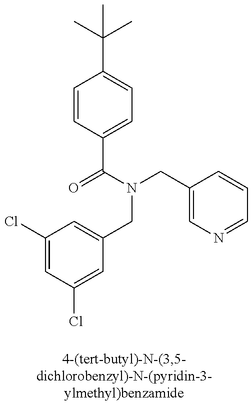 [SMILES: CC(C)(C)c1ccc(C(=O)N(Cc2cccnc2)Cc2cc(Cl)cc(Cl)c2)cc1] | 427.0 |
| Compound 62 | 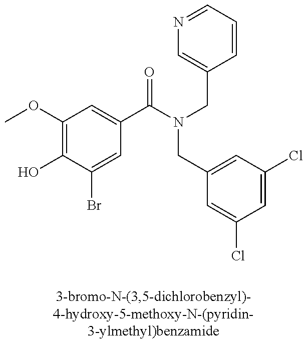 [SMILES: COc1cc(C(=O)N(Cc2cccnc2)Cc2cc(Cl)cc(Cl)c2)cc(Br)c1O] | 496.9 |
| Compound 63 | 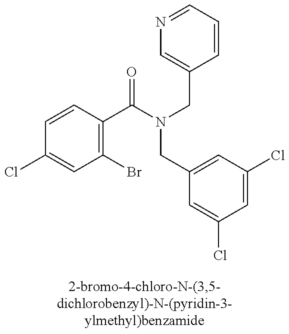 [SMILES: O=C(c1ccc(Cl)cc1Br)N(Cc1cccnc1)Cc1cc(Cl)cc(Cl)c1] | 484.8 |
| Compound 64 | 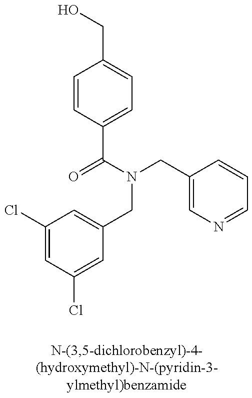 [SMILES: O=C(c1ccc(CO)cc1)N(Cc1cccnc1)Cc1cc(Cl)cc(Cl)c1] | 401.1 |
| Compound 65 | 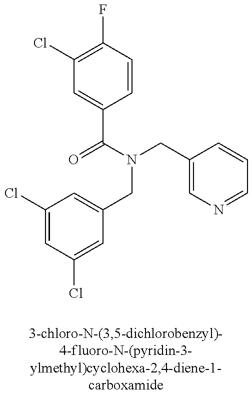 [SMILES: O=C(c1ccc(F)c(Cl)c1)N(Cc1cccnc1)Cc1cc(Cl)cc(Cl)c1] | 424.9 |
| Compound 66 | 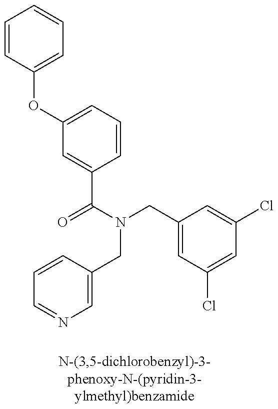 [SMILES: O=C(c1cccc(Oc2ccccc2)c1)N(Cc1cccnc1)Cc1cc(Cl)cc(Cl)c1] | 463.0 |
| Compound 67 | 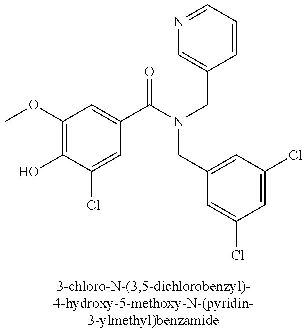 [SMILES: COc1cc(C(=O)N(Cc2cccnc2)Cc2cc(Cl)cc(Cl)c2)cc(Cl)c1O] | 451.0 |
| Compound 68 | 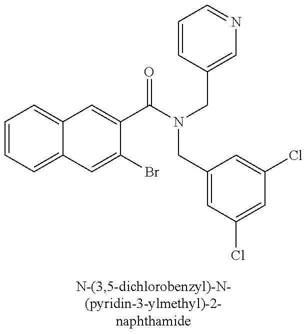 [SMILES: O=C(c1cc2ccccc2cc1Br)N(Cc1cccnc1)Cc1cc(Cl)cc(Cl)c1] | 421.0 |
| Compound 69 | 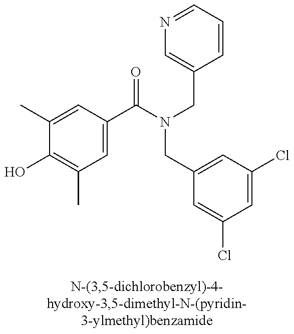 [SMILES: Cc1cc(C(=O)N(Cc2cccnc2)Cc2cc(Cl)cc(Cl)c2)cc(C)c1O] | 415.0 |
| Compound 70 | 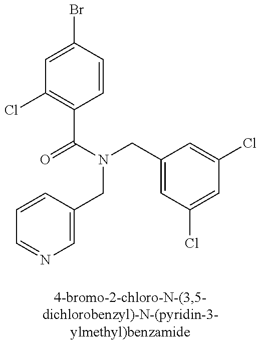 [SMILES: O=C(c1ccc(Br)cc1Cl)N(Cc1cccnc1)Cc1cc(Cl)cc(Cl)c1] | 484.9 |
| Compound 71 | 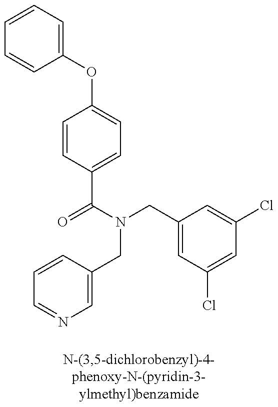 [SMILES: O=C(c1ccc(Oc2ccccc2)cc1)N(Cc1cccnc1)Cc1cc(Cl)cc(Cl)c1] | 463.0 |
| Compound 72 | 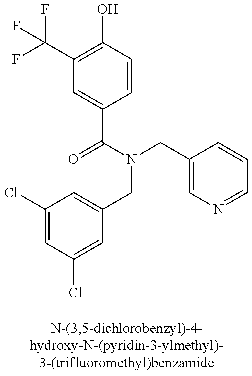 [SMILES: O=C(c1ccc(O)c(C(F)(F)F)c1)N(Cc1cccnc1)Cc1cc(Cl)cc(Cl)c1] | 455.0 |
| Compound 73 | 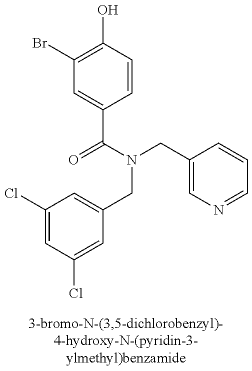 [SMILES: O=C(c1ccc(O)c(Br)c1)N(Cc1cccnc1)Cc1cc(Cl)cc(Cl)c1] | 466.9 |
| Compound 74 | 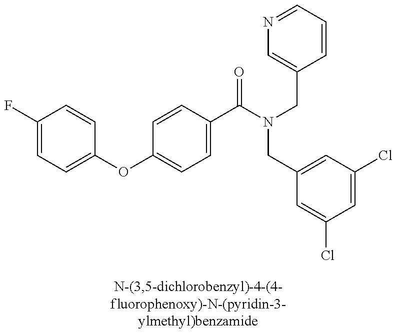 [SMILES: O=C(c1ccc(Oc2ccc(F)cc2)cc1)N(Cc1cccnc1)Cc1cc(Cl)cc(Cl)c1] | 481.0 |
| Compound 75 | 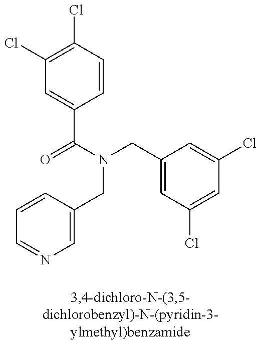 [SMILES: O=C(c1ccc(Cl)c(Cl)c1)N(Cc1cccnc1)Cc1cc(Cl)cc(Cl)c1] | 440.9 |
| Compound 76 | 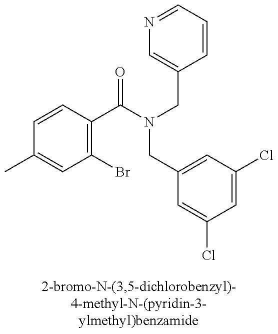 [SMILES: Cc1ccc(C(=O)N(Cc2cccnc2)Cc2cc(Cl)cc(Cl)c2)c(Br)c1] | 464.9 |
| Compound 77 | 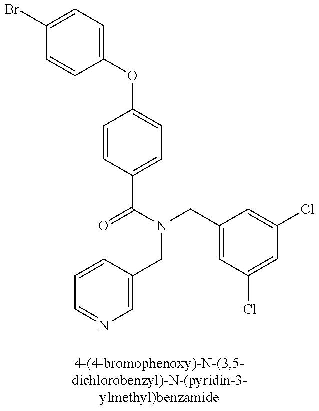 [SMILES: O=C(c1ccc(Oc2ccc(Br)cc2)cc1)N(Cc1cccnc1)Cc1cc(Cl)cc(Cl)c1] | 542.9 |
| Compound 78 | 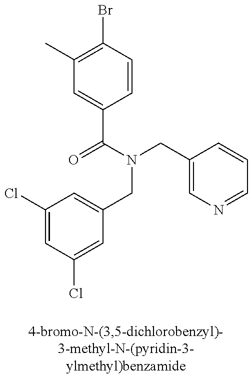 [SMILES: Cc1cc(C(=O)N(Cc2cccnc2)Cc2cc(Cl)cc(Cl)c2)ccc1Br] | 464.9 |
| Compound 79 | 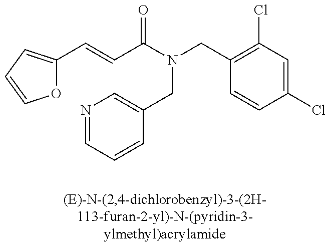 [SMILES: O=C(/C=C/c1ccco1)N(Cc1cccnc1)Cc1ccc(Cl)cc1Cl] | 387.0 |
| Compound 80 | 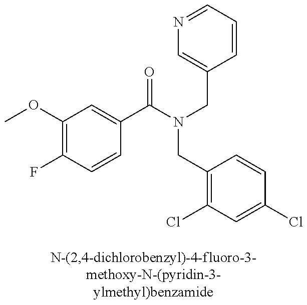 [SMILES: COc1cc(C(=O)N(Cc2cccnc2)Cc2ccc(Cl)cc2Cl)ccc1F] | 419.0 |
| Compound 81 | 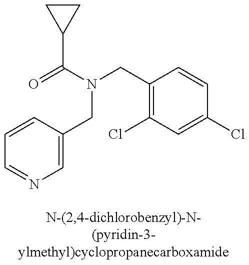 [SMILES: O=C(C1CC1)N(Cc1cccnc1)Cc1ccc(Cl)cc1Cl] | 335.0 |
| Compound 82 | 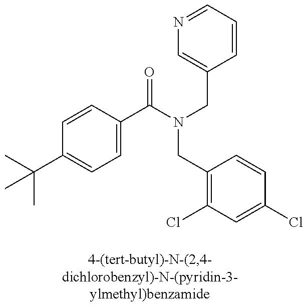 [SMILES: CC(C)(C)c1ccc(C(=O)N(Cc2cccnc2)Cc2ccc(Cl)cc2Cl)cc1] | 427.0 |
| Compound 83 | 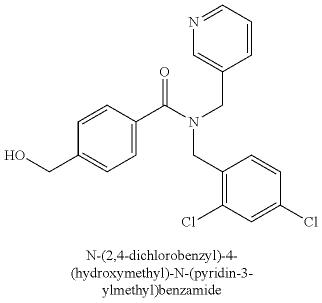 [SMILES: O=C(c1ccc(CO)cc1)N(Cc1cccnc1)Cc1ccc(Cl)cc1Cl] | 401.0 |
| Compound 84 | 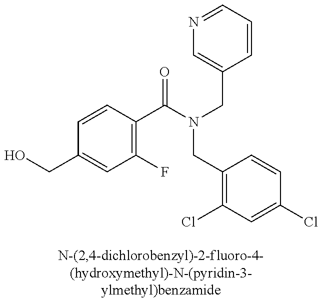 [SMILES: O=C(c1ccc(CO)cc1F)N(Cc1cccnc1)Cc1ccc(Cl)cc1Cl] | 419.0 |
| Compound 85 | 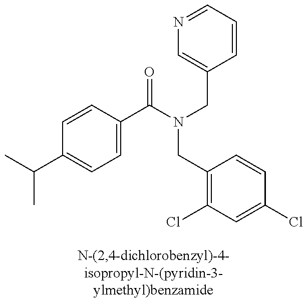 [SMILES: CC(C)c1ccc(C(=O)N(Cc2cccnc2)Cc2ccc(Cl)cc2Cl)cc1] | 413.0 |
| Compound 86 | 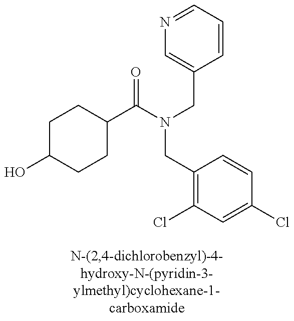 [SMILES: O=C(C1CCC(O)CC1)N(Cc1cccnc1)Cc1ccc(Cl)cc1Cl] | 393.0 |
| Compound 87 | 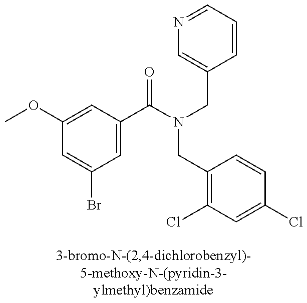 [SMILES: COc1cc(Br)cc(C(=O)N(Cc2cccnc2)Cc2ccc(Cl)cc2Cl)c1] | 480.9 |
| Compound 88 | 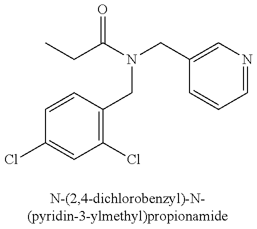 [SMILES: CCC(=O)N(Cc1cccnc1)Cc1ccc(Cl)cc1Cl] | 323.0 |
| Compound 89 | 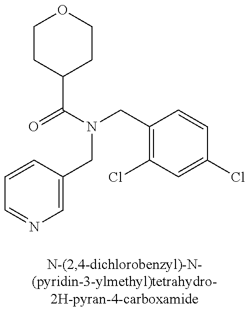 [SMILES: O=C(C1CCOCC1)N(Cc1cccnc1)Cc1ccc(Cl)cc1Cl] | 379.1 |
| Compound 90 | 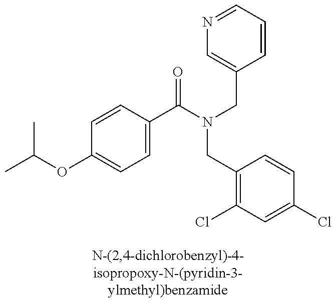 [SMILES: CC(C)Oc1ccc(C(=O)N(Cc2cccnc2)Cc2ccc(Cl)cc2Cl)cc1] | 429.1 |
| Compound 91 | 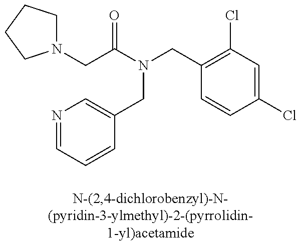 [SMILES: O=C(CN1CCCC1)N(Cc1cccnc1)Cc1ccc(Cl)cc1Cl] | 380.0 |
| Compound 92 | 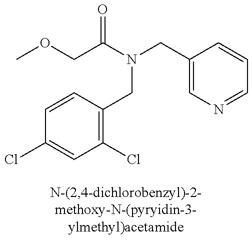 [SMILES: COCC(=O)N(Cc1cccnc1)Cc1ccc(Cl)cc1Cl] | 339.0 |
| Compound 93 | 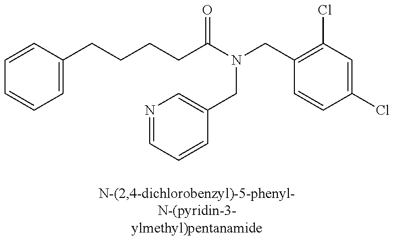 [SMILES: O=C(CCCCc1ccccc1)N(Cc1cccnc1)Cc1ccc(Cl)cc1Cl] | 427.2 |
| Compound 94 | 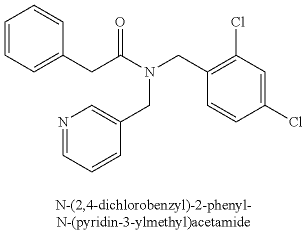 [SMILES: O=C(Cc1ccccc1)N(Cc1cccnc1)Cc1ccc(Cl)cc1Cl] | 385.0 |
| Compound 95 | 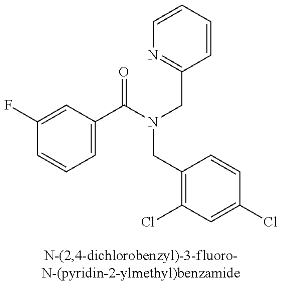 [SMILES: O=C(c1cccc(F)c1)N(Cc1ccccn1)Cc1ccc(Cl)cc1Cl] | 389.0 |
| Compound 96 | 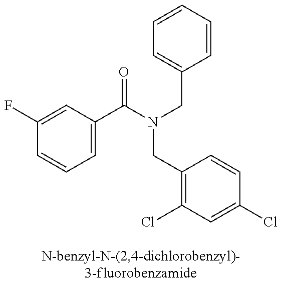 [SMILES: O=C(c1cccc(F)c1)N(Cc1ccccc1)Cc1ccc(Cl)cc1Cl] | 388.0 |
| Compound 97 | 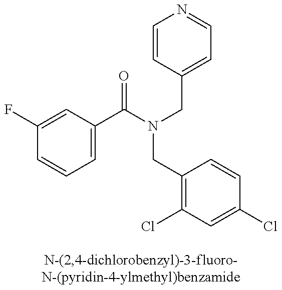 [SMILES: O=C(c1cccc(F)c1)N(Cc1ccncc1)Cc1ccc(Cl)cc1Cl] | 389.0 |
| Compound 98 | 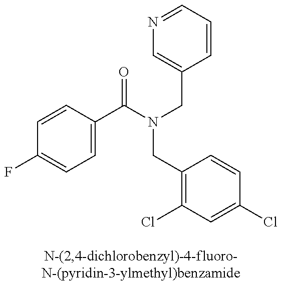 [SMILES: O=C(c1ccc(F)cc1)N(Cc1cccnc1)Cc1ccc(Cl)cc1Cl] | 389.6 |
| Compound 99 | 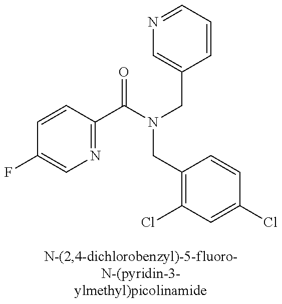 [SMILES: O=C(c1ccc(F)cn1)N(Cc1cccnc1)Cc1ccc(Cl)cc1Cl] | 390.5 |
| Compound 100 | 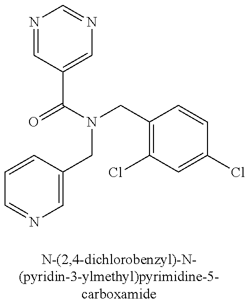 [SMILES: O=C(c1cncnc1)N(Cc1cccnc1)Cc1ccc(Cl)cc1Cl] | 373.6 |
| Compound 101 | 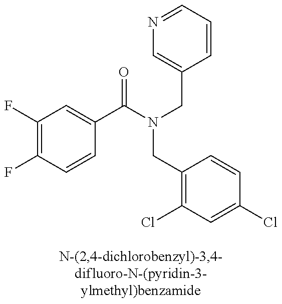 [SMILES: O=C(c1ccc(F)c(F)c1)N(Cc1cccnc1)Cc1ccc(Cl)cc1Cl] | 407.6 |
| Compound 102 | 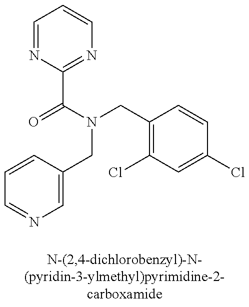 [SMILES: O=C(c1ncccn1)N(Cc1cccnc1)Cc1ccc(Cl)cc1Cl] | 373.6 |
| Compound 103 | 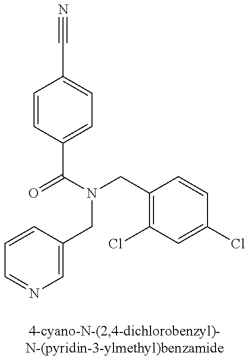 [SMILES: N#Cc1ccc(C(=O)N(Cc2cccnc2)Cc2ccc(Cl)cc2Cl)cc1] | 396.6 |
| Compound 104 | 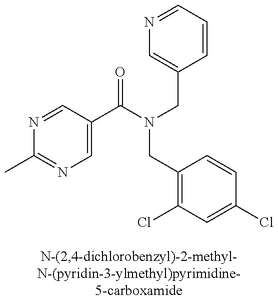 [SMILES: Cc1ncc(C(=O)N(Cc2cccnc2)Cc2ccc(Cl)cc2Cl)cn1] | 387.8 |
| Compound 105 | 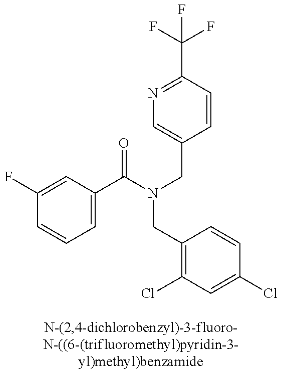 [SMILES: O=C(c1cccc(F)c1)N(Cc1ccc(C(F)(F)F)nc1)Cc1ccc(Cl)cc1Cl] | 457.1 |
| Compound 106 | 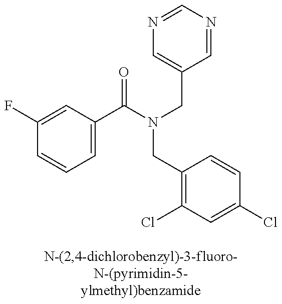 [SMILES: O=C(c1cccc(F)c1)N(Cc1cncnc1)Cc1ccc(Cl)cc1Cl] | 390.1 |
| Compound 107 | 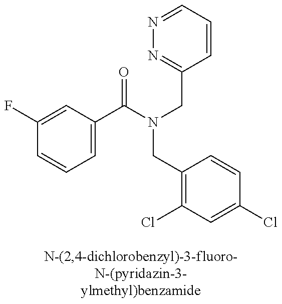 [SMILES: O=C(c1cccc(F)c1)N(Cc1cccnn1)Cc1ccc(Cl)cc1Cl] | 390.1 |
| Compound 108 | 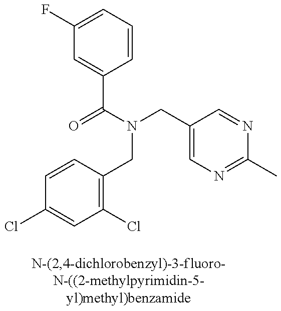 [SMILES: Cc1ncc(CN(Cc2ccc(Cl)cc2Cl)C(=O)c2cccc(F)c2)cn1] | 404.1 |
| Compound 109 | 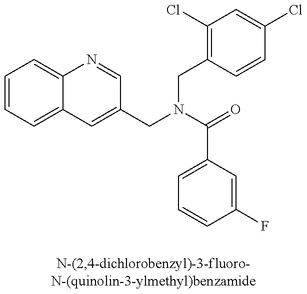 [SMILES: O=C(c1cccc(F)c1)N(Cc1cnc2ccccc2c1)Cc1ccc(Cl)cc1Cl] | 439.1 |
| Compound 110 | 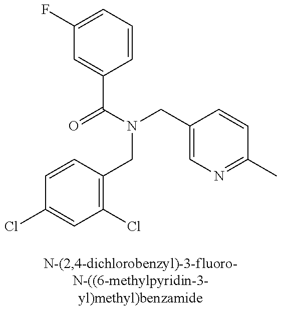 [SMILES: Cc1ccc(CN(Cc2ccc(Cl)cc2Cl)C(=O)c2cccc(F)c2)cn1] | 403.1 |
| Compound 111 | 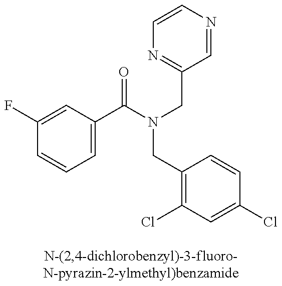 [SMILES: O=C(c1cccc(F)c1)N(Cc1cnccn1)Cc1ccc(Cl)cc1Cl] | 390.0 |
| Compound 112 | 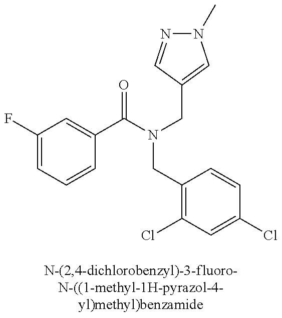 [SMILES: Cn1cc(CN(Cc2ccc(Cl)cc2Cl)C(=O)c2cccc(F)c2)cn1] | 392.0 |
| Compound 113 | 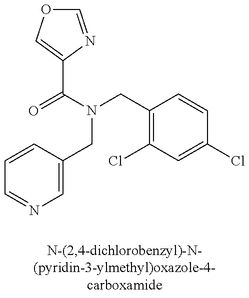 [SMILES: O=C(c1cocn1)N(Cc1cccnc1)Cc1ccc(Cl)cc1Cl] | 362.2 |
| Compound 114 | 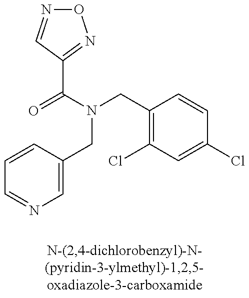 [SMILES: O=C(c1cnon1)N(Cc1cccnc1)Cc1ccc(Cl)cc1Cl] | 363.4 |
| Compound 115 | 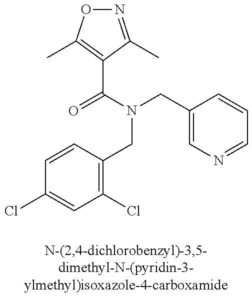 [SMILES: Cc1noc(C)c1C(=O)N(Cc1cccnc1)Cc1ccc(Cl)cc1Cl] | 390.5 |
| Compound 116 | 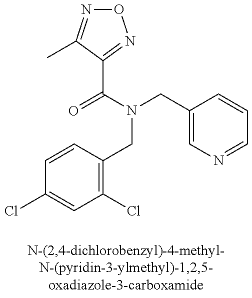 [SMILES: Cc1nonc1C(=O)N(Cc1cccnc1)Cc1ccc(Cl)cc1Cl] | 377.4 |
| Compound 117 | 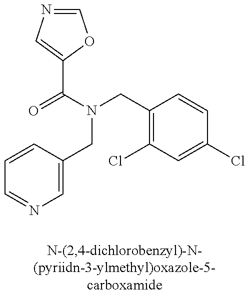 [SMILES: O=C(c1cnco1)N(Cc1cccnc1)Cc1ccc(Cl)cc1Cl] | 362.5 |
| Compound 118 | 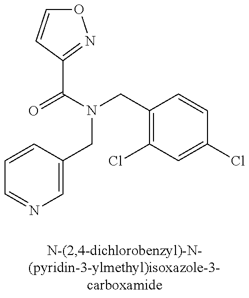 [SMILES: O=C(c1ccon1)N(Cc1cccnc1)Cc1ccc(Cl)cc1Cl] | 362.4 |
| Compound 119 | 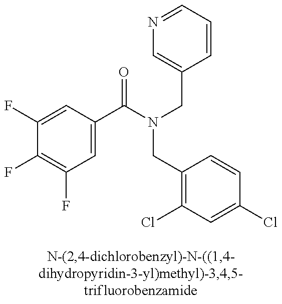 [SMILES: O=C(c1cc(F)c(F)c(F)c1)N(Cc1cccnc1)Cc1ccc(Cl)cc1Cl] | 424.9 |
| Compound 120 | 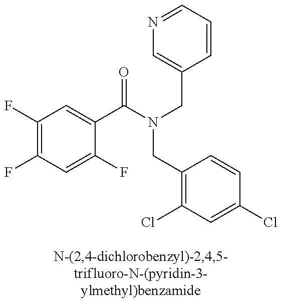 [SMILES: O=C(c1cc(F)c(F)cc1F)N(Cc1cccnc1)Cc1ccc(Cl)cc1Cl] | 424.9 |
| Compound 121 | 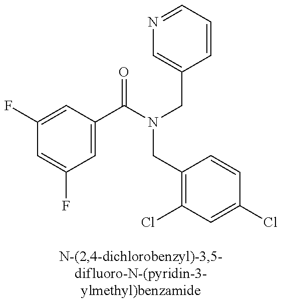 [SMILES: O=C(c1cc(F)cc(F)c1)N(Cc1cccnc1)Cc1ccc(Cl)cc1Cl] | 406.9 |
| Compound 122 | 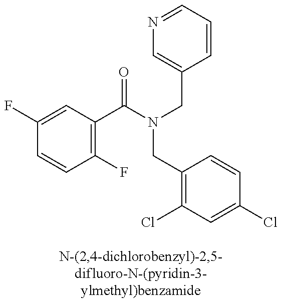 [SMILES: O=C(c1cc(F)ccc1F)N(Cc1cccnc1)Cc1ccc(Cl)cc1Cl] | 406.9 |
| Compound 123 | 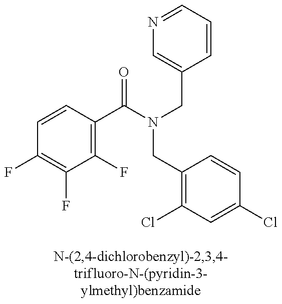 [SMILES: O=C(c1ccc(F)c(F)c1F)N(Cc1cccnc1)Cc1ccc(Cl)cc1Cl] | 424.9 |
| Compound 124 | 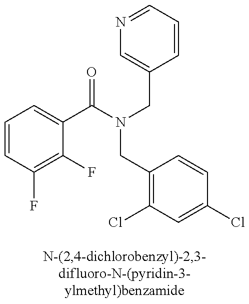 [SMILES: O=C(c1cccc(F)c1F)N(Cc1cccnc1)Cc1ccc(Cl)cc1Cl] | 406.9 |
| Compound 125 | 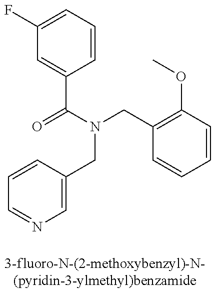 [SMILES: COc1ccccc1CN(Cc1cccnc1)C(=O)c1cccc(F)c1] | 351.5 |
| Compound 126 | 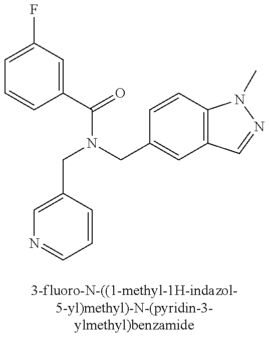 [SMILES: Cn1ncc2cc(CN(Cc3cccnc3)C(=O)c3cccc(F)c3)ccc21] | 375.5 |
| Compound 127 | 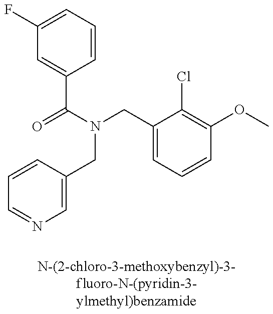 [SMILES: COc1cccc(CN(Cc2cccnc2)C(=O)c2cccc(F)c2)c1Cl] | 385.6 |
| Compound 128 | 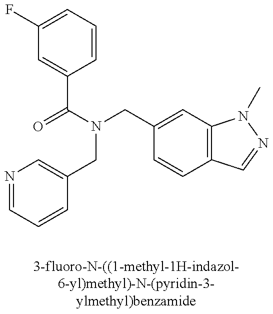 [SMILES: Cn1ncc2ccc(CN(Cc3cccnc3)C(=O)c3cccc(F)c3)cc21] | 375.4 |
| Compound 129 | 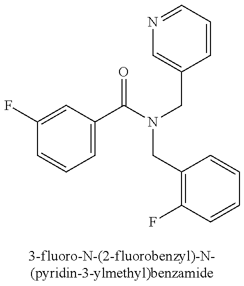 [SMILES: O=C(c1cccc(F)c1)N(Cc1cccnc1)Cc1ccccc1F] | 339.1 |
| Compound 130 | 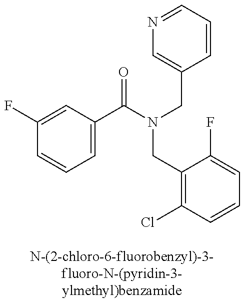 [SMILES: O=C(c1cccc(F)c1)N(Cc1cccnc1)Cc1c(F)cccc1Cl] | 373.1 |
| Compound 131 | 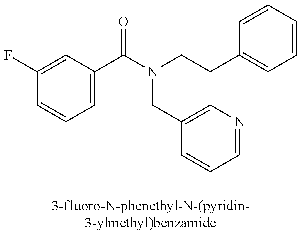 [SMILES: O=C(c1cccc(F)c1)N(CCc1ccccc1)Cc1cccnc1] | 335.0 |
| Compound 1332 | 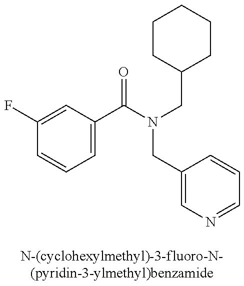 [SMILES: O=C(c1cccc(F)c1)N(Cc1cccnc1)CC1CCCCC1] | 327.1 |
| Compound 133 | 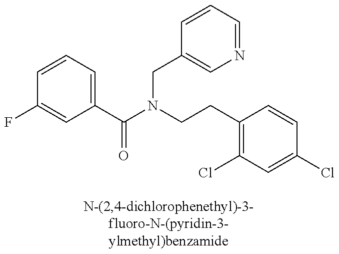 [SMILES: O=C(c1cccc(F)c1)N(CCc1ccc(Cl)cc1Cl)Cc1cccnc1] | 402.9 |
| Compound 134 | 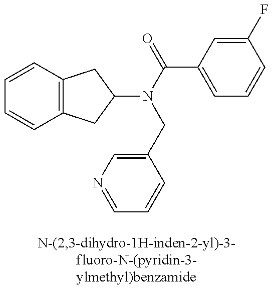 [SMILES: O=C(c1cccc(F)c1)N(Cc1cccnc1)C1Cc2ccccc2C1] | 347.0 |
| Compound 135 | 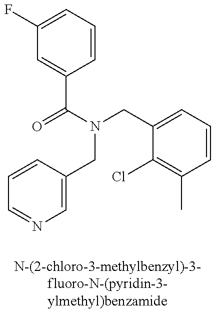 [SMILES: Cc1cccc(CN(Cc2cccnc2)C(=O)c2cccc(F)c2)c1Cl] | 369.0 |
| Compound 136 | 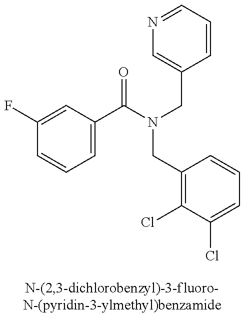 [SMILES: O=C(c1cccc(F)c1)N(Cc1cccnc1)Cc1cccc(Cl)c1Cl] | 388.9 |
| Compound 137 | 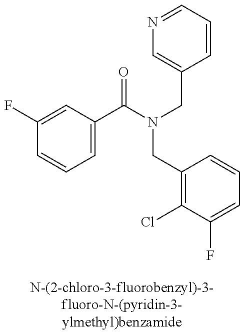 [SMILES: O=C(c1cccc(F)c1)N(Cc1cccnc1)Cc1cccc(F)c1Cl] | 372.9 |

General Procedure I

### Synthesis of N-chlorobenzyl)-3-fluoro-N-(pyridin-3-ylmethyl)benzamide (Compound 138)

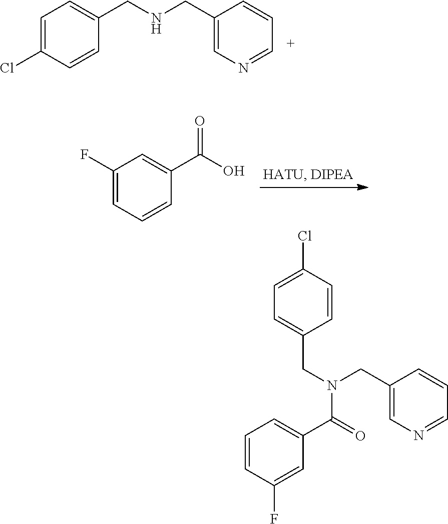 [SMILES: Clc1ccc(CNCc2cccnc2)cc1 ; O=C(O)c1cccc(F)c1 ; O=C(c1cccc(F)c1)N(Cc1ccc(Cl)cc1)Cc1cccnc1]

Step 1 Synthesis of N-(4-chlorobenzyl)-3-fluoro-N-(pyridin-3-ylmethyl)benzamnide. 3-fluorobenzoic acid (30 mg, 214 μmol) and HATU (97.3 mg, 256 μmol) were combined in DMF and stirred at room temperature for 10 minutes. This solution was then added to a flask containing [(4-chlorophenyl)methyl][(pyridin-3-yl)methyl]amine (59.5 mg, 256 μmol) and then N,N-diisopropylethylamine (185 μL, 1.07 mmol) was added. The reaction was stirred overnight at room temperature. The reaction mixture was directly subjected to mass directed Prep HPLC to obtain N-[(4-chlorophenyl)methyl]-3-fluoro-N-[(pyridin-3-yl)methyl]benzamide (46.9 mg, 60%). MS ES m/z: 355.1 [M+H]⁺.

The Following Compounds in Table 3 were Prepared in a Similar Manner to General Procedure I Described Above:

TABLE 3

| | | (ES, |
|---|---|---|
| Com- | | m/z) |
| pound | | [M + |
| ID | Structure | H]⁺ |
| Com- pound 139 | 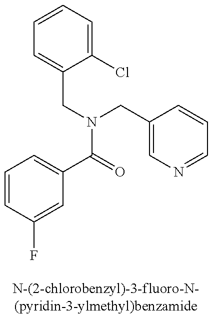 [SMILES: O=C(c1cccc(F)c1)N(Cc1cccnc1)Cc1ccccc1Cl] | 355.1 |
| Com- pound 140 | 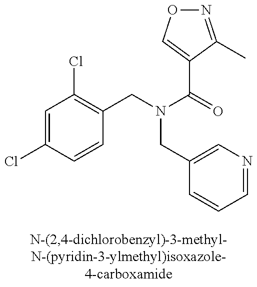 [SMILES: Cc1nocc1C(=O)N(Cc1cccnc1)Cc1ccc(Cl)cc1Cl] | 376.0 |
| Com- pound 141 | 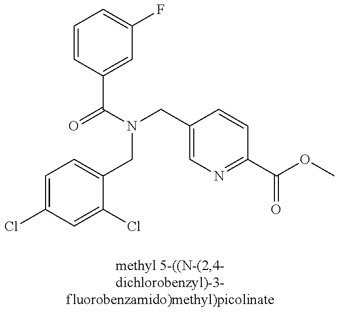 [SMILES: COC(=O)c1ccc(CN(Cc2ccc(Cl)cc2Cl)C(=O)c2cccc(F)c2)cn1] | 447.0 |
| Com- pound 176 | 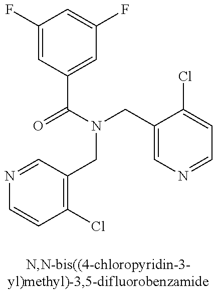 [SMILES: O=C(c1cc(F)cc(F)c1)N(Cc1cnccc1Cl)Cc1cnccc1Cl] | 408.5 |
| Com- pound 177 | 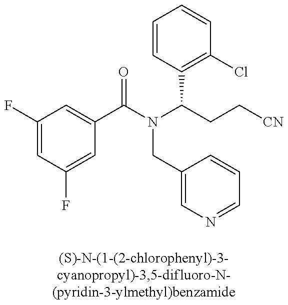 [SMILES: N#CCC[C@@H](c1ccccc1Cl)N(Cc1cccnc1)C(=O)c1cc(F)cc(F)c1] | 426.5 |
| Com- pound 178 | 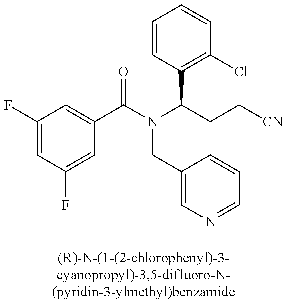 [SMILES: N#CCC[C@H](c1ccccc1Cl)N(Cc1cccnc1)C(=O)c1cc(F)cc(F)c1] | 426.5 |
| Com- pound 179 | 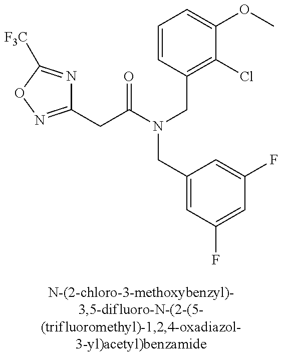 [SMILES: COc1cccc(CN(Cc2cc(F)cc(F)c2)C(=O)Cc2noc(C(F)(F)F)n2)c1Cl] | 462.1 |
| Com- pound 180 | 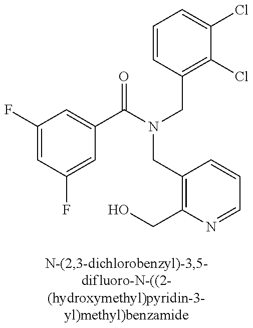 [SMILES: O=C(c1cc(F)cc(F)c1)N(Cc1cccnc1CO)Cc1cccc(Cl)c1Cl] | 437.3 |
| Com- pound 181 | 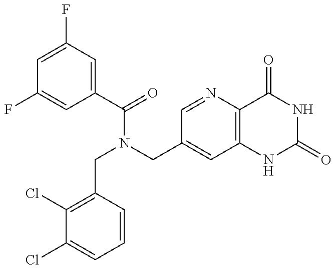 [SMILES: O=C(c1cc(F)cc(F)c1)N(Cc1cnc2c(=O)[nH]c(=O)[nH]c2c1)Cc1cccc(Cl)c1Cl] | 491.3 |
| Com- pound 182 | 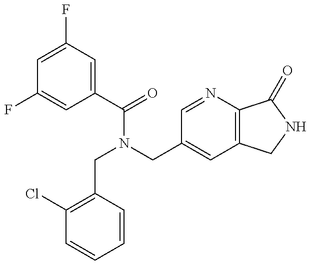 [SMILES: O=C1NCc2cc(CN(Cc3ccccc3Cl)C(=O)c3cc(F)cc(F)c3)cnc21] | 427.84 |
| Com- pound 183 | 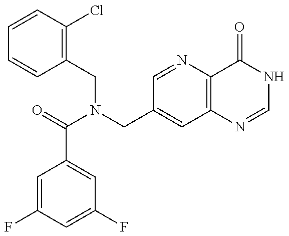 [SMILES: O=C(c1cc(F)cc(F)c1)N(Cc1cnc2c(=O)[nH]cnc2c1)Cc1ccccc1Cl] | 440.8 |

General Procedure II

### Synthesis of methyl 5-({[(2,4-dichlorophenyl)methyl]amino}methyl)pyridine-2-carboxylate (Compound 142)

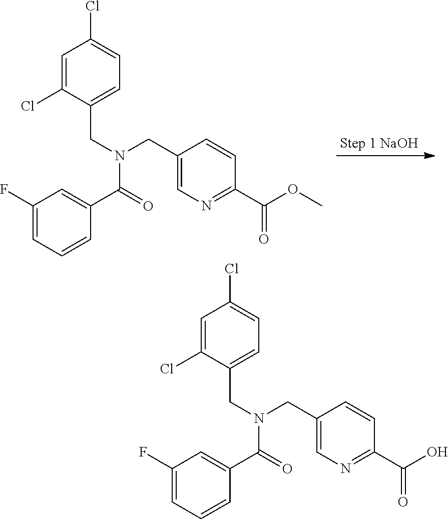

Step 1. methyl 5-({N-[(2,4-dichlorophenyl)methyl]-1-(3-fluorophenyl)formamido}methyl)pyridine-2-carboxylate (Compound 141, 64 mg, 143 umol) was dissolved in 5:1 THF:MeOH (500 uL). IN aq. NaOH (1.43 mL) was added and the reaction stirred at room temperature for 4 hours. The reaction was acidified to pH ˜3 with 1N HCl and extracted 4×5 mL CH₂Cl₂. The organic layers were combined and concentrated to afford 5-({N-[(2,4-dichlorophenyl)methyl]-1-(3-fluorophenyl)formamido}methyl)pyridine-2-carboxylic acid (56 mg, 82% yield) as a white solid. MS ES m/z: 433.0 [M+H]⁺.

The Following Compounds in Table 4 were Prepared in a Similar Manner to General Procedure II Described Above:

TABLE 4

| | | (ES, |
|---|---|---|
| Com- | | m/z) |
| pound | | [M + |
| ID | Structure | H]⁺ |
| Com- pound 147 | 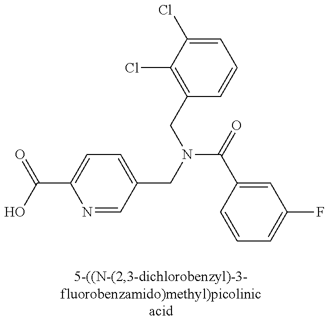 [SMILES: O=C(O)c1ccc(CN(Cc2cccc(Cl)c2Cl)C(=O)c2cccc(F)c2)cn1] | 434.9 |
| Com- pound 148 | 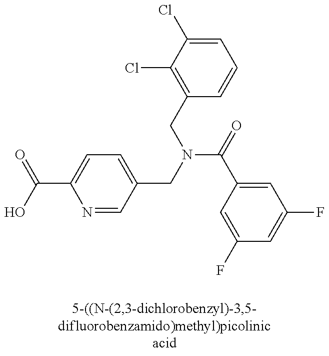 [SMILES: O=C(O)c1ccc(CN(Cc2cccc(Cl)c2Cl)C(=O)c2cc(F)cc(F)c2)cn1] | 450.9 |
| Com- pound 151 | 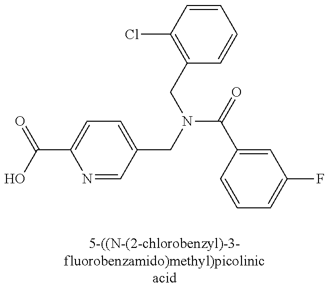 [SMILES: O=C(O)c1ccc(CN(Cc2ccccc2Cl)C(=O)c2cccc(F)c2)cn1] | 399.0 |
| Com- pound 152 | 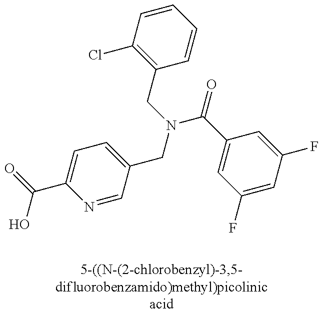 [SMILES: O=C(O)c1ccc(CN(Cc2ccccc2Cl)C(=O)c2cc(F)cc(F)c2)cn1] | 416.9 |
| Com- pound 184 | 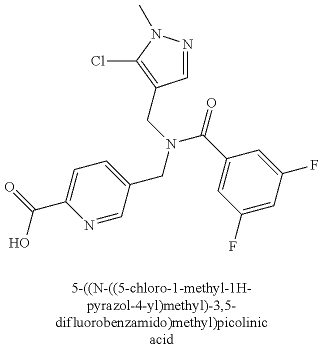 [SMILES: Cn1ncc(CN(Cc2ccc(C(=O)O)nc2)C(=O)c2cc(F)cc(F)c2)c1Cl] | 419.1 |
| Com- pound 185 | 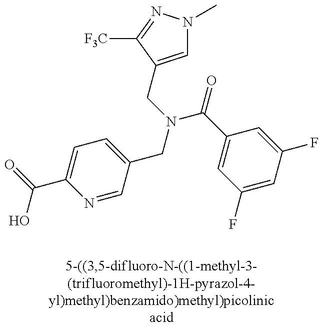 [SMILES: Cn1cc(CN(Cc2ccc(C(=O)O)nc2)C(=O)c2cc(F)cc(F)c2)c(C(F)(F)F)n1] | 453.0 |
| Com- pound 186 | 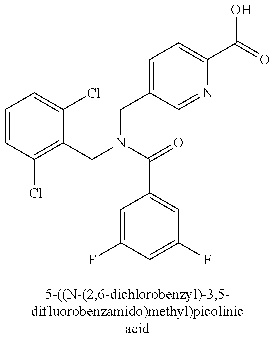 [SMILES: O=C(O)c1ccc(CN(Cc2c(Cl)cccc2Cl)C(=O)c2cc(F)cc(F)c2)cn1] | 452.8 |
| Com- pound 187 | 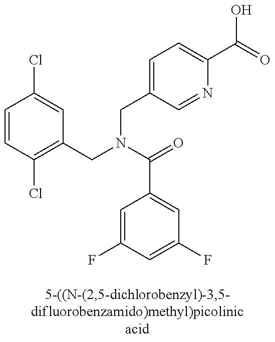 [SMILES: O=C(O)c1ccc(CN(Cc2cc(Cl)ccc2Cl)C(=O)c2cc(F)cc(F)c2)cn1] | 452.8 |
| Com- pound 188 | 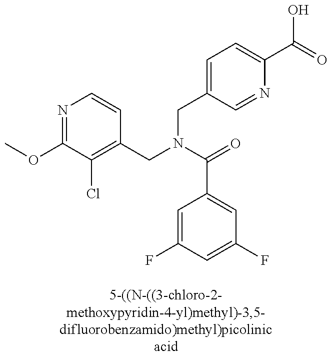 [SMILES: COc1nccc(CN(Cc2ccc(C(=O)O)nc2)C(=O)c2cc(F)cc(F)c2)c1Cl] | 447.8 |
| Com- pound 189 | 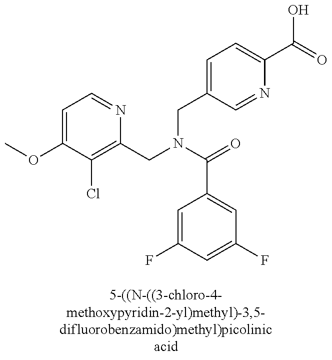 [SMILES: COc1ccnc(CN(Cc2ccc(C(=O)O)nc2)C(=O)c2cc(F)cc(F)c2)c1Cl] | 448.4 |
| Com- pound 190 | 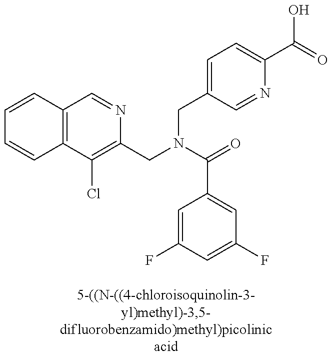 [SMILES: O=C(O)c1ccc(CN(Cc2ncc3ccccc3c2Cl)C(=O)c2cc(F)cc(F)c2)cn1] | 468.1 |
| Com- pound 191 | 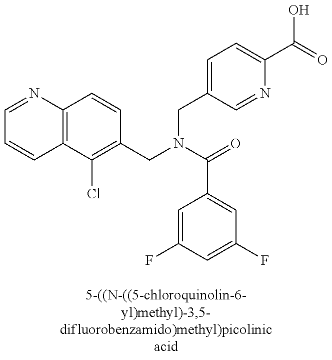 [SMILES: O=C(O)c1ccc(CN(Cc2ccc3ncccc3c2Cl)C(=O)c2cc(F)cc(F)c2)cn1] | 468.1 |
| Com- pound 192 | 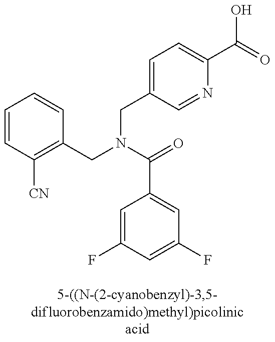 [SMILES: N#Cc1ccccc1CN(Cc1ccc(C(=O)O)nc1)C(=O)c1cc(F)cc(F)c1] | 408.5 |
| Com- pound 193 | 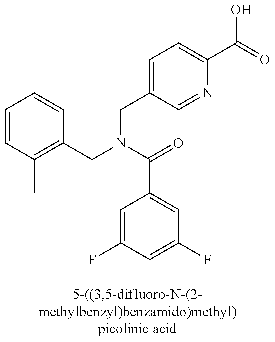 [SMILES: Cc1ccccc1CN(Cc1ccc(C(=O)O)nc1)C(=O)c1cc(F)cc(F)c1] | 397.2 |
| Com- pound 194 | 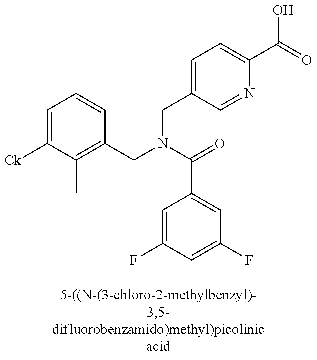 | 429.0 |
| Com- pound 195 | 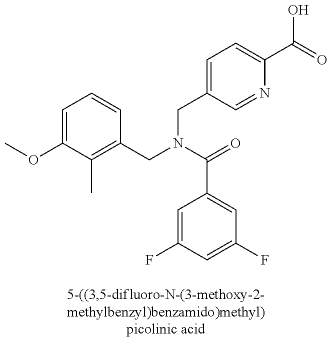 [SMILES: COc1cccc(CN(Cc2ccc(C(=O)O)nc2)C(=O)c2cc(F)cc(F)c2)c1C] | 427.5 |
| Com- pound 196 | 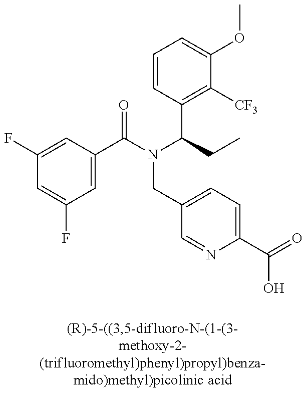 [SMILES: CC[C@H](c1cccc(OC)c1C(F)(F)F)N(Cc1ccc(C(=O)O)nc1)C(=O)c1cc(F)cc(F)c1] | 509.4 |
| Com- pound 197 | 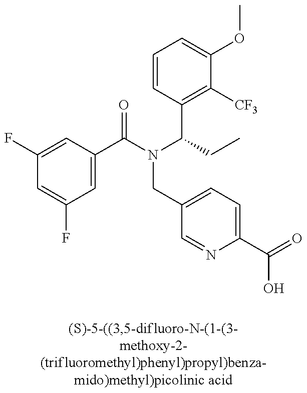 [SMILES: CC[C@@H](c1cccc(OC)c1C(F)(F)F)N(Cc1ccc(C(=O)O)nc1)C(=O)c1cc(F)cc(F)c1] | 509.4 |
| Com- pound 198 | 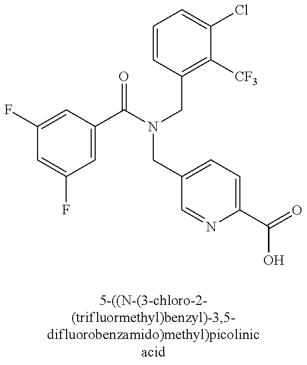 [SMILES: O=C(O)c1ccc(CN(Cc2cccc(Cl)c2C(F)(F)F)C(=O)c2cc(F)cc(F)c2)cn1] | 485.7 |
| Com- pound 199 | 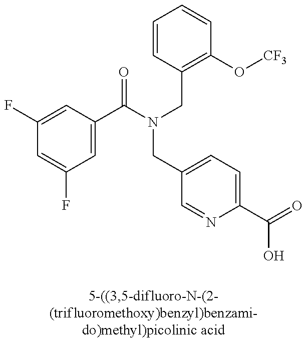 [SMILES: O=C(O)c1ccc(CN(Cc2ccccc2OC(F)(F)F)C(=O)c2cc(F)cc(F)c2)cn1] | 467.9 |
| Com- pound 200 | 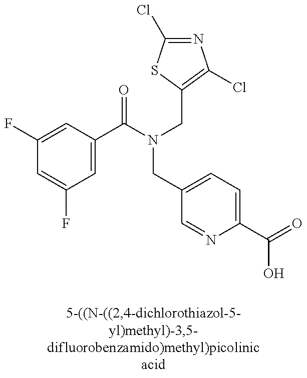 [SMILES: O=C(O)c1ccc(CN(Cc2sc(Cl)nc2Cl)C(=O)c2cc(F)cc(F)c2)cn1] | 456.1 |
| Com- pound 201 | 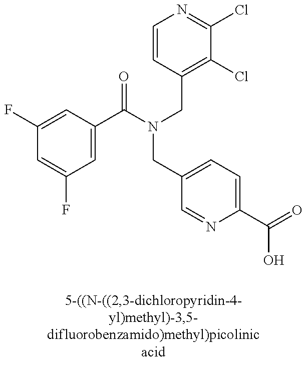 [SMILES: O=C(O)c1ccc(CN(Cc2ccnc(Cl)c2Cl)C(=O)c2cc(F)cc(F)c2)cn1] | 452.7 |
| Com- pound 202 | 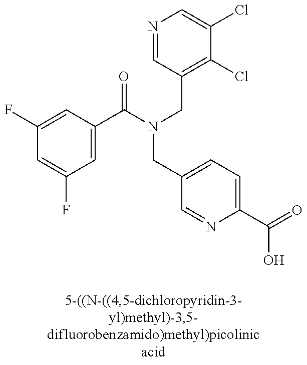 [SMILES: O=C(O)c1ccc(CN(Cc2cncc(Cl)c2Cl)C(=O)c2cc(F)cc(F)c2)cn1] | 454.0 |
| Com- pound 203 | 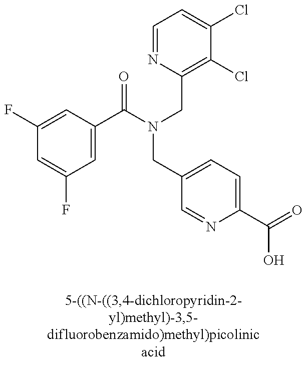 [SMILES: O=C(O)c1ccc(CN(Cc2nccc(Cl)c2Cl)C(=O)c2cc(F)cc(F)c2)cn1] | 452.4 |
| Com- pound 204 | 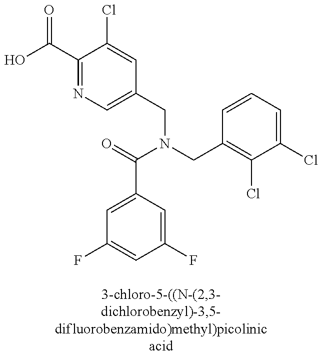 [SMILES: O=C(O)c1ncc(CN(Cc2cccc(Cl)c2Cl)C(=O)c2cc(F)cc(F)c2)cc1Cl] | 487.6 |
| Com- pound 205 | 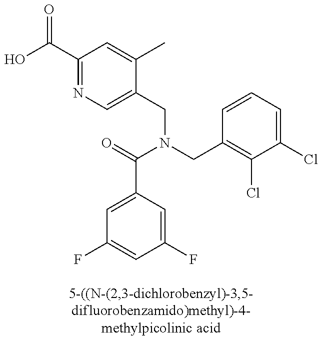 [SMILES: Cc1cc(C(=O)O)ncc1CN(Cc1cccc(Cl)c1Cl)C(=O)c1cc(F)cc(F)c1] | 465.0 |
| Com- pound 206 | 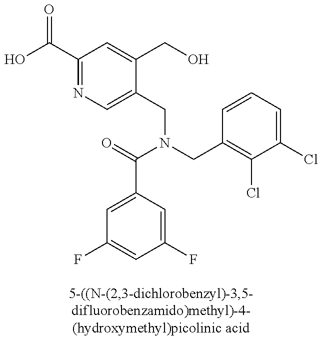 [SMILES: O=C(O)c1cc(CO)c(CN(Cc2cccc(Cl)c2Cl)C(=O)c2cc(F)cc(F)c2)cn1] | 480.8 |
| Com- pound 168 | 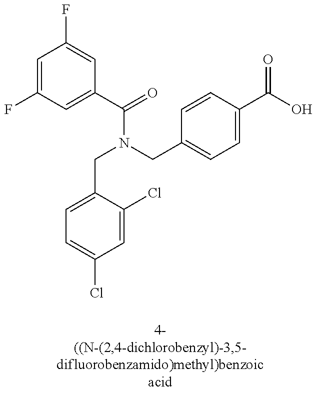 [SMILES: O=C(O)c1ccc(CN(Cc2ccc(Cl)cc2Cl)C(=O)c2cc(F)cc(F)c2)cc1] | 450.0 |
| Com- pound 172 | 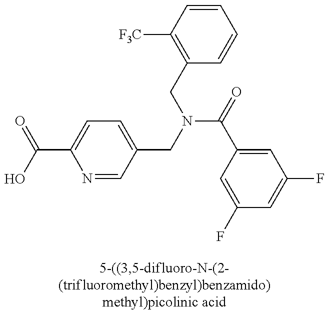 [SMILES: O=C(O)c1ccc(CN(Cc2ccccc2C(F)(F)F)C(=O)c2cc(F)cc(F)c2)cn1] | 451.1 |
| Com- pound 207 | 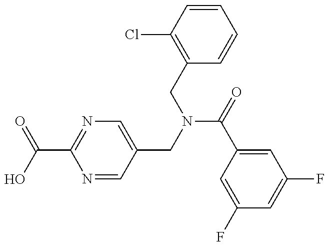 [SMILES: O=C(O)c1ncc(CN(Cc2ccccc2Cl)C(=O)c2cc(F)cc(F)c2)cn1] | 418.1 |
| Com- pound 208 | 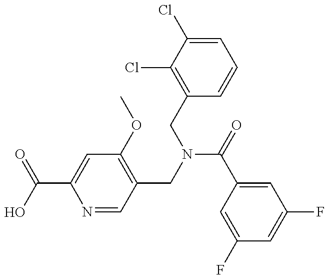 [SMILES: COc1cc(C(=O)O)ncc1CN(Cc1cccc(Cl)c1Cl)C(=O)c1cc(F)cc(F)c1] | 481.3 |
| Com- pound 209 | 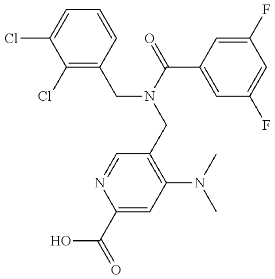 [SMILES: CN(C)c1cc(C(=O)O)ncc1CN(Cc1cccc(Cl)c1Cl)C(=O)c1cc(F)cc(F)c1] | 494.3 |
| Com- pound 210 | 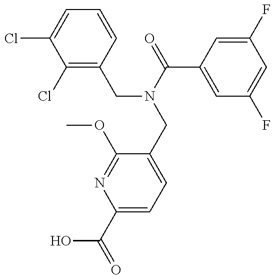 [SMILES: COc1nc(C(=O)O)ccc1CN(Cc1cccc(Cl)c1Cl)C(=O)c1cc(F)cc(F)c1] | 481.2 |
| Com- pound 247 | 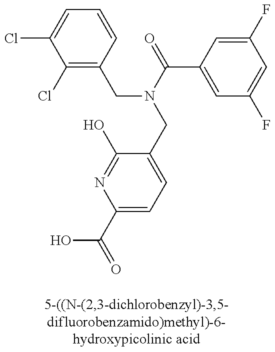 [SMILES: O=C(O)c1ccc(CN(Cc2cccc(Cl)c2Cl)C(=O)c2cc(F)cc(F)c2)c(O)n1] | 467.3 |
| Com- pound 258 | 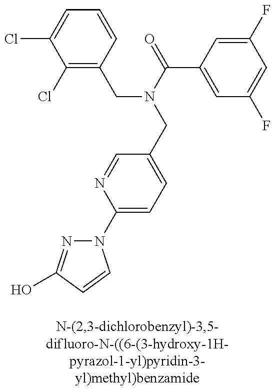 [SMILES: O=C(c1cc(F)cc(F)c1)N(Cc1ccc(-n2ccc(O)n2)nc1)Cc1cccc(Cl)c1Cl] | 488.1 |

General Procedure III

### Synthesis of 5-({N-[(2,4-dichlorophenyl)methyl]-1-(3-fluorophenyl)formamido}methyl)pyridine-2-carboxamide (Compound 143)

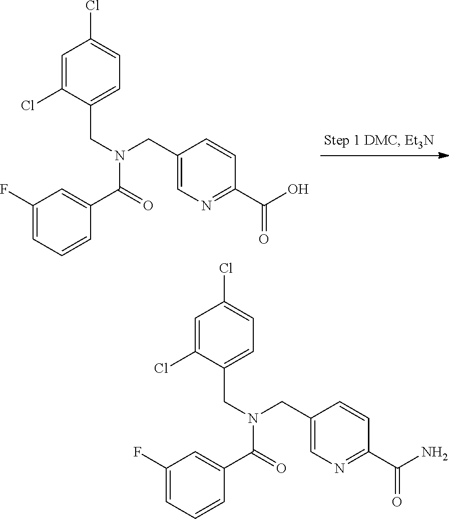 [SMILES: O=C(O)c1ccc(CN(Cc2ccc(Cl)cc2Cl)C(=O)c2cccc(F)c2)cn1 ; NC(=O)c1ccc(CN(Cc2ccc(Cl)cc2Cl)C(=O)c2cccc(F)c2)cn1]

Step 1. 5-({N-[(2,4-dichlorophenyl)methyl]-1-(3-fluorophenyl)formamido}methyl)pyridine-2-carboxylic acid (Compound 142, 15 mg, 34 umol), NH₄ (7M in MeOH; 10 uL, 69 umol), and Et₃N (14 uL, 69 umol) were combined in anhydrous DCE (1.5 mL). DMC (6 mg, 35 umol) was added, and the reaction was stirred at room temperature for 4 hours. The reaction was quenched with 1 mL H₂O and the organic layer was separated and concentrated to dryness. Purified by reverse phase chromatography using Biotage (Sfar C18 12 g, 90% H₂O/10% CH₃CN w/0.1% formic acid up to 10% H₂O/90% CH₃CN w/0.1% formic acid). Desired fractions were combined and concentrated to afford 5-({N-[(2,4-dichlorophenyl)methyl]-1-(3-fluorophenyl)formamido}methyl)pyridine-2-carboxamide (8.9 mg, 60% yield). MS ES m/z: 432.1 [M+H]⁺.

The Following Compounds in Table 5 were Prepared in a Similar Manner to General Procedure III Described Above:

TABLE 5

| | | (ES, |
|---|---|---|
| Com- | | m/z) |
| pound | | [M + |
| ID | Structure | H]⁺ |
| Com- pound 149 | 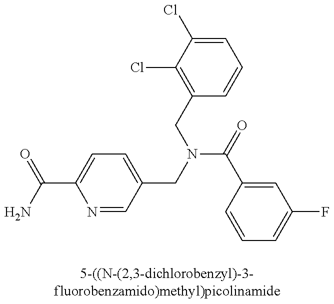 [SMILES: NC(=O)c1ccc(CN(Cc2cccc(Cl)c2Cl)C(=O)c2cccc(F)c2)cn1] | 432.0 |
| Com- pound 150 | 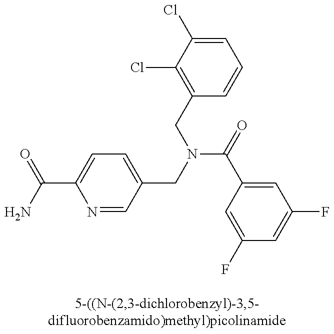 [SMILES: NC(=O)c1ccc(CN(Cc2cccc(Cl)c2Cl)C(=O)c2cc(F)cc(F)c2)cn1] | 450.0 |

General Procedure IV

### Synthesis of 5-({N-[(2,4-dichlorophenyl)methyl]-1-(3-fluorophenyl)formamido}methyl)-N-methylpyridine-2-carboxamide (Compound 144)

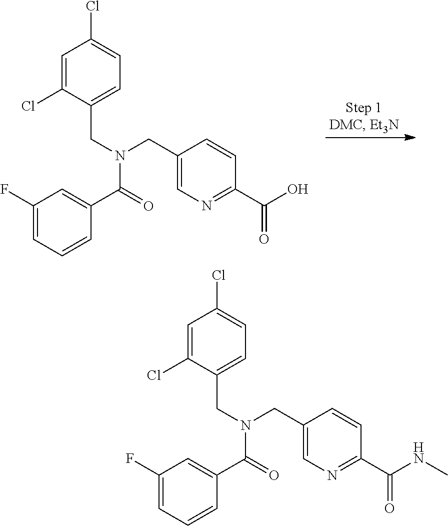 [SMILES: O=C(O)c1ccc(CN(Cc2ccc(Cl)cc2Cl)C(=O)c2cccc(F)c2)cn1 ; CNC(=O)c1ccc(CN(Cc2ccc(Cl)cc2Cl)C(=O)c2cccc(F)c2)cn1]

Step 1. 5-({N-[(2,4-dichlorophenyl)methyl]-1-(3-fluorophenyl)formamido}methyl)pyridine-2-carboxylic acid (Compound 142, 15 mg, 35 umol), Methylamine HCl (4.7 mg, 69 umol), and Et₃N (14 uL, 103 umol) were combined in DCE (1.5 mL). DMC (6 mg, 35 umol) was added, and the reaction was stirred at room temperature for 4 hours. The reaction was quenched with 1 mL H₂O and the organic layer was separated and concentrated to dryness. Purified by reverse phase chromatography using Biotage (Sfar C18 12 g, 90% H₂O/10% CH₃CN w/0.1% formic acid up to 10% H₂O/90% CH₃CN w/0.1% formic acid). Desired fractions were combined and concentrated to afford 5-({N-[(2,4-dichlorophenyl)methyl]-1-(3-fluorophenyl)formamido}methyl)-N-methylpyridine-2-carboxamide (12.5 mg, 81% yield). MS ES m/z: 446.1 [M+H]⁺.

General Procedure V

### Synthesis of N-(2,4-dichlorobenzyl)-3-fluoro-N-((6-(hydroxymethyl)pyridin-3-yl)methyl)benzamide (Compound 145)

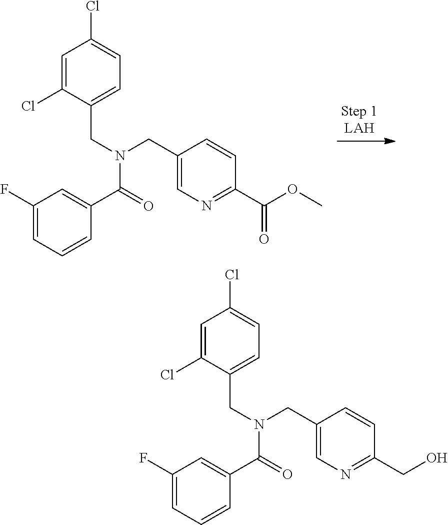 [SMILES: COC(=O)c1ccc(CN(Cc2ccc(Cl)cc2Cl)C(=O)c2cccc(F)c2)cn1 ; O=C(c1cccc(F)c1)N(Cc1ccc(CO)nc1)Cc1ccc(Cl)cc1Cl]

Step 1. A solution of methyl 5-({N-[(2,4-dichlorophenyl)methyl]-1-(3-fluorophenyl)formamido}methyl)pyridine-2-carboxylate (Compound 141, 48.7 mg, 109 umol) in anhydrous THF was added dropwise to a 2M solution of LAH (4.13 uL, 218 umol) in THF at 0° C. under N₂. The reaction was stirred at 0° C. and slowly warmed to room temperature over 90 minutes. The reaction was quenched with 1 mL water, diluted with 5 mL Et₂O, and another 1 mL water. The reaction was stirred at room temperature for 10 min. The organic layer was separated and concentrated to dryness. Purified by mass directed prep purification to afford N-(2,4-dichlorobenzyl)-3-fluoro-N-((6-(hydroxymethyl)pyridin-3-yl)methyl)benzamide (2.12 mg, 4.2% yield). 1H NMR (400 MHz, DMSO-d6) δ=8.21 (br s, 1H), 7.69 (br s, 1H), 7.56 (br s, 2H), 7.49-7.34 (m, 5H), 7.29 (br d, J=8.3 Hz, 3H), 5.37 (br s, 1H), 4.59 (br s, 3H), 4.52 (s, 4H), 3.29 (br s, 5H), 0.94 (t, J=7.1 Hz, 1H). MS: ES m/z: 419.0 [M+H]⁺.

General Procedure VI

### Synthesis of N-[(6-aminopyridin-3-yl)methyl]-N-[(2,4-dichlorophenyl)methyl]-3-fluorobenzamide (Compound 146)

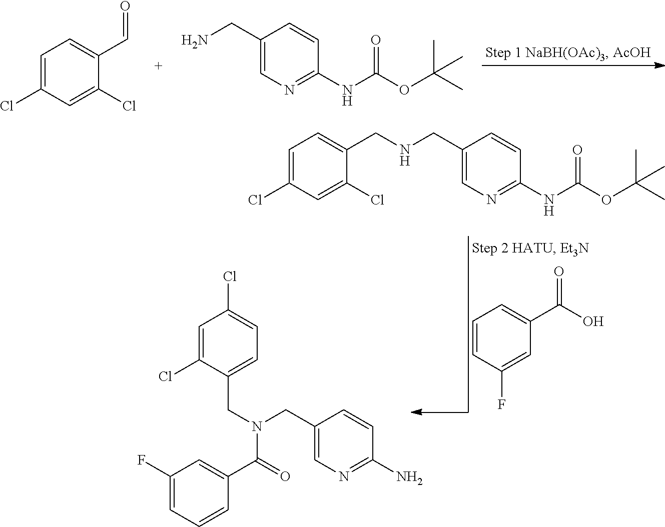 [SMILES: O=Cc1ccc(Cl)cc1Cl ; CC(C)(C)OC(=O)Nc1ccc(CN)cn1 ; CC(C)(C)OC(=O)Nc1ccc(CNCc2ccc(Cl)cc2Cl)cn1 ; Nc1ccc(CN(Cc2ccc(Cl)cc2Cl)C(=O)c2cccc(F)c2)cn1 ; O=C(O)c1cccc(F)c1]

### Synthesis of tert-butyl N-[5-({[(2,4-dichlorophenyl)methyl]amino}methyl)pyridin-2-yl]carbamate

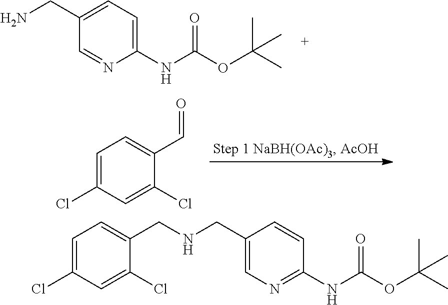

Step 1. 2,4-dichlorobenzaldehyde (194 mg, 1.11 mmol) was combined with tert-butyl (5-(aminomethyl)pyridin-2-yl)carbamate (250 mg, 1.11 mmol) and 10 uL Acetic Acid in DCE (20 mL). Lastly, NaBH(OAc)₃ (587 mg, 2.77 mmol) was added. The reaction was stirred at room temperature overnight. The reaction was quenched with Brine (15 mL). The organic layer was separated, dried over Na₂SO₄, filtered, and concentrated. Purified by reverse-phase chromatography using Biotage (Snap Ultra 30 g, 10% CH₃CN/90% H₂O w/0.1% formic acid up to 90% CH₃CN/10% H₂O w/0.1% formic acid gradient). Desired fractions were combined and lyophilized to afford tert-butyl N-[5-({[(2,4-dichlorophenyl)methyl]amino}methyl)pyridin-2-yl]carbamate (159.6 mg 38% yield).

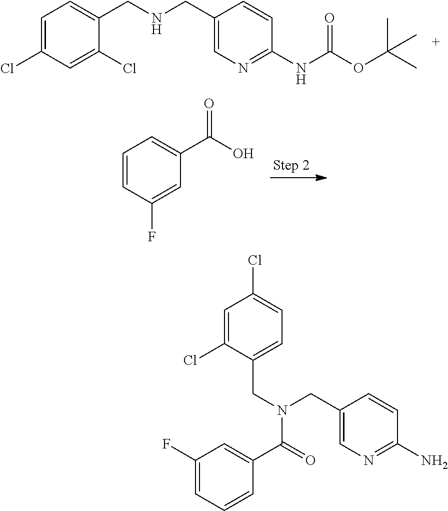 [SMILES: CC(C)(C)OC(=O)Nc1ccc(CNCc2ccc(Cl)cc2Cl)cn1 ; O=C(O)c1cccc(F)c1 ; Nc1ccc(CN(Cc2ccc(Cl)cc2Cl)C(=O)c2cccc(F)c2)cn1]

Step 2. tert-butyl N-[5-({[(2,4-dichlorophenyl)methyl]amino}methyl)pyridin-2-yl]carbamate (159.6 mg, 415 umol), 3-fluorobenzoic acid (64 mg, 456 umol), Et₃N (115 uL, 830 umol) and HATU (146 mg, 456 umol) were combined in DMA (4 mL). The reaction was stirred at room temperature overnight. The reaction was diluted with 5 mL H₂O and extracted 3×8 mL EtOAc. The organic layers were combined and concentrated. The residue was brought up in EtOAc (2 mL) and 5 eq. HCl (4M in 1,4-dioxane) and stirred at room temperature for 1 hour. The reaction was concentrated to dryness. Purified by normal-phase column chromatography using Biotage (ZIP Sphere 10 g, 10% EtOAc/Heptanes up to 100% EtOAc). Desired fractions were combined and concentrated to afford N-[(6-aminopyridin-3-yl)methyl]-N-[(2,4-dichlorophenyl)methyl]-3-fluorobenzamide as a white solid (32.1 mg, 17% yield). MS ES m/z: 404.0 [M+H]⁺.

The Following Compounds in Table 6 were Prepared in a Similar Manner to General Procedure VI Described Above:

TABLE 6

| | | (ES, |
|---|---|---|
| | | m/z) |
| Compound | | [M + |
| ID | Structure | H]⁺ |
| Compound 211 | 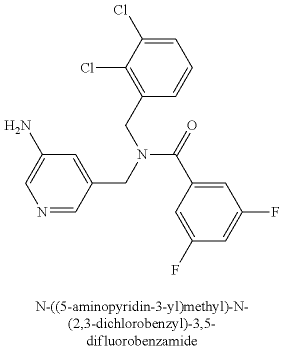 [SMILES: Nc1cncc(CN(Cc2cccc(Cl)c2Cl)C(=O)c2cc(F)cc(F)c2)c1] | 442.5 |
| Compound 212 | 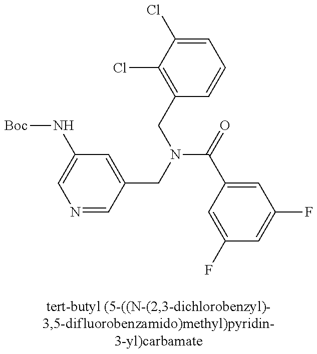 [SMILES: CC(C)(C)OC(=O)Nc1cncc(CN(Cc2cccc(Cl)c2Cl)C(=O)c2cc(F)cc(F)c2)c1] | 522.7 |

### Preparation of N-((6-acetamidopyridin-3-yl)methyl)-N-(2-chloro-3-methoxybenzyl)-3-fluorobenzamide (Compound 213)

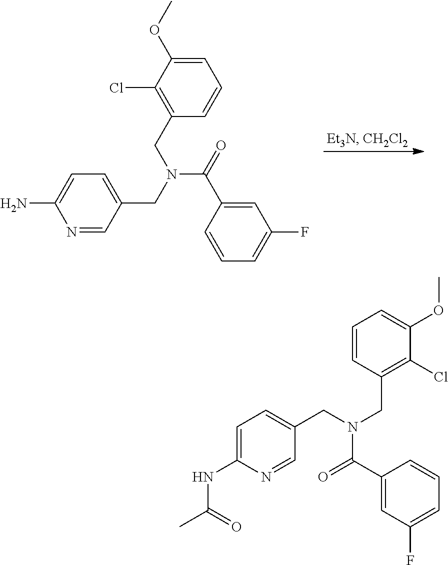 [SMILES: COc1cccc(CN(Cc2ccc(N)nc2)C(=O)c2cccc(F)c2)c1Cl ; COc1cccc(CN(Cc2ccc(NC(C)=O)nc2)C(=O)c2cccc(F)c2)c1Cl]

Step 1: N-((6-acetamidopyridin-3-yl)methyl)-N-(2-chloro-3-methoxybenzyl)-3-fluorobenzamide. To a stirred solution of 1-(pyridin-3-yl)ethan-1-amine (0.1 g, 239 umol) in CH₂Cl₂ (2 ml), Et₃N (20 uL, 263 umol) was added followed by the addition of acetyl chloride (7 uL, 263 umol) The reaction mixture was stirred at RT for 8 hours. After completion of reaction, the reaction mixture was washed with water (3 mL). The organic layer was removed, washed with Brine (3 ml), dried over Na₂SO₄. filtered, and concentrated under reduced pressure. The crude compound was purified by column chromatography to obtain the title compound (0.085 g, 77%). MS m/z 460.8 [M+1]⁺.

The Following Compounds in Table 7 were Prepared in a Similar Manner to Procedure Described Above:

TABLE 7

| | | (ES, m/z) |
|---|---|---|
| Compound ID | Structure | [M + H]⁺ |
| Compound 214 | 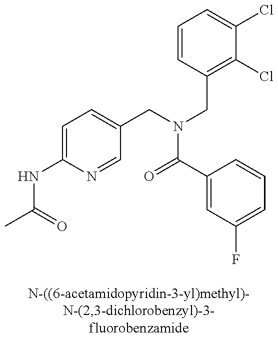 [SMILES: CC(=O)Nc1ccc(CN(Cc2cccc(Cl)c2Cl)C(=O)c2cccc(F)c2)cn1] | 446.1 |

General Procedures VII

### Preparation of (R)—N-(2,4-dichlorobenzyl)-3-fluoro-N-(1-(pyridin-3-yl)ethyl)benzamide (Compound 153) and (S)—N-(2,4-dichlorobenzyl)-3-fluoro-N-(1-(pyridin-3-yl)ethyl)benzamide (Compound 154)

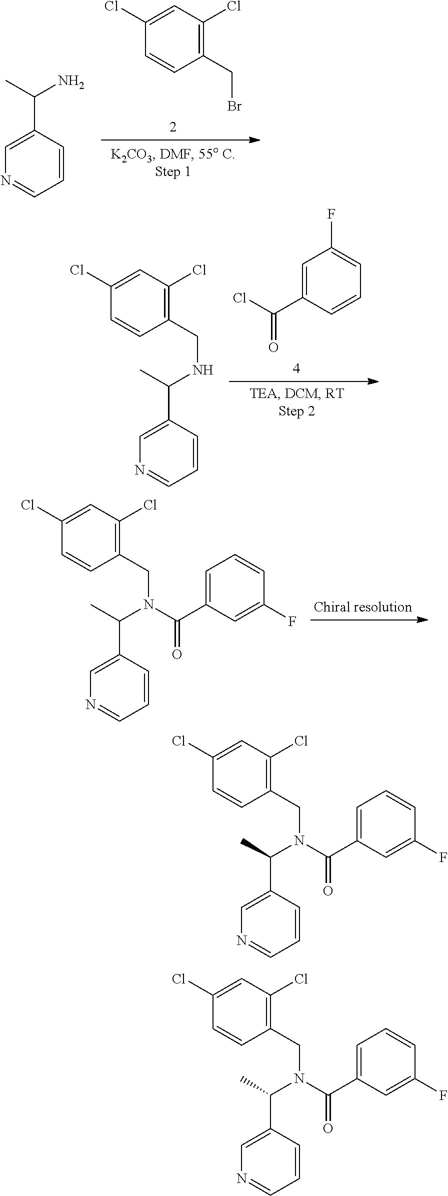

### Step 1: Synthesis of N-(2, 4-dichlorobenzyl)-1-(pyridin-3-yl)ethan-1-amine

To a stirred solution of 1-(pyridin-3-yl)ethan-1-amine (1 g, 8.18 mmol) in DMF (5 ml), Potassium carbonate (2.26 g, 16.4 mmol) was added. 1-(bromomethyl)-2,4-dichlorobenzene (2.55 g, 10.6 mmol) in DMF (5 ml) was added at room temperature. The reaction mixture was heated to 55° C. for 2 hours. After completion of reaction, the reaction mixture was diluted in Ethyl acetate (50 ml) and washed with cold water (3×50 ml). The organic layer was washed with Brine (50 ml), dried over Na₂SO₄. filtered, and concentrated under reduced pressure. The crude compound was purified by column chromatography to obtain the title compound (0.6 g, 26%). MS m/z 281.46 [M+1]⁺.

### Step 2: Synthesis of N-(2, 4-dichlorobenzyl)-3-fluoro-N-(1-(pyridin-3-yl)ethyl)benzamide

To a stirred mixture of N-(2, 4-dichlorobenzyl)-1-(pyridin-3-yl)ethan-1-amine (0.6 g, 2.13 mmol) in DCM (6 ml), Et₃N (0.54 g, 5.33 mmol) was added under nitrogen atmosphere. 3-Fluorobenzoyl chloride (0.51 g, 3.20 mmol) was added dropwise at room temperature and the reaction mixture was stirred at room temperature for 45 minutes. After completion of reaction, the reaction mixture was quenched with cold water (12 ml) and extracted with DCM (2×20 ml). The combined organic layer was washed with Brine (20 ml), dried over Na₂SO₄. filtered, and concentrated under reduced pressure. The crude compound was purified by combi flash chromatography to obtain the title compound (0.2 g, 23%) as a racemic mixture. MS: Racemic m/z 404.4 [M+H]⁺. Chiral Prep HPLC: Shimadzu LC-20AP with UV Detector; Column: CHIRALPAK IB-N (250*21) mm, 5 u; Mobile Phase A: 0.1% DEA in Hexane, Mobile Phase B: 0.1% DEA in Propan-2-ol; Isocratic gradient 85:15 (A:B), Column Flow: 18 mL/min. FR-1 (Isomer 1): R_{T}=18.98; FR-2 (Isomer 2): R_{T}=25.87.

Isomer 1 Arbitrarily assigned as (R)—N-(2,4-dichlorobenzyl)-3-fluoro-N-(1-(pyridin-3-yl)ethyl)benzamide (Compound 153) LCMS: m/z 404.3 [M+H]⁺. ¹H NMR (400 MHz, DMSO-d₆): δ 1.58 (d, J=6.8 Hz, 3H), 4.26 (bs, 1H), 4.59 (d, J=16.8 Hz, 1H), 5.15 (bs, 1H), 7.34-7.73 (m, 9H), 8.45 (d, J=4.0 Hz, 2H).

Isomer 2 Arbitrarily assigned as (S)—N-(2,4-dichlorobenzyl)-3-fluoro-N-(1-(pyridin-3-yl)ethyl)benzamide (Compound 154) LCMS: m/z 404.3 [M+H]⁺. ¹H NMR (400 MHz, DMSO-d₆): δ 1.58 (d, J=6.8 Hz, 3H), 4.27 (bs, 1H), 4.59 (d, J=16.8 Hz, 1H), 5.15 (bs, 1H), 7.33-7.72 (m, 9H), 8.46 (d, J=4.0 Hz, 2H).

General Procedures VIII

### Preparation of (R)—N-(1-(2,4-dichlorophenyl)propyl)-3-fluoro-N-(pyridin-3-ylmethyl)benzamide (Compound 157) and (S)—N-(1-(2,4-dichlorophenyl)propyl)-3-fluoro-N-(pyridin-3-ylmethyl)benzamide (Compound 158)

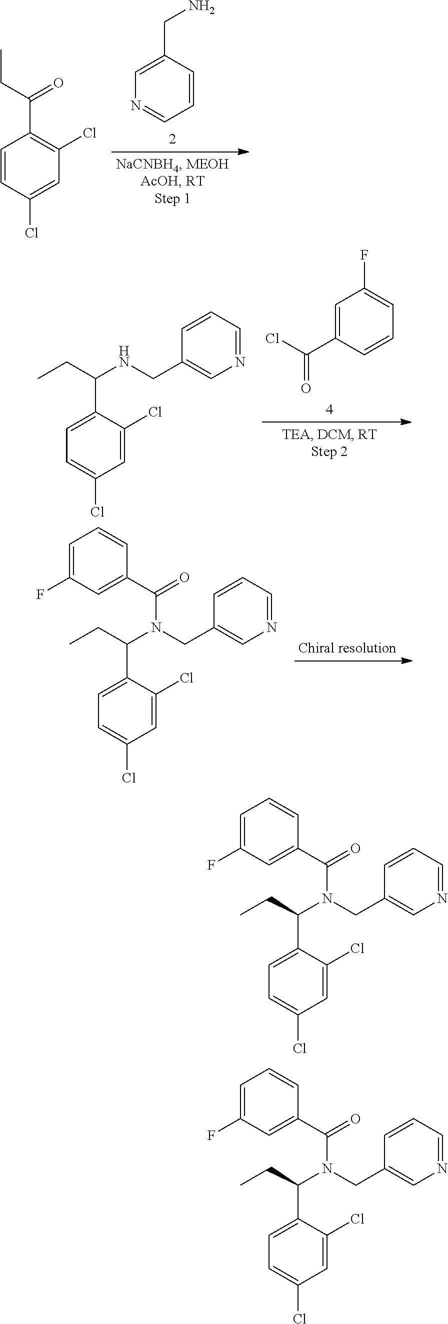

Step 1. Synthesis of 1-(2, 4-dichlorophenyl)-N-(pyridin-3-ylmethyl) propan-1-amine. A mixture of 1-(2,4-dichlorophenyl)propan-1-one (0.500 g, 2.46 mmol), Pyridin-3-ylmethanamine (0.32 g, 2.95 mmol) and Acetic acid (0.5 ml) in MeOH (5 ml) was allowed to stir at room temperature for 2 hours. Sodium cyanoborohydride (0.23 g, 3.69 mmol) and DCE (0.5 ml) were added, and the reaction mixture was stirred at room temperature for 16 hours. The reaction mixture was filtered through Celite. The filtrate was concentrated under reduced pressure to give title compound (0.730 g, crude) as an oil. LCMS: m/z 295.2 [M+H]⁺.

Step 2. Synthesis of N-(1-(2,4-dichlorophenyl)propyl)-3-fluoro-N-(pyridin-3-ylmethyl)benzamide. To a solution of 1-(2,4-dichlorophenyl)-N-(pyridin-3-ylmethyl)propan-1-amine (0.73 g, 2.47 mmol) in anhydrous DCM (7.6 ml), 3-fluorobenzoyl chloride (0.47 g, 2.96 mmol) was added under nitrogen atmosphere. Triethylamine (0.38 g, 3.71 mmol) was added drop wise at 0° C. The resulting reaction mixture was brought to room temperature and stirred for another 4 hours. After completion of reaction, water (30 ml) was slowly added, and the reaction mixture was extracted with DCM (2×100 ml). The combined organic layer was washed with Brine (20 ml), dried over Na₂SO₄. filtered, and concentrated under reduced pressure. The crude compound was purified by Combi-flash chromatography (RP Gold column) by using Acetonitrile and 0.1% formic acid in water to give the pure title compound (0.160 g, 15.53%) as solid. Chiral Prep HPLC: Water's PSFC-200 with UV Detector; Column: CHIRALCEL OX-H (250*21) mm, 5 u; Mobile Phase A: LIQUID·CO₂, Mobile Phase B: 0.1% DEA in Methanol; Isocratic gradient 82:18 (A:B), Column Flow: 80 mL/min (100 bar). FR-1 (Isomer 1): R_{T}=5.58; FR-2 (Isomer 2): R_{T}=7.97.

Isomer 1 Arbitrarily assigned as (R)—N-(1-(2,4-dichlorophenyl)propyl)-3-fluoro-N-(pyridin-3-ylmethyl)benzamide (Compound 157) LCMS: m/z 417.27 [M+H]⁺. ¹H NMR (400 MHz, DMSO-d₆): δ 0.80 (t, J=6.8 Hz, 3H), 2.07-2.15 (m, 1H), 2.21-2.23 (m, 1H), 4.44-4.55 (m, 2H), 5.35 (bs, 1H), 7.13-7.16 (m, 1H), 7.20-7.27 (m, 3H), 7.35 (d, J=7.2 Hz, 2H), 7.43-7.50 (m, 2H), 7.62 (d, J=8.8 Hz, 1H), 8.14 (s, 1H), 8.32 (d, J=4.0 Hz, 1H).

Isomer 2 Arbitrarily assigned as (S)—N-(1-(2,4-dichlorophenyl)propyl)-3-fluoro-N-(pyridin-3-ylmethyl)benzamide (Compound 158) LCMS: m/z 417.30 [M+H]⁺. ¹H NMR (400 MHz, DMSO-d₆): δ 0.80 (t, J=6.4 Hz, 3H), 2.07-2.15 (m, 1H), 2.21-2.23 (m, 1H), 4.43-4.55 (m, 2H), 5.35 (bs, 1H), 7.13-7.16 (m, 1H), 7.20-7.27 (m, 3H), 7.35 (d, J=8.0 Hz, 2H), 7.43-7.50 (m, 2H), 7.62 (d, J=8.8 Hz, 1H), 8.14 (s, 1H), 8.32 (d, J=4.4 Hz, 1H).

General Procedures IX

### Preparation of (R)—N-(1-(2,4-dichlorophenyl)-2-methylpropyl)-3-fluoro-N-(pyridin-3-ylmethyl)benzamide (Compound 159) and (S)—N-(1-(2,4-dichlorophenyl)-2-methylpropyl)-3-fluoro-N-(pyridin-3-ylmethyl)benzamide (Compound 160)

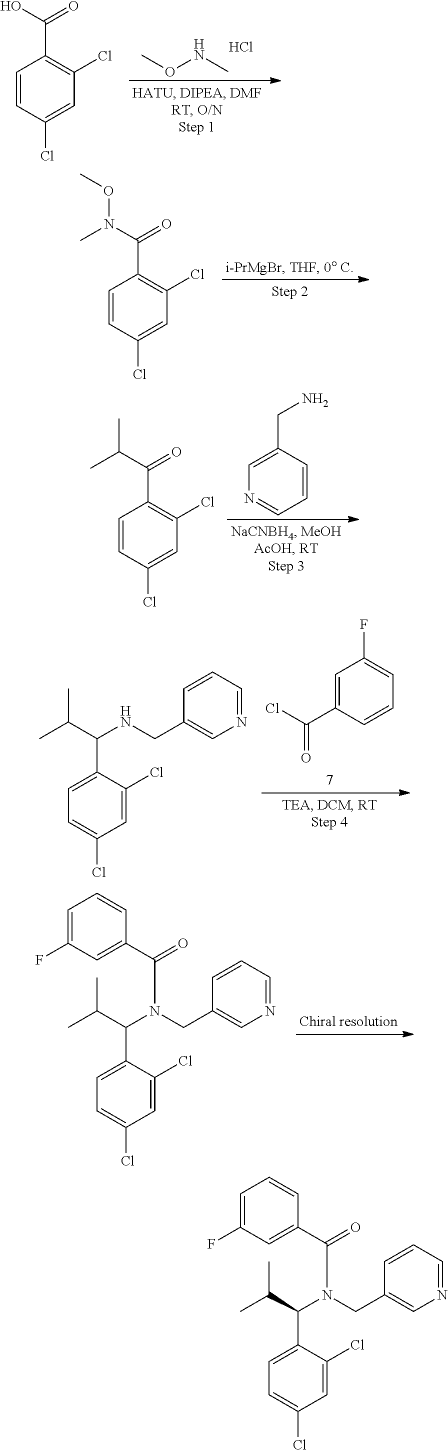

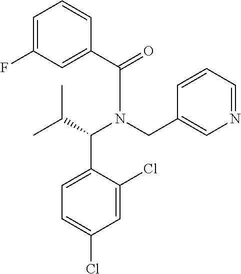 [SMILES: CC(C)[C@@H](c1ccc(Cl)cc1Cl)N(Cc1cccnc1)C(=O)c1cccc(F)c1]

Step 1. Synthesis of 2,4-dichloro-N-methoxy-N-methylbenzamide. To a stirred solution of 2,4-dichlorobenzoic acid (10 g, 52.35 mmol) in dry DMF (100 ml) was added N,O-dimethyl hydroxylamine hydrochloride (6.12 g, 62. 74 mmol) followed by the addition of HATU (29.0 g, 76.4 mmol) at 0° C. The reaction mixture was stirred at 0° C. for 1 hour. DIPEA (20.3 g, 157 mmol) was added dropwise at 0° C. and the resulting reaction mixture was stirred at room temperature for 4 hours. After completion of the reaction, water (250 ml) was slowly added, and the reaction was extracted with Ethyl acetate (2×100 ml). The combined organic layer was washed with Brine (200 ml), dried over Na₂SO₄. filtered, and concentrated under reduced pressure. The crude compound was purified by column chromatography to give title compound (8.0 g, 65%) as an oil. LCMS: m/z 234.1 [M+H]⁺.

Step 2. Synthesis of 1-(2, 4-dichlorophenyl)-2-methylpropan-1-one. A solution of 2, 4-dichloro-N-methoxy-N-methylbenzamide (0.47 g, 2.01 mmol) in dry THF (5 ml) was cooled to 0° C. under Nitrogen atmosphere. Isopropylmagnesium bromide (5 ml, 5.04 mmol) in THF was added dropwise at 0° C. The resulting reaction mixture was brought to room temperature and heated at 60° C. for 8 hours. After completion of reaction, saturated Ammonium chloride solution (5 ml) was slowly added, and the reaction mixture was extracted with Ethyl acetate (2×25 ml). The combined organic layer was washed with Brine (10 ml), dried over Na₂SO₄. filtered, and concentrated under reduced pressure. The crude compound was purified by column chromatography to give title compound (0.04 g, 9%) as an oil. ¹H NMR (400 MHz, DMSO-d₆): δ 1.065 (d, J=6.8 Hz, 6H), 3.25-3.35 (m, 1H), 7.55 (dd, J=8.4 Hz, 1.6 Hz, 1H), 7.67 (d, J=8.4 Hz, 1H), 7.75 (d, J=1.6 Hz, 1H).

Step 3. Synthesis of 1-(2, 4-dichlorophenyl)-2-methyl-N-(pyridin-3-ylmethyl) propan-1-amine. A mixture of 1-(2,4-dichlorophenyl)-2-methylpropan-1-one (1 g, 4.6 mmol), Pyridin-3-ylmethanamine (2 g, 18.5 mmol) and Acetic acid (1 ml) in DCE (10 ml) was stirred at room temperature for 2 hours. Sodium cyanoborohydride (0.43 g, 6.84 mmol) and Methanol (1 ml) were added, and the reaction mixture was heated at 60° C. for 24 hours. After completion of reaction, the reaction mixture was filtered through Celite and the filtrate was concentrated under reduced pressure. The crude compound was purified by Combiflash column chromatography (RP-Gold column) using Acetonitrile and 0.1% formic acid in water to give a pure title compound (0.260 g, 18%) as solid. LCMS: m/z 309.2 [M+H]⁺. ¹H NMR (400 MHz, DMSO-d₆): δ 0.75 (d, J=6.8 Hz, 3H), 0.95 (d, J=6.8 Hz 3H), 1.79-1.84 (m, 1H), 3.28-3.31 (m, 1H), 3.57 (d, J=14 Hz, 1H), 3.73-3.74 (m, 1H), 7.30-7.33 (m, 1H), 7.48 (d, J=7.2 Hz, 1H), 7.55 (s, 1H), 7.65-7.69 (m, 2H), 8.44 (s, 2H).

Step 4. Synthesis of N-(1-(2,4-dichlorophenyl)-2-methylpropyl)-3-fluoro-N-(pyridin-3-ylmethyl) benzamide. A solution of 1-(2,4-dichlorophenyl)-2-methyl-N-(pyridin-3-ylmethyl)propan-1-amine (0.26 g, 0.84 mmol) in DCM (2.6 ml) was cooled to 0° C. 3-Fluorobenzoyl chloride (0.198 g, 1.25 mmol) was added under nitrogen atmosphere followed by the drop wise addition of Triethylamine (0.35 g, 3.43 mmol) at 0° C. The resulting reaction mixture was brought to room temperature and stirred for another 4 hours. After completion of reaction, water (5 ml) was slowly added, and the reaction mixture was extracted with Ethyl acetate (2×15 ml). The combined organic layer was washed with Brine (15 ml), dried over Na₂SO₄. filtered, and concentrated under reduced pressure. The crude compound was purified by Combiflash column chromatography (RP Gold column) using Acetonitrile and 0.1% formic acid in water to give the pure title compound (0.040 g, 11%) as solid. Chiral Prep HPLC: Shimadzu LC-20AP with UV Detector: CHIRALPAK IH 250*21 mm, 5 u; Mobile Phase A: 0.1% DEA in Hexanes, Mobile Phase B: 0.1% DEA in Propan-2-ol: Methanol (50:50); Isocratic gradient 90:10 (A:B), Column Flow: 18 mL/min (100 bar). FR-1 (Isomer 1): R_{T}=10.71; FR-2 (Isomer 2): R_{T}=17.03.

Isomer 1 Arbitrarily assigned as (R)—N-(1-(2,4-dichlorophenyl)-2-methylpropyl)-3-fluoro-N-(pyridin-3-ylmethyl)benzamide (Compound 159) LCMS: m/z 431.1 [M+H]⁺. ¹H NMR (400 MHz, DMSO-d₆): δ 0.81 (d, J=6.4 Hz, 3H), 1.20 (d, J=6.4 Hz, 3H), 2.80-2.86 (m, 1H), 4.55-4.65 (m, 2H), 5.55 (bs, 1H), 7.10-7.19 (m, 3H), 7.23-7.29 (m, 3H), 7.43-7.48 (m, 1H), 7.53 (s, 1H), 7.77 (d, J=8.4 Hz, 1H), 8.03 (bs, 1H), 8.30 (d, J=3.6 Hz, 1H).

Isomer 2 Arbitrarily assigned as (S)—N-(1-(2,4-dichlorophenyl)-2-methylpropyl)-3-fluoro-N-(pyridin-3-ylmethyl)benzamide (Compound 160) LCMS: m/z 431.4 [M+H]⁺. ¹H NMR (400 MHz, DMSO-d₆): δ 0.81 (d, J=6.4 Hz, 3H), 1.20 (d, J=6.4 Hz, 3H), 2.80-2.86 (m, 1H), 4.55-4.65 (m, 2H), 5.55 (bs, 1H), 7.10-7.19 (m, 3H), 7.23-7.29 (m, 3H), 7.43-7.48 (m, 1H), 7.53 (s, 1H), 7.77 (d, J=8.4 Hz, 1H), 8.03 (bs, 1H), 8.30 (d, J=3.6 Hz, 1H).

General Procedures X

### Preparation of (R)—N-(1-(2,4-dichlorophenyl)-2-hydroxyethyl)-3-fluoro-N-(pyridin-3-ylmethyl)benzamide (Compound 161) and (S)—N-(1-(2,4-dichlorophenyl)-2-hydroxyethyl)-3-fluoro-N-(pyridin-3-ylmethyl)benzamide (Compound 162)

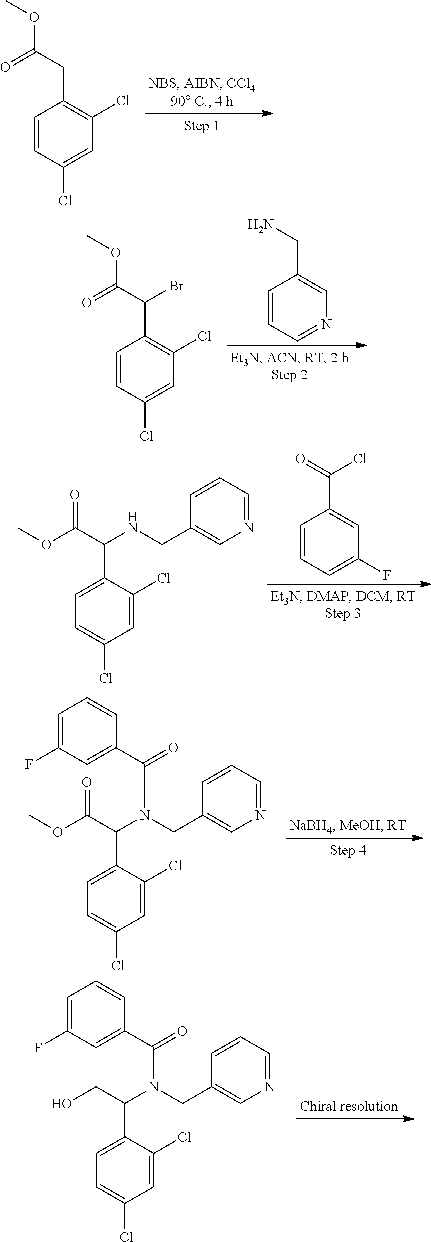

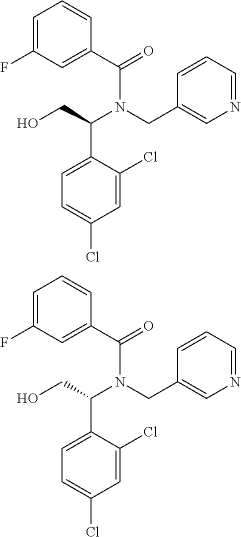

Step 1. Synthesis of Methyl 2-bromo-2-(2, 4-dichlorophenyl)acetate. To a stirred solution of Methyl 2-(2,4-dichlorophenyl)acetate (0.5 g, 2.28 mmol) in Carbon tetrachloride (5 ml) was added N-Bromosuccinimide (0.41 g, 2.28 mmol) and AIBN (0.074 g, 0.45 mmol). The reaction mixture was heated at 90° C. for 4 hours. After completion of the reaction, the solvent was evaporated and the crude product was purified by column chromatography to give title compound (0.6 g, 88%). ¹H NMR (400 MHz, CDCl₃): δ 3.83 (s, 3H), 5.88 (s, 1H), 7.34 (d, J=8.8 Hz, 1H), 7.44 (s, 1H), 7.76 (d, J=8.4 Hz, 1H).

Step 2. Synthesis of Methyl 2-(2, 4-dichlorophenyl)-2-((pyridin-3-ylmethyl)amino)acetate. To a solution of methyl 2-bromo-2-(2, 4-dichlorophenyl)acetate (0.4 g, 1.34 mmol) in anhydrous Acetonitrile (4 ml) under an atmosphere of Nitrogen, was added triethylamine (0.26 ml, 1.85 mmol) and 3-Picolylamine (0.116 mg, 1.07 mmol). The resulting reaction mixture was brought to room temperature and stirred for 5 hours. After completion of reaction, water (15 ml) was added slowly, and the reaction mixture was extracted with Ethyl acetate (3×10 ml). The combined organic layers were washed with Brine (10 ml), dried over Na₂SO₄. filtered, and concentrated under reduced pressure. The crude compound was purified by combi-flash chromatography to give the title compound (0.280 g, 64%) as gum. LCMS: m/z 325.2 [M⁺+1]. ¹H NMR (400 MHz, CDCl₃): δ 3.74 (s, 3H), 3.82 (s, 2H), 4.86 (s, 1H), 7.28-7.30 (bs, 2H), 7.39 (d, J=8.0 Hz, 1H), 7.45 (s, 1H), 7.71 (d, J=6.8 Hz, 1H), 8.56 (s, 2H).

Step 3. Synthesis of Methyl 2-(2,4-dichlorophenyl)-2-(3-fluoro-N-(pyridin-3-ylmethyl)benzamido) acetate. Methyl 2-(2, 4-dichlorophenyl)-2-((pyridin-3-ylmethyl)amino)acetate (0.28 g, 0.86 mmol), Triethylamine (0.66 ml, 2.06 mmol), DMAP (0.02 g, 0.16 mmol) and DCM (3 ml) was stirred for 5 minutes at room temperature under Nitrogen atmosphere. 3-Fluorobenzoyl chloride (0.204 g, 1.28 mmol) was added dropwise to the reaction mixture. The reaction mixture allowed to stir at room temperature for 16 hours. After completion of reaction, the reaction mixture was poured into solution of saturated Sodium bicarbonate (10 ml) and extracted with Dichloromethane (3×10 ml). The combined organic layer was washed with Brine (10 ml), dried over Na₂SO₄. filtered, and concentrated under reduced pressure. The crude compound was purified by column chromatography to give title compound (0.23 g, 59%) as gum. LCMS: m/z 447.3 [M+H]⁺.

Step 4. Synthesis of N-(1-(2,4-dichlorophenyl)-2-hydroxyethyl)-3-fluoro-N-(pyridin-3-ylmethyl) benzamide. To a solution of Methyl 2-(2,4-dichlorophenyl)-2-(3-fluoro-N-(pyridin-3-ylmethyl)benzamido)acetate (0.17 g, 0.39 mmol) in Methanol (6 ml), Sodium borohydride (0.298 g, 7.9 mmol) was added portion wise at 0° C. The resulting reaction mixture was brought to room temperature and stirred for another 16 hours. After completion of reaction, saturated ammonium chloride solution (50 ml) was added slowly, and the reaction mixture was extracted with Dichloromethane (2×30 ml). The combined organic layers were washed with Brine (20 ml), dried over Na₂SO₄. filtered, and concentrated under reduced pressure. The crude product was purified by column chromatography to give the title compound (0.12 g, 75%) as gum. Chiral Prep HPLC: Water's PSFC-200 with UV Detector; Column: CHIRALPAK IH (250*21) mm, 5 u; Mobile Phase A: Liquid CO₂, Mobile Phase B: 0.1% DEA in IPA: ACN (50:50); Isocratic gradient 80:20 (A:B), Column Flow: 80 mL/min (100 bar). FR-1 (Isomer 1): R_{T}=4.51 min; FR-2 (Isomer 2): R_{T}=9.80 min.

Isomer 1 Arbitrarily assigned as (R)—N-(1-(2,4-dichlorophenyl)-2-hydroxyethyl)-3-fluoro-N-(pyridin-3-ylmethyl)benzamide (Compound 161) LCMS: m/z 419.1 [M+H]⁺. ¹H NMR (400 MHz, DMSO-d₆): δ 3.92-3.97 (m, 1H), 4.07-4.11 (m, 1H), 4.49 (d, J=16.4 Hz, 1H), 4.62-4.66 (m, 1H), 5.10 (br s, 1H), 5.40 (br s, 1H), 7.15-7.18 (m, 1H), 7.25-7.47 (m, 7H), 7.62 (d, J=8.4 Hz, 1H), 8.20 (s, 1H), 8.32 (d, J=4.0 Hz, 1H).

Isomer 2 Arbitrarily assigned as (S)—N-(1-(2,4-dichlorophenyl)-2-hydroxyethyl)-3-fluoro-N-(pyridin-3-ylmethyl)benzamide (Compound 162) LCMS: m/z 419.1 [M+H]⁺. ¹H NMR (400 MHz, DMSO-d₆): δ 3.92-3.98 (m, 1H), 4.08-4.10 (m, 1H), 4.49 (d, J=16.0 Hz, 1H), 4.62-4.66 (m, 1H), 5.11 (br s, 1H), 5.40 (br s, 1H), 7.15-7.18 (m, 1H), 7.22-7.47 (m, 7H), 7.62 (d, J=8.0 Hz, 1H), 8.20 (s, 1H), 8.32 (d, J=4.4 Hz, 1H).

General Procedures XI

### Preparation of (R)—N-(1-(4-carbamoyl-2-chlorophenyl)ethyl)-3-fluoro-N-(pyridin-3-ylmethyl)benzamide (Compound 163) and (S)—N-(1-(4-carbamoyl-2-chlorophenyl)ethyl)-3-fluoro-N-(pyridin-3-ylmethyl)benzamide (Compound 164)

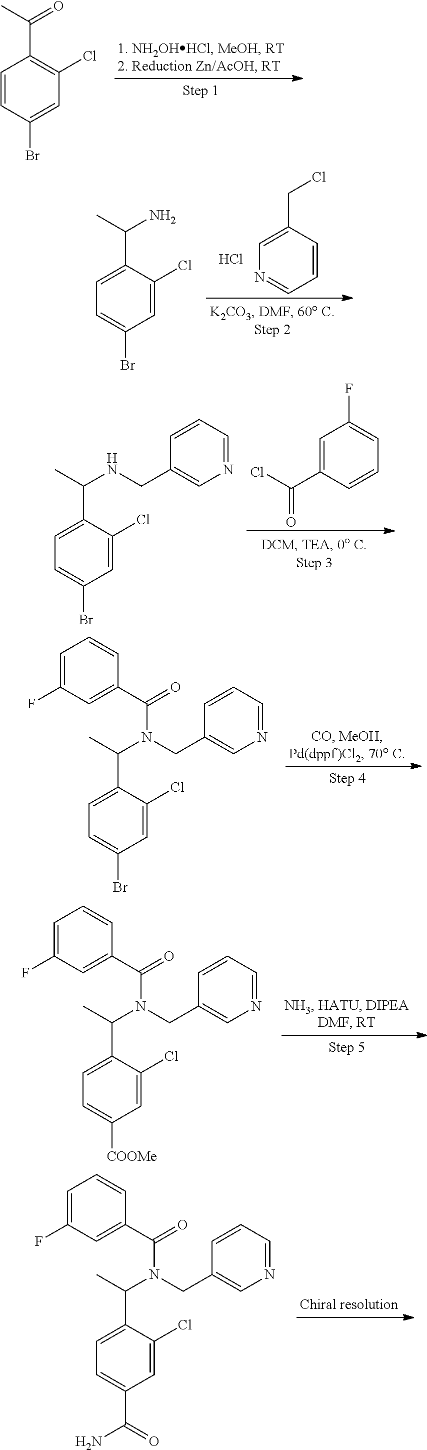

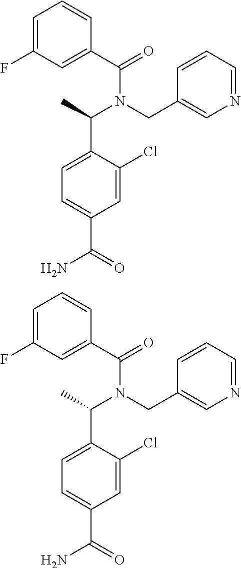

Step 1. Synthesis of 1-(4-bromo-2-chlorophenyl)ethan-1-amine. To a stirred solution of 1-(4-bromo-2-chlorophenyl)ethan-1-one (10 g, 42.82 mmol) in Methanol (200 ml), Molecular sieves (20 g) were added followed by the addition of hydroxylamine hydrochloride (8.93 g, 128.48 mmol). The reaction mixture was stirred at room temperature for 16 hours. After completion of reaction, the reaction mixture was filtered through Celite and the filtrate was evaporated to dryness. The crude solid was triturated with 10% Ethyl Acetate in Hexanes (20 ml) followed by n-pentane (20 ml) and decanted to obtain the 1-(4-bromo-2-chlorophenyl)ethan-1-one oxime (10 g). LCMS: m/z 249.7 [M+H]⁺.

1-(4-bromo-2-chlorophenyl)ethan-1-one oxime (10 g, 40.24 mmol) was dissolved in EtOH:AcOH (1:1, 200 ml) and Zinc powder (21.05 g, 322 mmol) was added portionwise at room temperature (caution: exothermic reaction). The reaction was stirred at room temperature for 18 hours. After completion of reaction, the reaction mixture was filtered through Celite and the filtrate was evaporated to dryness. The crude residue was neutralized with sodium bicarbonate solution to adjust pH to 8. The product was extracted with EtOAc (3×100 ml). Combined organic layer was dried over Na₂SO₄. filtered, and concentrated under reduced pressure. The crude compound was triturated with n-pentane (2×50 ml) and decanted to obtain the title compound (5.05 g, 50%, 2 steps). LCMS: m/z 235.8 [M+H]⁺.

Step 2. Synthesis 1-(4-bromo-2-chlorophenyl)-N-(pyridin-3-ylmethyl)ethan-1-amine. To a stirred mixture of 1-(4-bromo-2-chlorophenyl)ethan-1-amine (5.0 g, 21.32 mmol) in DMF (50 ml), Potassium carbonate (8.84 g, 64.0 mmol) was added. 3-(chloromethyl)pyridine. HCl (4.54 g, 27.7 mmol) was added at room temperature. The reaction mixture was heated to 60° C. for 16 hours. After completion of reaction, the reaction mixture was quenched with cold water 150 ml and extracted with Ethyl acetate (3×50 ml). The combined organic layer was washed with cold water (2×50 ml), dried over Na₂SO₄. filtered, and concentrated under reduced pressure. The crude compound was purified by RP Gold column chromatography using Acetonitrile and 0.1% formic acid in water to give the pure title compound (3.5 g, 50%). LCMS: m/z 326.0 [M+H]⁺.

Step 3. Synthesis of N-(1-(4-bromo-2-chlorophenyl)ethyl)-3-fluoro-N-(pyridin-3-ylmethyl)benzamide. To a stirred mixture of 1-(4-bromo-2-chlorophenyl)-N-(pyridin-3-ylmethyl)ethan-1-amine (3.5 g, 10.7 mmol) in DCM (35 ml), Et₃N (2.72 g, 26.9 mmol) was added at room temperature under nitrogen atmosphere. The reaction mixture was cooled to 0° C. and 3-Fluorobenzoylchloride (2.55 g, 16.1 mmol) was added dropwise. The reaction mixture was stirred at 15° C. for 1 hour. After completion of reaction, the reaction mixture was quenched with Sodium bicarbonate solution (35 ml) and extracted with DCM (2×35 ml). The combined organic layer was washed with water (2×50 ml) followed by Brine (50 ml), dried over Na₂SO₄. filtered, and concentrated under reduced pressure. The crude compound was purified by Combiflash column chromatography to give the title compound (3.0 g, 62%). LCMS: m/z 447.0 [M+H]⁺.

Step 4. Synthesis of Methyl 3-chloro-4-(1-(3-fluoro-N-(pyridin-3-ylmethyl)benzamido) ethyl)benzoate. To a stirred mixture of N-(1-(4-bromo-2-chlorophenyl)ethyl)-3-fluoro-N-(pyridin-3-ylmethyl)benzamide (1 g, 2.23 mmol) in MeOH (20 ml) were added Sodium acetate (0.531 g, 6.48 mmol), Palladium acetate (0.050 g, 0.23 mmol) and Pd(dppf)Cl₂ (0.163 g, 0.23 mmol) at room temperature in hydrogenator. Carbon monoxide (g) pressure (125 psi) was applied and hydrogenator was heated to 70° C. for 16 hours. After completion of reaction, reaction mixture was filtered through Celite and the filtrate was concentrated. The crude compound was purified by Combiflash column chromatography to give title compound (0.625 g, 65%). LCMS: m/z 427.0 [M+H]⁺.

Step 5. N-(1-(4-carbamoyl-2-chlorophenyl)ethyl)-3-fluoro-N-(pyridin-3-ylmethyl) benzamide. A stirred solution of Methyl 3-chloro-4-(1-(3-fluoro-N-(pyridin-3-ylmethyl)benzamido) ethyl)benzoate (0.625 g, 1.46 mmol) in MeOH (12.5 ml) was purged with Ammonia gas for 15-30 minutes at −78° C. The reaction mixture was stirred at room temperature for 16 hours. After completion of reaction, the reaction mixture was concentrated. The crude compound was purified by RP Gold column chromatography using Acetonitrile and 0.1% formic acid in water to give the pure title compound (0.160 g, 26%). Chiral Prep HPLC: Water's PSFC-200 with UV Detector; Column: CHIRALPAK IB-N (250*21) mm, 5 u; Mobile Phase A: LIQUID·CO₂, Mobile Phase B: 0.1% DEA in IPA: ACN (50:50); Isocratic gradient 78:22 (A:B), Column Flow: 80 mL/min (100 bar). FR-1 (Isomer 1): R_{T}=5.93 min; FR-2 (Isomer 2): R_{T}=6.28 min.

Isomer 1 Arbitrarily assigned as (R)—N-(1-(4-carbamoyl-2-chlorophenyl)ethyl)-3-fluoro-N-(pyridin-3-ylmethyl)benzamide (Compound 163) LCMS: m/z 412.0 [M+H]. ¹H NMR (400 MHz, DMSO-d₆): δ 1.64 (d, J=7.2 Hz, 3H), 4.49459 (, 2H), 5.50 (bs, 1H), 7.17-7.29 (m, 4H), 7.44-7.50 (i, 2H), 7.67 (d, J=8.0 Hz, 1H), 7.80-7.83 (i, 2H), 8.23 (bs, 1H), 8.33 (d, J=4.0 Hz, 1H).

Isomer 2 Arbitrarily assigned as (S)—N-(1-(4-carbamoyl-2-chlorophenyl)ethyl)-3-fluoro-N-(pyridin-3-ylmethyl)benzamide (Compound 164) LCMS: m/z 412.0 [M+H]⁺. ¹H NMR (400 MHz, DMSO-d₆): δ 1.64 (d, J=7.2 Hz, 3H), 4.49458 (m, 2H), 5.50 (bs, 1H), 7.17-7.28 (i, 4H), 7.45-7.50 (7, 2H), 7.67 (d, J=8.0 Hz, 1H), 7.80-7.83 (m, 2H), 8.23 (bs, 1(H), 8.33 (d, J=4.0 Hz, 1H).

The Following Compounds in Table 8 were Prepared in a Similar Manner to General Procedures VII-XI Described Above:

TABLE 8

| | | ¹H NMR (400 | (ES, m/z) |
|---|---|---|---|
| Compound ID | Structure | MHz, DMSO) δ | [M + H]⁺ |
| Compound 155 | 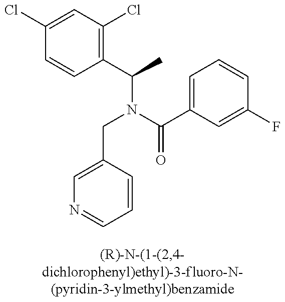 [SMILES: C[C@H](c1ccc(Cl)cc1Cl)N(Cc1cccnc1)C(=O)c1cccc(F)c1] | 1.66 (bs, 3H), 4.40 (d, J = 16.0 Hz, 1H), 4.67 (bs, 1H), 5.02-5.04 (m, 1H), 7.16-7.19 (m, 1H), 7.19-7.75 (m, 8H), 8.21 (bs, 1H), 8.32 (d, J = 4.4 Hz, 1H). | 403.58 |
| Compound 156 | 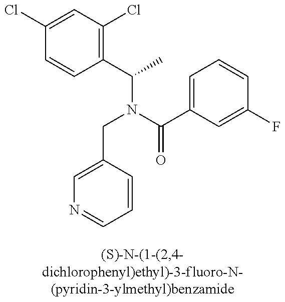 [SMILES: C[C@@H](c1ccc(Cl)cc1Cl)N(Cc1cccnc1)C(=O)c1cccc(F)c1] | 1.66 (bs, 3H), 4.40 (d, J = 15.6 Hz, 1H), 4.68 (bs, 1H), 5.04 (bs, 1H), 7.16- 7.19 (m, 1H), 7.19- 7.64 (m, 8H), 8.22 (bs, 1H), 8.32 (d, J = 4.0 Hz, 1H). | 403.57 |
| Compound 166 | 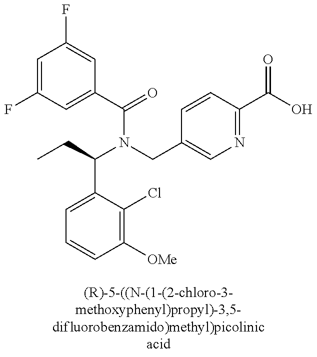 [SMILES: CC[C@H](c1cccc(OC)c1Cl)N(Cc1ccc(C(=O)O)nc1)C(=O)c1cc(F)cc(F)c1] | | 475.4 |
| Compound 167 | 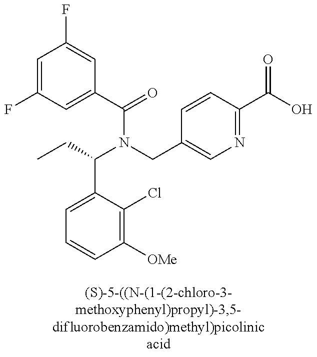 [SMILES: CC[C@@H](c1cccc(OC)c1Cl)N(Cc1ccc(C(=O)O)nc1)C(=O)c1cc(F)cc(F)c1] | | 475.1 |
| Compound 173 | 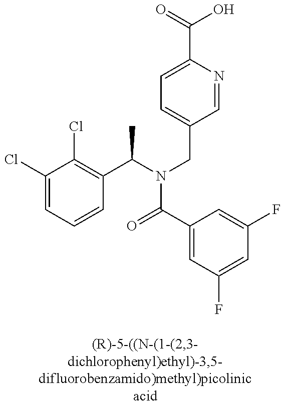 [SMILES: C[C@H](c1cccc(Cl)c1Cl)N(Cc1ccc(C(=O)O)nc1)C(=O)c1cc(F)cc(F)c1] | 8.32 (s, 1H), 7.84 (d, J = 8.0 Hz, 1H), 7.61 (td, J = 7.8, 1.9 Hz, 2H), 7.48 (dd, J = 8.0, 1.5 Hz, 1H), 7.35 (t, J = 7.9 Hz, 1H), 7.31- 7.21 (m, 1H), 7.17 (d, J = 6.1, 2H), 5.49 (s, 1H), 4.72-4.56 (m, 2H), 1.66 (d, J = 7.0 Hz, 3H) | 465.0 |
| Compound 174 | 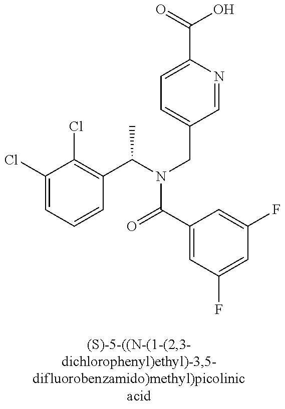 [SMILES: C[C@@H](c1cccc(Cl)c1Cl)N(Cc1ccc(C(=O)O)nc1)C(=O)c1cc(F)cc(F)c1] | 8.32 (s, 1H), 7.84 (d, J = 8.0 Hz, 1H), 7.61 (td, J = 8.1, 1.9 Hz, 2H), 7.48 (dd, J = 8.0, 1.5 Hz, 1H), 7.35 (t, J = 7.9 Hz, 1H), 7.27 (tt, J = 9.4, 2.4 Hz, 1H), 7.17 (dt, J = 6.1, 2.1 Hz, 2H), 5.49 (s, 1H), 4.64 (q, J = 16.7 Hz, 2H), 1.66 (d, J = 7.0 Hz, 3H). | 465.0 |
| Compound 254 | 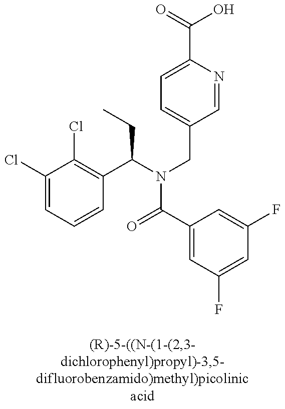 [SMILES: CC[C@H](c1cccc(Cl)c1Cl)N(Cc1ccc(C(=O)O)nc1)C(=O)c1cc(F)cc(F)c1] | | 479.3 |
| Compound 255 | 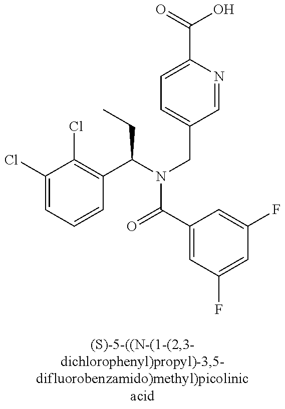 [SMILES: CC[C@H](c1cccc(Cl)c1Cl)N(Cc1ccc(C(=O)O)nc1)C(=O)c1cc(F)cc(F)c1] | | 479.3 |

### Synthesis of 2-(4-((N-(2,4-dichlorobenzyl)-3,5-difluorobenzamido)methyl)-1H-pyrazol-1-yl)acetic acid (Compound 165)

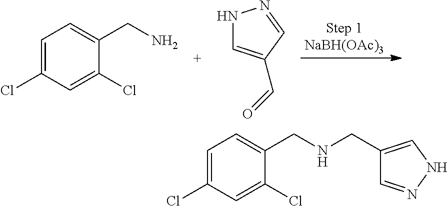

Step 1. 1H-pyrazole-4-carbaldehyde (1 g, 10.4 mmol) and 1-(2,4-dichlorophenyl)methanamine (2.00 g, 11.4 mmol) were combined in DCM (25 mL) and stirred at room temperature before the addition of sodium triacetoxyborohydride (3.30 g, 15.6 mmol). The mixture was stirred overnight under an atmosphere of nitrogen. The reaction was quenched with saturated NaHCO₃ solution and the product was extracted with DCM. The combined organic extracts were dried over Na₂SO₄, filtered, and concentrated. The residue was purified by reverse phase chromatography (Biotage 60 g C18 cartridge; 5-50% CH₃CN/H₂O with 0.1% formic acid gradient) to afford N-((1H-pyrazol-4-yl)methyl)-1-(2,4-dichlorophenyl)methanamine. ES m/z 256.0 [M+H]⁺.

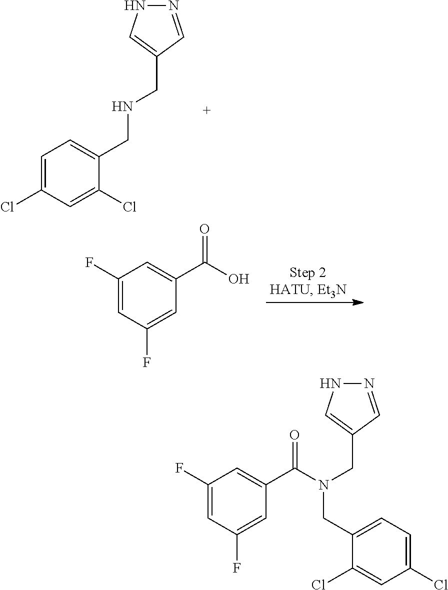 [SMILES: Clc1ccc(CNCc2cn[nH]c2)c(Cl)c1 ; O=C(O)c1cc(F)cc(F)c1 ; O=C(c1cc(F)cc(F)c1)N(Cc1cn[nH]c1)Cc1ccc(Cl)cc1Cl]

Step 2. N-((1H-pyrazol-4-yl)methyl)-1-(2,4-dichlorophenyl)methanamine (925 mg, 3.6 mmol) and 3,5-difluorobenzoic acid (573 mg, 3.6 mmol), Et3N (1.00 mL, 7.2 mmol), and HATU (1.64 g, 4.32 mmol) were combined in DMF (25 mL) and stirred at room temperature overnight. The reaction was quenched with saturated NaHCO₃ solution and the product was extracted with DCM. The combined organic extracts were dried over Na₂SO₄, filtered, and concentrated. The residue was purified by reverse phase chromatography (Biotage 60 g C18 cartridge; 5-50% CH₃CN/H₂O with 0.1% formic acid gradient) to afford N-((1H-pyrazol-4-yl)methyl)-N-(2,4-dichlorobenzyl)-3,5-difluorobenzamide. ES m/z 396.0 [M+H]⁺.

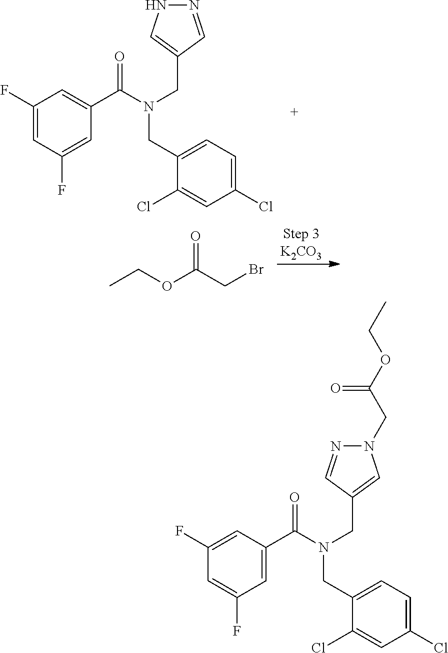

Step 3. N-((1H-pyrazol-4-yl)methyl)-N-(2,4-dichlorobenzyl)-3,5-difluorobenzamide (461 mg, 1.1 mmol) and K2CO3 (322 mg, 2.2 mmol) were combined in DMF (15 mL). After 15 minutes, ethyl 2-bromoacetate (183 mg, 1.1 mmol) was added. The reaction was stirred at room temperature overnight. The reaction quenched with saturated Brine solution and the product was extracted with DCM. The combined organic extracts were dried over Na₂SO₄, filtered, and concentrated. The residue was purified by reverse phase chromatography (Biotage 60 g C18 cartridge; 5-50% CH₃CN/H₂O with 0.1% formic acid gradient) to afford ethyl 2-(4-((N-(2,4-dichlorobenzyl)-3,5-difluorobenzamido)methyl)-1H-pyrazol-1-yl)acetate. ES m/z 482.1 [M+H]⁺.

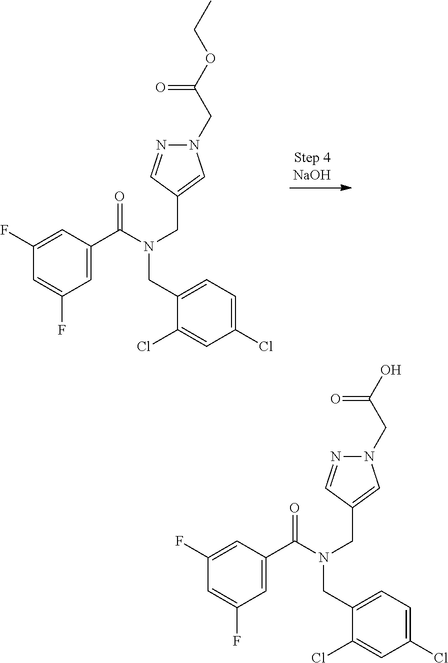 [SMILES: CCOC(=O)Cn1cc(CN(Cc2ccc(Cl)cc2Cl)C(=O)c2cc(F)cc(F)c2)cn1 ; O=C(O)Cn1cc(CN(Cc2ccc(Cl)cc2Cl)C(=O)c2cc(F)cc(F)c2)cn1]

Step 4. 40 mg ester 1 eq., 2 eq. 1M NaOH. ethyl 2-(4-((N-(2,4-dichlorobenzyl)-3,5-difluorobenzamido)methyl)-1H-pyrazol-1-yl)acetate (40 mg, 0.083 mmol) was dissolved in MeOH (1 mL) and 1M aq. NaOH (166 uL, 0.166 mmol) was added. The reaction was stirred at room temperature for 6 hours. The residue was purified by reverse phase chromatography (Biotage 60 g C18 cartridge; 5-50% CH₃CN/H₂O with 0.1% formic acid gradient) to afford 2-(4-((N-(2,4-dichlorobenzyl)-3,5-difluorobenzamido)methyl)-1H-pyrazol-1-yl)acetic acid. ES m/z 455.0 [M+H]⁺.

The Following Compounds in Table 9 were Prepared in a Similar Manner to Methods and Procedures Described Above:

TABLE 9

| | | ¹H NMR (400 | |
|---|---|---|---|
| | | MHz, DMSO) | (ES, m/z) |
| ID | Structure/Name | δ | [M + H]⁺ |
| Compound 169 | 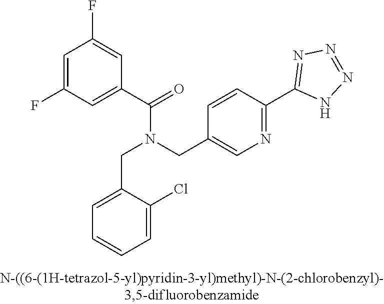 [SMILES: O=C(c1cc(F)cc(F)c1)N(Cc1ccc(-c2nnn[nH]2)nc1)Cc1ccccc1Cl] | | 441.4 |
| Compound 170 | 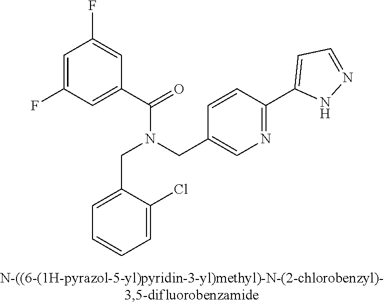 [SMILES: O=C(c1cc(F)cc(F)c1)N(Cc1ccc(-c2ccn[nH]2)nc1)Cc1ccccc1Cl] | | 439.4 |
| Compound 171 | 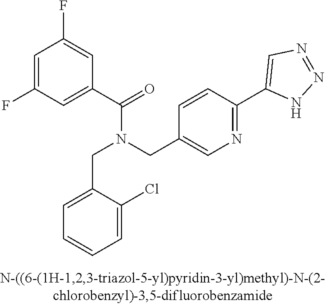 [SMILES: O=C(c1cc(F)cc(F)c1)N(Cc1ccc(-c2cnn[nH]2)nc1)Cc1ccccc1Cl] | | 440.5 |
| Compound 175 | 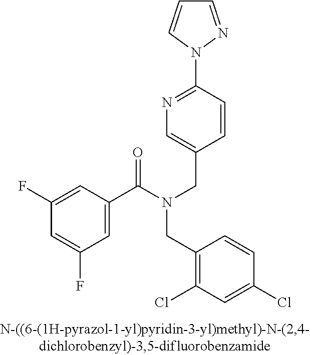 [SMILES: O=C(c1cc(F)cc(F)c1)N(Cc1ccc(-n2cccn2)nc1)Cc1ccc(Cl)cc1Cl] | | 473.0 |
| Compound 215 | 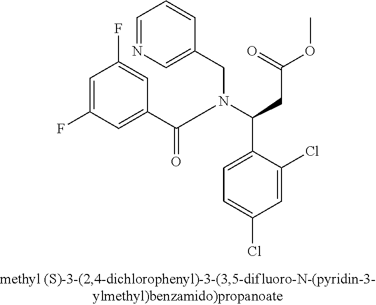 [SMILES: COC(=O)C[C@@H](c1ccc(Cl)cc1Cl)N(Cc1cccnc1)C(=O)c1cc(F)cc(F)c1] | 8.32 (d, J = 4.7 Hz, 1H), 8.15 (s, 1H), 7.64 (d, J = 8.4 Hz, 1H), 7.44 (s, 1H), 7.36 (t, J = 6.6 Hz, 2H), 7.28 (s, 1H), 7.15 (dd, J = 8.0, 4.4 Hz, 3H), 5.86 (s, 1H), 4.55 (s, 2H), 3.52 (s, 3H), 3.31 (d, J = 6.1 Hz, 2H). | 480.9 |
| Compound 216 | 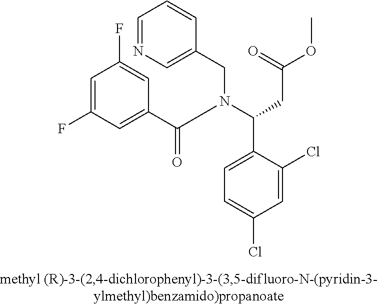 [SMILES: COC(=O)C[C@H](c1ccc(Cl)cc1Cl)N(Cc1cccnc1)C(=O)c1cc(F)cc(F)c1] | | 481.0 |
| Compound 217 | 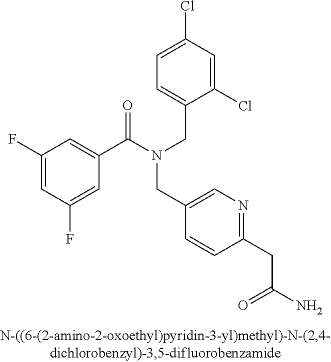 [SMILES: NC(=O)Cc1ccc(CN(Cc2ccc(Cl)cc2Cl)C(=O)c2cc(F)cc(F)c2)cn1] | 8.32 (s, 1H), 7.62 (d, J = 8.1 Hz, 1H), 7.54 (s, 1H), 7.42 (d, J = 2.7 Hz, 2H), 7.35-7.20 (m, 5H), 6.74 (s, 1H), 4.60 (d, J = 15.2 Hz, 4H), 3.59 (s, 2H), 3.10 (s, 1H). | 466.0 |
| Compound 218 | 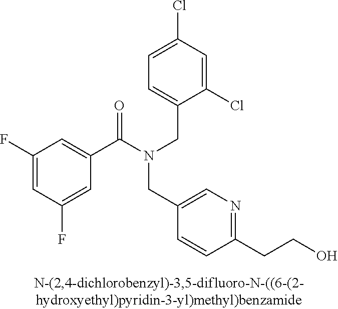 [SMILES: O=C(c1cc(F)cc(F)c1)N(Cc1ccc(CCO)nc1)Cc1ccc(Cl)cc1Cl] | 8.30 (s, 1H), 7.54 (d, J = 11.7 Hz, 2H), 7.41 (s, 2H), 7.29 (t, J = 9.4 Hz, 1H), 7.23 (d, J = 7.5 Hz, 3H), 4.62 (s, 2H), 4.57 (s, 2H), 4.41 (d, J = 5.5 Hz, 1H), 3.76 (q, J = 6.4 Hz, 2H), 2.89 (t, J = 6.8 Hz, 2H). | 452.9 |
| Compound 219 | 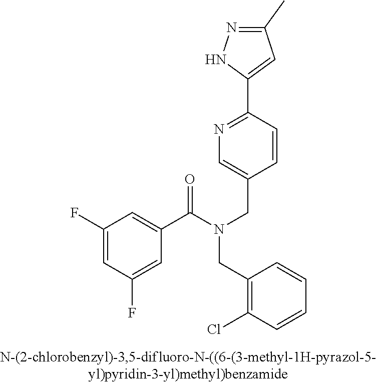 [SMILES: Cc1cc(-c2ccc(CN(Cc3ccccc3Cl)C(=O)c3cc(F)cc(F)c3)cn2)[nH]n1] | 12.52 (s, 1H), 8.38 (s, 1H), 7.83 (s, 1H), 7.68 (s, 1H), 7.45-7.24 (m, 4H), 7.28-7.20 (m, 2H), 6.57 (s, 1H), 4.69 (s, 2H), 4.62 (s, 2H), 2.29 (s, 3H), 1.31 (d, J = 17.3 Hz, 1H), 0.90 (t, J = 6.8 Hz, 1H). | 453.1 |
| Compound 220 | 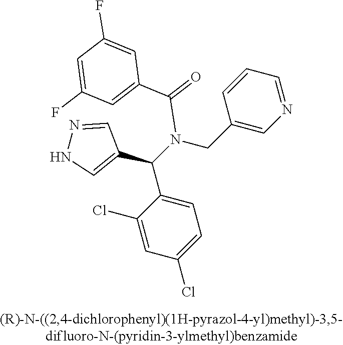 [SMILES: O=C(c1cc(F)cc(F)c1)N(Cc1cccnc1)[C@H](c1cn[nH]c1)c1ccc(Cl)cc1Cl] | 12.80 (s, 1H), 8.32 (d, J = 4.8 Hz, 1H), 8.10 (s, 1H), 7.53 (s, 2H), 7.46 (d, J = 8.4 Hz, 1H), 7.42-7.33 (m, 3H), 7.27 (t, J = 9.5 Hz, 1H), 7.14 (dd, J = 7.9, 4.7 Hz, 1H), 7.03 (d, J = 6.5 Hz, 2H), 6.47 (s, 1H), 4.90 (d, J = 16.3 Hz, 1H), 4.68 (d, J = 16.3 Hz, 1H). | 473.3 |
| Compound 221 | 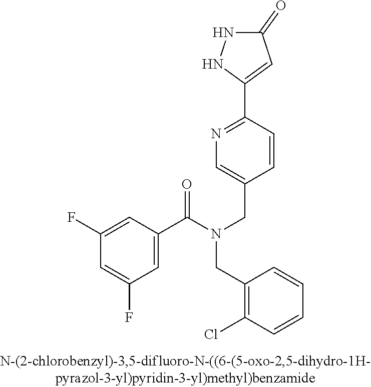 [SMILES: O=C(c1cc(F)cc(F)c1)N(Cc1ccc(-c2cc(=O)[nH][nH]2)nc1)Cc1ccccc1Cl] | 8.39 (s, 1H), 7.70 (s, 2H), 7.45-7.19 (m, 6H), 6.03 (s, 1H), 4.71-4.60 (m, 4H). | 455.6 |
| Compound 222 | 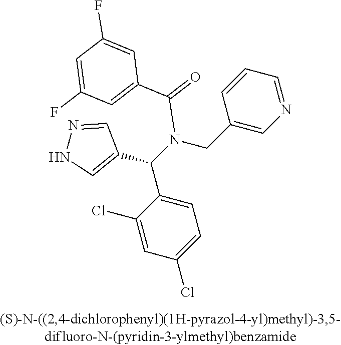 [SMILES: O=C(c1cc(F)cc(F)c1)N(Cc1cccnc1)[C@@H](c1cn[nH]c1)c1ccc(Cl)cc1Cl] | 12.77 (s, 1H), 8.31 (d, J = 4.7 Hz, 1H), 8.10 (s, 1H), 7.52 (s, 2H), 7.46 (d, J = 8.5 Hz, 1H), 7.39 (dd, J = 8.6, 2.2 Hz, 1H), 7.35 (s, 2H), 7.31-7.22 (m, 1H), 7.14 (dd, J = 7.9, 4.7 Hz, 1H), 7.06-6.99 (m, 2H), 6.46 (s, 1H), 4.89 (d, J = 16.3 Hz, 1H), 4.68 (d, J = 16.3 Hz, 1H). | 473.1 |
| Compound 223 | 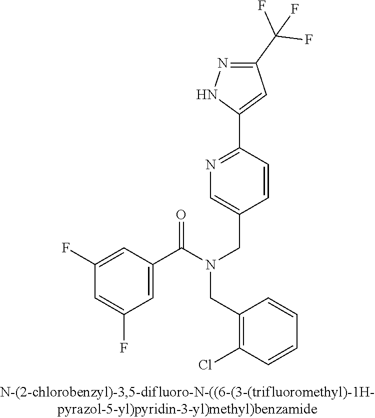 [SMILES: O=C(c1cc(F)cc(F)c1)N(Cc1ccc(-c2cc(C(F)(F)F)n[nH]2)nc1)Cc1ccccc1Cl] | 14.12 (s, 1H), 8.48 (s, 1H), 7.88 (d, J = 8.1 Hz, 1H), 7.82 (s, 1H), 7.42-7.30 (m, 2H), 7.33-7.10 (m, 3H), 4.69 (d, J = 15.6 Hz, 4H). | 507.3 |
| Compound 224 | 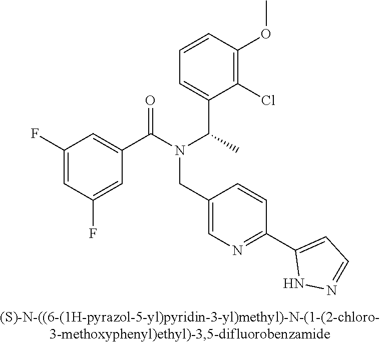 [SMILES: COc1cccc([C@H](C)N(Cc2ccc(-c3ccn[nH]3)nc2)C(=O)c2cc(F)cc(F)c2)c1Cl] | 8.19 (s, 1H), 7.70 (d, J = 16.5 Hz, 2H), 7.48 (d, J = 8.2 Hz, 1H), 7.37-7.23 (m, 2H), 7.21 (dd, J = 7.9, 1.4 Hz, 1H), 7.18-7.10 (m, 2H), 7.09-7.02 (m, 1H), 6.77 (d, J = 2.1 Hz, 1H), 4.54 (s, 2H), 3.77 (s, 3H), 1.63 (d, J = 7.0 Hz, 3H), 1.29 (d, J = 5.4 Hz, 2H), 0.90 (dt, J = 10.1, 7.1 Hz, 1H) | 483.3 |
| Compound 225 | 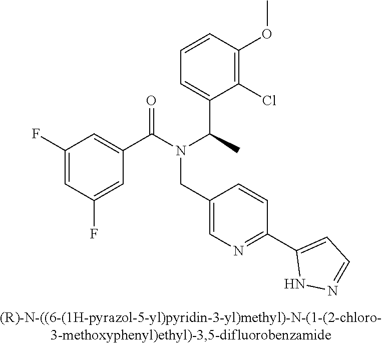 [SMILES: COc1cccc([C@@H](C)N(Cc2ccc(-c3ccn[nH]3)nc2)C(=O)c2cc(F)cc(F)c2)c1Cl] | 8.19 (s, 1H), 7.70 (d, J = 16.5 Hz, 2H), 7.48 (d, J = 8.2 Hz, 1H), 7.37-7.23 (m, 2H), 7.21 (dd, J = 7.9, 1.4 Hz, 1H), 7.18-7.10 (m, 2H), 7.09-7.02 (m, 1H), 6.77 (d, J = 2.1 Hz, 1H), 4.54 (s, 2H), 3.77 (s, 3H), 1.63 (d, J = 7.0 Hz, 3H), 1.29 (d, J = 5.4 Hz, 2H), 0.90 (dt, J = 10.1, 7.1 Hz, 1H) | 483.3 |
| Compound 226 | 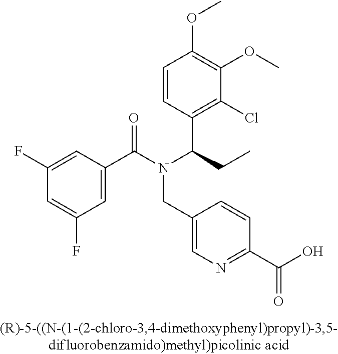 [SMILES: CC[C@H](c1ccc(OC)c(OC)c1Cl)N(Cc1ccc(C(=O)O)nc1)C(=O)c1cc(F)cc(F)c1] | 8.19 (s, 1H), 7.72 (s, 2H), 7.67 (s, 1H), 7.48 (d, J = 8.3 Hz, 1H), 7.37-7.09 (m, 5H), 7.06 (dd, J = 8.2, 1.3 Hz, 1H), 6.77 (d, J = 2.2 Hz, 1H), 5.50 (s, 1H), 4.53 (s, 2H), 3.77 (s, 3H), 2.61 (s, 1H), 1.63 (d, J = 7.0 Hz, 3H), 1.28 (s, 2H), 0.90 (q, J = 6.7 Hz, 1H). | |
| Compound 227 | 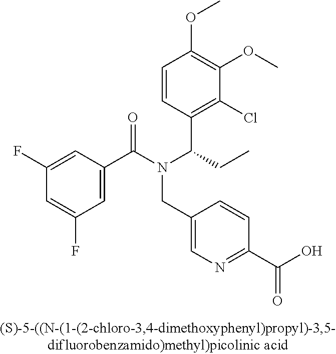 [SMILES: CC[C@@H](c1ccc(OC)c(OC)c1Cl)N(Cc1ccc(C(=O)O)nc1)C(=O)c1cc(F)cc(F)c1] | 8.22 (s, 1H), 7.77 (d, J = 8.0 Hz, 1H), 7.49 (d, J = 7.9 Hz, 1H), 7.28 (d, J = 8.7 Hz, 2H), 7.17 (s, 2H), 6.95 (d, J = 8.7 Hz, 1H), 4.66 (s, 1H), 4.47 (d, J = 16.7 Hz, 1H), 3.79 (s, 3H), 3.63 (s, 3H), 2.09 (dt, J = 14.5, 7.0 Hz, 1H), 0.79 (s, 4H). | |
| Compound 228 | 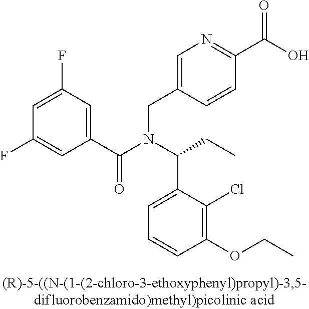 [SMILES: CCOc1cccc([C@@H](CC)N(Cc2ccc(C(=O)O)nc2)C(=O)c2cc(F)cc(F)c2)c1Cl] | 8.23 (s, 1H), 7.79 (d, J = 8.0 Hz, 1H), 7.52 (d, J = 8.1 Hz, 1H), 7.33-7.15 (m, 4H), 7.18-7.13 (m, 1H), 6.99 (dd, J = 8.1, 1.6 Hz, 1H), 5.35 (s, 1H), 4.64 (s, 1H), 4.53 (d, J = 16.6 Hz, 1H), 4.04 (dddd, J = 16.8, 13.9, 8.4, 4.9 Hz, 2H), 2.27 (s, 2H), 2.12 (ddd, J = 13.8, 7.5, 5.8 Hz, 1H), 1.33 (t, J = 6.9 Hz, 3H), 0.81 (t, J = 7.2 Hz, 3H). | 489.7 |
| Compound 229 | 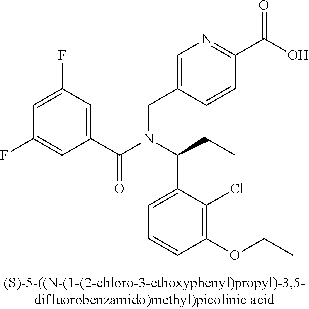 [SMILES: CCOc1cccc([C@H](CC)N(Cc2ccc(C(=O)O)nc2)C(=O)c2cc(F)cc(F)c2)c1Cl] | 8.23 (s, 1H), 7.79 (d, J = 8.0 Hz, 1H), 7.52 (d, J = 8.2 Hz, 1H), 7.25 (dd, J = 15.1, 7.2 Hz, 2H), 7.21-7.14 (m, 3H), 6.99 (dd, J = 8.1, 1.5 Hz, 1H), 5.35 (s, 1H), 4.63 (s, 1H), 4.53 (d, J = 16.6 Hz, 1H), 4.13-3.95 (m, 2H), 2.27 (s, 2H), 2.12 (ddd, J = 13.8, 7.5, 5.8 Hz, 1H), 1.32 (t, J = 6.9 Hz, 3H), 0.81 (t, J = 7.4 Hz, 3H). | 489.4 |
| Compound 230 | 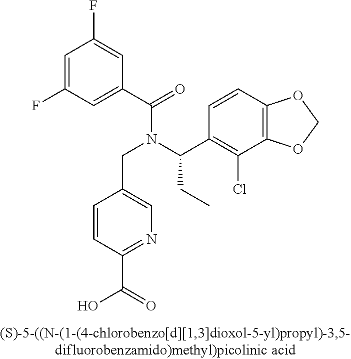 [SMILES: CC[C@@H](c1ccc2c(c1Cl)OCO2)N(Cc1ccc(C(=O)O)nc1)C(=O)c1cc(F)cc(F)c1] | 8.22 (s, 1H), 7.82 (d, J = 8.1 Hz, 1H), 7.53 (d, J = 8.2 Hz, 1H), 7.28 (t, J = 9.4 Hz, 1H), 7.18 (s, 2H), 7.08 (d, J = 8.2 Hz, 1H), 6.82 (d, J = 8.2 Hz, 1H), 6.10 (s, 1H), 5.97 (s, 1H), 4.61 (s, 1H), 4.49 (d, J = 16.7 Hz, 1H), 2.24 (s, 2H), 2.09 (dt, J = 13.6, 6.7 Hz, 1H), 0.82 (t, J = 7.4 Hz, 3H). | 489.3 |
| Compound 231 | 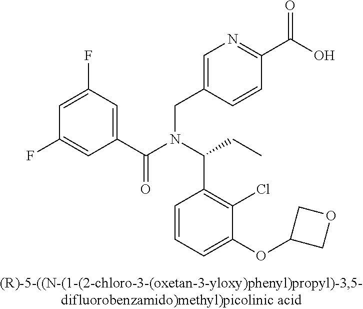 [SMILES: CC[C@H](c1cccc(OC2COC2)c1Cl)N(Cc1ccc(C(=O)O)nc1)C(=O)c1cc(F)cc(F)c1] | 8.21 (s, 1H), 7.79 (d, J = 8.1 Hz, 1H), 7.52 (d, J = 8.1 Hz, 1H), 7.34-7.15 (m, 5H), 6.65 (dd, J = 7.9, 1.6 Hz, 1H), 5.37 (s, 1H), 5.23 (p, J = 5.5 Hz, 1H), 4.90 (q, J = 6.0 Hz, 2H), 4.67 (s, 1H), 4.61-4.46 (m, 3H), 2.29 (s, 3H), 2.14 (dq, J = 13.8, 7.1 Hz, 1H), 0.82 (t, J = 7.1 Hz, 3H). | 517.4 |
| Compound 232 | 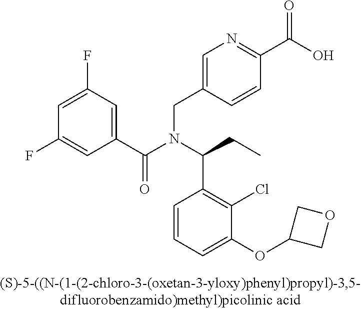 [SMILES: CC[C@@H](c1cccc(OC2COC2)c1Cl)N(Cc1ccc(C(=O)O)nc1)C(=O)c1cc(F)cc(F)c1] | 8.21 (s, 1H), 7.79 (d, J = 8.0 Hz, 1H), 7.52 (d, J = 8.1 Hz, 1H), 7.34-7.15 (m, 6H), 6.65 (d, J = 7.8 Hz, 1H), 5.37 (s, 1H), 5.23 (p, J = 5.5 Hz, 1H), 4.90 (q, J = 6.0 Hz, 2H), 4.66 (s, 1H), 4.61-4.46 (m, 3H), 2.29 (s, 2H), 2.13 (dt, J = 14.0, 6.9 Hz, 1H), 0.82 (t, J = 7.0 Hz, 3H). | 517.4 |
| Compound 233 | 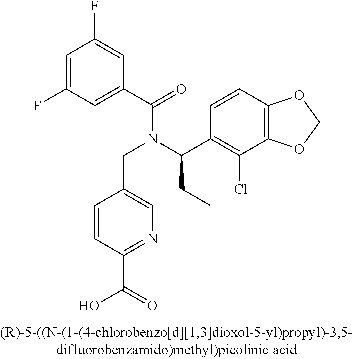 [SMILES: CC[C@H](c1ccc2c(c1Cl)OCO2)N(Cc1ccc(C(=O)O)nc1)C(=O)c1cc(F)cc(F)c1] | 8.22 (s, 1H), 7.82 (d, J = 8.1 Hz, 1H), 7.53 (d, J = 8.1 Hz, 1H), 7.28 (t, J = 9.5 Hz, 1H), 7.18 (s, 2H), 7.08 (d, J = 8.2 Hz, 1H), 6.82 (d, J = 8.2 Hz, 1H), 6.10 (d, J = 1.0 Hz, 1H), 5.97 (s, 1H), 5.28 (s, 1H), 4.62 (s, 1H), 4.49 (d, J = 16.6 Hz, 1H), 2.23 (s, 1H), 2.09 (dq, J = 13.7, 6.2 Hz, 1H), 0.83 (d, J = 8.4 Hz, 3H). | 489.3 |
| Compound 234 | 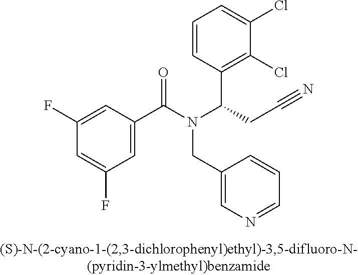 [SMILES: N#CC[C@@H](c1cccc(Cl)c1Cl)N(Cc1cccnc1)C(=O)c1cc(F)cc(F)c1] | 8.29 (dd, J = 4.8, 1.7 Hz, 1H), 8.05 (d, J = 2.5 Hz, 1H), 7.72 (dd, J = 7.9, 1.5 Hz, 1H), 7.55 (dd, J = 8.1, 1.4 Hz, 1H), 7.36 (t, J = 8.0 Hz, 1H), 7.29 (tt, J = 6.9, 3.8 Hz, 2H), 7.19 (d, J = 6.3 Hz, 2H), 7.12 (dd, J = 7.9, 4.7 Hz, 1H), 6.00 (s, 1H), 4.57 (d, J = 16.9 Hz, 1H), 4.44 (d, J = 16.7 Hz, 1H), 3.68-3.52 (m, 2H). | 446.3 |
| Compound 235 | 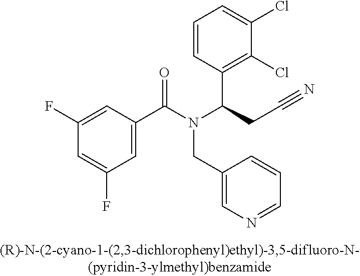 [SMILES: N#CC[C@H](c1cccc(Cl)c1Cl)N(Cc1cccnc1)C(=O)c1cc(F)cc(F)c1] | 8.32-8.26 (m, 1H), 8.05 (s, 1H), 7.72 (d, J = 7.8 Hz, 1H), 7.55 (dd, J = 8.1, 1.3 Hz, 1H), 7.36 (t, J = 8.0 Hz, 1H), 7.29 (d, J = 8.6 Hz, 2H), 7.19 (d, J = 6.2 Hz, 2H), 7.12 (dd, J = 7.9, 4.8 Hz, 1H), 6.00 (s, 1H), 4.62-4.49 (m, 1H), 4.44 (d, J = 16.8 Hz, 1H), 3.60 (d, J = 7.5 Hz, 2H). | 446.3 |
| Compound 248 | 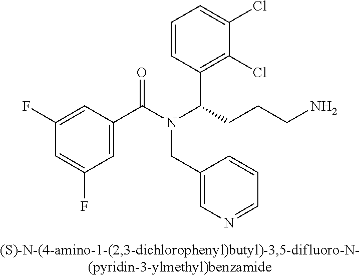 [SMILES: NCCC[C@@H](c1cccc(Cl)c1Cl)N(Cc1cccnc1)C(=O)c1cc(F)cc(F)c1] | | 464.3 |
| Compound 249 | 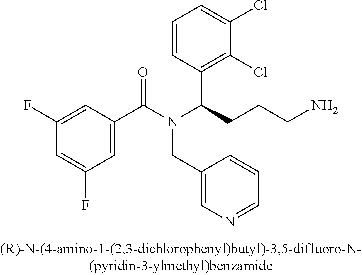 [SMILES: NCCC[C@H](c1cccc(Cl)c1Cl)N(Cc1cccnc1)C(=O)c1cc(F)cc(F)c1] | | 464.3 |
| Compound 236 | 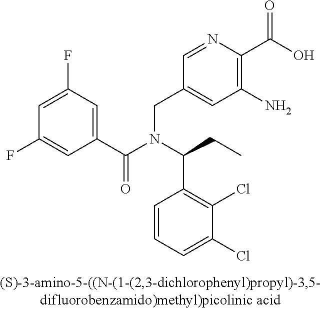 [SMILES: CC[C@@H](c1cccc(Cl)c1Cl)N(Cc1cnc(C(=O)O)c(N)c1)C(=O)c1cc(F)cc(F)c1] | 7.58 (dd, J = 7.9, 1.5 Hz, 1H), 7.52 (dd, J = 8.1, 1.5 Hz, 1H), 7.44 (s, 1H), 7.39-7.24 (m, 2H), 7.17 (d, J = 6.4 Hz, 2H), 6.92 (s, 1H), 5.43 (s, 1H), 4.46 (s, 2H), 2.21 (s, 2H), 2.13 (dt, J = 13.9, 6.9 Hz, 1H), 0.84 (t, J = 7.4 Hz, 3H). | 494.2 |
| Compound 237 | 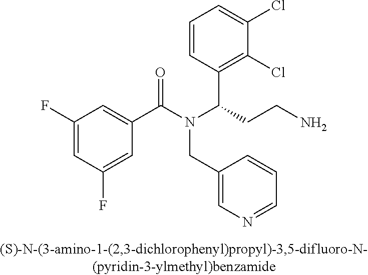 [SMILES: NCC[C@@H](c1cccc(Cl)c1Cl)N(Cc1cccnc1)C(=O)c1cc(F)cc(F)c1] | 8.50-8.39 (m, 3H), 7.69 (ddt, J = 7.7, 5.5, 2.3 Hz, 2H), 7.52 (dt, J = 7.8, 2.8 Hz, 1H), 7.48 (dd, J = 4.1, 2.2 Hz, 1H), 7.49-7.25 (m, 4H), 4.25 (d, J = 6.4 Hz, 1H), 3.61 (t, J = 8.8 Hz, 1H), 3.55 (s, 1H), 3.38 (s, 2H), 2.88 (s, 1H), 1.92 (qd, J = 7.4, 4.7 Hz, 2H), 1.28 (q, J = 6.1 Hz, 2H). | 450.3 |
| Compound 238 | 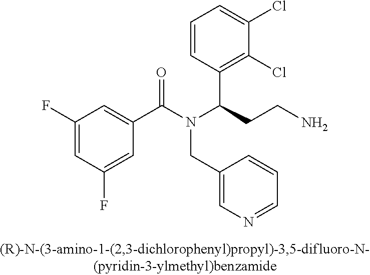 [SMILES: NCC[C@H](c1cccc(Cl)c1Cl)N(Cc1cccnc1)C(=O)c1cc(F)cc(F)c1] | 8.45 (s, 1H), 8.49-8.39 (m, 2H), 7.69 (ddd, J = 7.8, 6.0, 1.8 Hz, 2H), 7.60-7.40 (m, 3H), 7.43-7.25 (m, 2H), 4.26 (q, J = 6.8 Hz, 1H), 3.62 (dd, J = 13.8, 5.9 Hz, 1H), 3.53 (dd, J = 13.8, 6.3 Hz, 1H), 3.38 (q, J = 7.3 Hz, 2H), 1.28 (s, 3H), 2.88 (d, J = 7.2 Hz, 1H), 1.93 (qd, J = 7.5, 3.5 Hz, 2H). | 450.0 |
| Compound 239 | 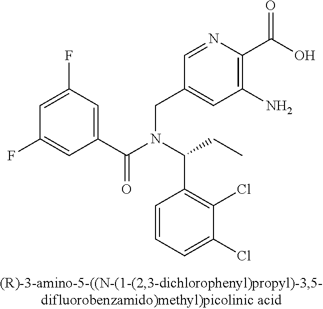 [SMILES: CC[C@H](c1cccc(Cl)c1Cl)N(Cc1cnc(C(=O)O)c(N)c1)C(=O)c1cc(F)cc(F)c1] | 7.58 (dd, J = 7.9, 1.6 Hz, 1H), 7.52 (dd, J = 8.1, 1.4 Hz, 1H), 7.44 (s, 1H), 7.39-7.24 (m, 2H), 7.17 (d, J = 6.4 Hz, 2H), 6.92 (s, 1H), 5.43 (s, 1H), 4.47 (s, 2H), 2.21 (s, 2H), 2.13 (dt, J = 13.8, 6.8 Hz, 1H), 0.84 (t, J = 7.2 Hz, 3H). | 494.2 |
| Compound 240 | 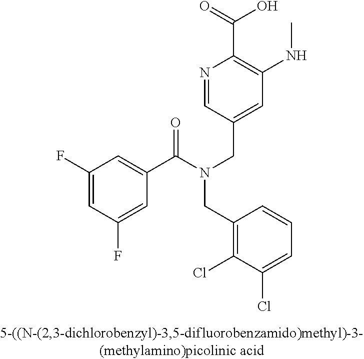 [SMILES: CNc1cc(CN(Cc2cccc(Cl)c2Cl)C(=O)c2cc(F)cc(F)c2)cnc1C(=O)O] | 7.72 (s, 1H), 7.56 (dt, J = 8.1, 4.0 Hz, 1H), 7.42-7.35 (m, 2H), 7.35-7.22 (m, 1H), 7.23 (d, J = 6.0 Hz, 2H), 6.96 (s, 1H), 4.75 (s, 2H), 4.64 (s, 2H), 2.86 (s, 3H). | 480.1 |
| Compound 241 | 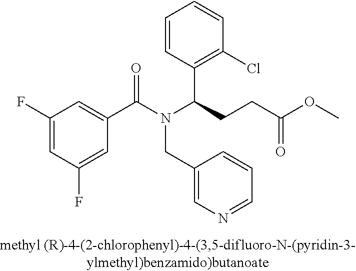 [SMILES: COC(=O)CC[C@H](c1ccccc1Cl)N(Cc1cccnc1)C(=O)c1cc(F)cc(F)c1] | 8.30 (dd, J = 4.7, 1.6 Hz, 1H), 8.10 (s, 1H), 7.62 (dd, J = 7.3, 2.1 Hz, 1H), 7.41-7.19 (m, 5H), 7.18-7.06 (m, 4H), 5.59 (s, 1H), 4.51 (s, 2H), 3.55 (s, 3H), 2.96 (s, 1H), 2.39 (dt, J = 14.0, 7.1 Hz, 1H), 1.20 (t, J = 7.3 Hz, 1H). | 459.4 |
| Compound 242 | 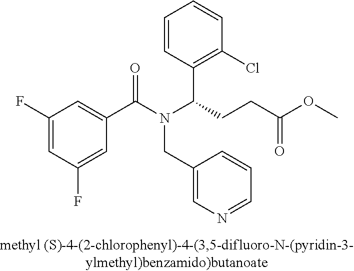 [SMILES: COC(=O)CC[C@@H](c1ccccc1Cl)N(Cc1cccnc1)C(=O)c1cc(F)cc(F)c1] | 8.30 (d, J = 4.8 Hz, 1H), 8.09 (s, 1H), 7.62 (dd, J = 7.4, 2.1 Hz, 1H), 7.41-7.19 (m, 5H), 7.14 (dd, J = 7.9, 4.8 Hz, 1H), 7.09 (d, J = 6.5 Hz, 2H), 5.59 (s, 1H), 4.51 (s, 2H), 3.55 (s, 3H), 2.45-2.23 (m, 1H), 1.27 (s, 1H), 1.27 (d, J = 12.3 Hz, 0H). | 459.4 |
| Compound 243 | 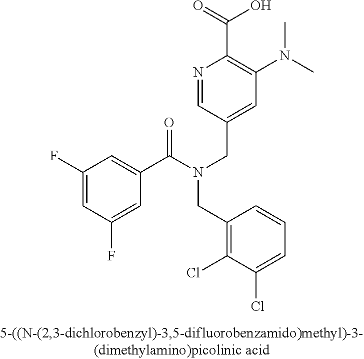 [SMILES: CN(C)c1cc(CN(Cc2cccc(Cl)c2Cl)C(=O)c2cc(F)cc(F)c2)cnc1C(=O)O] | 7.71 (s, 1H), 7.60-7.53 (m, 1H), 7.44-7.37 (m, 2H), 7.35-7.25 (m, 4H), 7.21-7.16 (m, 1H), 7.02 (s, 1H), 4.73 (d, J = 6.2 Hz, 3H), 4.23 (d, J = 4.3 Hz, 4H), 4.65 (d, J = 9.1 Hz, 3H), 2.88-2.78 (m, 3H). | 494.4 |
| Compound 244 | 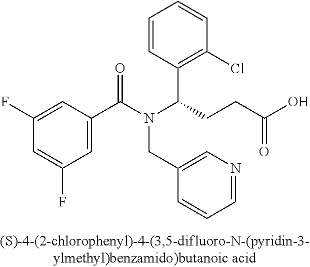 [SMILES: O=C(O)CC[C@@H](c1ccccc1Cl)N(Cc1cccnc1)C(=O)c1cc(F)cc(F)c1] | 8.31 (dd, J = 4.7, 1.7 Hz, 1H), 8.10 (s, 1H), 7.64 (dd, J = 7.4, 2.0 Hz, 1H), 7.42-7.18 (m, 6H), 7.18-7.06 (m, 4H), 5.64 (s, 2H), 4.52 (s, 3H), 2.41 (t, J = 7.1 Hz, 2H), 2.25 (dt, J = 14.3, 6.9 Hz, 1H), 2.19 (s, 2H). | 445.1 |
| Compound 245 | 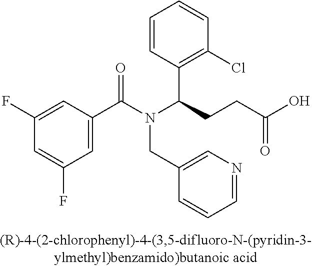 [SMILES: O=C(O)CC[C@H](c1ccccc1Cl)N(Cc1cccnc1)C(=O)c1cc(F)cc(F)c1] | 8.31 (dd, J = 4.7, 1.7 Hz, 1H), 8.10 (s, 1H), 7.64 (dd, J = 7.4, 2.0 Hz, 1H), 7.42-7.19 (m, 5H), 7.18-7.07 (m, 3H), 5.64 (s, 1H), 4.52 (s, 2H), 2.45-2.37 (m, 1H), 2.24 (q, J = 8.2 Hz, 1H), 2.18 (s, 1H). | 445.1 |
| Compound 246 | 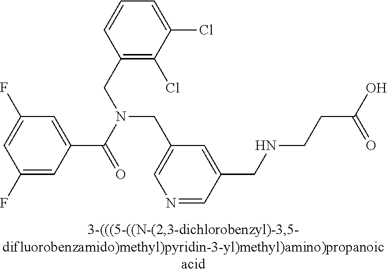 [SMILES: O=C(O)CCNCc1cncc(CN(Cc2cccc(Cl)c2Cl)C(=O)c2cc(F)cc(F)c2)c1] | | 508.19 |
| Compound 250 | 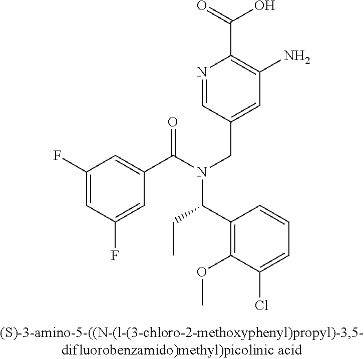 [SMILES: CC[C@@H](c1cccc(Cl)c1OC)N(Cc1cnc(C(=O)O)c(N)c1)C(=O)c1cc(F)cc(F)c1] | | 489.9 |
| Compound 251 | 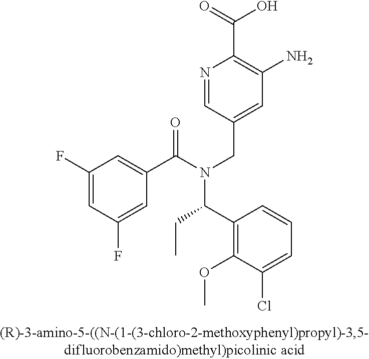 [SMILES: CC[C@@H](c1cccc(Cl)c1OC)N(Cc1cnc(C(=O)O)c(N)c1)C(=O)c1cc(F)cc(F)c1] | | 489.9 |
| Compound 253 | 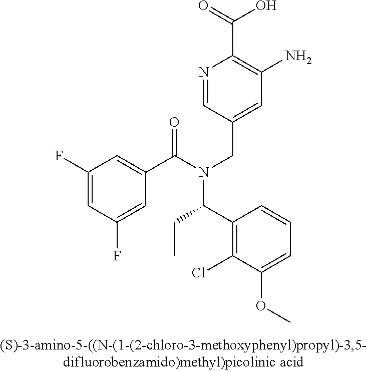 [SMILES: CC[C@@H](c1cccc(OC)c1Cl)N(Cc1cnc(C(=O)O)c(N)c1)C(=O)c1cc(F)cc(F)c1] | | 489.9 |
| Compound 252 | 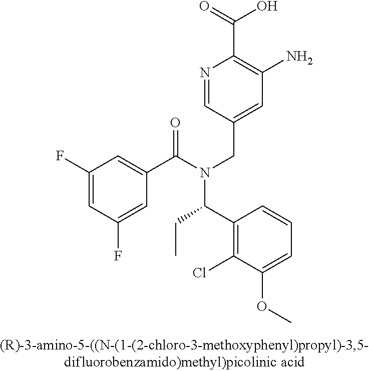 [SMILES: CC[C@@H](c1cccc(OC)c1Cl)N(Cc1cnc(C(=O)O)c(N)c1)C(=O)c1cc(F)cc(F)c1] | | 489.9 |
| Compound 256 | 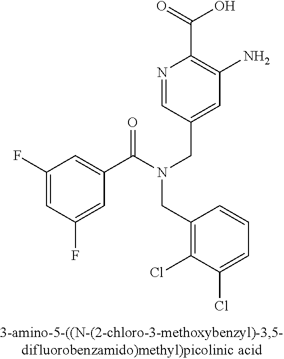 [SMILES: Nc1cc(CN(Cc2cccc(Cl)c2Cl)C(=O)c2cc(F)cc(F)c2)cnc1C(=O)O] | | 465.1 |
| Compound 257 | 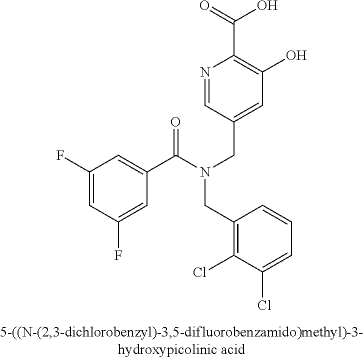 [SMILES: O=C(O)c1ncc(CN(Cc2cccc(Cl)c2Cl)C(=O)c2cc(F)cc(F)c2)cc1O] | | 466.0 |
| Compound 259 | 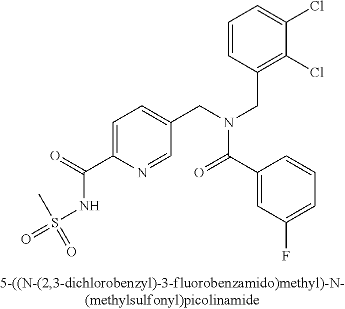 [SMILES: CS(=O)(=O)NC(=O)c1ccc(CN(Cc2cccc(Cl)c2Cl)C(=O)c2cccc(F)c2)cn1] | | 509.1 |
| Compound 260 | 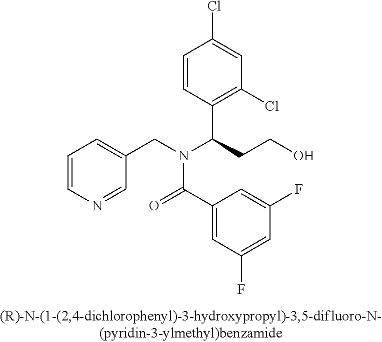 [SMILES: O=C(c1cc(F)cc(F)c1)N(Cc1cccnc1)[C@H](CCO)c1ccc(Cl)cc1Cl] | | 450.1 |
| Compound 261 | 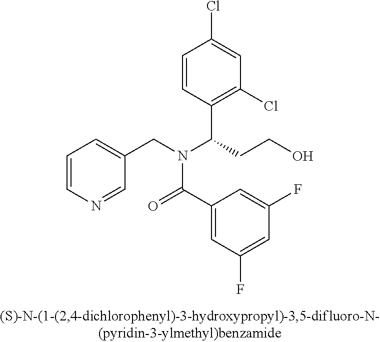 [SMILES: O=C(c1cc(F)cc(F)c1)N(Cc1cccnc1)[C@@H](CCO)c1ccc(Cl)cc1Cl] | | 450.1 |
| Compound 262 | 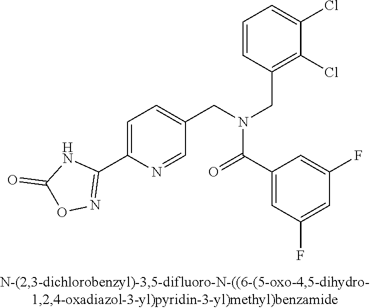 [SMILES: O=C(c1cc(F)cc(F)c1)N(Cc1ccc(-c2noc(=O)[nH]2)nc1)Cc1cccc(Cl)c1Cl] | | 490.0 |

Biochemical Assays

1. Silencing TREX1 in Tumor Cells

Activation of the cGAS/STING pathway upon sensing of cytosolic DNA and subsequent type I IFN production can occur in both tumor cells and innate immune cells, particularly dendritic cells. To evaluate whether TREX1 keeps in check the production of type I IFN by a well described cold syngeneic tumor model that undergoes immune-mediated rejection upon activation of type I IFN by STING agonists, TREX1 was knocked down in B16F10 tumor cells using CRISPR (FIG. 1A). Accumulation of cytosolic DNA via DNA transfection of the tumor cells resulted in an about 5-fold increase in IFNβ production by the TREX1 knockout B16F10 cells relative to the parental tumor cells, demonstrating that TREX1 attenuated the activation of the cGAS/STING pathway in B16F10 tumor cells (FIG. 1B).

2. Growth of TREX1-Competent and -Deficient B16F10 Tumor Cells In Vivo The growth of TREX1-competent and -deficient B16F10 tumor cells in vivo was evaluated. C57BL/6J mice were inoculated subcutaneously on the right flank with 300,000 parental or TREX1 knockout B16F10 tumor cells. Body weights were collected two times per week, and tumor measurements, two to three times per week, starting when tumors became measurable and for the remaining duration of the study. Tumors in which TREX1 had been silenced presented with remarkably smaller volumes than the parental B16F10 tumors (FIG. 2).

Tumors were harvested on day 19, upon termination of the study, and digested into single cell suspensions to enable flow cytometry quantification of tumor-infiltrating immune populations. TREX1 knockout B16F10 tumors were found to exhibit a significant increase in overall immune cells, which reflected an increase in the number of tumor infiltrating CD4 and CD8 T cells as well as in plasmacytoid dendritic cells (pDCs) (FIG. 3). pDCs are known to play a central role in the induction of antigen-specific anti-tumor immune responses whereas T cells are known to be major effectors of anti-tumor efficacy in mice and humans. The profound change in the immune infiltrate of the tumors deficient in TREX1 thus suggest that the inhibition of the growth of the latter tumors is at least in part immune-mediated.

### TREX1 Biochemical Assay

Compound potency was assessed through a fluorescence assay measuring degradation of a custom dsDNA substrate possessing a fluorophore-quencher pair on opposing strands. Degradation of the dsDNA liberates free fluorophore to produce a fluorescent signal. Specifically, 7.5 μL of N-terminally His-Tev tagged full length human TREX1 (expressed in E. coli and purified in house) in reaction buffer (50 mM Tris (pH 7.4), 150 mM NaCl, 2 mM DTT, 0.1 mg/mL BSA, 0.01% (v/v) Tween-20 and 100 mM MgCl₂) was added to a 384-well Black ProxiPlate Plus (Perkin Elmer) which already contained compound (150 nL) at varying concentrations as a 10-point dose-response in DMSO. To this was added 7.5 μL of dsDNA substrate (Strand A: 5′ TEX615/GCT AGG CAG 3′; Strand B: 5′ CTG CCT AGC/IAbRQSp (Integrated DNA Technologies)) in reaction buffer. Final concentrations were 150 pM TREX1, 60 nM dsDNA substrate in reaction buffer with 1.0% DMSO (v/v). After 25 minutes at room temperature, reactions were quenched by the addition of 5 μL of stop buffer (same as reaction buffer plus 200 mM EDTA). Final concentrations in the quenched reaction were 112.5 pM TREX1, 45 nM DNA and 50 mM EDTA in a volume of 20 μL. After a 5-minute incubation at room temperature, plates were read in a laser sourced Envision (Perkin-Elmer), measuring fluorescence at 615 nm following excitation w/570 nm light. IC₅₀ values were calculated by comparing the measured fluorescence at 615 nm ratio relative to control wells pre-quenched w/stop buffer (100% inhibition) and no inhibitor (0% inhibition) controls as using non-linear least square four parameter fits and either Genedata or GraphPad Prism (GraphPad Software, Inc.).

### TREX2 Biochemical Assay

Compound potency was assessed through a fluorescence assay measuring degradation of a custom dsDNA substrate possessing a fluorophore-quencher pair on opposing strands. Degradation of the dsDNA liberates free fluorophore to produce a fluorescent signal. Specifically, 7.5 μL of N-terminally His-Tev tagged human TREX2 (residues M44-A279, expressed in E. coli and purified in house) in reaction buffer (50 mM Tris (pH 7.4), 150 mM NaCl, 2 mM DTT, 0.1 mg/mL BSA, 0.01% (v/v) Tween-20 and 100 mM MgCl₂) was added to a 384-well Black ProxiPlate Plus (Perkin Elmer) which already contained compound (150 nL) at varying concentrations as a 10-point dose-response in DMSO. To this was added 7.5 μL of dsDNA substrate (Strand A: 5′ TEX615/GCT AGG CAG 3′; Strand B: 5′ CTG CCT AGC/IAbRQSp (IDT)) in reaction buffer. Final concentrations were 2.5 nM TREX2, 60 nM dsDNA substrate in reaction buffer with 1.0% DMSO (v/v). After 25 minutes at room temperature, reactions were quenched by the addition of 5 μL of stop buffer (same as reaction buffer plus 200 mM EDTA). Final concentrations in the quenched reaction mixture were 1.875 pM TREX2, 45 nM DNA and 50 mM EDTA in a volume of 20 μL. After a 5-minute incubation at room temperature, plates were read in a laser sourced Envision (Perkin-Elmer), measuring fluorescence at 615 nm following excitation w/570 nm light. IC₅₀ values were calculated by comparing the measured fluorescence at 615 nm ratio relative to control wells pre-quenched w/stop buffer (100% inhibition) and no inhibitor (0% inhibition) controls as using non-linear least square four parameter fits and either Genedata or GraphPad Prism (GraphPad Software, Inc.).

Results are shown in Table 10. TREX1 EC₅₀: A=<0.1 μM; B=0.1 to 1 μM; C=1 to 10 μM; D=>10 μM. TREX2 EC₅₀: A=<1 μM; B=1 to 10 μM; C=10 to 100 μM; D=>100 μM.

TABLE 10

| | hTREX1 | hTREX2 |
|---|---|---|
| Example | EC₅₀ | EC₅₀ |
| Compound 1 | D | D |
| Compound 2 | C | D |
| Compound 3 | D | |
| Compound 4 | D | |
| Compound 5 | C | D |
| Compound 6 | C | D |
| Compound 7 | D | |
| Compound 8 | D | |
| Compound 9 | D | |
| Compound 10 | D | |
| Compound 11 | C | C |
| Compound 12 | C | D |
| Compound 13 | C | |
| Compound 14 | D | |
| Compound 15 | D | |
| Compound 16 | D | |
| Compound 17 | D | |
| Compound 18 | D | |
| Compound 19 | C | D |
| Compound 20 | D | |
| Compound 21 | D | |
| Compound 22 | D | D |
| Compound 23 | D | D |
| Compound 24 | D | D |
| Compound 25 | C | D |
| Compound 26 | D | D |
| Compound 27 | D | D |
| Compound 28 | D | D |
| Compound 29 | D | D |
| Compound 30 | D | D |
| Compound 31 | D | D |
| Compound 32 | D | D |
| Compound 33 | D | D |
| Compound 34 | D | D |
| Compound 35 | D | D |
| Compound 36 | D | D |
| Compound 37 | D | D |
| Compound 38 | D | D |
| Compound 39 | D | D |
| Compound 40 | D | D |
| Compound 41 | D | D |
| Compound 42 | D | D |
| Compound 43 | D | D |
| Compound 44 | D | D |
| Compound 45 | D | D |
| Compound 46 | D | D |
| Compound 47 | D | D |
| Compound 48 | D | D |
| Compound 49 | D | D |
| Compound 50 | D | D |
| Compound 51 | D | D |
| Compound 52 | D | D |
| Compound 53 | D | C |
| Compound 54 | D | D |
| Compound 55 | D | D |
| Compound 56 | D | D |
| Compound 57 | D | D |
| Compound 58 | D | D |
| Compound 59 | D | D |
| Compound 60 | D | D |
| Compound 61 | D | D |
| Compound 62 | D | D |
| Compound 63 | D | D |
| Compound 64 | D | D |
| Compound 65 | C | D |
| Compound 66 | D | D |
| Compound 67 | D | D |
| Compound 68 | D | D |
| Compound 69 | D | D |
| Compound 70 | D | D |
| Compound 71 | D | D |
| Compound 72 | D | D |
| Compound 73 | D | D |
| Compound 74 | D | D |
| Compound 75 | D | D |
| Compound 76 | D | D |
| Compound 77 | D | D |
| Compound 78 | D | D |
| Compound 79 | D | D |
| Compound 80 | D | D |
| Compound 81 | D | D |
| Compound 82 | D | D |
| Compound 83 | D | D |
| Compound 84 | D | D |
| Compound 85 | D | D |
| Compound 86 | D | D |
| Compound 87 | D | D |
| Compound 88 | D | D |
| Compound 89 | D | D |
| Compound 90 | D | D |
| Compound 91 | D | D |
| Compound 92 | D | D |
| Compound 93 | D | D |
| Compound 94 | D | D |
| Compound 95 | D | D |
| Compound 96 | D | D |
| Compound 97 | D | D |
| Compound 98 | D | D |
| Compound 99 | D | D |
| Compound 100 | D | D |
| Compound 101 | C | D |
| Compound 102 | D | D |
| Compound 103 | D | D |
| Compound 104 | D | D |
| Compound 105 | D | D |
| Compound 106 | C | D |
| Compound 107 | D | D |
| Compound 108 | D | D |
| Compound 109 | D | D |
| Compound 110 | D | D |
| Compound 111 | C | D |
| Compound 112 | D | D |
| Compound 113 | D | D |
| Compound 114 | D | D |
| Compound 115 | D | D |
| Compound 116 | D | D |
| Compound 117 | D | D |
| Compound 118 | D | D |
| Compound 119 | B | C |
| Compound 120 | D | D |
| Compound 121 | B | C |
| Compound 122 | D | D |
| Compound 123 | C | D |
| Compound 124 | C | D |
| Compound 125 | D | D |
| Compound 126 | D | D |
| Compound 127 | C | D |
| Compound 128 | C | D |
| Compound 129 | D | D |
| Compound 130 | C | C |
| Compound 131 | D | D |
| Compound 132 | D | D |
| Compound 133 | D | D |
| Compound 134 | D | D |
| Compound 135 | C | C |
| Compound 136 | B | C |
| Compound 137 | B | C |
| Compound 138 | D | D |
| Compound 139 | C | D |
| Compound 140 | D | D |
| Compound 141 | D | D |
| Compound 142 | B | C |
| Compound 143 | D | D |
| Compound 144 | C | D |
| Compound 145 | C | C |
| Compound 146 | C | D |
| Compound 147 | A | B |
| Compound 148 | A | A |
| Compound 149 | D | D |
| Compound 150 | C | D |
| Compound 151 | B | C |
| Compound 152 | A | B |
| Compound 153 | D | D |
| Compound 154 | D | D |
| Compound 155 | B | C |
| Compound 156 | D | D |
| Compound 157 | B | C |
| Compound 158 | C | C |
| Compound 159 | C | C |
| Compound 160 | C | C |
| Compound 161 | D | C |
| Compound 162 | B | C |
| Compound 163 | B | C |
| Compound 164 | D | C |
| Compound 165 | C | D |
| Compound 166 | A | B |
| Compound 167 | A | B |
| Compound 168 | C | C |
| Compound 169 | B | C |
| Compound 170 | A | C |
| Compound 171 | B | C |
| Compound 172 | A | B |
| Compound 173 | B | C |
| Compound 174 | A | A |
| Compound 175 | B | D |
| Compound 215 | C | C |
| Compound 216 | C | C |
| Compound 260 | A | B |
| Compound 261 | B | C |
| Compound 213 | D | D |
| Compound 214 | C | D |
| Compound 262 | B | B |
| Compound 219 | C | C |
| Compound 193 | B | C |
| Compound 195 | A | B |
| Compound 194 | A | B |
| Compound 254 | A | B |
| Compound 255 | A | A |
| Compound 179 | D | C |
| Compound 220 | B | C |
| Compound 221 | B | C |
| Compound 222 | C | C |
| Compound 205 | A | A |
| Compound 181 | B | B |
| Compound 199 | C | C |
| Compound 223 | D | D |
| Compound 210 | C | C |
| Compound 256 | A | A |
| Compound 192 | D | D |
| Compound 247 | B | C |
| Compound 224 | C | C |
| Compound 200 | B | C |
| Compound 225 | B | C |
| Compound 226 | A | B |
| Compound 227 | A | B |
| Compound 228 | A | B |
| Compound 229 | A | B |
| Compound 201 | A | B |
| Compound 198 | B | C |
| Compound 202 | A | B |
| Compound 203 | A | B |
| Compound 204 | B | B |
| Compound 208 | A | A |
| Compound 176 | B | B |
| Compound 230 | A | B |
| Compound 231 | B | B |
| Compound 232 | A | C |
| Compound 233 | A | B |
| Compound 197 | A | B |
| Compound 196 | A | A |
| Compound 212 | B | B |
| Compound 211 | A | B |
| Compound 234 | C | C |
| Compound 235 | A | B |
| Compound 180 | B | B |
| Compound 236 | A | A |
| Compound 237 | D | C |
| Compound 238 | D | C |
| Compound 239 | A | B |
| Compound 240 | A | C |
| Compound 178 | C | C |
| Compound 177 | B | C |
| Compound 209 | A | C |
| Compound 241 | C | C |
| Compound 242 | A | C |
| Compound 243 | C | C |
| Compound 248 | A | C |
| Compound 244 | A | C |
| Compound 249 | C | C |
| Compound 245 | C | C |
| Compound 257 | A | C |
| Compound 206 | A | C |
| Compound 250 | A | C |
| Compound 251 | A | C |
| Compound 252 | A | C |
| Compound 253 | A | C |
| Compound 217 | B | D |
| Compound 207 | B | C |
| Compound 218 | B | C |
| Compound 258 | C | C |
| Compound 259 | C | D |
| Compound 184 | B | C |
| Compound 186 | A | B |
| Compound 185 | B | C |
| Compound 190 | A | B |
| Compound 187 | A | B |
| Compound 188 | A | B |
| Compound 182 | C | C |
| Compound 183 | B | C |
| Compound 191 | A | B |
| Compound 189 | B | C |

While we have described a number of embodiments, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A compound having the Formula I: 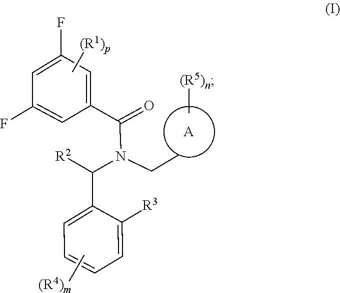
or a pharmaceutically acceptable salt thereof, wherein
R¹ is halo, hydroxy, (C₁-C₄)alkyl, halo(C₁-C₄)alkyl, (C₁-C₄)alkoxy, or halo(C₁-C₄)alkoxy;
R² is hydrogen, (C₁-C₄)alkyl, halo(C₁-C₄)alkyl, —(C₁-C₄)alkylOR^{a}, —C(O)R^{b}, —C(O)OR^{b}, —C(O)NR^{b}R^{c}, —C(O)NR^{b}R^{c}, 5- to 7-membered heteroaryl, or 4- to 7-membered heterocyclyl, wherein said heteroaryl and heterocyclyl are each optionally substituted with 1 to 3 groups selected from R⁶;
R³ is halo, (C₁-C₄)alkyl, halo(C₁-C₄)alkyl, (C₁-C₄)alkoxy, halo(C₁-C₄)alkoxy, —NR^{b}R^{c}, or CN;
R⁴ is halo, hydroxy, (C₁-C₄)alkyl, halo(C₁-C₄)alkyl, (C₁-C₄)alkoxy, halo(C₁-C₄)alkoxy, CN, —NR^{b}R^{c}, —C(O)R^{b}, —C(O)OR^{b}, —C(O)NR^{b}R^{c}, —SR^{b}, —C(O)NR^{b}R^{c}, —S(O)₂R^{b}, —S(O)R^{b}, —NR^{b}C(O)R^{c}, —NR^{b}C(O)OR^{c}, —NR^{b}C(S)OR^{c}, and —NR^{b}C(O)N^{c}R^{d};
Ring A is nitrogen-containing 5- to 7-membered heteroaryl;
R⁵ is halo, hydroxy, (C₁-C₄)alkyl, halo(C₁-C₄)alkyl, (C₁-C₄)alkoxy, halo(C₁-C₄)alkoxy, oxo, —(C₁-C₄)alkylOR^{d}, —(C₁-C₄)alkylC(O)R^{d}, —(C₁-C₄)alkylC(O)OR^{e}, —(C₁-C₄)alkylC(O)NR^{d}R^{e}, —C(O)R^{d}, —C(O)OR^{d}, —C(O)NR^{d}R^{e}, —C(O)NR^{d}SO₂(C₁-C₄)alkyl, CN, —NR^{d}R^{e}, —C(O)NR^{d}SO₃H, —NR^{d}C(O)R^{d}, —NR^{d}C(O)OR^{e}, —NR^{d}C(S)OR^{d}, —NR^{d}C(O)N^{e}R^{f}, —NR^{d}C(S)NR^{e}R^{f}, —N^{d}S(O)₂NR^{e}R^{f}, —C(S)R^{d}, —S(O)₂R^{d}, —S(O)R^{d}, —C(S)OR^{d}, —C(S)NR^{d}R^{e}, —NR^{d}C(S)R^{e}, —SR^{d}, —(C₁-C₄)alkylC(O)NR^{d}R^{e}, —(C₁-C₄)alkylC(O)NR^{d}SO₂(C₁-C₄)alkyl, —(C₁-C₄)alkylNR^{d}R^{e}, —(C₁-C₄)alkylC(O)NR^{d}SO₃H, —(C₁-C₄)alkylNR^{d}C(O)R^{e}, —(C₁-C₄)alkylNR^{d}C(O)OR^{e}, —(C₁-C₄)alkylNR^{d}C(S)OR^{e}, —(C₁-C₄)alkylNR^{d}C(O)N^{e}R^{f}, —(C₁-C₄)alkylNR^{d}C(S)NR^{e}R^{f}, —(C₁-C₄)alkylNR^{d}S(O)₂NR^{e}R^{f}, —(C₁-C₄)alkylC(S)R^{d}, —(C₁-C₄)alkylS(O)₂R^{d}, —(C₁-C₄)alkylS(O)R^{d}, —(C₁-C₄)alkylC(S)OR^{d}, —(C₁-C₄)alkylC(S)NR^{d}R^{e}, —(C₁-C₄)alkylNRd^{b}C(S)R^{c}, —(C₁-C₄)alkylSR^{d}, 5- to 7-membered heteroaryl, or 4- to 7-membered heterocyclyl, wherein said heteroaryl and heterocyclyl are each optionally substituted with 1 to 3 groups selected from R⁷;
R^{a}, R^{b}, R^{c}, R^{d}, R^{e}, and R^{f} are each independently hydrogen, (C₁-C₄)alkyl, or halo(C₁-C₄)alkyl;
R⁶ and R⁷ are each independently halo, hydroxy, (C₁-C₄)alkyl, halo(C₁-C₄)alkyl, (C₁-C₄)alkoxy, halo(C₁-C₄)alkoxy, CN, or oxo;
m and n are each independently 0, 1, 2 or 3; and
p is 0 or 1.

2. The compound of claim 1, or a pharmaceutically acceptable salt thereof, wherein
R² is hydrogen, (C₁-C₄)alkyl, halo(C₁-C₄)alkyl, —(C₁-C₄)alkylOR^{a}, 5- to 7-membered heteroaryl, or 4- to 7-membered heterocyclyl, wherein said heteroaryl and heterocyclyl are each optionally substituted with 1 to 3 groups selected from R⁶;
R³ is halo, (C₁-C₄)alkyl, halo(C₁-C₄)alkyl, or CN; and
m and n are each independently 0, 1, or 2.

3. The compound of claim 1, or a pharmaceutically acceptable salt thereof, wherein R³ is halo, halo(C₁-C₄)alkyl, (C₁-C₄)alkoxy, or halo(C₁-C₄)alkoxy.

4-6. (canceled)

7. The compound of claim 1, wherein the compound is of the Formula II: 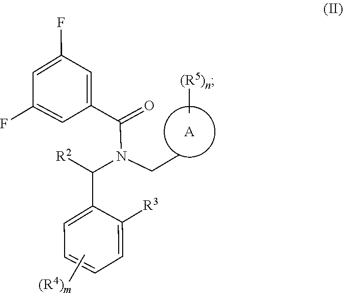
or a pharmaceutically acceptable salt thereof.

8. The compound of claim 1, or a pharmaceutically acceptable salt thereof, wherein R² is hydrogen or (C₁-C₄)alkyl.

9. The compound of claim 1, wherein the compound is of the Formula III: 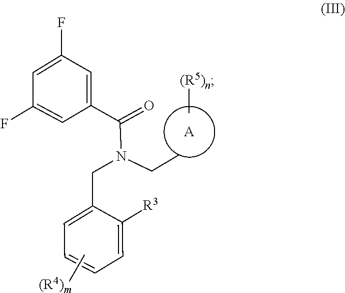
or a pharmaceutically acceptable salt thereof.

10. The compound of claim 1, or a pharmaceutically acceptable salt thereof, wherein R⁴ is halo, (C₁-C₄)alkyl, halo(C₁-C₄)alkyl, (C₁-C₄)alkoxy, or halo(C₁-C₄)alkoxy or CN.

11-13. (canceled)

14. The compound of claim 1, or a pharmaceutically acceptable salt thereof, wherein m is 0, 1 or 2.

15. (canceled)

16. The compound of claim 1, wherein the compound is of the Formula IV: 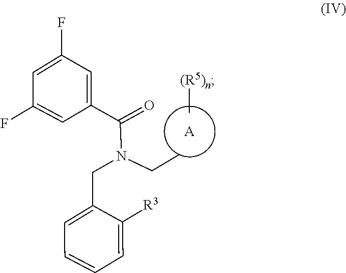
or a pharmaceutically acceptable salt thereof.

17. The compound of claim 1, or a pharmaceutically acceptable salt thereof, wherein Ring A is pyridyl, pyrimidinyl, pyridazinyl, pyrazinyl, or pyrazolyl.

18. The compound of claim 1, or a pharmaceutically acceptable salt thereof, wherein Ring A is pyridyl.

19. The compound of claim 1, wherein the compound is of the Formula V: 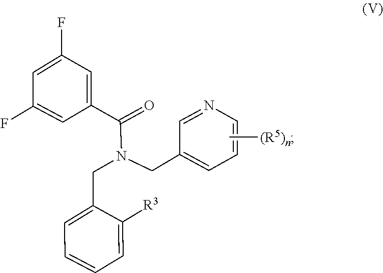
or a pharmaceutically acceptable salt thereof.

20. The compound of claim 1, wherein R⁵ is in the para position with respect to the connection point for ring A.

21. The compound of claim 1, or a pharmaceutically acceptable salt thereof, wherein R⁵ is halo, oxo, (C₁-C₄)alkyl, halo(C₁-C₄)alkyl, —C(O)OR^{b}, —NR^{b}R^{c}, —C(O)NR^{b}R^{c}, —(C₁-C₄)alkylOR^{b}, —(C₁-C₄)alkylC(O)R^{b}, —(C₁-C₄)alkylC(O)NR^{b}R^{c}, —C(O)NR^{b}SO2(C₁-C₄)alkyl, 5- to 6-membered heteroaryl, wherein said heteroaryl is optionally substituted with 1 to 3 groups selected from R⁷.

22. The compound of claim 1, or a pharmaceutically acceptable salt thereof, wherein R⁵ is oxo, CF₃, CH₃, C(O)OCH₃, C(O)OH, CH₂COOH, NH₂, CONH₂, CH₂CONH₂, CONHCH₃, CH₂OH, CH₂CH₂OH, CONHSO₂CH₃, tetrazolyl, pyrazolyl, triazolyl, or pyrazolyl, wherein said pyrazolyl is optionally substituted with hydroxy.

23. The compound of claim 1, or a pharmaceutically acceptable salt thereof, wherein n is 0, 1 or 2.

24. (canceled)

25. A pharmaceutical composition comprising a compound of claim 1, or a pharmaceutically acceptable salt thereof, and a pharmaceutically acceptable carrier.

26. A method of treating a disease responsive to the inhibition of TREX1 in a subject, comprising administering to the subject, a therapeutically effective amount of a compound of claim 1, or a pharmaceutically acceptable salt thereof.

27. The method of claim 26, wherein the disease is cancer.

28-55. (canceled)

56. The compound of claim 1, wherein the compound is selected from any one of the following or a pharmaceutically acceptable salt thereof: 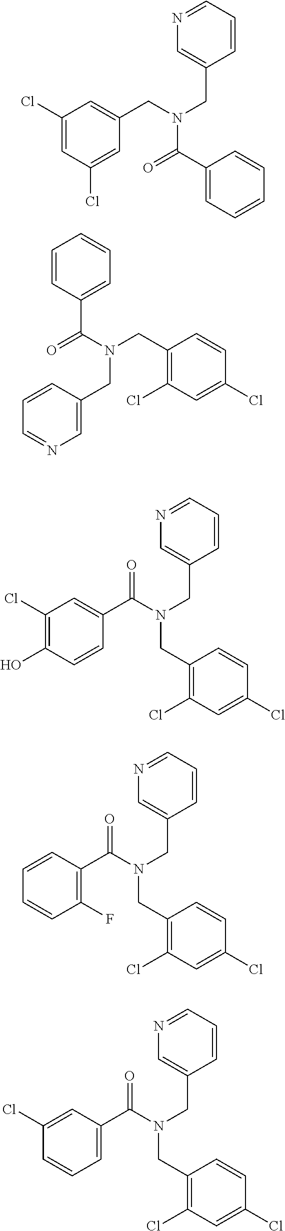 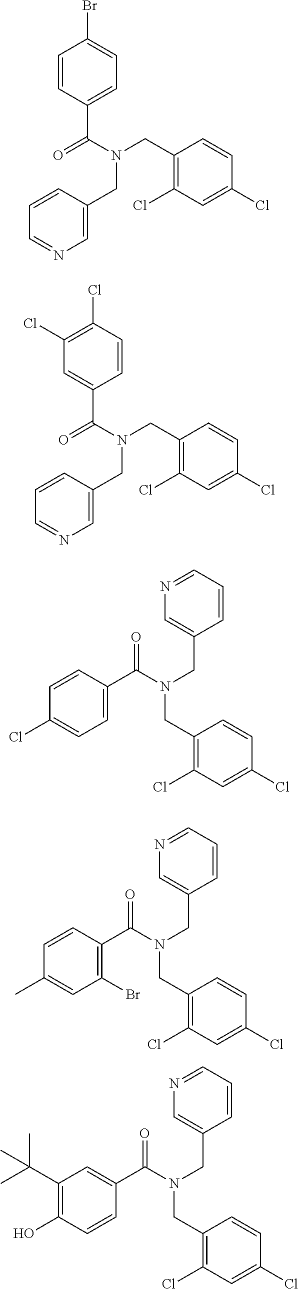 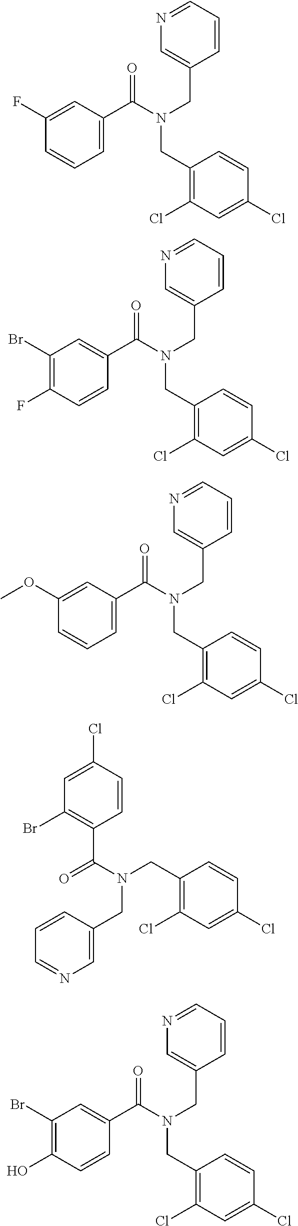 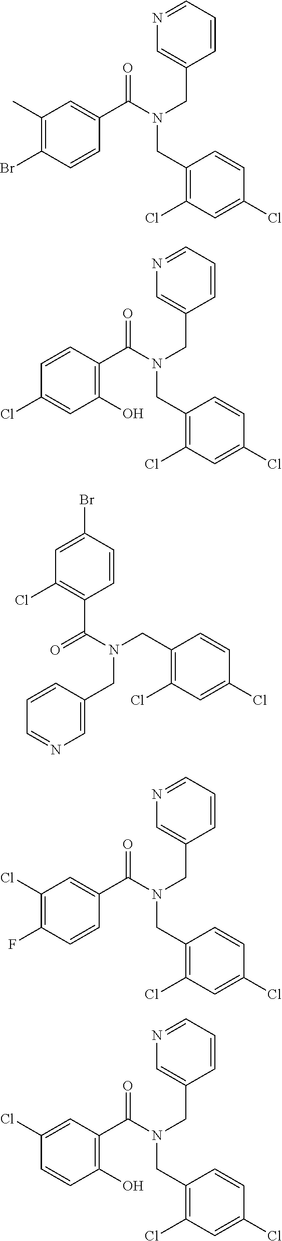 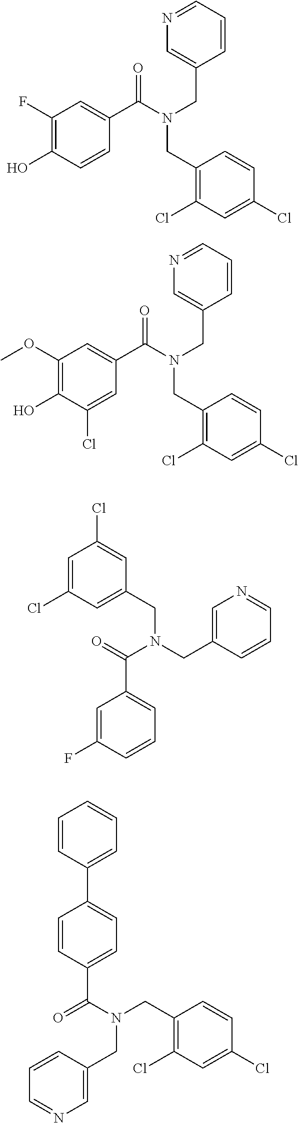 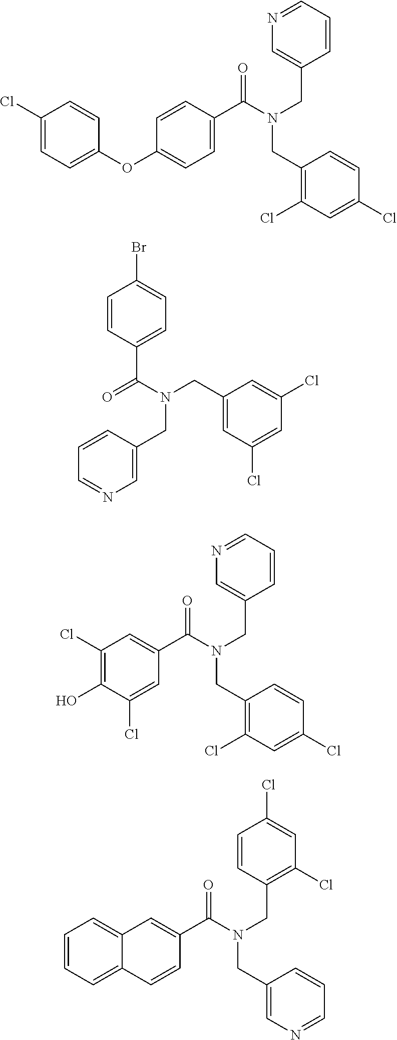 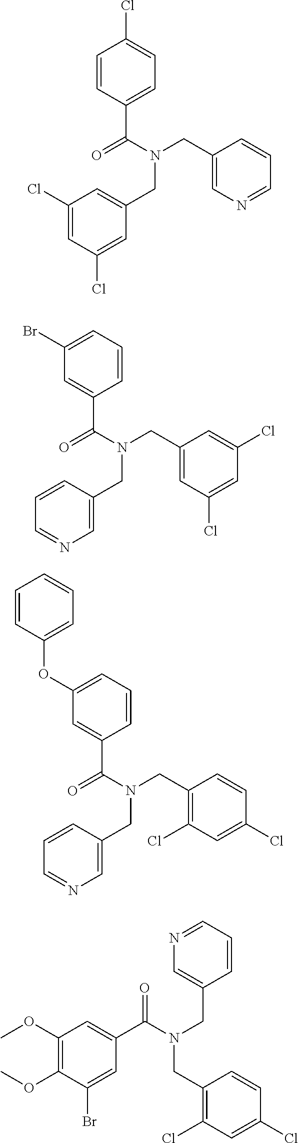 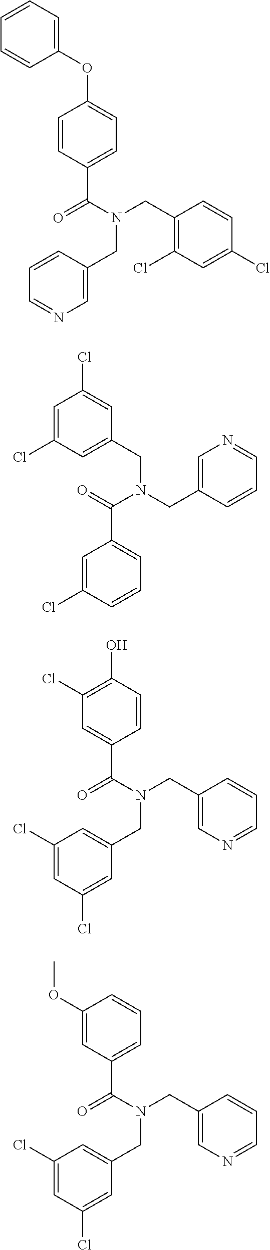 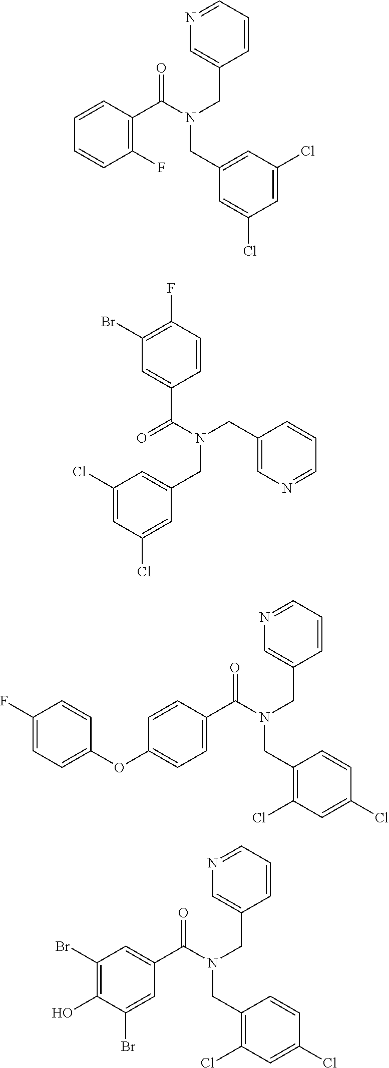 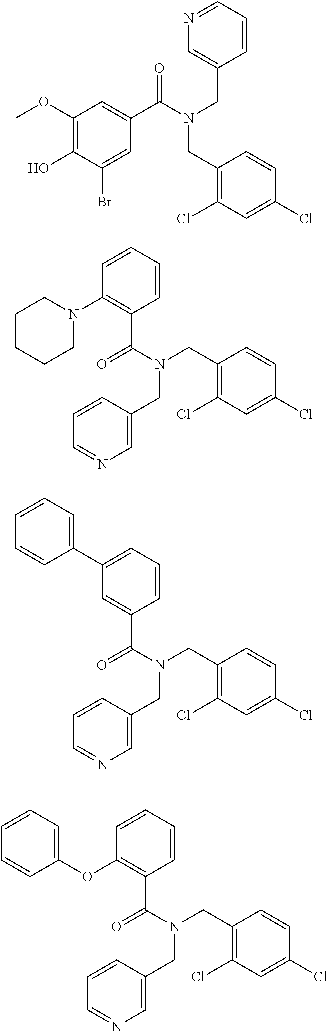 [SMILES: COc1cc(C(=O)N(Cc2cccnc2)Cc2ccc(Cl)cc2Cl)cc(Br)c1O ; O=C(c1ccccc1N1CCCCC1)N(Cc1cccnc1)Cc1ccc(Cl)cc1Cl ; O=C(c1cccc(-c2ccccc2)c1)N(Cc1cccnc1)Cc1ccc(Cl)cc1Cl ; O=C(c1ccccc1Oc1ccccc1)N(Cc1cccnc1)Cc1ccc(Cl)cc1Cl] 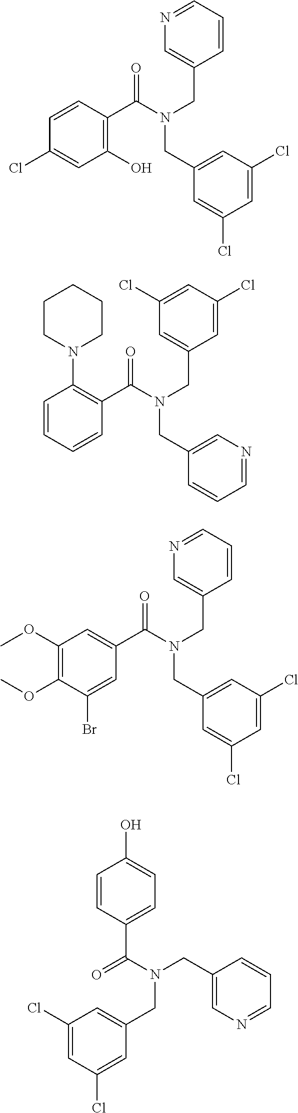 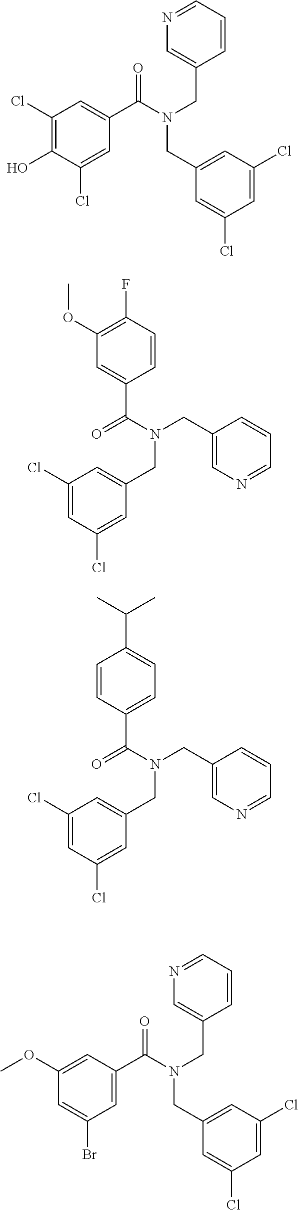 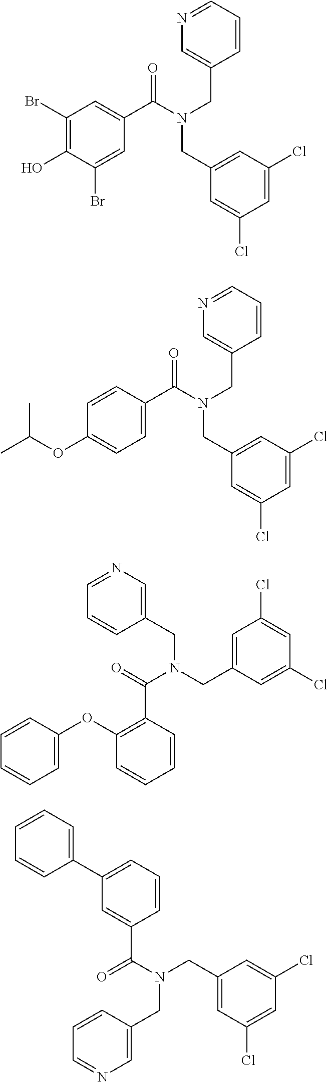 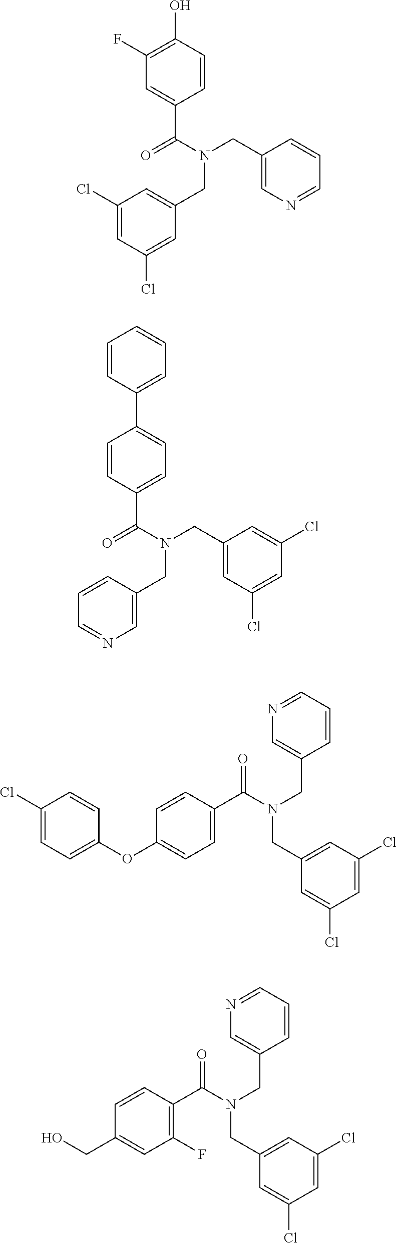 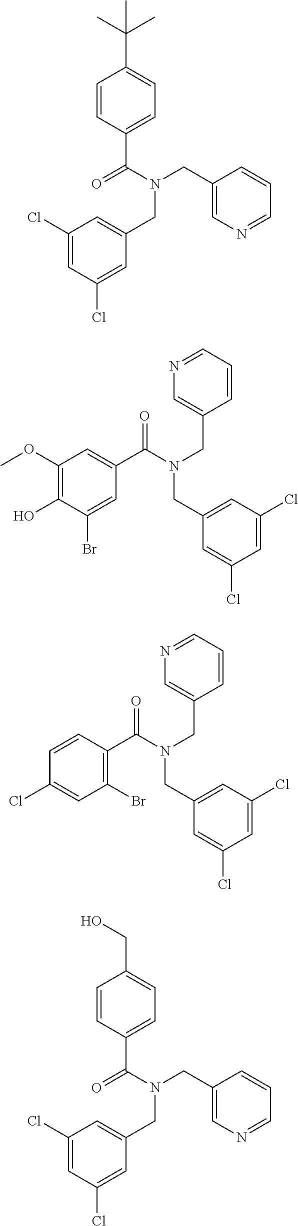 [SMILES: CC(C)(C)c1ccc(C(=O)N(Cc2cccnc2)Cc2cc(Cl)cc(Cl)c2)cc1 ; COc1cc(C(=O)N(Cc2cccnc2)Cc2cc(Cl)cc(Cl)c2)cc(Br)c1O ; O=C(c1ccc(Cl)cc1Br)N(Cc1cccnc1)Cc1cc(Cl)cc(Cl)c1 ; O=C(c1ccc(CO)cc1)N(Cc1cccnc1)Cc1cc(Cl)cc(Cl)c1] 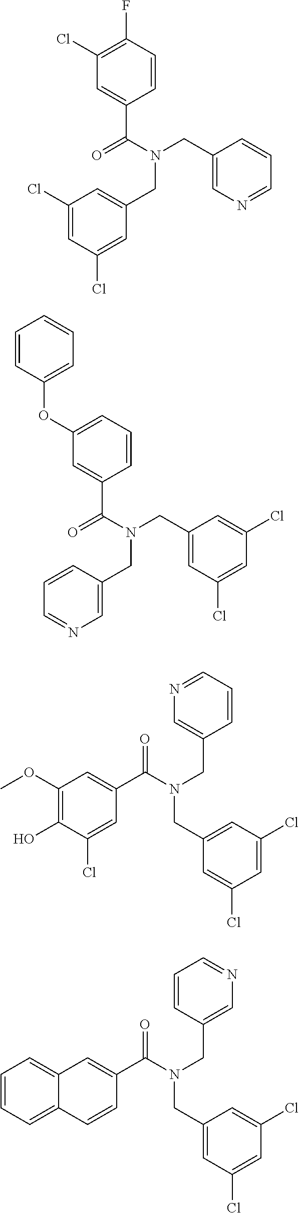 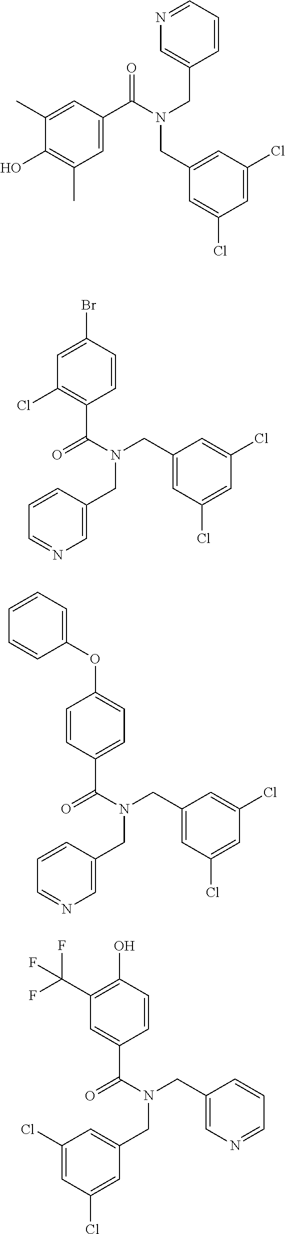 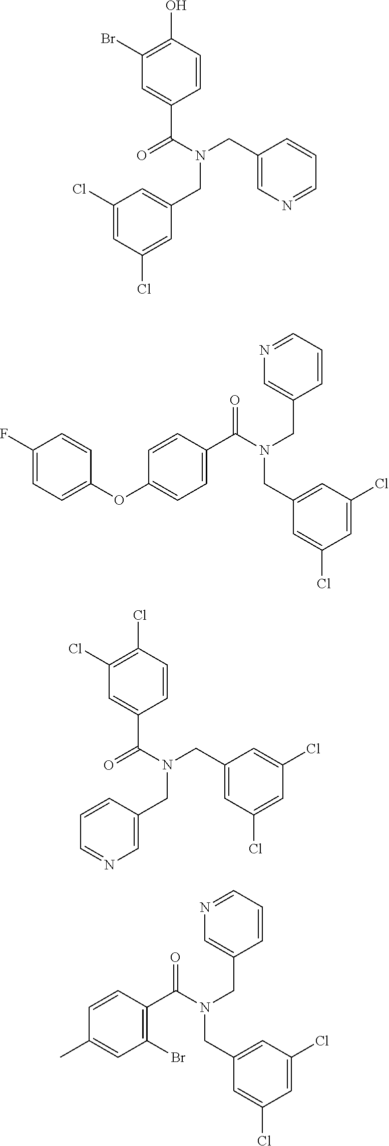 [SMILES: O=C(c1ccc(O)c(Br)c1)N(Cc1cccnc1)Cc1cc(Cl)cc(Cl)c1 ; O=C(c1ccc(Oc2ccc(F)cc2)cc1)N(Cc1cccnc1)Cc1cc(Cl)cc(Cl)c1 ; O=C(c1ccc(Cl)c(Cl)c1)N(Cc1cccnc1)Cc1cc(Cl)cc(Cl)c1 ; Cc1ccc(C(=O)N(Cc2cccnc2)Cc2cc(Cl)cc(Cl)c2)c(Br)c1] 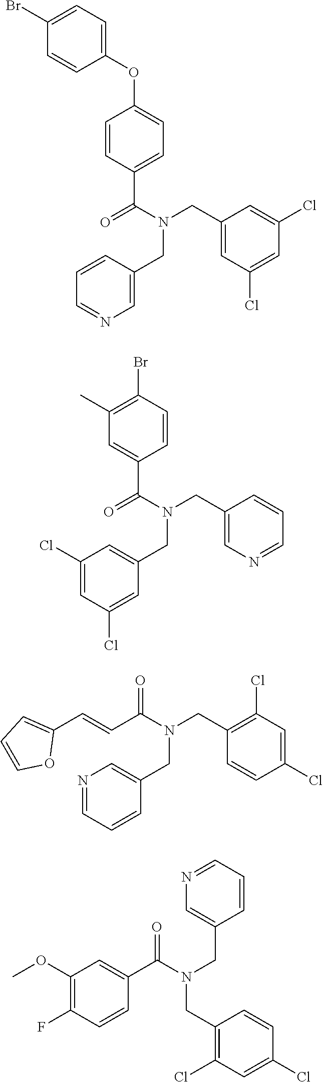 [SMILES: O=C(c1ccc(Oc2ccc(Br)cc2)cc1)N(Cc1cccnc1)Cc1cc(Cl)cc(Cl)c1 ; Cc1cc(C(=O)N(Cc2cccnc2)Cc2cc(Cl)cc(Cl)c2)ccc1Br ; O=C(/C=C/c1ccco1)N(Cc1cccnc1)Cc1ccc(Cl)cc1Cl ; COc1cc(C(=O)N(Cc2cccnc2)Cc2ccc(Cl)cc2Cl)ccc1F] 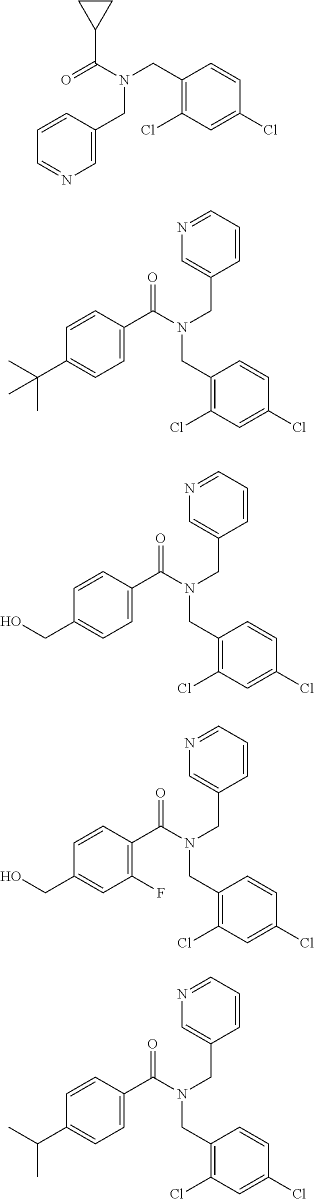 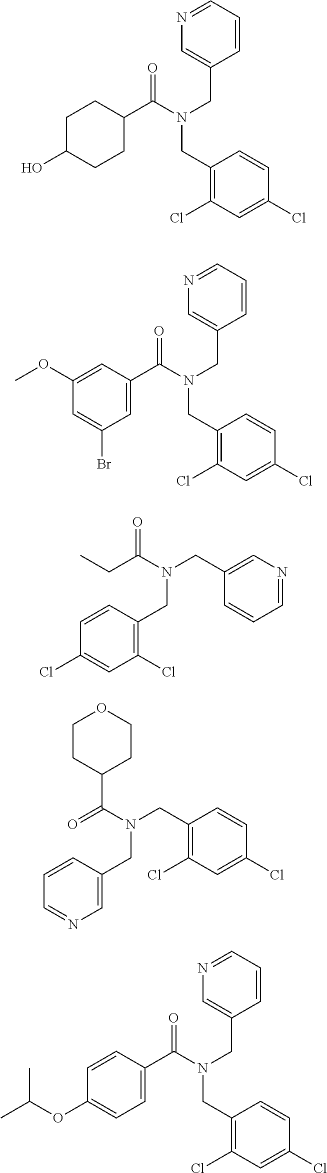 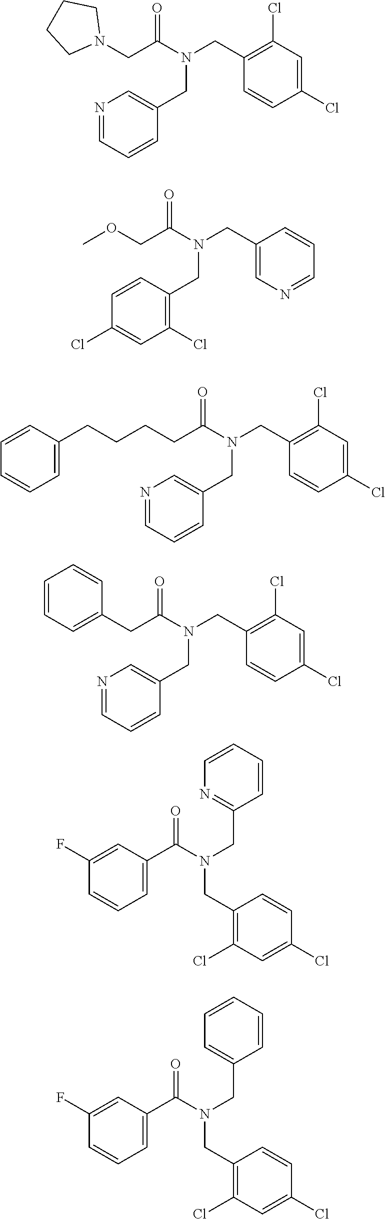 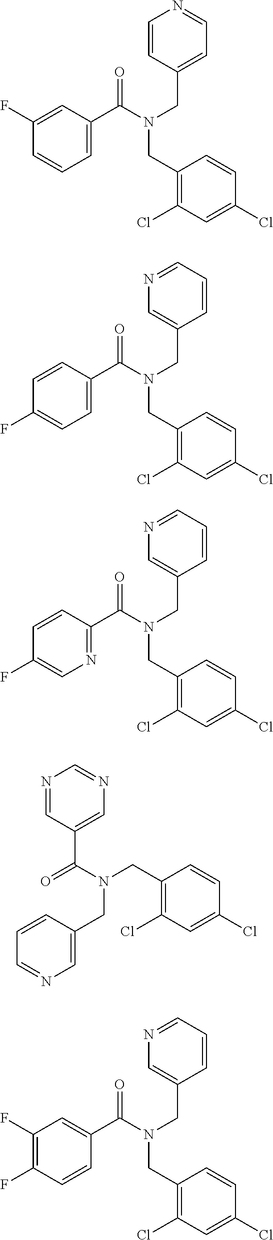 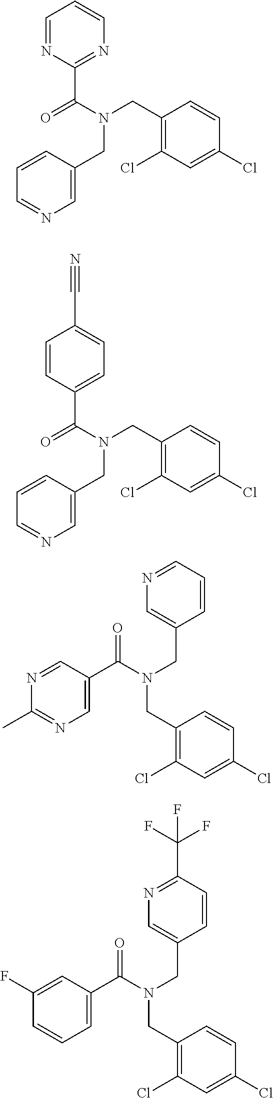 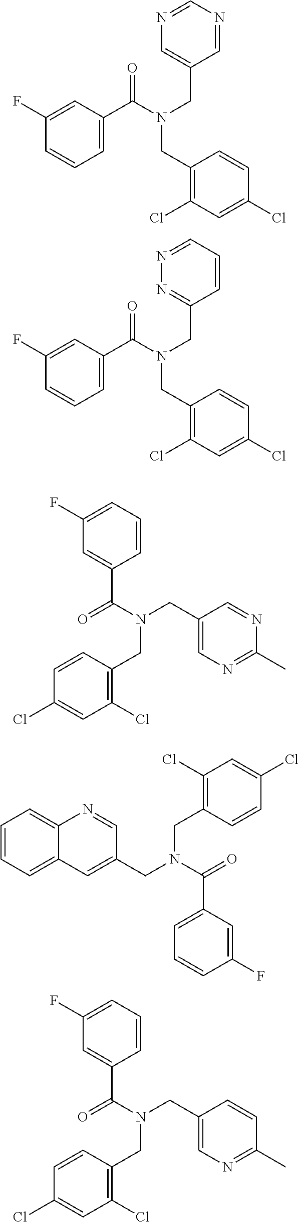 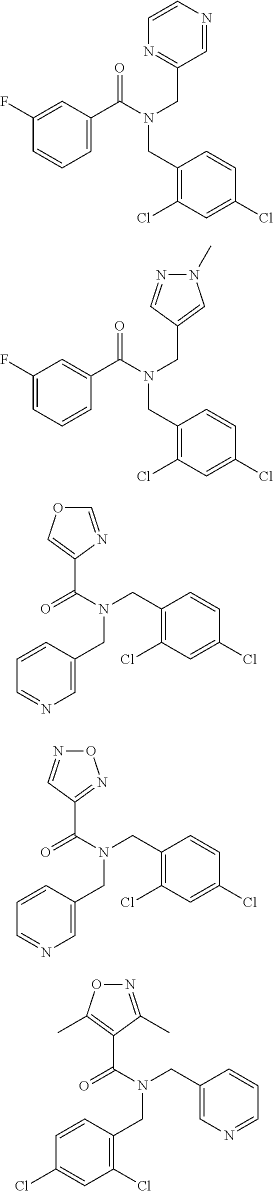 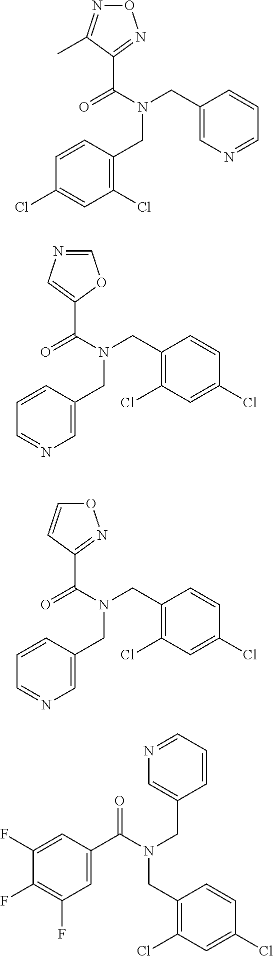 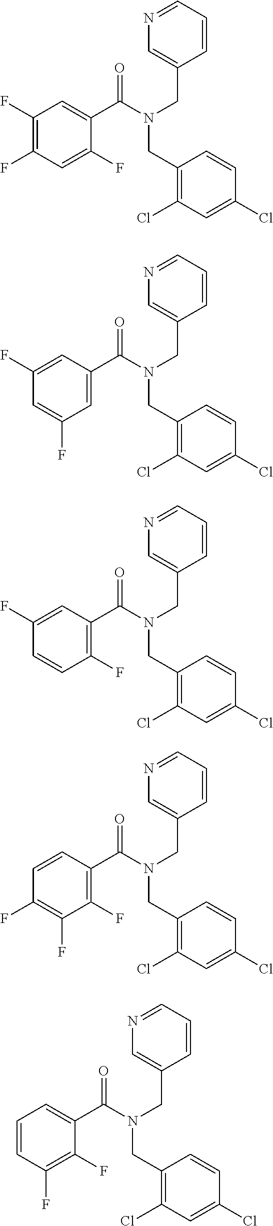 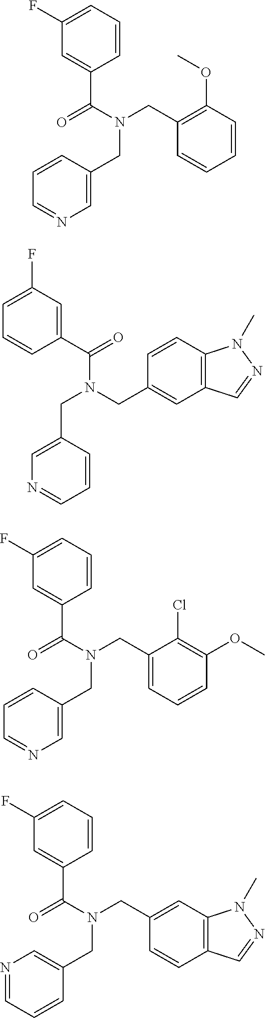 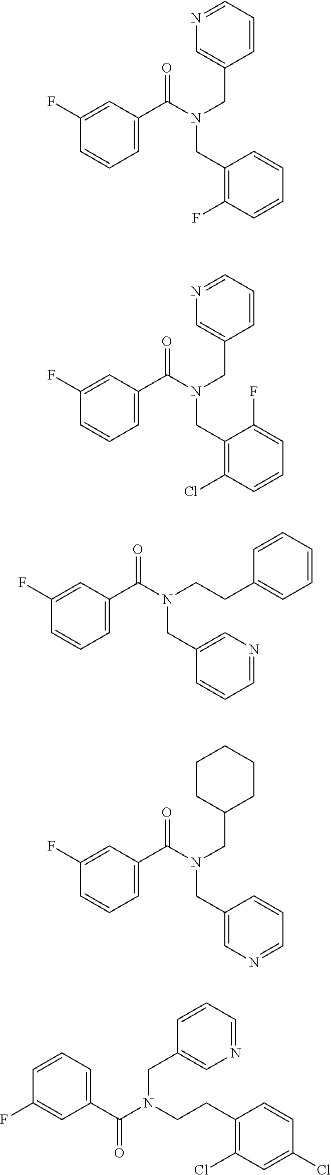 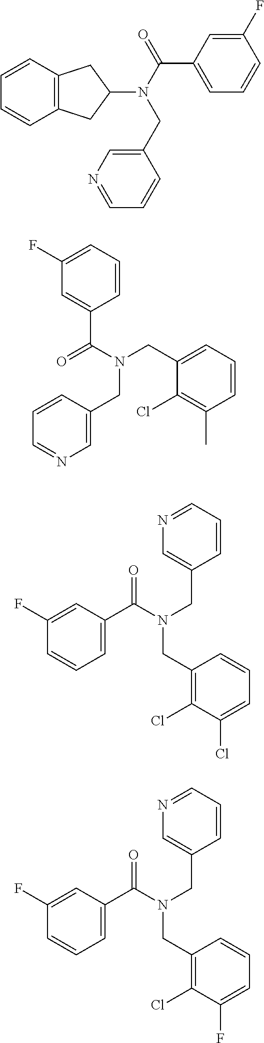 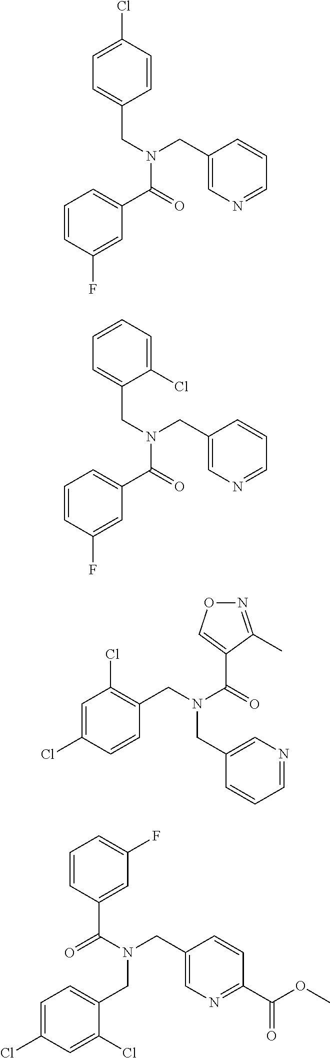 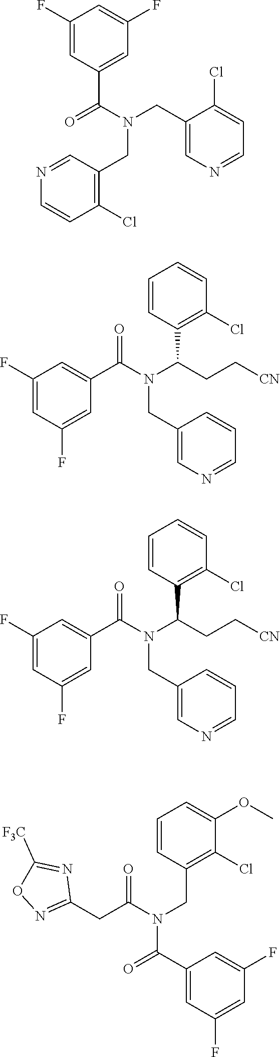 [SMILES: O=C(c1cc(F)cc(F)c1)N(Cc1cnccc1Cl)Cc1cnccc1Cl ; N#CCC[C@@H](c1ccccc1Cl)N(Cc1cccnc1)C(=O)c1cc(F)cc(F)c1 ; N#CCC[C@H](c1ccccc1Cl)N(Cc1cccnc1)C(=O)c1cc(F)cc(F)c1 ; COc1cccc(CN(C(=O)Cc2noc(C(F)(F)F)n2)C(=O)c2cc(F)cc(F)c2)c1Cl] 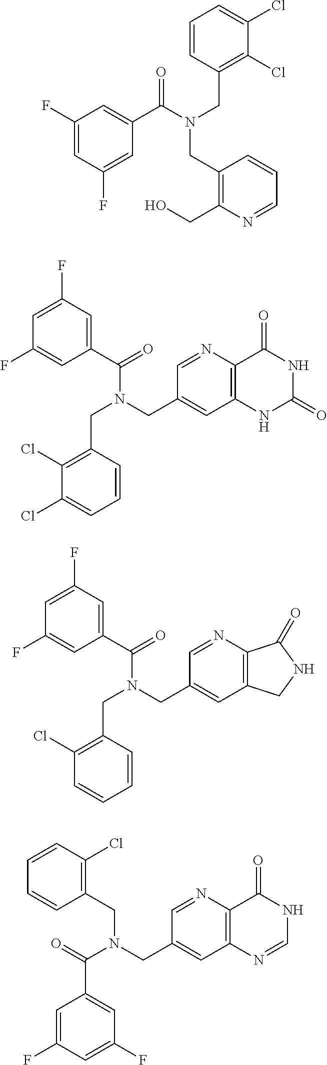 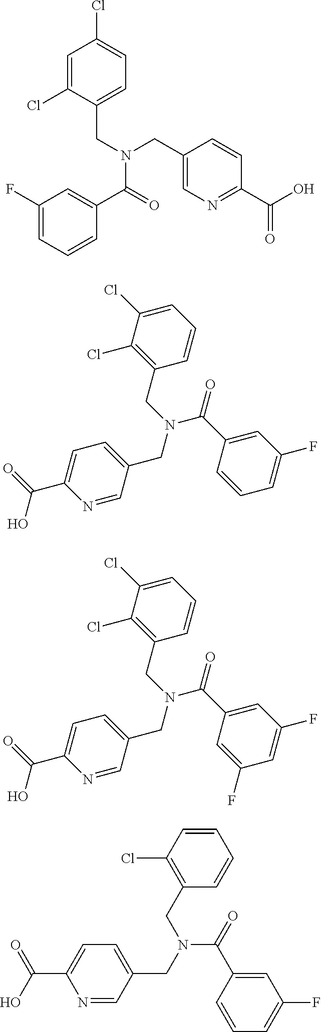 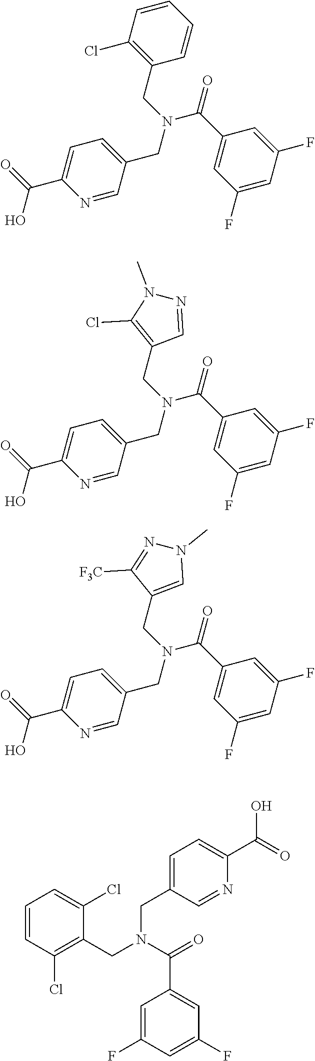 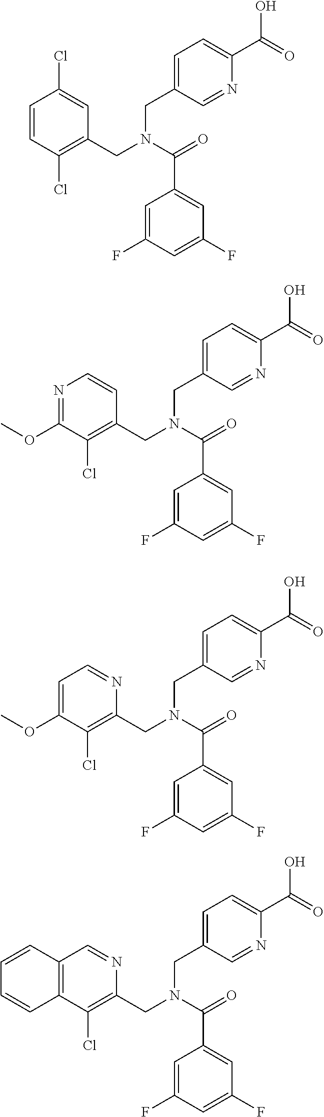 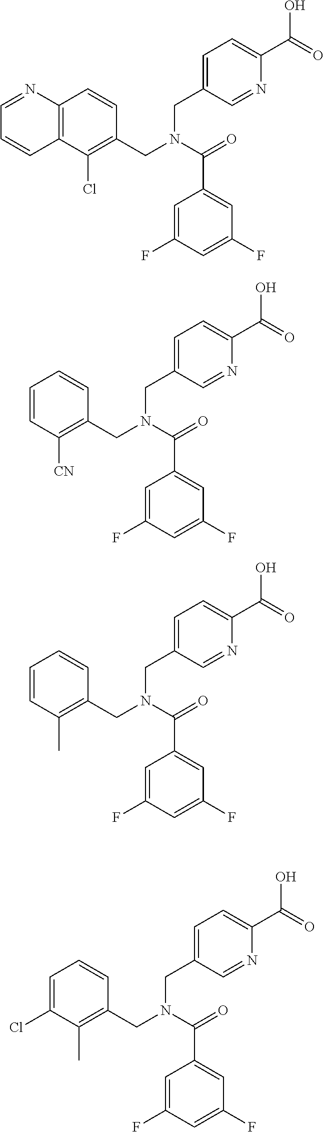 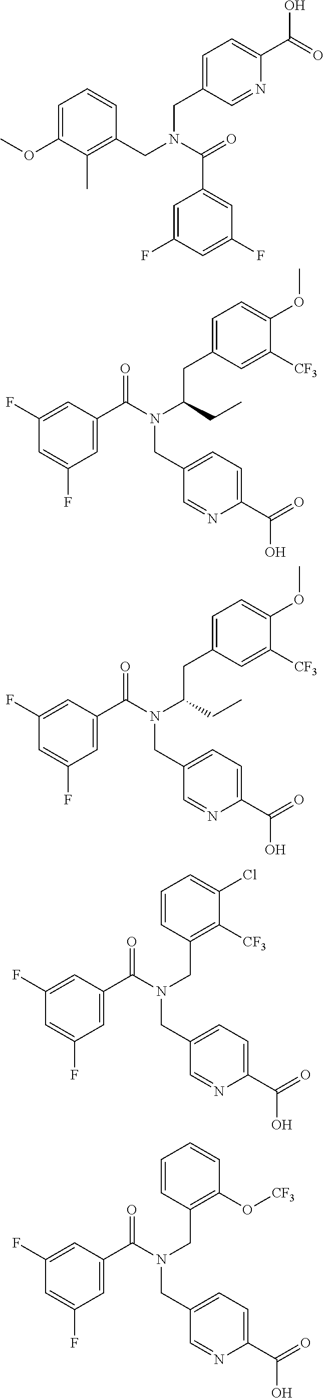 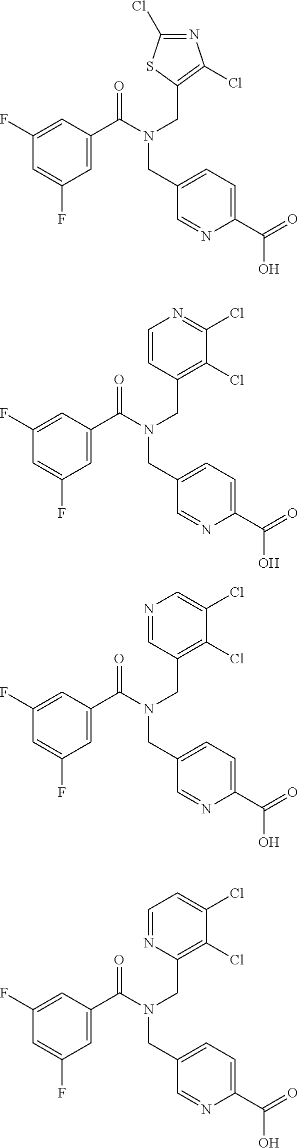 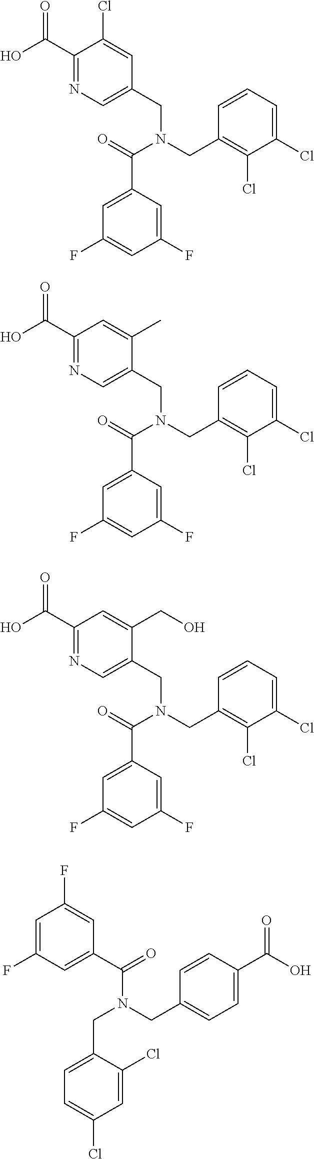 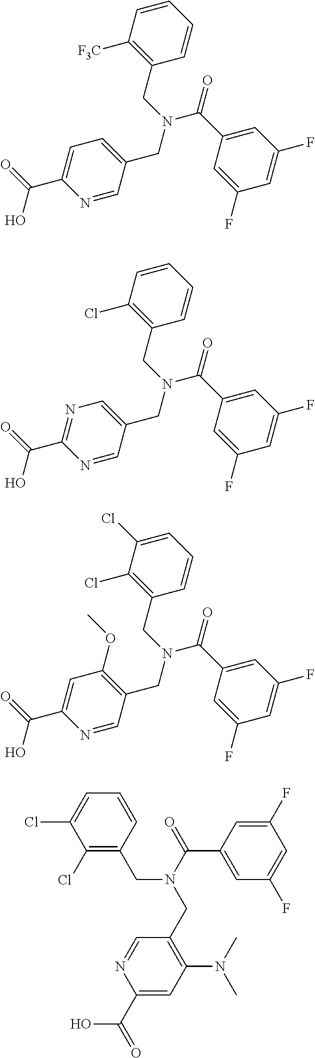 [SMILES: O=C(O)c1ccc(CN(Cc2ccccc2C(F)(F)F)C(=O)c2cc(F)cc(F)c2)cn1 ; O=C(O)c1ncc(CN(Cc2ccccc2Cl)C(=O)c2cc(F)cc(F)c2)cn1 ; COc1cc(C(=O)O)ncc1CN(Cc1cccc(Cl)c1Cl)C(=O)c1cc(F)cc(F)c1 ; CN(C)c1cc(C(=O)O)ncc1CN(Cc1cccc(Cl)c1Cl)C(=O)c1cc(F)cc(F)c1] 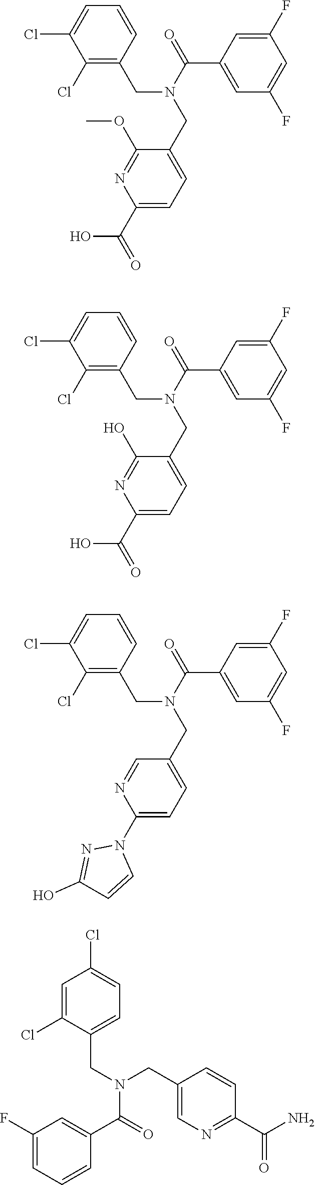 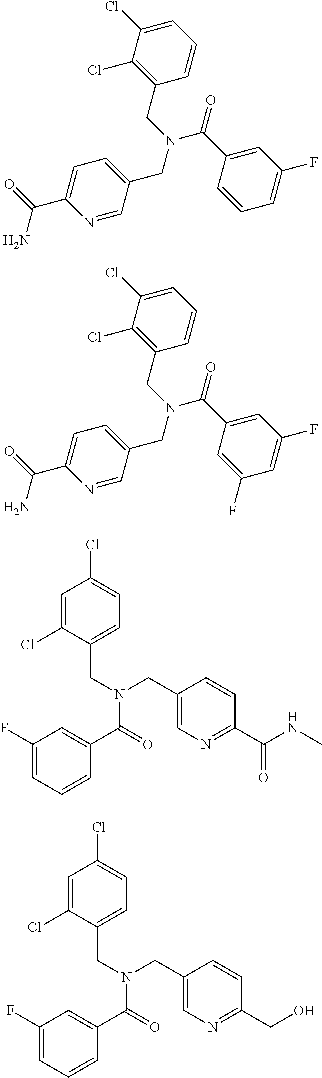 [SMILES: NC(=O)c1ccc(CN(Cc2cccc(Cl)c2Cl)C(=O)c2cccc(F)c2)cn1 ; NC(=O)c1ccc(CN(Cc2cccc(Cl)c2Cl)C(=O)c2cc(F)cc(F)c2)cn1 ; CNC(=O)c1ccc(CN(Cc2ccc(Cl)cc2Cl)C(=O)c2cccc(F)c2)cn1 ; O=C(c1cccc(F)c1)N(Cc1ccc(CO)nc1)Cc1ccc(Cl)cc1Cl] 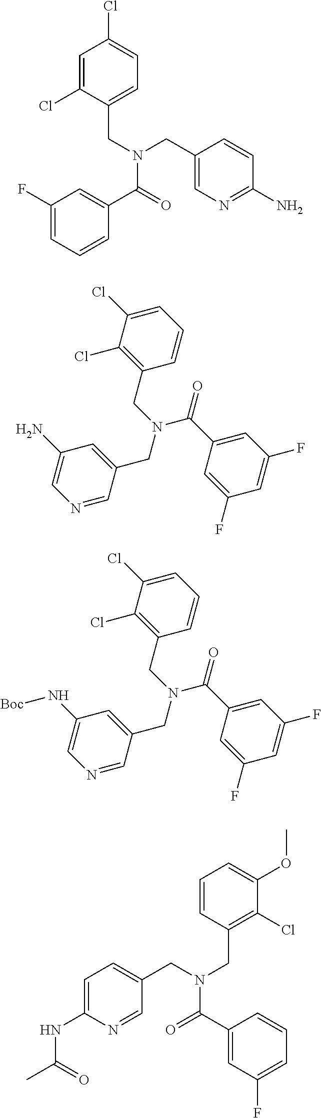 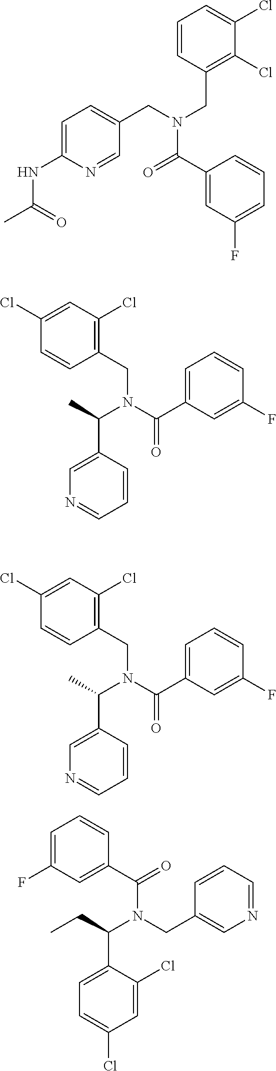 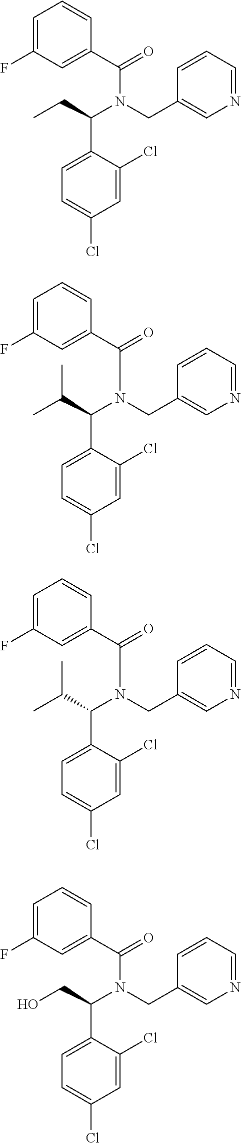 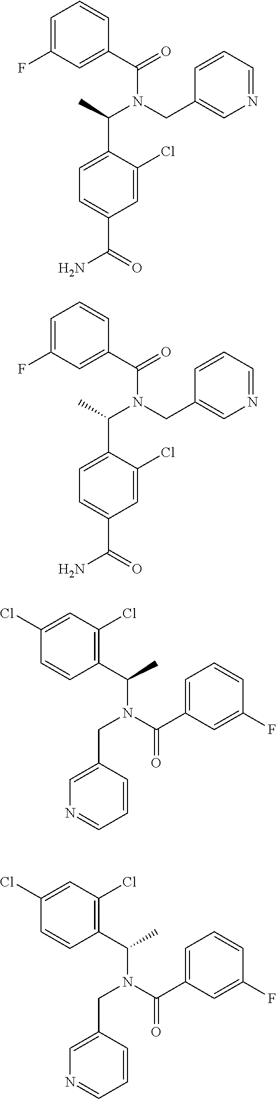 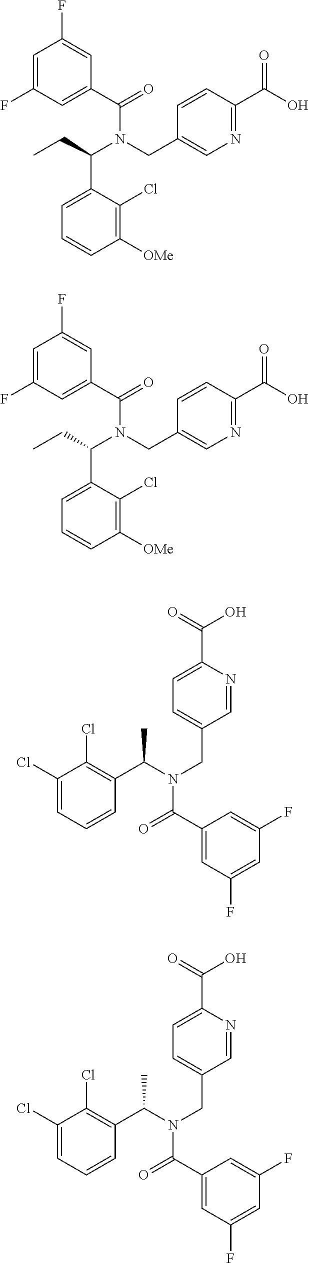 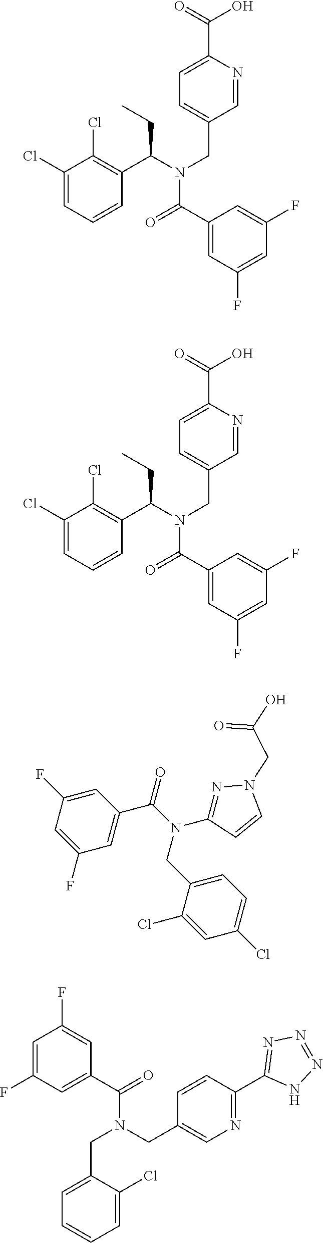 [SMILES: CC[C@H](c1cccc(Cl)c1Cl)N(Cc1ccc(C(=O)O)nc1)C(=O)c1cc(F)cc(F)c1 ; CC[C@H](c1cccc(Cl)c1Cl)N(Cc1ccc(C(=O)O)nc1)C(=O)c1cc(F)cc(F)c1 ; O=C(O)Cn1ccc(N(Cc2ccc(Cl)cc2Cl)C(=O)c2cc(F)cc(F)c2)n1 ; O=C(c1cc(F)cc(F)c1)N(Cc1ccc(-c2nnn[nH]2)nc1)Cc1ccccc1Cl] 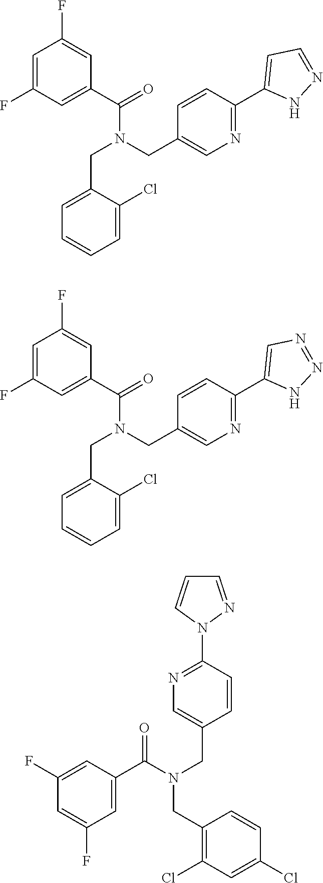 [SMILES: O=C(c1cc(F)cc(F)c1)N(Cc1ccc(-c2ccn[nH]2)nc1)Cc1ccccc1Cl ; O=C(c1cc(F)cc(F)c1)N(Cc1ccc(-c2cnn[nH]2)nc1)Cc1ccccc1Cl ; O=C(c1cc(F)cc(F)c1)N(Cc1ccc(-n2cccn2)nc1)Cc1ccc(Cl)cc1Cl] 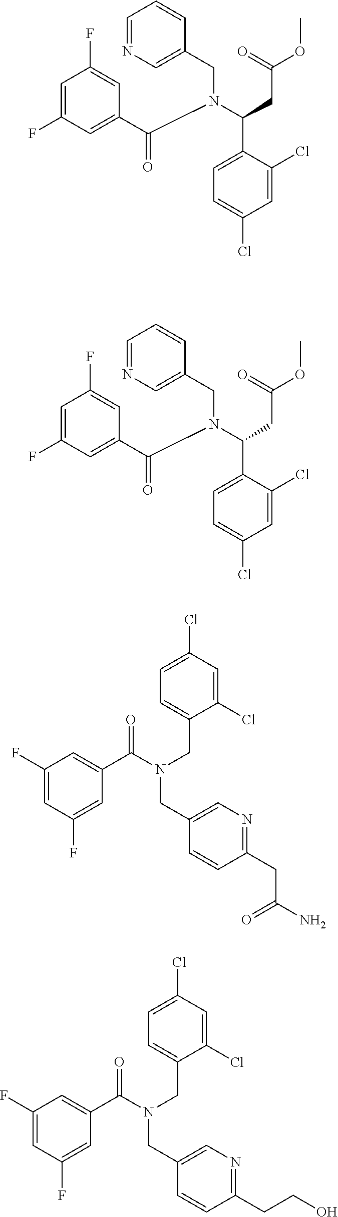 [SMILES: COC(=O)C[C@@H](c1ccc(Cl)cc1Cl)N(Cc1cccnc1)C(=O)c1cc(F)cc(F)c1 ; COC(=O)C[C@H](c1ccc(Cl)cc1Cl)N(Cc1cccnc1)C(=O)c1cc(F)cc(F)c1 ; NC(=O)Cc1ccc(CN(Cc2ccc(Cl)cc2Cl)C(=O)c2cc(F)cc(F)c2)cn1 ; O=C(c1cc(F)cc(F)c1)N(Cc1ccc(CCO)nc1)Cc1ccc(Cl)cc1Cl] 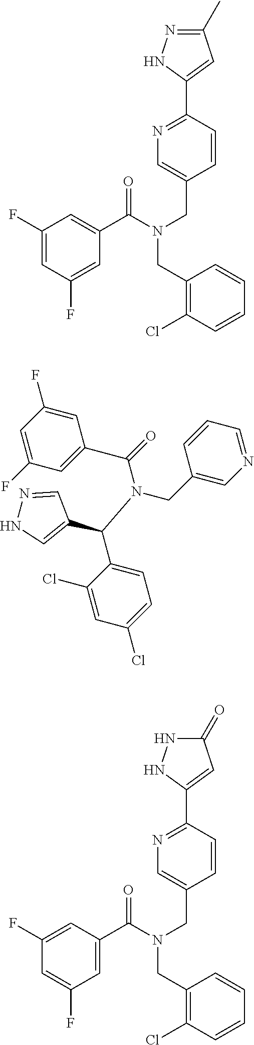 [SMILES: Cc1cc(-c2ccc(CN(Cc3ccccc3Cl)C(=O)c3cc(F)cc(F)c3)cn2)[nH]n1 ; O=C(c1cc(F)cc(F)c1)N(Cc1cccnc1)[C@H](c1cn[nH]c1)c1ccc(Cl)cc1Cl ; O=C(c1cc(F)cc(F)c1)N(Cc1ccc(-c2cc(=O)[nH][nH]2)nc1)Cc1ccccc1Cl] 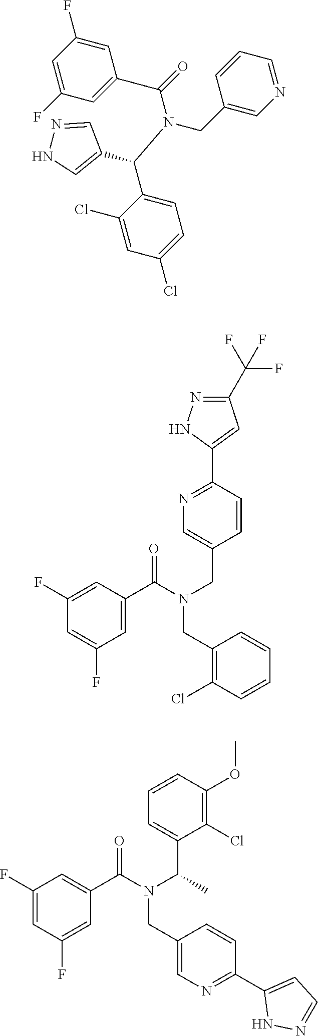 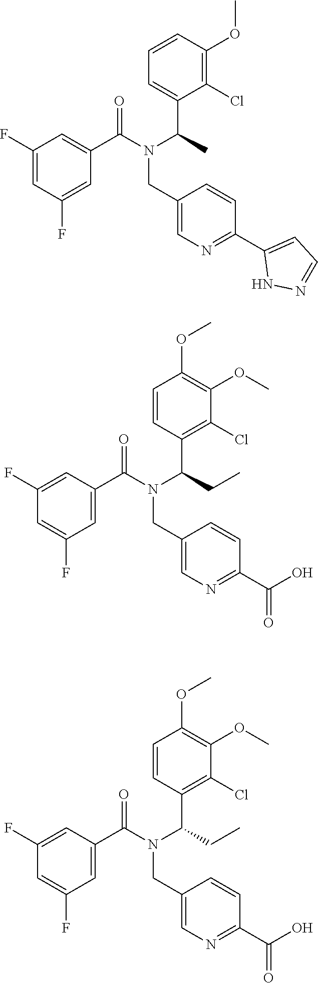 [SMILES: COc1cccc([C@@H](C)N(Cc2ccc(-c3ccn[nH]3)nc2)C(=O)c2cc(F)cc(F)c2)c1Cl ; CC[C@H](c1ccc(OC)c(OC)c1Cl)N(Cc1ccc(C(=O)O)nc1)C(=O)c1cc(F)cc(F)c1 ; CC[C@@H](c1ccc(OC)c(OC)c1Cl)N(Cc1ccc(C(=O)O)nc1)C(=O)c1cc(F)cc(F)c1] 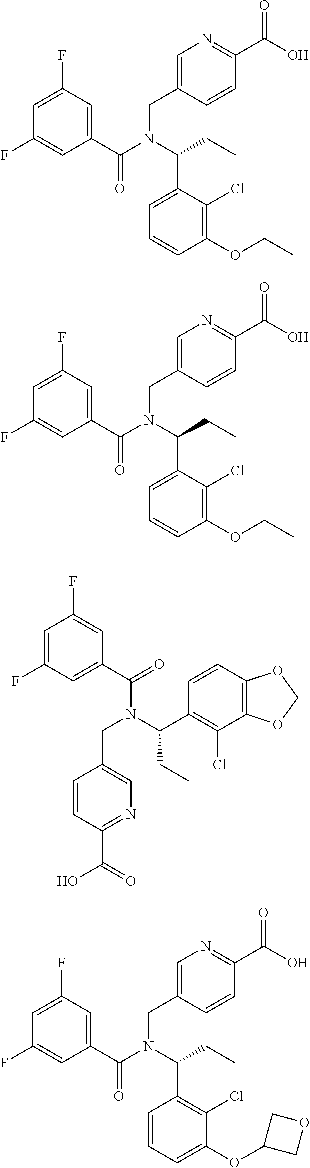 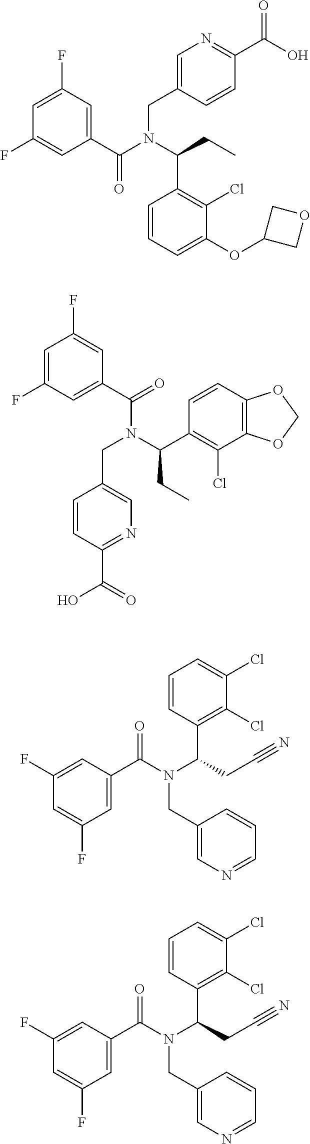 [SMILES: CC[C@@H](c1cccc(OC2COC2)c1Cl)N(Cc1ccc(C(=O)O)nc1)C(=O)c1cc(F)cc(F)c1 ; CC[C@H](c1ccc2c(c1Cl)OCO2)N(Cc1ccc(C(=O)O)nc1)C(=O)c1cc(F)cc(F)c1 ; N#CC[C@@H](c1cccc(Cl)c1Cl)N(Cc1cccnc1)C(=O)c1cc(F)cc(F)c1 ; N#CC[C@H](c1cccc(Cl)c1Cl)N(Cc1cccnc1)C(=O)c1cc(F)cc(F)c1] 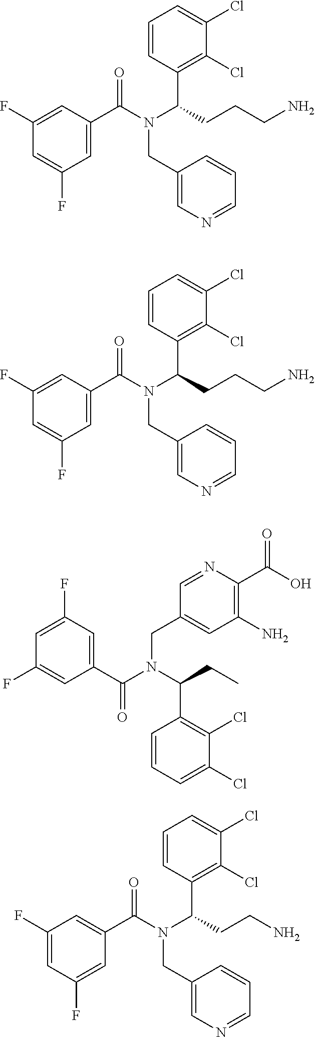 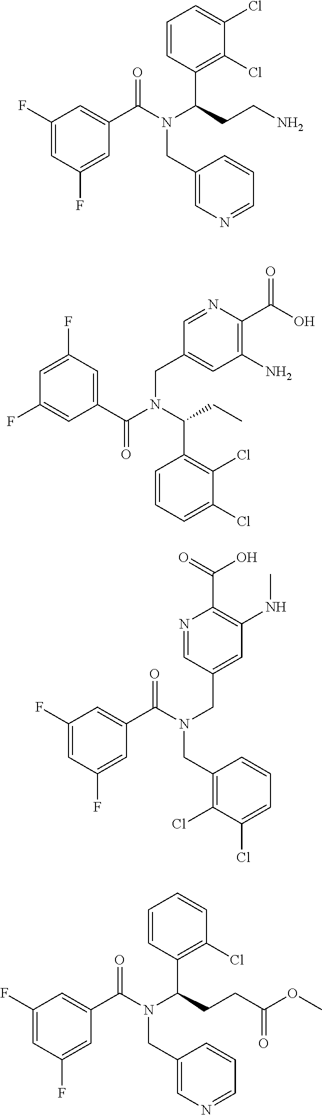 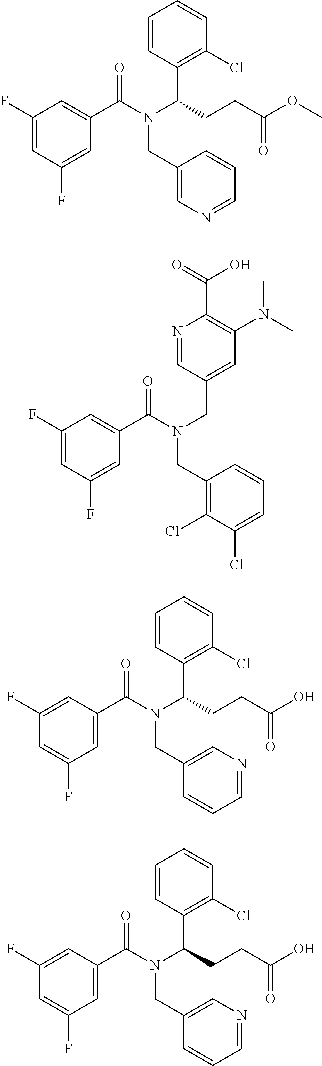 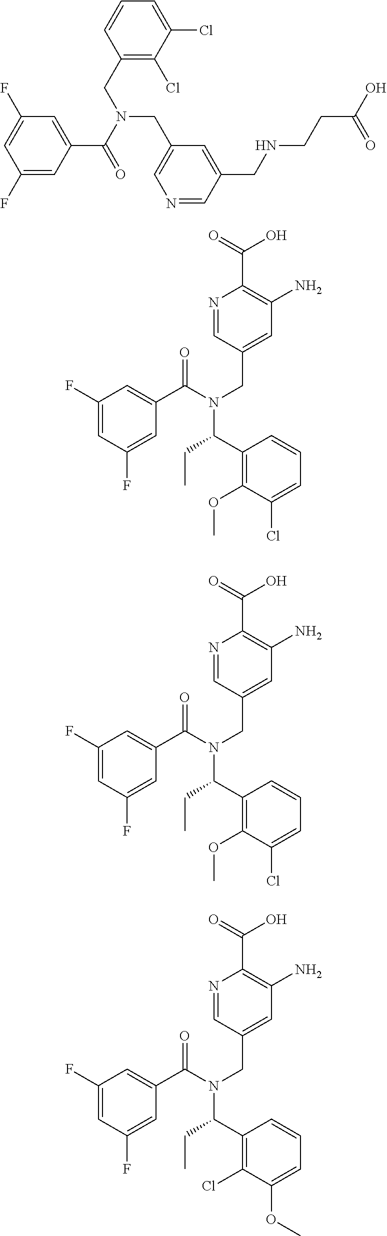 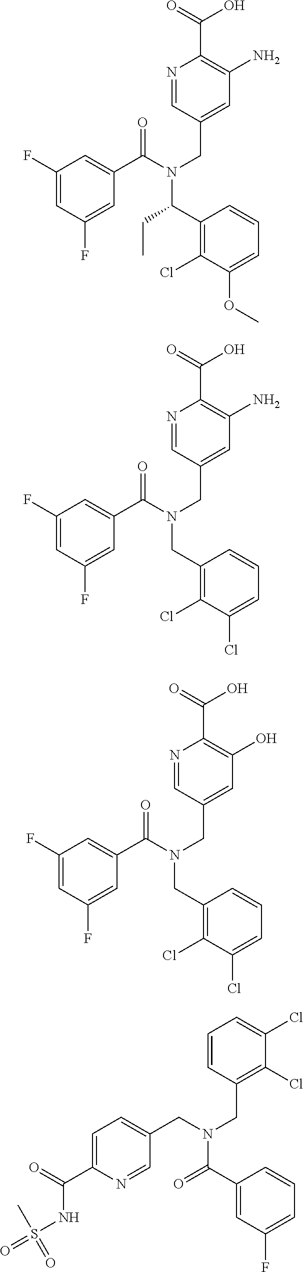 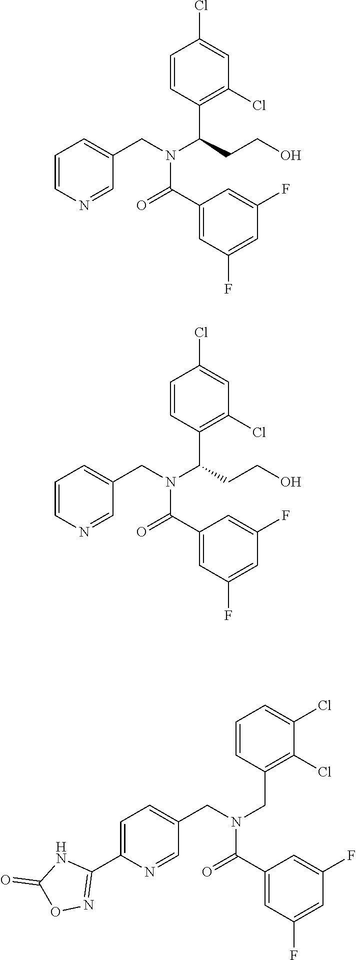 [SMILES: O=C(c1cc(F)cc(F)c1)N(Cc1cccnc1)[C@H](CCO)c1ccc(Cl)cc1Cl ; O=C(c1cc(F)cc(F)c1)N(Cc1cccnc1)[C@@H](CCO)c1ccc(Cl)cc1Cl ; O=C(c1cc(F)cc(F)c1)N(Cc1ccc(-c2noc(=O)[nH]2)nc1)Cc1cccc(Cl)c1Cl]
